(19) United States
(12) Patent Application Publication
(10) Pub. No.: US 2005/0272055 A1
(43) Pub. Date: Dec. 8, 2005

(54) Method of treating lethal shock induced by toxic agents and diagnosing exposure to toxic agents by measuring distinct pattern in the levels of expression of specific genes

(76) Inventors: Rina Das, Rockville, MD (US); Marti Jett, Washington, DC (US); Chanaka Mendis, Platteville, WI (US); Roger Neill, Rockville, MD (US)

Correspondence Address: Caroline Nash;Nash & Titus, LLC, 21402 Unison Road, Middleburg, VA, 20117, US

(21) Appl. No.: 11/000,615

(22) Filed: Dec. 1, 2004

Related U.S. Application Data
(63) Continuation-in-part of application No. 10/007,806, filed on Nov. 9, 2001.
Application No. 10/007,806 is a continuation-in-part of application No. 09/495,724, filed on Feb. 1, 2000, now Pat. No. 6,316,197.

(51) Int. Cl. C12Q 1/68; A61K 31/55

(52) U.S. Cl. 435/6

(57) ABSTRACT

A method for administering a therapeutic agent which inhibits the mechanistic pathways necessary to maintain the progression of lethal shock. The therapeutic agent is administered in the form of a drug, antisense or protein depending on the gene expression.

This application is a CIP of U.S. Ser. No. 10/007,806 filed Nov. 9, 2001 which is a CIP of U.S. Ser. No. 09/495,724 filed Feb. 1, 2000, both incorporated in their entirety by reference.

### GOVERNMENT INTEREST

The invention described herein may be manufactured, used and licensed by or for the U.S. Government.

### FIELD OF THE INVENTION

The present invention relates to methods of treating lethal shock using compositions and/or anitisense to turn off the expression of genes that are up-regulated by exposure to toxic agents or by increasing the amount of proteins or their products when genes that produce those proteins are down regulated by exposure to toxic agents.

### BACKGROUND OF THE INVENTION

The threat of terrorist action using biological warfare (BW), chemical or infectious agents has occurred throughout the world. These acts of terrorism are unpredictable and counter efforts have been aimed at rapid, accurate diagnosis and speedy treatment. Determination of the exact toxin that a subject has been exposed to is critical to treatment.

Current methods for pathogen or toxin identification require specialized reagents that are structural-based probes. For bio-engineered toxic agents, those probes may prove to be ineffective. The increased sophistication available for design of potential biological weapons will require reliance on better approaches to adequately identify such threats. Simple identification of toxins or infectious agents may be complicated by the fact that genetic manipulations could (1) make BW agents unrecognizable by structural-based technologies, or (2) enhance their devastating effects, making them toxic at undetectable levels. Furthermore, small amounts of common bacterial products, such as protein A or endotoxin, have been shown to markedly potentate activities of biological warfare threat toxins. The difficulties of identifying toxins experienced in the past could lead to potentially disastrous delays in responding appropriately to the threat or to the possibility of inappropriate treatment based on inadequate information. Thus far, diagnoses could only be made based on symptoms, which may take 4-24 hours or more to appear, and by that time, the damage is irreversible and death may result.

Description of a Selected Group of Toxic Agents:

There are many toxic agents that are a threat to humans in situations of biological warfare. For example, SEB: Staphyloccocal enterotoxin B is a potent bacterial toxin known to cause lethal shock. The mode of exposure could be aerosol, food or water contamination. It interacts with the lymphoid cells, proximal tubule (PT) kidney and other cells initiating cascades of reactions ultimately leading to lethal shock. The initial symptoms for SEB-induced intoxication are vertigo, muscle weakness (vasoconstriction in the extremities) within 1-8 hrs of exposure to the toxin. The symptoms that follow are nausea, vomiting and diarrhea, along with hypotension and vasodilation of blood vessels in kidney and other organs (1-24 h). Respiratory distress and pathological hypotension eventually lead to irreversible shock and death at about 40-60 hrs post exposure, although very early incidents (ca. 6 h) have been observed. The mechanism of its action is not clear, nor is it understood how SEB is massively potententated by trace levels of contaminants such as Protein A or endotoxin. In short, there is no system available to determine host exposure or individual responses and the toxin is rapidly (30 min) removed from the blood stream to the kidney PT (75%), liver and spleen.

Anthrax is another highly toxic agent. Anthrax is a natural disease of herbivorous animals that can be transmitted to humans. The causative agent Bacillus anthracis, can form spores which are extremely hardy and can remain alive for a very long time. After inhalation of a heavy dose of anthrax spores, however, the onset of the disease may occur within a day and death may follow rapidly in a couple of days. The molecular changes caused by this agent in the host is totally unknown, therefore identifying genes altered by this agent is very crucial for rapid and effective detection and for designing better treatments for this deadly pathogen. Anthrax is known to cause lethal shock.

Brucella is a highly infectious bacteria that causes disabling symptomatology (fever, chills, fatigue) in humans. Bacteria can be acquired through inhalation, ingestion, or penetration of damaged skin. As facultative intracellular parasites of macrophages, they primarily localize in the reticuloendothelial system. Bacteremia and symptoms occur from several days to several weeks after infection, presumably as a result of amplification of bacterial numbers in spleen, liver and bone marrow. Host response involves both Th1 and Th2 immune mechanisms, but is generally tilted toward Th1. In murine models of brucellosis, both antibody and T cells transfer immunity. Brucella LPS is relatively nonpyrogenic compared to LPS from Enterobacteriaceae. This property may explain the relative paucity of immune and inflammatory response early in infection.

Brucella has also been found to induce a cytokyne storm in humans which causes illness. The present invention includes treating a patient with anticytokyne therapy to reverse the harmful effects of the cytokine storm.

Plague is still another threatening toxic agent to man. The Y. pestis is an organism that causes plague. Plague symptoms include fever, chills, headache, hemoplysis and toxemia. This eventually leads to respiratory failure and death. Until now, diagnosis has been made by symptom analysis. This means that the progress of the illness can go unchecked before treatment is sought and is therefore, unsuccessful. A faster test is needed for plague. Plague also causes shock.

Botulinum toxin is extremely potent neurotoxins produced by different strains of the bacterium Clostridium botulinum. There are seven serotypes of botulinum toxins, which share the same functional mechanism: they have an endopeptidase activity that cleaves a protein in synaptic vesicles thereby inhibiting release of acetycholine. The resulting block in neurotransmitter release causes general skeletal muscle paralysis with death occurring due to respiratory failure. Following inhalation or ingestion of botulinum toxin, symptoms may appear within 24 to 36 hours or may take several days to appear. This toxin causes weakness, dizziness, dry mouth and throat, blurred vision and diplopia, dysarthria, disphonia, dysphasia and respiratory failure. A faster test for exposure to the botulinum toxin is needed.

Cholera Toxin (CT) causes vomiting, headache, diarrhea resulting in death. Mortality is as high as 80%. Diagnosis is done by symptoms of diarrhea and dehydration. The Cholera Toxin is a very difficult toxin to spot in a blood sample. Therefore, a faster, non-symptom related test is needed to prevent death.

There is no easy or fast detection method to confirm the exposure to these and other toxic agents. The deadly symptoms of lethal shock appear before they are diagnosed so the important life-saving treatment is delayed which results in deaths that could be prevented if an earlier test were available. Current methods for pathogen identification using structural-based probes may not be useful for early diagnosis for the reasons stated above.

One of the most harmful symptoms that are related to exposure to most toxic agents is the appearance of lethal shock. It is important to note that treatment of lethal shock initiated by multiple causes, has been an intractable medical problem that has been studied for (at least) decades. Clinical trials of therapies aimed at blocking/sequestering inflammatory mediators and involving huge numbers of patients, have not shown statistically significant benefits relative to no treatment.

Therefore, an object of the present invention is to provide for a method of treating patents that have been exposured to toxic agents by measuring distinct patterns in the levels of expression of specific genes and treating the patient based on the distinct patterns.

It is a further object of the invention to select a panel of genes, the altered pattern of expression of which will provide a fingerprint that is indicative of exposure to a particular toxic agent. This panel of genes will also indicate whether an exposed individual will develop the symptom of lethal shock. This panel of genes can show the potential to reveal the severity of exposure and the individual susceptibility to the agent, and can provide indicators of course of impending illness for even unknown toxic agents that leads to enlightenment of how to treat an exposed patient.

A still further object of the invention is to provide a method of early treatment of subjects exposed to threat agents, with the intervention of drugs or with agents, such as antisense oligos, which turn off the expression of genes that react detrimentally to toxins or by the addition of turned off advantageous proteins, based on the newly found gene changes.

A still further object of the present invention is to provide a method of treatment that is dependant upon the time of exposure to a toxic agent, wherein a particular treatment is effective at a particular time period after exposure.

### SUMMARY OF THE INVENTION

With the method of the present invention, the problems experienced in the past are solved. With the present invention both known and presently unknown or bio-engineered biological warfare (BW) agents can be identified based on early host functional responses to exposure and the patient that has been exposed may be treated. The present method also has the benefit of revealing the presence of low-level potentiating contaminants, such as LPS and Protein A which cause the toxins to have a more potent effect on an exposed subject. The present invention provides early information regarding individual exposure and susceptibility which is useful for determining proper treatment. This approach offers the benefits of immediate diagnosis, and the ability to identify those who have been exposed to toxic agents but have not yet developed signs or symptoms. This approach also offers a viable and successful treatment for lethal shock to prevent the symptoms from occurring.

The present invention solves the problems of the past with a method whereby an individual's exposure and his/her response to a toxic agent based on alterations in gene expression in their peripheral blood lymphoid cells (also referred to as human lymphoid cells) can be determined. These cells are readily available from personnel. These cells serve as a reservoir of historical information; although they may not, themselves, be the pathogenic target of a toxic agent, the toxic agents can indirectly activate lymphoid cells to produce a unique gene expression patterns typical of the impending illness. In addition to diagnostics, the gene expression profile potentially provides a regimen for specially designed, stage dependent, appropriate treatment.

The present invention is thus, directed to a method of treating a patient that has been exposed to a toxic agent based on amounts and time of protein/gene expression present in a sample of mammalian tissue or mammalian body fluids that has been exposed to a toxic agent. The present invention is particularly useful because it can provide an early treatment based on diagnosis of exposure to a toxic agent before the onslaught of any symptoms.

The present invention also permits a determination of time of exposure based on measurement of amounts of up regulation and/or down regulation of certain genes at particular intervals after exposure. By determining time of exposure, lethal shock can be prevented by the administering of protein products of genes that are down regulated or the administering of antisense in the case where genes are upregulated by the toxic agent.

### BRIEF DESCRIPTION OF THE FIGURES

FIG. 1 is a graph showing a time dependent expression of CTAP-III through RT-PCR wherein the levels of the CTAP-III gene go down upon SEB exposure;

FIG. 2 is a graph showing a time dependent expression of proteoglycan V1;

FIG. 3 is a graph showing a time dependent expression of GBP;

FIG. 4 is a graph showing a time dependent expression of HIF-1;

FIG. 5 is a graph showing a time dependent expression of IL-6;

FIG. 6 is a graph showing a time dependent expression of Ferritin heavy chain;

FIG. 7 is a graph showing a time dependent expression of IL-6 in response to SEB in monkey samples;

FIG. 8 is a graph showing a time dependent expression of GBP in response to SEB in monkey samples;

FIG. 9 is a graph showing a time dependent expression of CTAP in response to SEB in monkey samples;

FIG. 10 is a graph showing a comparison of expression of a cDNA, which codes for CTAP-III induced by SEB or LPS;

FIG. 11 is a graph showing a comparison of expression of IL-6 induced by SEB and LPS;

FIG. 12 is a graph showing a comparison of expression of a cDNA, which codes for GBP-2;

FIG. 13 is a graph showing a comparison of expression of a cDNA, which codes for HIF-1;

FIG. 14 is a graph showing expression pattern of RhoE in Human Kidney Cells in Response to SEB;

FIG. 15 is a graph showing a differential expression pattern of Interleukin-6 in Human Kidney Cells in response to SEB;

FIG. 16 is a graph showing differential expression pattern of Interleukin-6 in Human Kidney Cells in response to LPS;

FIG. 17a is a graph showing the expression pattern of Ferretin Heavy chain in kidney cells in response to LPS;

FIG. 17b is a graph showing comparison of Ferritin Gene Expression in human kidney cells in response to LPS and SEB;

FIG. 18 is a graph showing comparison of GBP gene expression in human kidney cells in response to LPS and SEB;

FIG. 19 is a graph showing comparison of Myosin Heavy chain Gene Expression in human kidney cells in response to LPS and SEB;

FIG. 20 is a graph showing a comparison of HIF-1 gene expression in human kidney cells in response to LPS and SEB;

FIG. 21 is a graph showing the effect of P-38 in SEB induced cell proliferation;

FIG. 22 is a graph showing the effect of P-38 on TNF-alpha induction;

FIG. 23 is a graph showing the effect of P-38 inhibitor on CD-69 expression;

FIG. 24 is a graph showing SEB-induced proliferation: inhibition by HPA-Na;

FIG. 25 is a graph showing SEB-induced proliferation: protein kinase C inhibitors;

FIG. 26 is graph showing SEB-induced TNF-a production: effects of PKC inhibitors;

FIG. 27 is a model of HPA-Na molecule;

FIG. 28 is a graph showing expression of Ferretin heavy chain in response to Anthrax;

FIG. 29 is a graph showing expression of HIF-1 after Anthrax exposure;

FIG. 30 is a graph showing expression of GBP in Anthrax treated cells;

FIG. 31 is a graph of expression of IL-6 after Anthrax exposure in human lymphoid cells;

FIG. 32 is a graph of expression of ILT6 in Anthrax treated cells;

FIG. 33a is a graph of expression of cathepsin L in Anthrax treated cells;

FIG. 33b is a graph of expression of HCI and EIF3 upon exposure to Anthrax;

FIG. 34 is a graph of the changes in expression of a GBP in response to SEB vs Anthrax;

FIG. 35 is a graph of the change in expression of IL-6 mRNA in response to SEB vs Anthrax;

FIG. 36 is a graph of the changes in expression of HIF-1 in response to SEB vs Anthrax;

FIG. 37 is a digital differential display gel profile showing gene profiles of SEB exposed samples as compared to gene profiles of a control;

FIG. 38 is a digital differential display gel profile showing gene profiles of SEB exposed sample and Anthrax exposed samples as compared to gene profiles of a control;

FIG. 39 is a digital differential display gel profile showing gene profiles of plague exposed samples as compared to gene profiles of a control;

FIG. 40 is a digital differential display gel profile showing gene profiles of cholera toxin exposed samples as compared to gene profiles of a control;

FIG. 40b is a digital differential display gel profile showing comparison of changes in gene expression in response to SEB and Cholera Toxin; and

FIG. 41 is a graph showing the expression of GBP in peripheral blood lymphoid cells of monkeys challenged with SEB;

FIG. 42 is a flow diagram of a putative signaling pathway induced in RPTEC by SEB;

FIG. 43 is a gel picture of expression of EPO in control pig and SEB treated kidneys;

FIG. 44 is a graph showing B-Lymphocyte Activation Antigen CD86 (B7-2 Antigen);

FIG. 45 is a graph showing lymphocyte adaptor protein (LNK);

FIG. 46 is a graph showing expression of Na/H exchanger and Ferrochelatase;

FIG. 47 is a graph showing expression of kinases upon anthrax exposure;

FIG. 48 is a graph showing fold changes of proteasome components;

FIG. 49 is a graph showing growth arrest and DNA-damage-inducible protein GADD153;

FIG. 50 is a graph showing fold change of ADP-ribosylation factor-1;

FIG. 51 is a graph showing fold change of Cathepsin H.

FIG. 52 is a graph showing fold changes of expression of HIF1 and RAB-2 genes;

FIG. 53 is a graph showing expression of IL-18 and IL-10 upon anthrax exposure;

FIG. 54 is a graph showing fold change of C-MYC oncogene upon anthrax exposure;

FIG. 55A is a graph showing expression of TNF alpha and beta upon anthrax exposure;

FIG. 55B is a graph showing expression of Acyl-CoA and DAD-1 gene upon anthrax exposure;

FIG. 56 is a graphic representation of some of the symptoms from exposure of piglets to incapacitating doses of SEB;

FIG. 57 is a graphic representation of some of the symptoms from exposure of piglets to lethal doses of SEB;

FIG. 58 is a graph showing the time course of the effect of SEB on the expression levels of serotonin (5-HT);

FIG. 59 is a graph showing the effect of Zofran on the symptoms of piglets exposed to SEB;

FIG. 60 is a digital image showing gene expression for EPO vs 18s in kidneys from piglets lethally challenged with SEB 48 hour post exposure, showing down regulation of the-EPO gene in SEB challenged piglets;

FIG. 61 is a graph showing the effect of EPO on body temperature, wherein the body temperature raised during SEB toxication and EPO treatment was able to bring the temperature down;

FIG. 62 is a graph showing the effect of EPO on blood pressure for SEB challenged pigs;

FIG. 63 is a graph showing administration of IV SEB to piglets and the effects;

FIG. 64A is a graph of piglet temperature after SEB exposure;

FIG. 64B is a graph of piglet systolic blood pressure after SEB exposure;

FIG. 65 is a digital image of a normal and a SEB treated piglet spleens;

FIGS. 66A-E are digital images of various organs and tissues showing the results of SEB exposure in piglets;

FIG. 67 is a digital image of Payer's patches in in SEB treated piglets;

FIGS. 68 A-F are digital images of microscopic findings of histological examination of selected tissues in SEB treated piglets;

FIGS. 69A-D are digital images of a periarteriolar lymphoid sheath in SEB treated piglets;

FIGS. 70 A-F are digital images of histological findings of SEB treated piglets;

FIG. 71 is a graph of microarray results of five genes at selected times after SEB exposure;

FIG. 72 is a digital image of a histological cross section of lymphoid tissues at 48 hours after SEB administration showing diffuse expansion of the lymphoid tissue and variable congestion;

FIG. 73 is a digital image of a histological cross section of lymnphoid tissue at 72 hours after SEB administration showing extensive congestion, hemorrhage and edema with areas of lymphocytolysis;

FIG. 74 is a digital image of a histological cross section of normal lymphoid tissue;

FIG. 75 is a graph showing systolic blood pressure changes correlated with host response genes that can be diagnostic indicators of stage of illness;

FIG. 76 is a table showing stage appropriate diagnostic markers or therapeutic targets;

FIG. 77 is a graph showing plasma serotonin levels in sham or SEB challenged monkeys;

FIG. 78 is a graph showing stage appropriate therapy for edema in animals.

FIG. 79A is a three dimensional graph showing global gene expression profiles of progression of SEB 2° and 3° effects with time;

FIG. 79B is a three dimensional graph using predictive modeling invitro to 14 biothreats successfully selected sets of genes to identify SEB in vivo.

### DETAILED DESCRIPTION

Discussion of the Figures and Tables:

It has been found that the host gene expression patterns act as diagnostic markers. The present inventors have compiled a library of genes altered by different toxic agents. These libraries consist of hundreds of genes altered upon exposure to a particular agent. These discoveries and method of diagnosing exposure to a toxic agent are set forth in U.S. Pat. No. 6,316,197, incorporated herein in its entirety by reference. Excerpts from U.S. Pat. No. 6,316,197 are provided below for convenience.

A gene library has been generated for each biological warfare agent in the present invention. This list gives the name of the gene and the ratio or fold difference of genes from the control values. These libraries allow the determination of the gene changes induced by each agent. The genes that are 2 fold and higher in ratio are good candidates for marker genes for determining exposure to each specific agent.

The inventors have identified a list of more than 200 genes per agent that change upon exposure to a toxic agent. These genes are important for not only early detection before the symptoms appear but also provide therapeutic targets that can be used for treatment of patients.

The gene lists provided in the following tables for each agent, provide the first glimpse ever at observing the molecular changes induced in the host upon exposure to toxic agents. No one has looked at the molecular events in the host before in such a global way.

The library of genes is a useful tool for developing a diagnostic chip that will contain all the disclosed gene names on one slide. These DNA chips are useful for confirmation of gene expression patterns upon exposure to toxic agents. The specific genes that are altered upon exposure serve as diagnostic markers and help predict the course of illness. A DNA chip containing specific genes for each agent, all in the same chip, which is used for diagnostic purposes.

With blood samples from exposed individuals to any of the above mentioned toxic agents, RNA is isolated and hybridized to the chip by methods known in the art to determine the gene changes. We have developed an extensive database of these gene changes with all the mentioned agents that can be used to identify the type of exposure. Targeting these genes for therapeutic intervention at various stages of illness is the key to this invention.

Effect of SEB on the Expression of Different Genes: Table 1

RT-PCR was performed on RNA samples from human lymphoid cells treated with SEB for different time periods. Several changes in expression of genes were observed that were up regulated or down regulated in response to the toxin in a time dependent manner as summarized in Table 1.

Differential display was used to identify various genes that are altered upon SEB exposure to human lymphoid cells in vitro. The differential display (DD-PCR) procedure has been completed using all the possible anchored and arbitrary primer combination (220) that has covered the entire RNA population. We have identified more than 900 genes that are altered upon SEB exposure. See Tables 1a and 1b.

Description of Gene Changes Induced by each Threat Agent that can be used for Diagnostic Tests:

Gene lists were obtained after screening of several gene arrays. Each agent was exposed to the cells and RNA isolated for gene array experiments. The untreated and treated samples were then labeled with 33P and hybridized to the arrays. The signals were obtained by scanning in a BIORAD scanner and the intensities of each spot was normalized with the housekeeping genes. Global normalization was also performed after the 16 bit Tiff image was aligned to the grid for each spot.

Each table represents columns showing first the function of the gene, the name of the gene, and the numbers represent the fold change at indicated time points. Fold change, was calculated after normalization of signals and was obtained by dividing the treated number with the untreated control. The ratio obtained after this is designated as fold change.

Use of Gene Array for Identification of Altered Genes in the Host:

The inventors have used gene array, a powerful tool, for identification of altered genes in the host upon exposure to the toxic agents. Libraries of genes were generated for each agent. The gene names are listed with each agent separately. The gene names are listed with each agent separately. The results of Tables 2-9 were obtained using gene array. These genes are altered specifically by each pathogen in a human upon exposure.

Table 2: Gene Library from Brucella Exposure.

Human lymphoid cells were exposed to Brucella Melitensis in vitro for different time periods, RNA isolated and gene screening performed using Gene Array blots. Table 2 shows the differences in expression pattern of untreated and treated samples. Many genes are upregulated and many genes are downregulated. They act as marker genes to predict exposure to Brucella.

Table 3: Gene Library for Yersinia Exposure.

Human monocytes were exposed to Yersinia pestis in vitro for different time periods, RNA isolated and gene screening performed using Gene Array blots. Table 3 shows the differences in expression patterns of untreated and treated samples. Each of these genes that are up regulated or down regulated 2 fold and higher can act as marker genes for Plague (Yersinia) exposure and also be used as therapeutic targets.

Table 4: Gene Library for SEB Exposure.

Human lymphoid cells were exposed to SEB in vitro for different time periods, RNA isolated and gene screening performed using Gene Array blots. Table 4 shows the differences in expression patterns of untreated and treated samples. Each of these genes that are up regulated or down regulated 2 fold and higher can act as marker genes for SEB exposure. These genes can be also targeted for therapy.

Table 5: Gene Library for Anthrax Exposure in Vitro.

Human lymphoid cells were exposed to Anthrax spores in vitro for different time periods, RNA isolated and gene screening performed using Gene Array blots. Table 5 shows the differences in expression pattern of untreated and treated samples. Each of the genes that are up regulated or down regulated 2 fold and higher act as marker genes for Anthrax exposure. These specific genes can be targeted for therapy and gives us much more choices other than using CIPRO which is the most common antibiotic treatment available today.

Table 6: Gene Changes Induced by Anthrax in Vivo in Monkeys

In vivo monkeys were exposed to Anthrax spores, whole blood collected at different time periods (24hr, 48hr, 72hr), RNA isolated and hybridized to Gene Array blots. Table 6 shows the ratio of treated over control samples. Each of the genes that are up regulated or down regulated 2 fold and higher act as marker genes for Anthrax exposure. A pattern of gene expression is also seen during these time points. Some of the early genes are upregulated by 24h and they disappear by 72h. However, some of the damaging genes causing cell death appear at later time points and they stay up regulated. These genes act as diagnostic markers and therapeutic targets for exposure to each of these BW agents.

Table 7a-7d: Gene Library for Venezuelan Equine Encephalitis (VEE) Virus Exposure in Vitro.

Human lymphoid cells were exposed to VEE virus in vitro for different time periods, RNA isolated and gene screening performed using Gene Array blots. Tables 7a and 7b shows the differences in expression patterns of untreated and treated samples. Many genes are upregulated and many genes are downregulated, acting as marker genes to predict exposure to VEE virus. Table 7a shows Array I and Table 7b shows Array II. Table 7c is a table showing gene changes induced by VEE virus invitro in human lymphoid cells for a cancer array. Table 7d is a comprehensive table showing gene changes induced by VEE virus in vitro in human lymphoid cells.

Table 8: Gene Library for Dengue Virus Exposure in Vitro.

Human lymphoid cells were exposed to Dengue virus in vitro for different time periods, RNA isolated and gene screening performed using Gene Array blots. Table 8 is a comprehensive table that shows the differences in expression patterns of untreated and treated samples. Many genes are upregulated and many genes are downregulated acting as marker genes to predict exposure to Dengue virus. These genes can be targeted specifically to combat the disease progression.

Table 9: Baseline Gene List

Approximately 244 genes were selected that were never expressed in 24 untreated control human lymphoid samples. The expression level of these genes were below the background levels in all these 24 samples. However, upon treatment with various agents, the expression of these genes was significantly altered. This leads us to believe that these genes can be used as specific diagnostic markers to identify exposure to the biological threat agents that we have tested. These genes since are never expressed in unexposed individuals, upregulation of these particular genes will indicate exposure to some agent. Only after exposure to a bacterial pathogen, or virus or toxin will alter the expression of these genes, thus these sets of genes are very important for diagnostic tests. These genes are also useful for targeting them after exposure to these BW agents for effective treatment.

### Discussion of Figures

Genes Identified Using Differential Display PCR: A Few Genes that were Identified Using DD-PCR to be Altered by SEB Exposure Were Selected and Confirmed their Level of Expression Using RT-PCR.

Effect of SEB on the Expression of CTAP-III Gene:

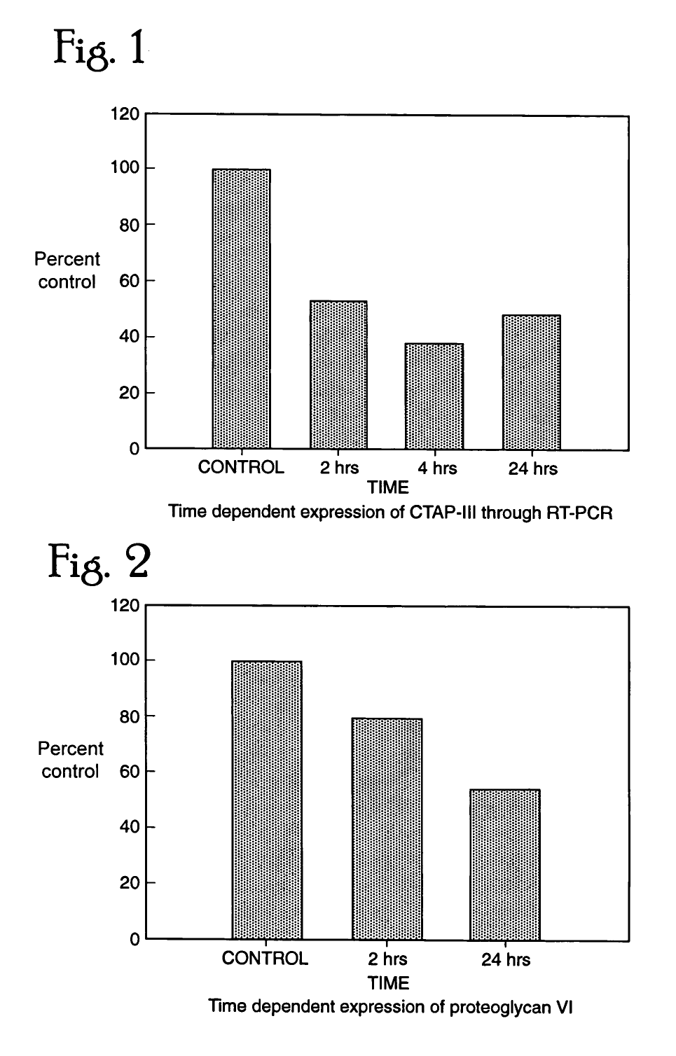

The CTAP III gene was identified to be down regulated by SEB, which was confirmed by RT-PCR, and by Northern blot analysis. FIG. 1 shows the levels of CTAP-III going down upon SEB exposure. A down regulation of the expression of CTAP III was observed as early as 2 hours (40-60%), and this activity was retained even at 24 hours (40-60%).

Effect of SEB on the Expression of Proteoglycan Gene:

Primers were designed for proteoglycan V₁ (Vimentin) gene and RT-PCR performed on RNA samples from different time periods of SEB exposure. There was a dramatic decrease in expression upon SEB exposure (FIG. 2). Within 2 hrs the expression of this gene was down regulated to 70-85% of control levels and the expression levels were further decreased to 45-60% by 24 hours.

Effect of SEB on Gene Expression of GBP:

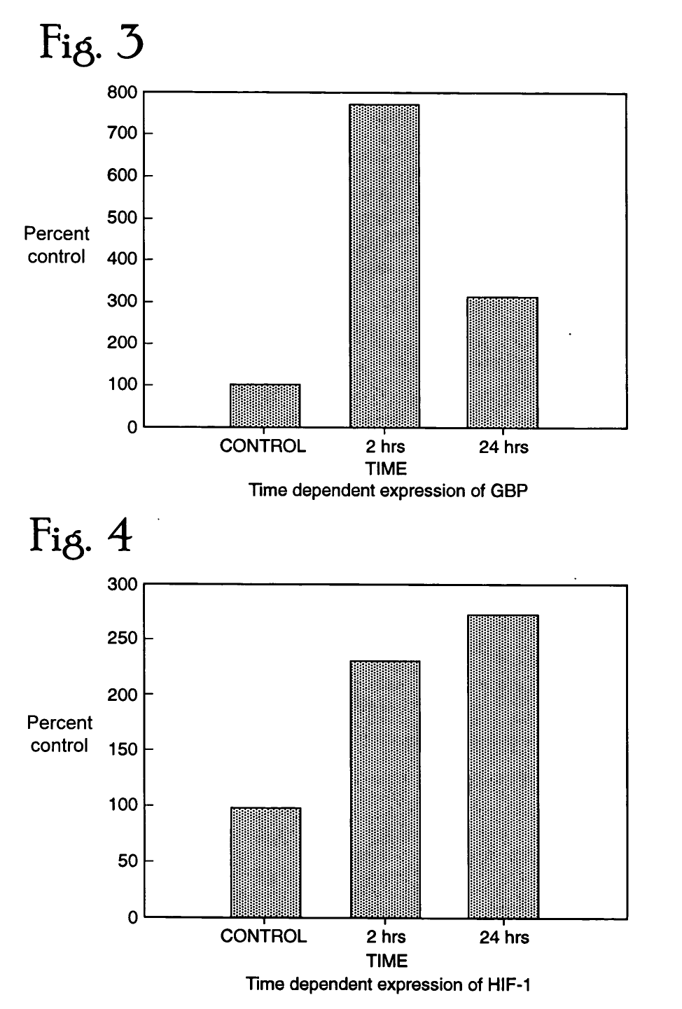

SEB exposure caused a significant increase in expression of this gene that is involved in Guanylate cyclase regulation (FIG. 3). A clear induction of the expression of GBP was evident as early as 2 hours (7.5 - 8 fold). Even though its activity gradually decreased by 24 hours, the levels were still well over control levels (3-3.5 fold).

Effect of SEB on Gene Expression of Hypoxia Inducible Factor (HIF-1):

The expression of the gene HIF-1 was also up regulated in response to SEB in a time dependant manner (FIG. 4). Increase of the expression of this gene was observed at 2 hrs (2-2.5 fold), and the expression continued to increase even at 24 hrs (2.5-3 fold).

Effect of SEB on gene expression of IL6:

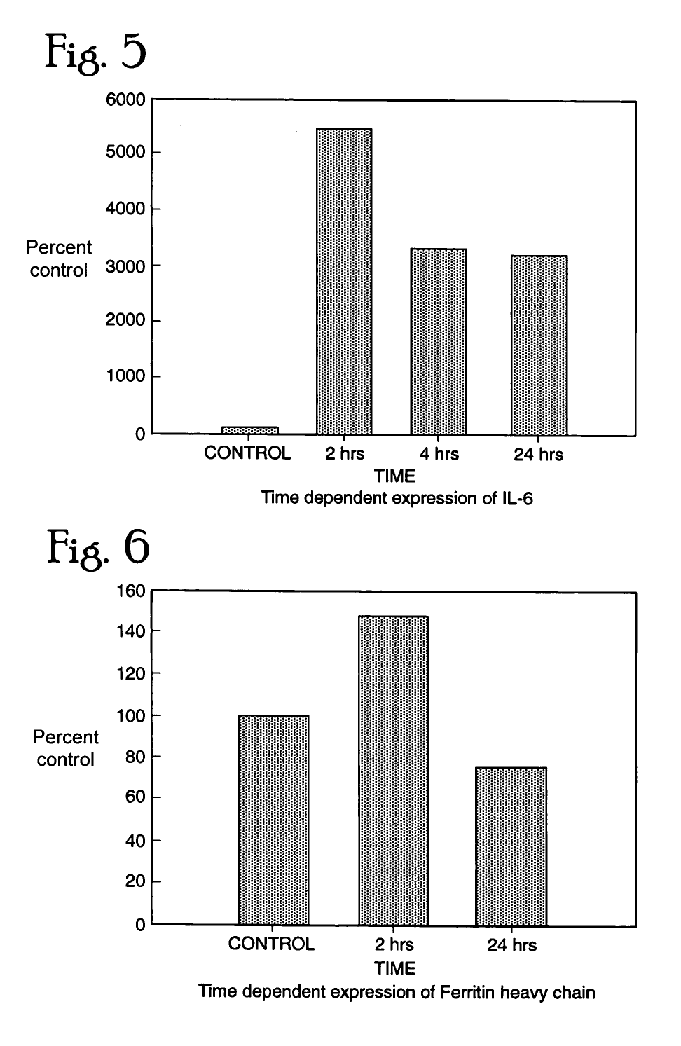

IL6 gene expression was significantly up regulated upon SEB exposure within 2 hrs of exposure (FIG. 5). A significant enhancement of the expression of IL-6 was detected as early as 2 hrs (50-55 fold), and this enhanced expression was evident around 24 hours (30-35 fold).

Effect of SEB Exposure on Gene Expression of Ferritin Heavy Chain:

SEB exposure caused a decrease in the expression of Human Ferritin gene as shown in FIG. 6 by 24 hours. Even though ferritin expression was about 1.4-1.5 fold by 2 hrs, it gradually decreased by 24 hrs reaching 70-80% of control levels.

Confirmation of Gene Changes in Monkey Blood Samples Exposed to SEB:

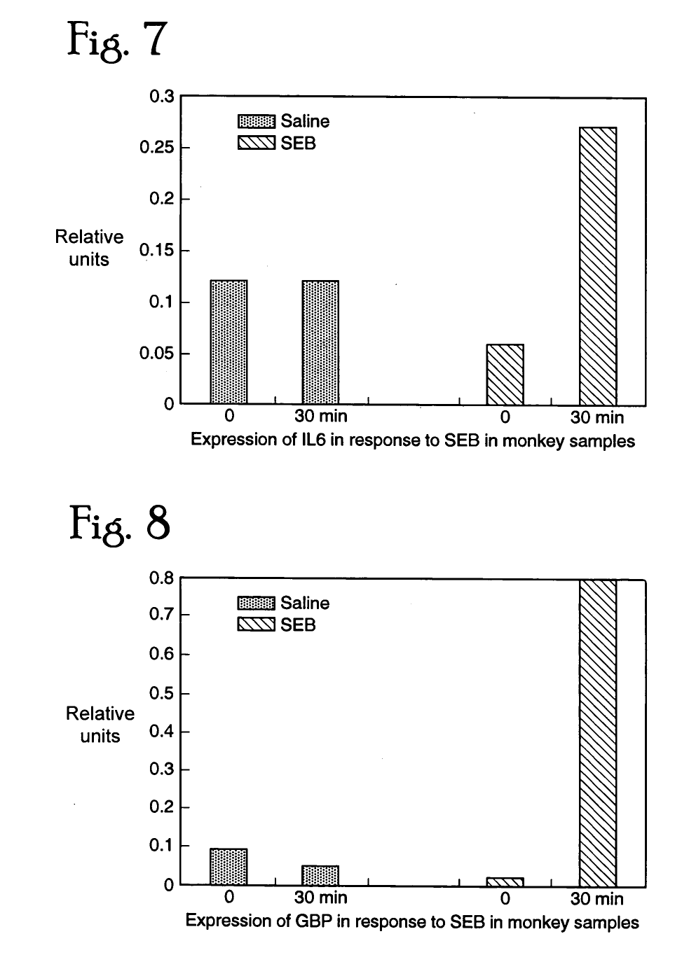

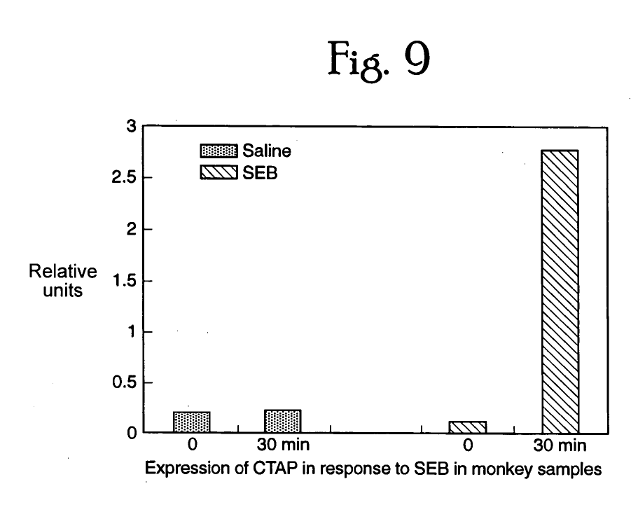

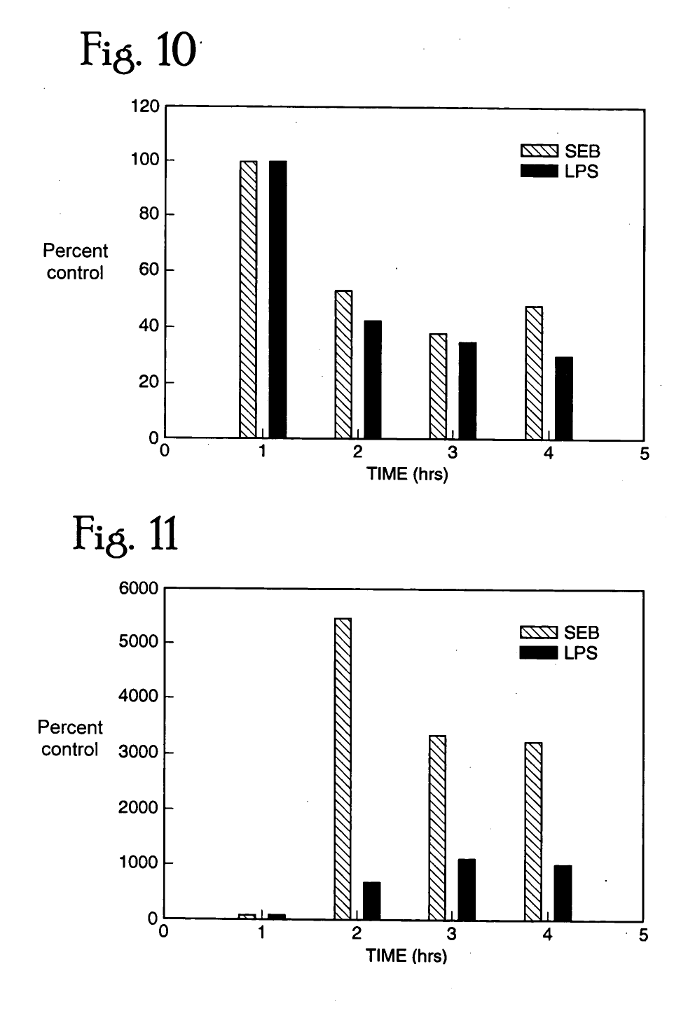

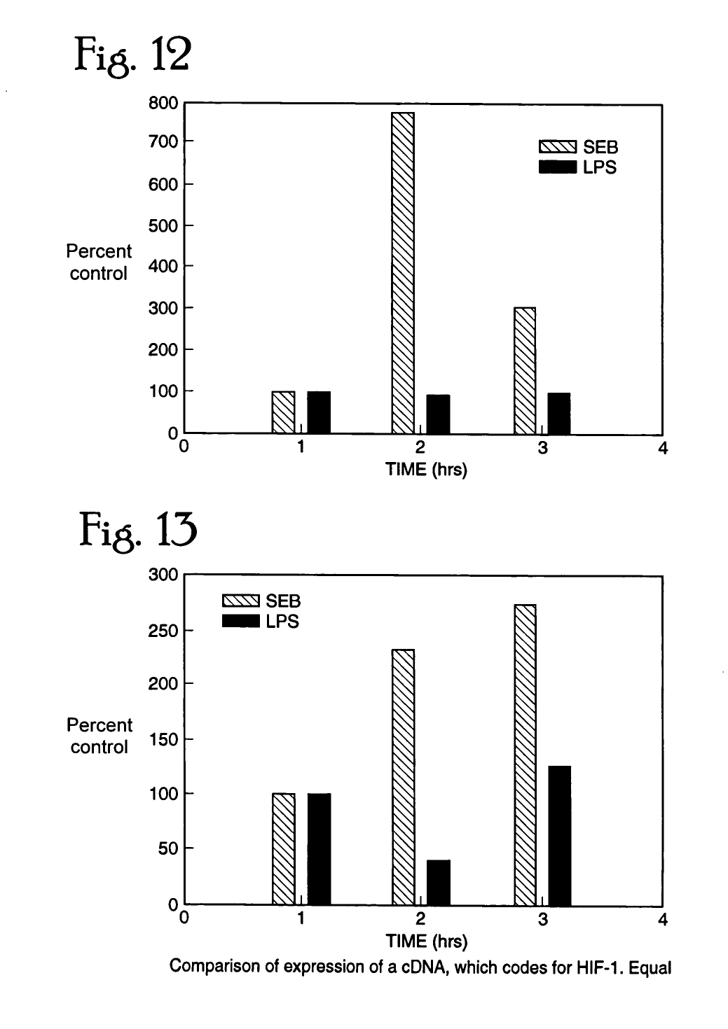

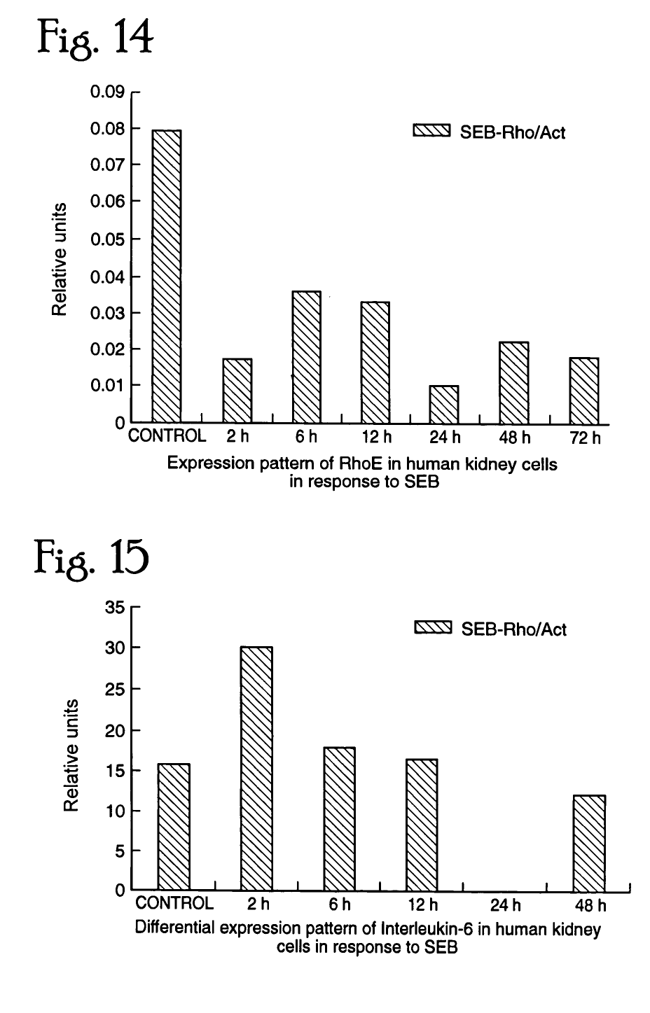

We verified these findings in lymphocytes of monkeys challenged with SEB. Using PCR primers designed for the selected genes, we have found unique patterns in alteration of gene expression as early as 30 minutes post-aerosol challenge. We tested three genes in lymphocytes from monkey blood samples after exposure to SEB (FIG. 7-9).

The expression of IL6 and GBP was up regulated by 30 minutes of SEB challenge in monkey samples. This was a sub-lethal dose given to the monkeys so the expression of CTAP-III was also shown to be up regulated in these samples by 30 minutes of exposure. Similar results were obtained with human cells in vitro when exposed to SEB.

Summary of Changes: Table 2a summarizes all the changes that were observed that were induced by these toxins in human lymphoid cells.

Comparison of the Effects of SEB and LPS on Expression of CTAP-III

Equal amount of the RNA samples treated with SEB and LPS along with proper controls were reverse transcribed as described elsewhere and amplified using custom designed primers of CTAP-III. Equal volumes of samples were resolved on a 1% agarose gel, visualized by ethidium bromide staining and quantitated by NIH image program 1.61. #1, Control; #2-4 were treated with 100 ng/ml SEB or LPS for different time periods and were normalized with expression of β-actin. #2; 2 hrs, #3, 4 hrs; #4, 24 hrs. Both SEB and LPS toxins were capable of down regulating the CTAP-III gene while showing a similar activation pattern. Effect of LPS was prominent compared to SEB. Down regulation of the CTAP III gene was visible as early as 2 hrs (SEB 50% of control levels and LPS 45% of control levels). After 24hrs of treatment expression of the CTAP-III gene induced by SEB was about 33-45% of control levels while LPS was 25-35% (FIG. 10). In FIG. 10, for each pair of results shown comparing SEB to LPS, the left band is SEB and the right band is LPS.

Comparison of the Effect of SEB and LPS on the Expression of the IL-6 GENE

Equal amount of the RNA samples treated with SEB and LPS along with proper controls were reverse-transcribed as described elsewhere and amplified using custom designed primers of IL-6. Equal volumes of samples were run on 1% agarose gel in a gel loading buffer, subjected to electrophoresis at IOOV for 40 min., visualized by ethidium bromide staining and quantitated by the NIH image program 1.61. #1, Control; #2-4 were treated with 100 ng/ml SEB or LPS for different time periods and were normalized with β-actin. #2; 2 hrs, #3, 4 hrs; #4, 24 hrs. Both toxins up regulated the expression of the IL-6 gene in a time dependent manner while the effect of SEB in human lymphoid cells was more prominent. An up regulation was seen as early as 2 hrs by both toxins (SEB 52-57 fold, LPS 7-8 fold), and was still up regulated at 24 hrs (SEB 30-35 fold, LPS 10-12 fold). SEB had a pronounced effect on IL-6 gene expression but with LPS it was not very significant (FIG. 11). In FIG. 11, for each pair of results shown comparing SEB to LPS, the left band is SEB and the right band is LPS.

Comparison of the Effects of SEB and LPS on Expression of GBP-2

Equal amount of the RNA samples treated with SEB and LPS along with proper controls were reverse-transcribed as described elsewhere and amplified using custom designed primers of GBP-2. Equal volumes of samples were resolved on a 1% agarose gel, visualized by ethidium bromide staining and quantitated by the NIH image program 1.61. #1, Control; #2-3 were treated with 100 ng/ml SEB or LPS for different time periods and were normalized with P-actin. #2; 2 hrs, #3, 24 hrs. GBP was clearly up regulated by SEB by 2hrs (7-8 fold), and was seen even after 24 hrs (3-3.5 fold). LPS had no effect on the expression of GBP-2 (FIG. 12). ). In FIG. 12, for each pair of results shown comparing SEB to LPS, the left band is SEB and the right band is LPS.

Comparison of the Effects of SEB and LPS on Expression of HIF-1

The HIF-1 gene expression was up regulated by SEB in a time dependent manner reaching an optimum value by 24 hrs (2.5-3 fold). Expression pattern of the HIF-1 gene by LPS was different to that observed for SEB. There was no significant change observed even after 24 hrs (FIG. 13). In FIG. 13, for each pair of results shown comparing SEB to LPS, the left band is SEB and the right band is LPS.

Summary of Unique Changes Induced by SEB and LPS:

Table A summarizes the changes induced by SEB and LPS. The time dependent changes are also noted in this table.

Differential Gene Expression Patterns in Human Kidney Cells Induced by SEB

The RhoE gene was identified by differential display (DD)—polymerase chain reaction (PCR) as one of the genes that was down regulated by SEB in renal proximal tubule epithelial cells (RPTEC). Two- to eight-fold reduction in expression, depending on the length of cell exposure to SEB, was confirmed by reverse transcription (RT)—PCR with specific primers (FIG. 14). Expression of RhoE gene was down regulated by SEB as early as 2 hrs (¼ th of control levels) and this was seen even after 72 hrs (¼^{th} of control levels).

Comparison of Gene Expression Patterns Induced by LPS and SEB in Human Kidney Cells.

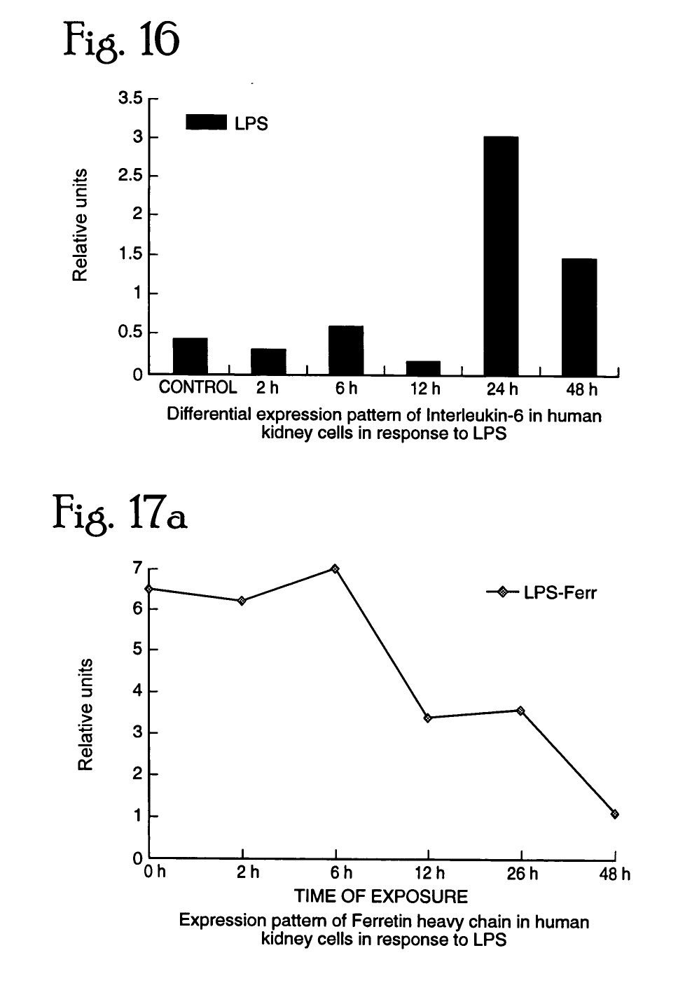

A) Genes encoding ferritin, Guanylate binding protein (GBP) and interleukin-6 (IL-6) were differentially expressed in RPTEC (renal proximal tuble epithelial cell) stimulated with LPS. The peak expression of ferritin and GBP occurred at approximately 6 h of exposure, while the IL-6 did not show significant levels of expression until 24 h of the toxin stimulation. None of these genes were known to be differentially expressed in cells stimulated with SEB, as compared to the control cells (FIG. 15-18).

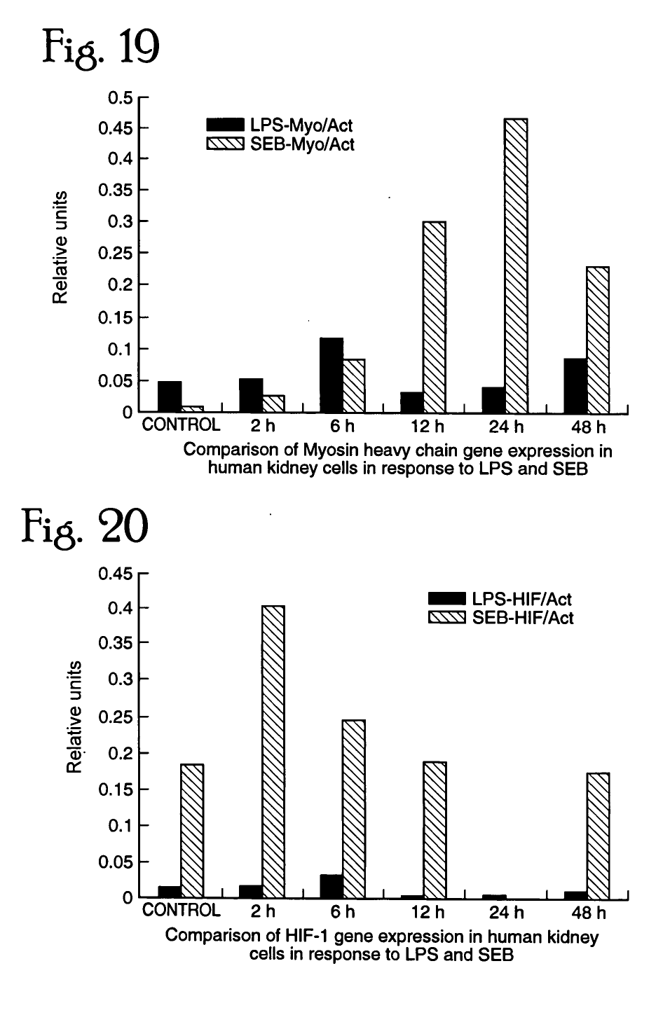

B) Genes encoding hypoxia-inducible factor-1 (HIF-1) and myosin heavy chain showed no significant differences in expression patterns in LPS-stimulated RPTEC. However, both of these genes were up regulated in SEB-stimulated cells, with peak expression of HIF-1 and myosin occurring at approximately 2 h (greater than two-fold increase over control) and 24 h (greater than 20-fold difference increase over control), respectively (FIG. 19-20).

In FIG. 17b, for each pair of results shown comparing Ferritin gene expression in response to SEB and LPS, the left band is LPS-FER/Act and the right band is SEB-Fer/Act.

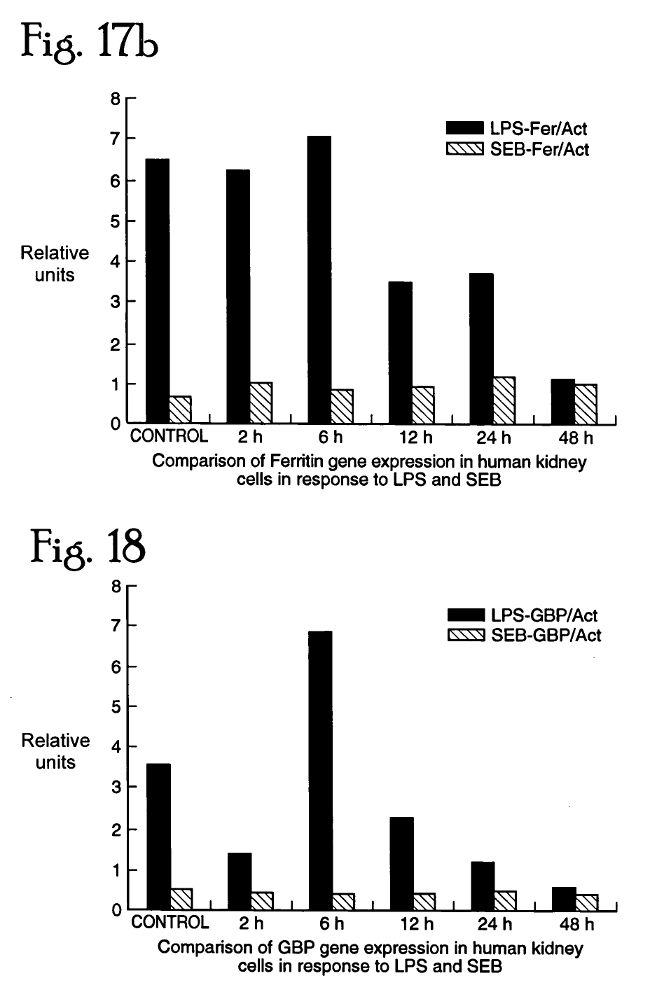

In FIG. 18, for each pair of results shown comparing GBP gene expression in response to LPS and SEB, the left band is LPS-GBP/ACT and the right band is SEB-GBP/Act.

In FIG. 19, for each pair of results showing the comparison of myosin heavy chain gene expression in human kidney cells in response to LPS and SEB, the left band is LPS-Myo/Act and the right band is SEB-Myo/Act.

In FIG. 20, for each pair of results showing the comparison of HIF-1 gene expression in human kidney cells in response to LPS and SEB, the left band is LPS-HIF/Act and the right band is SEB-HIF/Act.

Summary of Gene Changes in Human Kidney Cells in Response to SEB:

Table B summarizes all the 32 genes that were altered in kidney cells in response to SEB exposure. There were 14 genes that were up regulated and 18 genes that were down regulated.

### Treatment

Effect of Drugs to Block SEB Induced Responses:

We have tested three different drugs and have found them to be effective blockers of SEB induced responses. P-38 inhibitor is an inhibitor of a kinase that is crucial for signal transduction of SEB in human lymphocytes. It is preferred to administer P-38 within 2 hours of exposure to SEB. HPA-Na is a heteropolyanion that is a free radical scavenger that is also very effective in blocking the SEB effects. It is preferred to administer HPA-Na within 2-3 hours of exposure to SEB

Effect of P-38 Inhibitor on SEB Induced Cellular Events:

The drug known as P-38 was obtained from Smith Klien Beecham, NJ. Human TNF-α can either be as a membrane associated (26 kDa) or secreted (17 kDa) form (Kriegler, et al., cell, 53, 45-53, 1988). TNF-α induced by SEB is in the secreted form. TNF-alpha induces hemorrhagic necrosis and regression of tumors in animals, is cytotoxic to transformed cells, and promotes immunity, inflammation, insulin resistance, hypertension, shock and some cases chronic diseases (Tracey, et al., Annu. Rev. Cell Biol., 9, 317-343, 1993; Sidhu, et al., Pharmacol. Ther., 57, 79-128, 1993). Ability of P-38 inhibitor to block the induction of TNF-alpha makes this a solid therapeutic target.

Cells of the immune system utilize surface molecules for selective trafficking and focused cellular responses to a variety of inflammatory stimuli (Hogg, et al., Curr. Opin. Immunol., 5, 383-589, 1993; Mackay, et al., Immunol. Today, 1, 99-104, 1993). CD69 is a surface molecule that is rapidly expressed in response to various interleukins such as IL-2, IL-13 and is closely linked to the activation to protein kinase C in human T lymphocytes (Bjorndahl, et al., J. Immunol., 1, 4094-4098, 1988; Cebrian, et al., Eur. J. Immunol., 19, 809-816, 1989; Hamann, et al., J. Immunol., 150, 4920-4928, 1993; Testi, et al., J. Immunol., 150, 4920-4924, 1989). Flow cytometry is used for assessing surface molecule expression on selected cell populations. Ability of P-38 kinase inhibitor SB-203580 to reduce the production of CD69 induced by SEB increases the importance of P-38 inhibitor as a therapeutic target.

Effect of P-38 Inhibitor on SEB Induced Responses:

Effect of p-38 Inhibitor on Cell Proliferation.

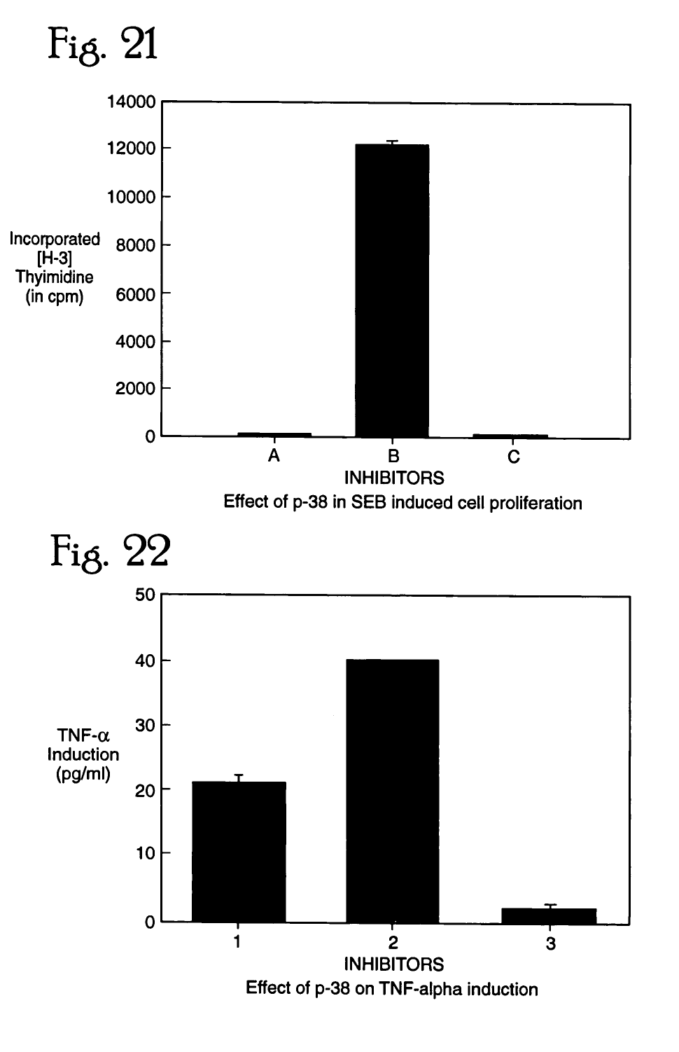

P-38 inhibitor was administered at a concentration of 10 uM. P-38 inhibitor was able to block the growth of T-cells as shown in FIG. 21. Induction of cell proliferation by SEB as shown in FIG. 21 (12,000 cpm) was clearly inhibited with the treatment of SEB induced cells with 10 uM of p-38 inhibitor SB-203580 (FIG. 21).

Effect of P-38 Inhibitor on the Induction of TNF-alpha

Human lymphoid cells were treated with P-38 inhibitor followed by SEB exposure.

Upon the treatment of SEB with P-38, the TNF-α gene expression in human lymphoid cells almost doubled compared to untreated samples. When treated with 10 μg of P-38 inhibitor, the previously observed induction of the TNF-α gene by SEB was brought down back to control levels (FIG. 22).

Effect of p-38 Inhibitor on the Induction of CD-69

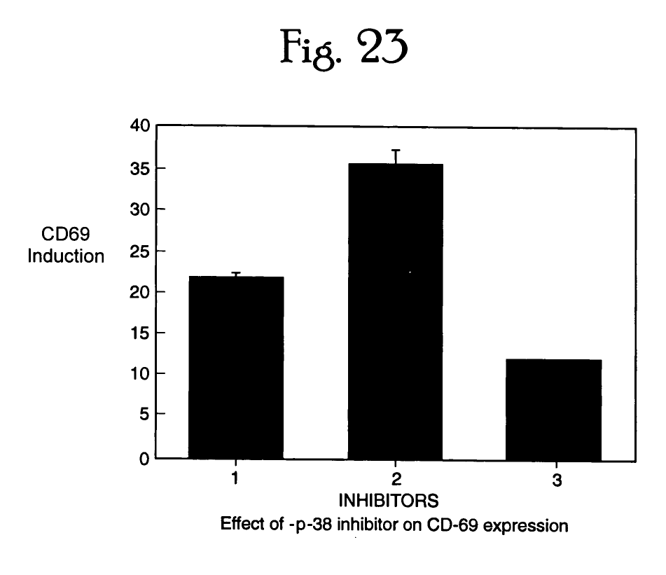

When human lymphoid cells were treated with 100 ng/ml of SEB, we observed a clear stimulation of CD69 production in human lymphoid cells (15-20 pg/ml over control levels). This induction with SEB was clearly blocked upon the treatment with 10 uM of P-38 inhibitor (FIG. 23). Therefore blocking the enzyme activity of P-38 kinase was bale to block several key steps involved in SEB toxicity, whereby ultimately blocking cell proliferation induced by SEB, suggesting a potential therapeutic agent for treating lethal shock induced by SEB.

Effect of HPA-Na and PKC Inhibitors on SEB Induced Responses:

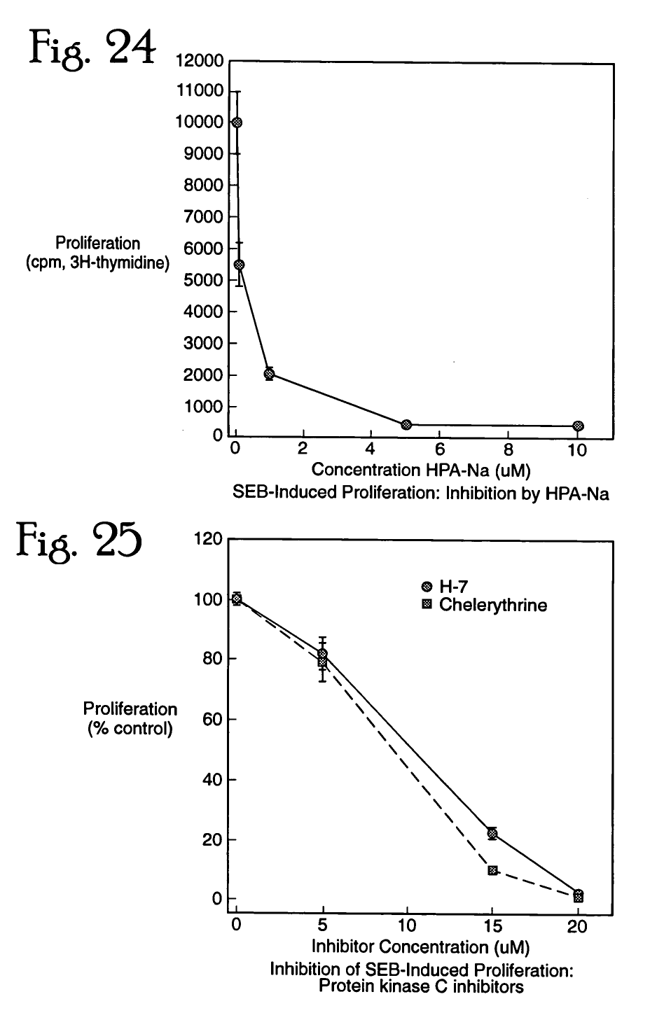

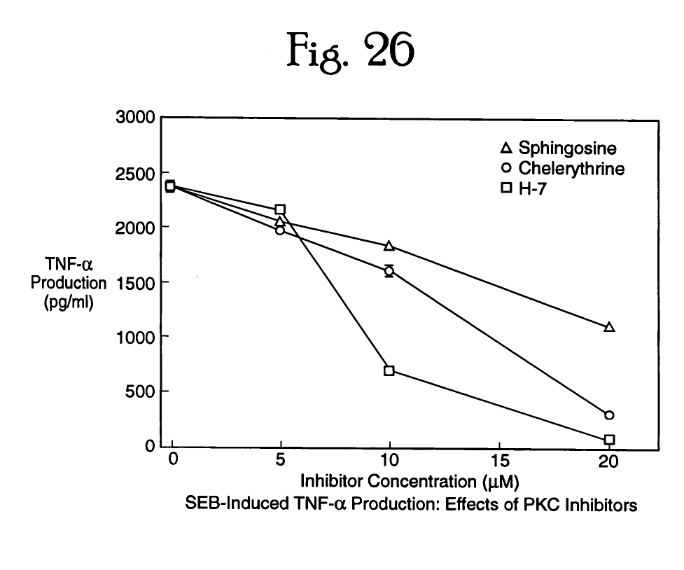

SEB is known to induce rapid proliferation of the T cells, we tested different concentrations of the drugs on this assay and showed a definite decrease in proliferation. (FIG. 24, 25) We also showed that the PKC inhibitor was able to block effectively TNF-alpha production upon exposure of human peripheral blood lymphoid cells (isolated monocytes/lymphocytes: 1/4) to SEB as well. H7 was the inhibitor that was most effective among the other PKC inhibitors (FIG. 26). These class of inhibitors can have therapeutic potential for treatment of early symptoms induced by the toxin.

Effect of HPA on Proliferation Assay Induced by SEB:

The drug HPA-Na was given to human lymhpoid cells in the amount shown in FIG. 24 and was able to block the SEB induced T cell proliferation which was tested by thymidine incorporation. The drug HPA-Na was chosen in the laboratory out of a panel of several of these group of heteropolyanions. As shown in FIG. 24, HPA-Na was effective at very low concentrations to block the proliferation of lymphoid cells induced by SEB. The super antigenic properties of this toxin were blocked by this drug, thus can be a potential treatment for lethal shock.

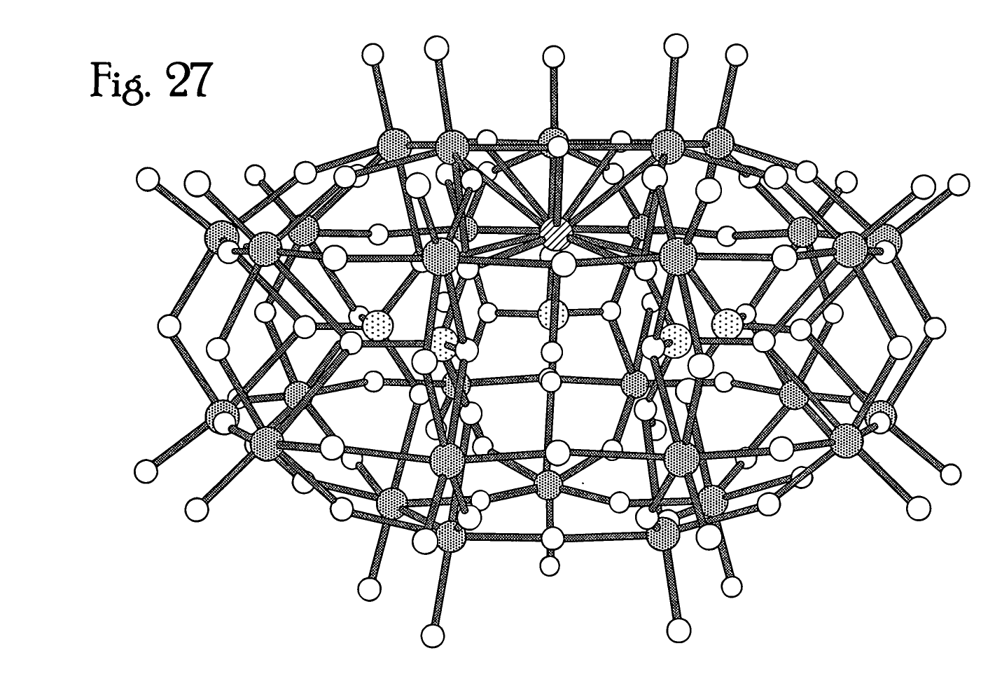

The drug HPA-Na (a heteropolyanion which is a metal ion derivative of polyoxotungstate) was synthesized in the laboratory using methods outlined in Heteropoly and Isopoly Oxometalateds, Michael Thor Pope, Springer Verlag, Berlin, Germany 1983. This drug is water soluble and stable at room temperature. Its structure is shown in FIG. 27.

Effect of Inhibitors of Protein Kinase C:

As shown in FIG. 25, these inhibitors (H-7 and Chelerythrine) effectively blocked SEB-induced proliferation. As shown in FIG. 26, these inhibitors also blocked production of TNF-α and also blocked production of eicosanoids and neuropeptides. The dose was 10 uM.

Treatment of Toxin Induced Illness with Antisense:

A new technique for treating patients is to prevent expression of specific genes by administering antisense to the mRNA for that particular gene. For the situation described in this application, persons exposed to toxic agents, in addition to classical drugs that target specific metabolic pathways, can be treated with antisense to mRNA coding for specific genes that we have determined to be critical for toxicity induced by the specific toxic agent. An example is that staphylococcal enterotoxin B illness is characterized by rapid drop in blood pressure, likely due to loss of regulation of vascular tone especially in organs. We have identified several genes, with altered expression in response to SEB that are involved in various aspects of regulation of vascular tone (Table 1b; FIG. 3, 12). Another example of a more severe progression of SEB-induced illness is respiratory distress (leading to death); we have also found SEB-induced alterations in expression of genes that have been related to respiratory distress (Table 1b FIG. 4).

First one would determine, based on gene array analysis or conventional structural-based probes, that the patient had been exposed to a toxic agent. The probes used were designed to identify the agent such as SEB toxin gene or Anthrax genes, or genes specific for the pathogen itself.) If gene array analysis had been performed, detection of expressed genes known to be critical for the progression of the intoxication would be apparent by comparing the expression patterns with the gene libraries set forth in this description.

Dose of antisense: Typically patients have been treated and tolerate a dose of 0.5- 3 mg/kg/day delivered by continuous intravenous infusion. Antisense is easily designed for any gene based on methods well known in the art. Saline is an example of a carrier used to deliver it intravenously. In most cases for the toxic agents, there is a critical time period of the illness that lasts for 2-4 days. Treatment with antisense therapy for this length of time would not present a problem. One study treated ovarian cancer patients for 21 days on/ 7 days off (Yuen, et al., Yuen AR, et al., Phase I study of an antisense oligonucleotide to protein kinase C-alpha (ISIS 3521/CGP 64128A) in patients with cancer, Clin Cancer Res 1999 Nov., 5(11): 3357-63 (1999). Other methods of administration are also under study including intraperitoneal, intramuscular and oral administration.

Antisense (complementary base pairs to the desired sequence) is typically constructed beginning with the 3 base “start code” for a specific mRNA and proceeding with the nucleotide sequence of the mRNA for the gene in question. Using Blast and other Gene search engines, one continues down the sequence of the desired gene until one determines that the sequence targets only the mRNA for the desired gene. An example in our laboratory is that for liver-fatty acid binding protein (L-FABP), a 19 base oligonucleotide sequence was specific for L-FABP. Hammameih, FASEB J. in press. (Das et al., Clin. Cancer Res., 7:1706-1715, 2001). This antisense was able to block the effects of L-FABP in cancer cells.

In general, this approach is successful because the antisense fragment binds to the complementary region of the selected gene. At that point, several theories exist such as that RNases are activated due to the complementary oligonucleotide bound to the mRNA or that blocking the “start code”, along with binding of the complementary oligonucleotide to the selected gene, prevents mRNA synthesis. Never the less, extensive studies indicate that directed antisense blocks synthesis of the gene in question. Shi Q, et al., Constitutive and inducible interleukin 8 expression by hypoxia and acidosis renders human pancreatic cancer cells more tumorigenic and metastatic. Clin Cancer Res 5(11):3711-21 (1999); Cho-Chung YS, Antisense DNA-targeting protein kinase A-RIA subunit: a novel approach to cancer treatment, Front Biosci 4:D898-907 (1999); Tian XX et al, Altered expression of the suppressors PML and p53 in glioblastoma cells with the antisense-EGF-receptor. Br J Cancer 81(6):994-1001 (1999). Additionally, some of the genes (and their corresponding proteins) found to be altered in response to toxic agents have already been studied for other reasons and specific inhibitors exist to treat the toxic agent-induced illness. Respiratory distress induced by SEB is an example (see Table 1b). (Table 1b. is a table showing a list of genes that have been identified to be altered upon SEB exposure using DD-PCR.)

Although no one knew previously that these genes and their corresponding proteins were altered in response to SEB or other listed bio-threat agents, these mediators were well known to be involved in asthma-induced respiratory distress. As such, specific inhibitors have been and are being designed to target these products, such as antisense to specific genes or inhibiting agents of an enzyme or a signaling pathway.

Intravenous administration of antisense therapy is likely to be the most successful route since most of the action of toxic agents might be expected to be associated with lymphoid and endothelial cells. In addition, IV could be distributed to the kidney, liver and spleen.

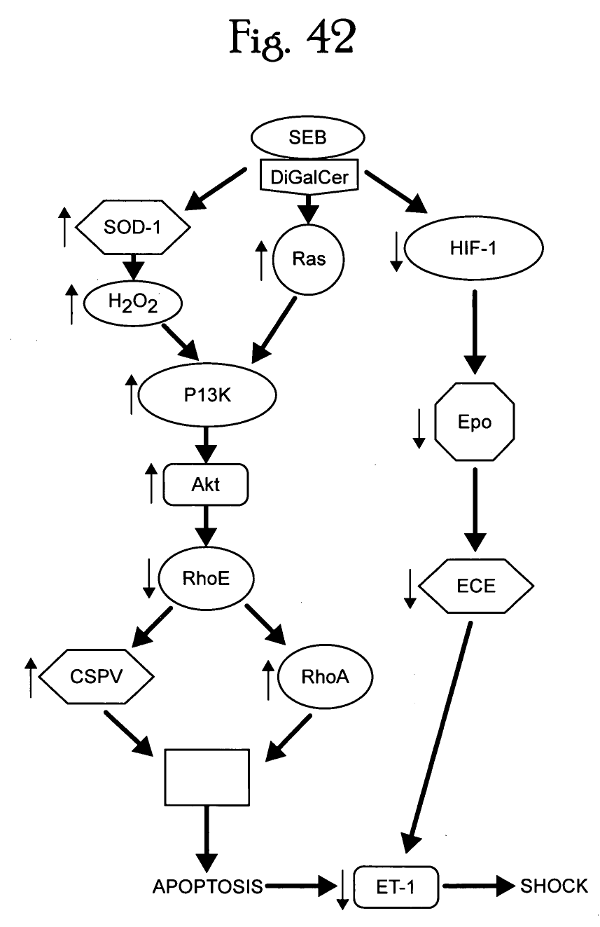

For example, FIG. 42 shows a putative signaling pathway induced in RPTEC (RPTEC is Renal proximal tubule epithelial cells i.e. kidney cells) by SEB. In the diagram, SEB is presented to kidney cells which sets off a sequences of events. On the right side of the flow diagram, HIF-1is down regulated causing EPO to be down regulated, causing ECE to be down regulated, causing ET-1 to be down regulated leading to shock. If a gene is down regulated, the corresponding protein can be administered to prevent the chain reaction just described that leads to shock. If a gene is up regulated in the sequence of events, the antisense to that gene can be administered to shut it down to prevent the oncoming chain reaction that leads to shock or other symptoms. In FIG. 42, the thin arrows indicate either up or down regulation of the gene expression, or activation or inhibition of the protein.

The genes that are disclosed as upregulated can be found in public gene libraries. The preparation of antisense to these known genes is easily accomplished by known techniques to those of ordinary skill in the art. Likewise, the preparation of proteins for known genes is easily accomplished by known techniques to those of ordinary skill in the art.

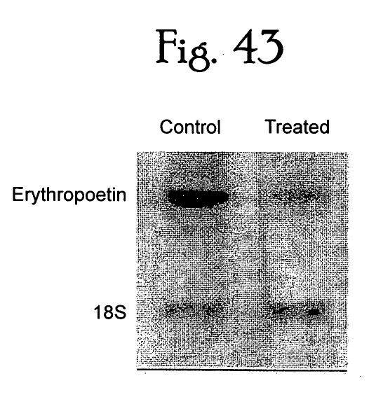

We have shown that when SEB was given to pigs and the kidney was analyzed for levels of EPO, there was a downregulation of EPO upon SEB exposure (FIG. 43). Treatment with EPO for lethal shock has never been tested before here the inventors have now successfully used EPO for treatment of shock induced by SEB. In the event protein is given, it would be given in a dose of 50 U-500 Unit/kg body weight, by IV administration. Procrit has been approved to increase red blood cell production in cancer patients who undergo chemotherapy. Another function of EPO is its role as a stimulator of endothelin-1 production and it is that function that has the potential to provide intervention for lethal shock.

Effect of Anthrax on Expression of Different Genes in Human Lymphoid Cells in Vitro:

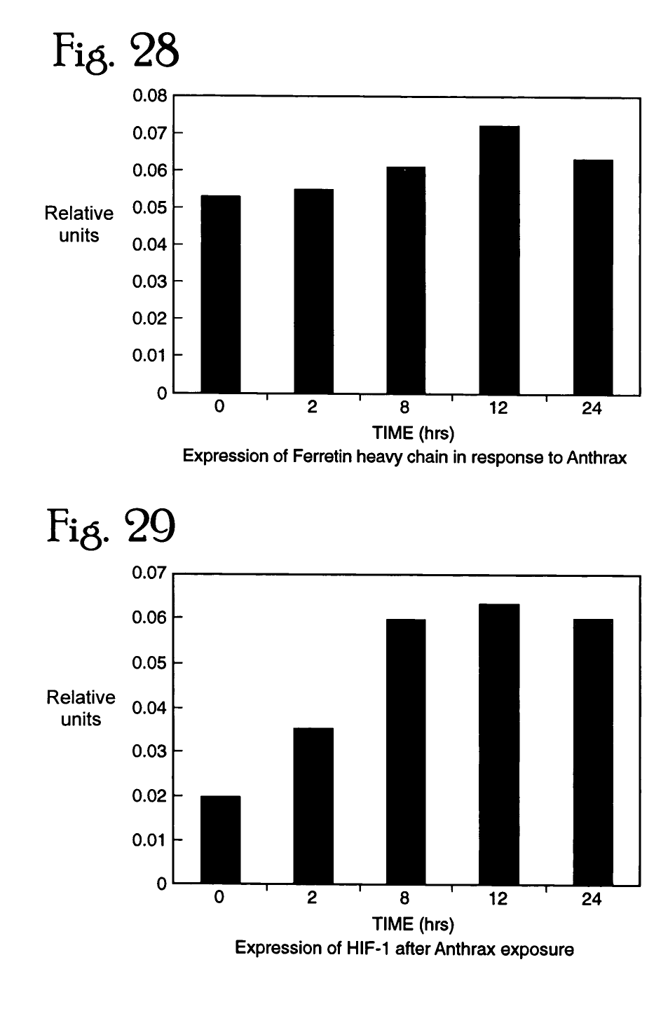

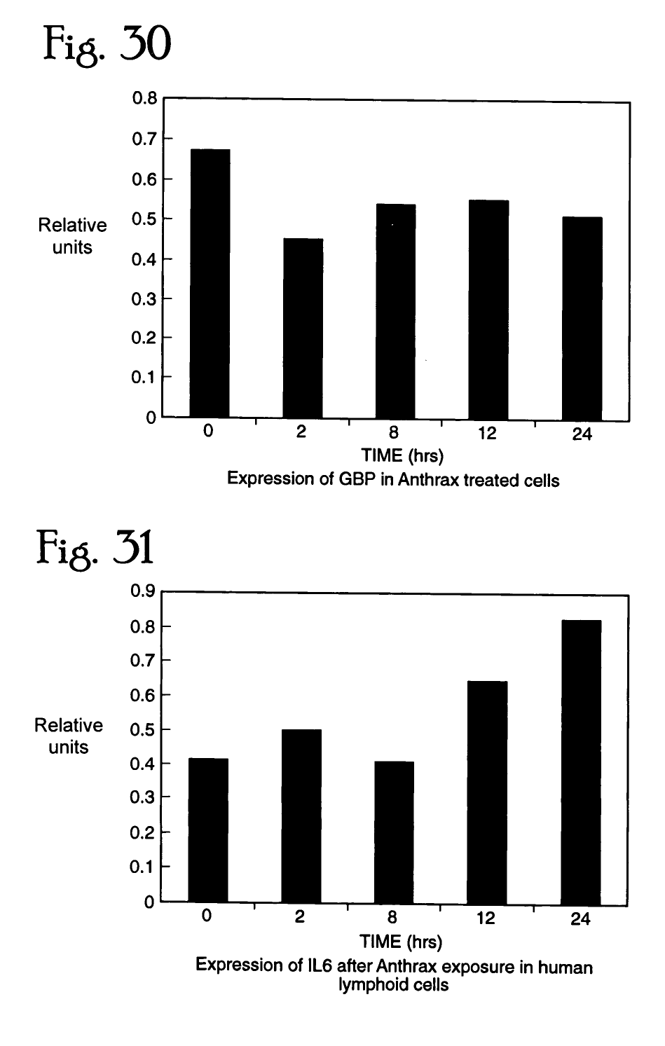

Cells were exposed to anthrax spores for different time periods and RNA isolated from the cells. Primers were designed for each gene and RT-PCR performed on RNA samples from different time periods of Anthrax exposure. Gene expression of Ferritin heavy chain and GBP did not alter in response to Anthrax (FIG. 28, 30). However expression of HIF-1 was up regulated within two hours and reached its peak by 8 hrs and was constant till 24 hrs (FIG. 29). Expression of IL6 was increased moderately doubling by 24 hrs (FIG. 31) in anthrax treated cells.

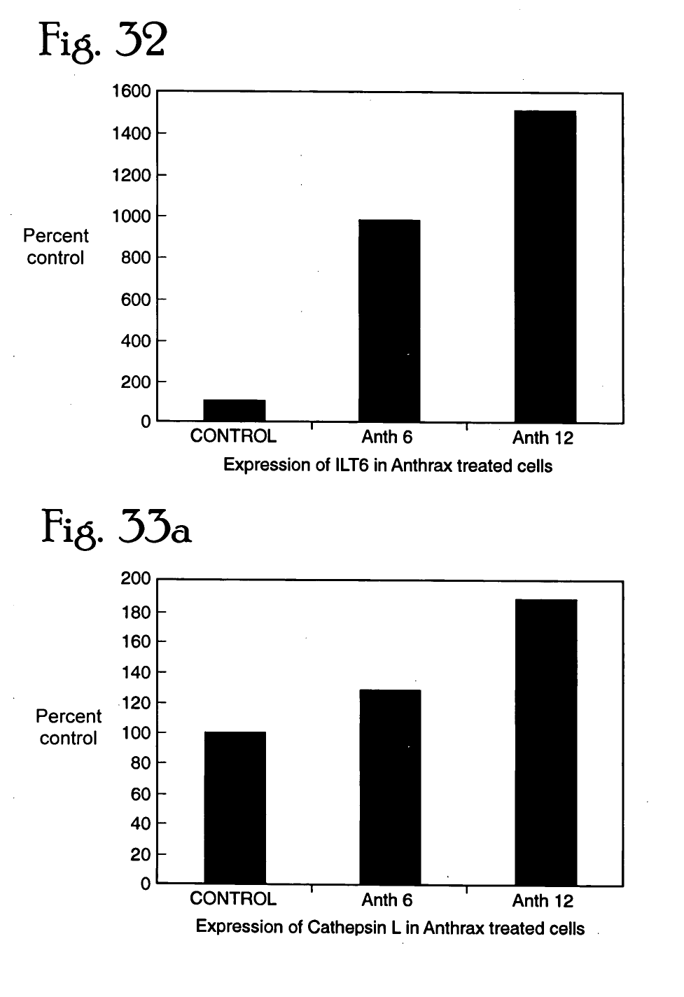

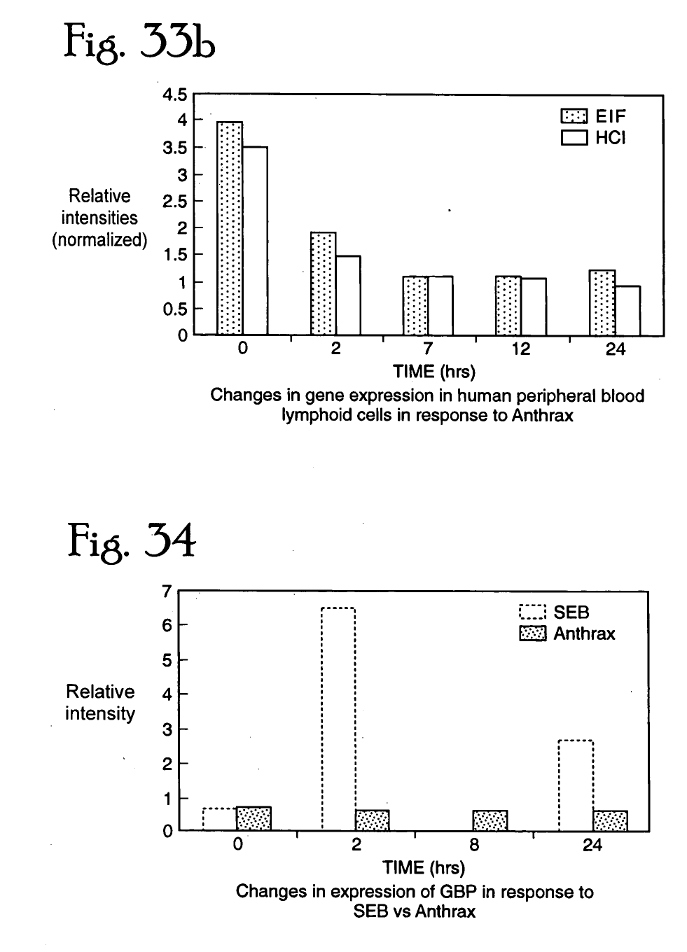

Genes identified from differential display in anthrax treated cells were also tested for the level of expression by RT-PCR. FIG. 32 shows the expression of ILT6 (immunoglobulin-like transcript) to be significantly up regulated by 6 hrs and it reaches its peak at 12 hrs of anthrax exposure. The expression of cathepsin-L (a lysosomal enzyme) was also shown to be up regulated in FIG. 33a. There was a dramatic decrease in expression of HCI (Human collagenase inhibitor) and EIF3 (eukaryotic translation initiation factor) upon Anthrax exposure (FIG. 33b). These specific genes have not been ever reported to be altered by anthrax and targeting these genes opens up new opportunities for treatment of this new deadly threat today.

Comparison of Gene Expression Pattern in SEB and Anthrax Treated Cells:

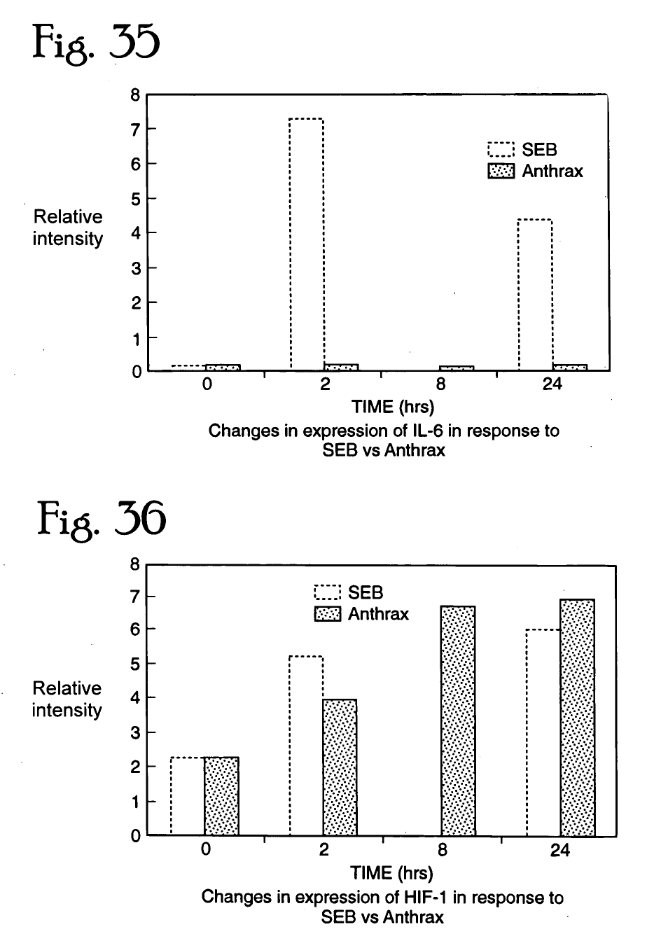

The expression of GBP was compared in SEB and anthrax treated cells. There was a significant difference in response in these two sets. SEB showed an up regulation of the gene however there was no change in expression of the gene in anthrax treated cells (FIG. 34). This suggests that there is a pattern of changes in gene expression, which will be specific for each agent.

Expression of IL6 was compared in cells exposed to these two BW agents. IL6 showed a 50-fold increase by two hours of SEB exposure and it remained high even after 24 hrs. There was no change of IL6 expression in two hours of Anthrax exposure however there was only a two fold increase by 24 hrs (FIG. 35). This suggests a distinct pattern of gene expression induced by each agent in a host cell.

Expression of HIF- 1 was up regulated in both the groups with SEB and Anthrax treated cells (FIG. 36). It is not surprising that certain genes are elevated in response to several, but not necessarily all the various toxins. We expect that these genes, while less specific for a particular agent, may still be useful to establish a pattern of alterations in gene expression by the various toxic agents.

In FIGS. 34-36, for each pair of results shown, the left band is SEB and the right band is Anthrax.

Differential Display Gel Profiles of Each BW Agent:

RNA was isolated from lymphoid cells after treatment with each agent. RNA was processed using differential display kits (obtained from Beckman-Coulter, Calif.) using 33P to label the PCR products and was resolved on a long-read gel. The gels were dried and exposed to X-ray films.

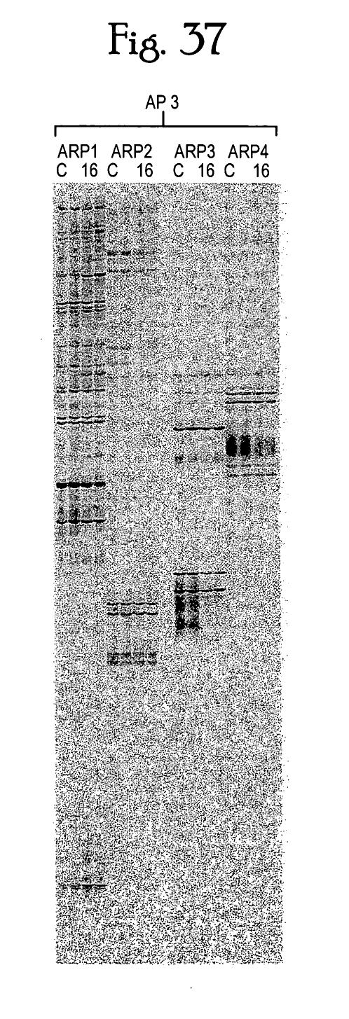

Cells were treated with SEB for 16 hrs and different AP (anchored primers) and ARPs (arbitary primers) primers were used for the DD-PCR reaction (FIG. 37). Each reaction was performed in duplicate and the samples were resolved on a 4.6% acrylamide gel. Bands that were altered were cut, cloned and analyzed for their sequence. On the digital display, the ‘C’ represents a control and the 16h represents a sample exposed to SEB.

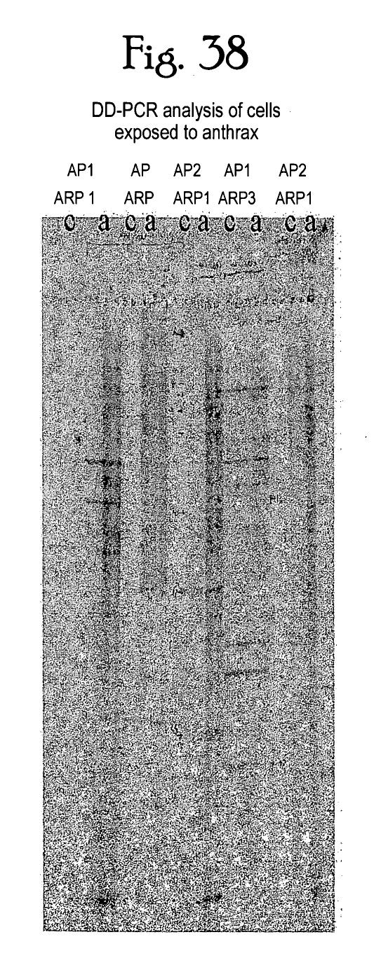

Cells were treated with anthrax spores for 12 hrs and RNA isolated and compared to the control at 12 hrs. The comparison of SEB and anthrax is shown in FIG. 38. Bands of interest were cut out and identified for gene sequences.

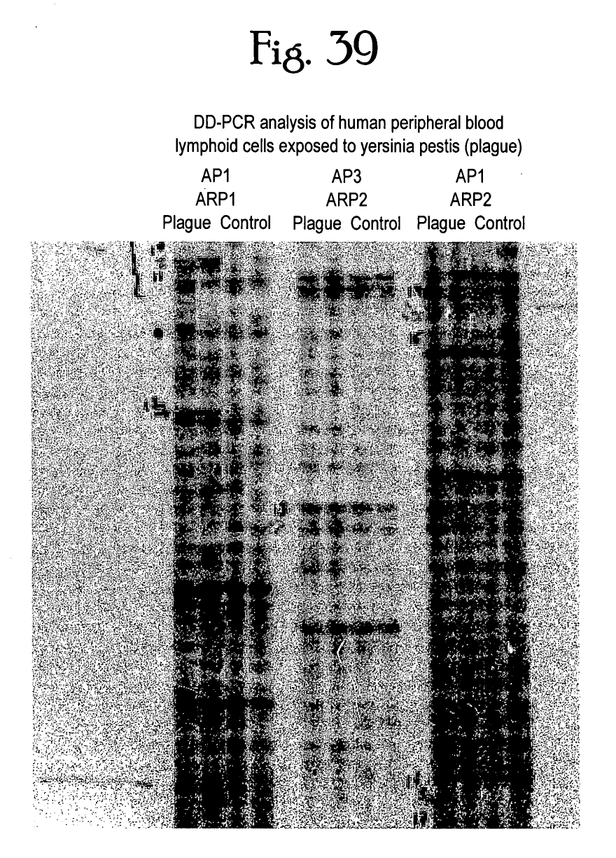

Monocytes were exposed to Yersinia pestis for 30 mins. and were inactivated in gentamycin for two hours prior to RNA isolation. Combination of different APs and ARPs were used on these RNA samples in duplicate and resolved on a long gel. Bands that showed changes were cut out for further analysis (FIG. 39).

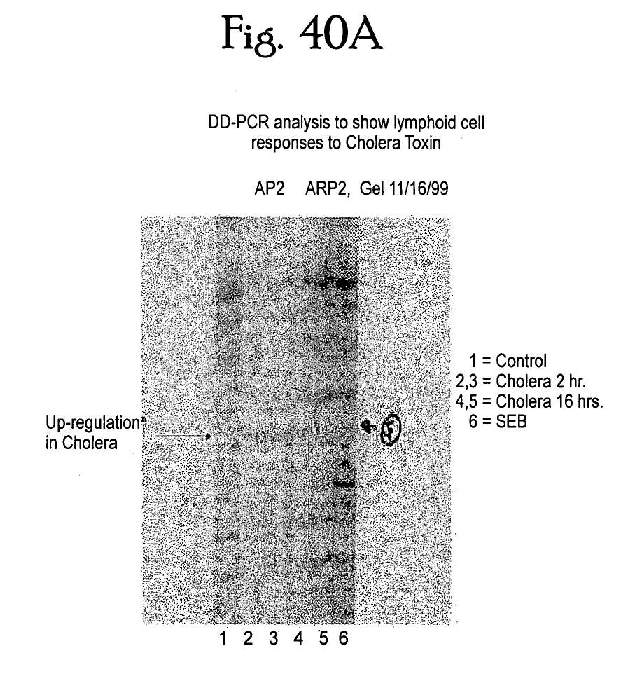

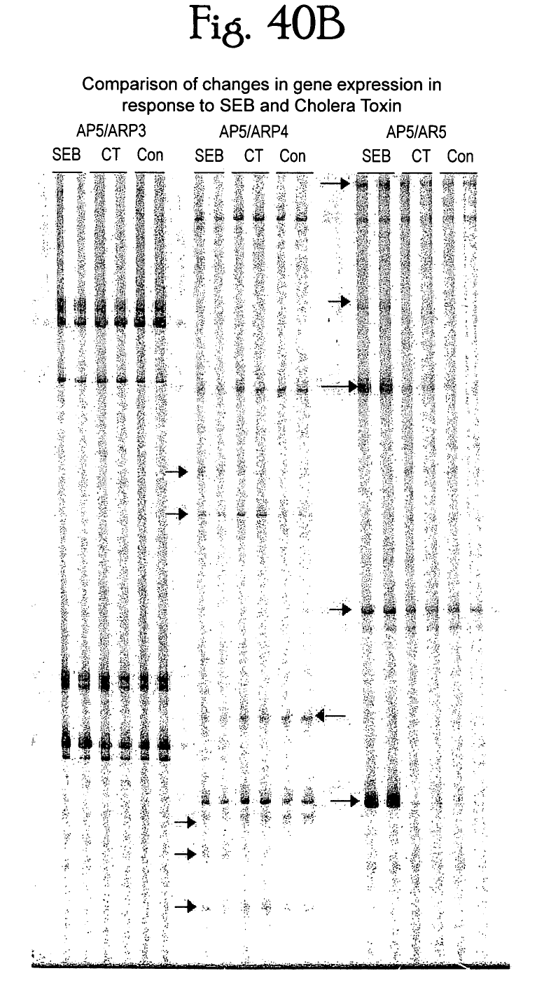

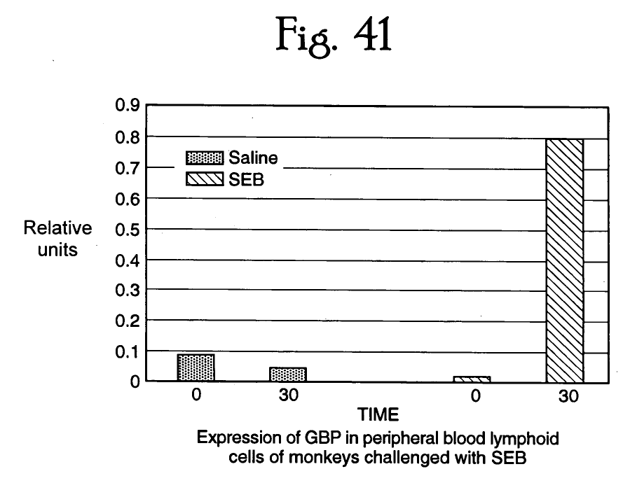

Lymphoid cells were exposed to Cholera toxin for 12 hrs prior to RNA isolation. DD-PCR reaction was performed and resolved on a long gel. Bands of interest were isolated and purified for sequencing (FIG. 40).

A prototype example is described using 2 shock-inducing toxins, staphylococcal enterotoxin B (SEB) and endotoxin, of which lipopolysaccharide (LPS) is the smallest active unit.
a) Gene profile for diagnostics: We determined the changes in gene expression in response to two shock-inducing toxins, staphylococcal enterotoxin B (SEB) and lipopolysaccharide (LPS), the smallest active unit of endotoxin. For these two agents which result in lethal shock using different mechanisms, we found several alterations in lymphoid cell gene expression which are common to both. However, we have also found genes that are specifically altered by each agent. We found that patterns of gene expression in lymphoid cells could be categorized to indicate likely course/outcome (such as shock, neurological toxicity, etc) very early after exposure to a toxic agent.
b) Gene identification for treatment: Lethal shock has proven to be elusive in successful treatment because so many cascades of cellular mediators are activated; the techniques we have used, differential display (DD)-PCR have identified genes never before thought to be involved in shock. Several of these genes contribute to regulation of vascular tone (hypotension is one of the major problems with lethal shock). We realize that manipulation of the expression of the corresponding proteins offers new targets for treatment of shock.
c) Novel previously unknown genes: We have found many genes responding to SEB which are not yet in the databases although they show up as gene bands on polyacrylamide gel. These novel genes have been sequenced and present additional possibilities for treatment.
d) We have tested this approach using peripheral blood lymphoid cells isolated from monkeys challenged with SEB. We selected genes to verify based on the experiments using DD-PCR with SEB exposure. Indeed, as early as 30 min. post-SEB exposure, we observed that the in vivo response reflected the pattern of altered gene expression that we had seen in vitro. With in vivo vs in vitro anthrax exposure, similarities are seen through out the course of illness in monkeys (72 h).

At the present time we have now found about 829 genes with altered expression, which have been observed upon SEB exposure to peripheral blood human lymphoid cells. Of these genes, the identity of 120 genes has been determined by comparing their sequences to known sequences in GENBANK databases. Those genes have never previously been associated with SEB-induced lethal shock.

We have also identified 85 genes appearing as bands on gel in anthrax exposure to peripheral blood human lymphoid cells and 28 bands on gel in Plague exposure to peripheral blood human lymphoid cells and about 30 bands on gel in Cholera exposure to peripheral blood human lymphoid cells, each band indicating a specific gene. See FIGS. 37 -40b.

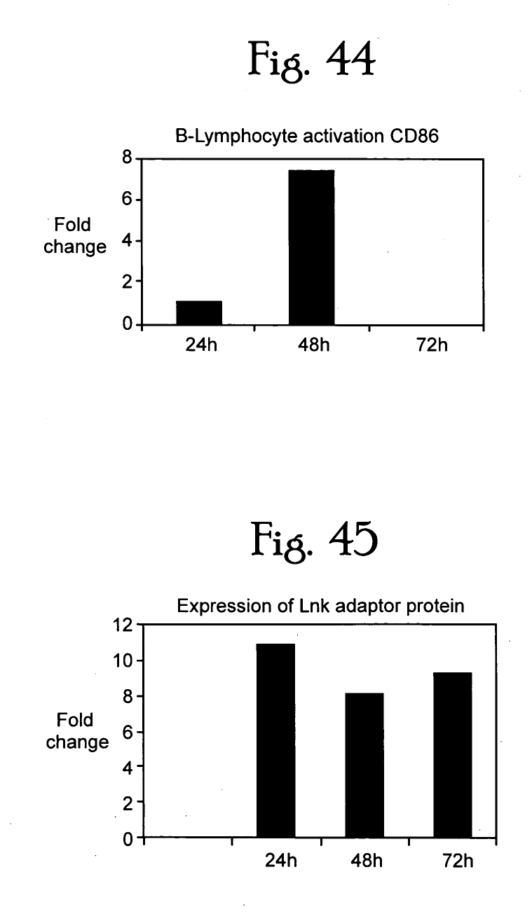

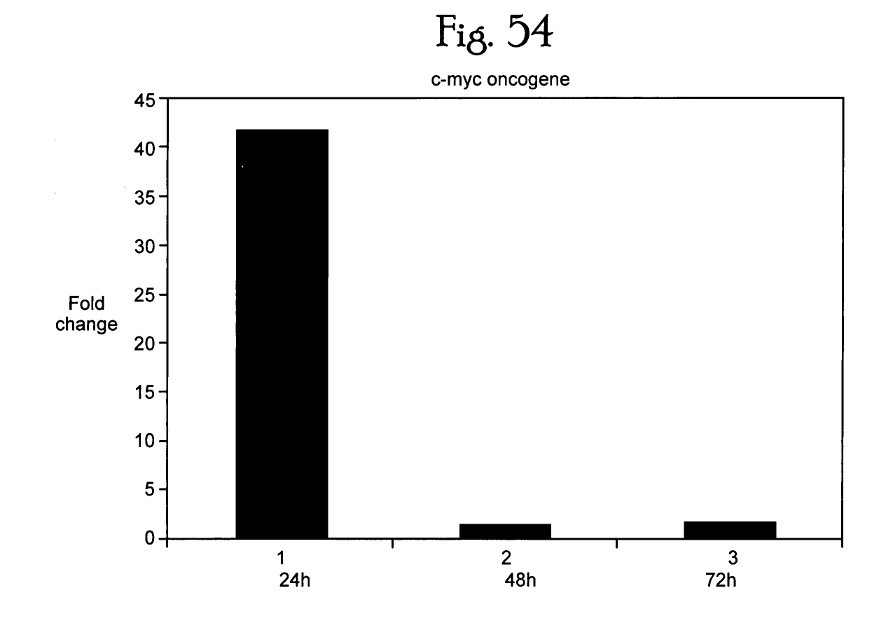

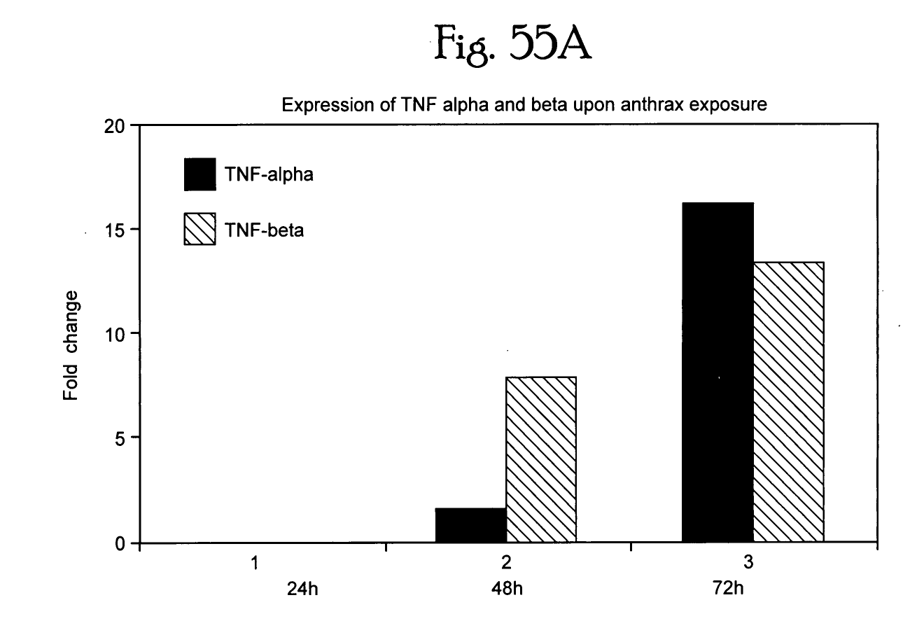

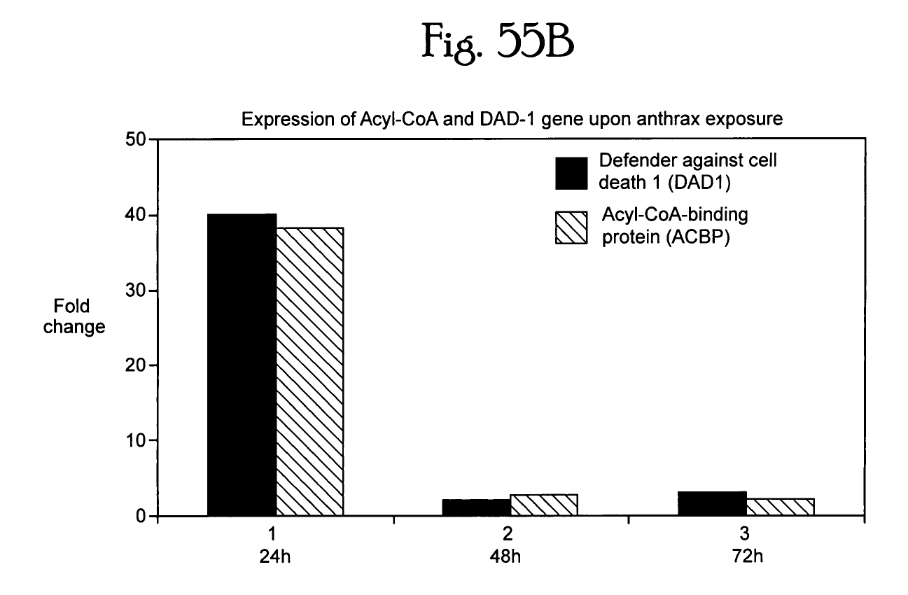

Gene Changes for Anthrax n Monkey (see FIGS. 44-56):

As shown in FIG. 44, B-lymphocyte activation antigen CD86 (B7-2 antigen) DC86 (B70/ B7.2) is a type 1 membrane glycoprotein and is expressed earlier during an immune response. CD86 takes an important role in the interaction between T lymphocytes and antigen presenting cells (APC's) as co-stimulatory molecules. Dysregulation of expression of B7 may be implicated in the pathogenesis of autoimmune disease. CD86 expressed on Langerhans cells may play an important part in the pathogenesis of atopic dermatitis.

As shown in FIG. 45, stimulation of the T cell antigen receptor TCR activates a set of non-receptor protein tyrosine kinases that assist in delivering signals to the cell interior Lnk, a 38-kDa protein consisting of a single SH2 domain and a region containing potential tyrosine phosphorylation sites, might serve to join Grb2, phospholipase C-gammal, and phosphatidylinositol 3-kinase to the TCR.

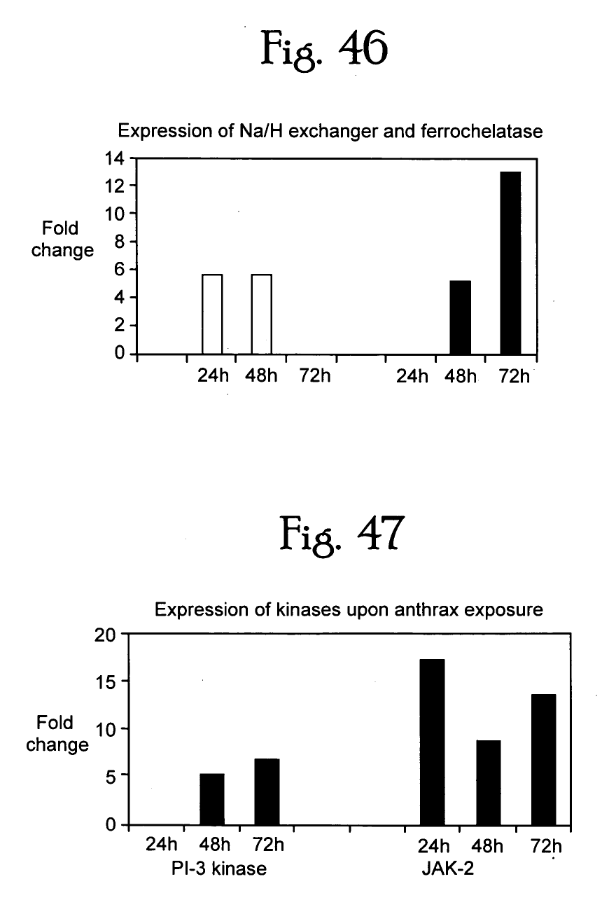

As shown in FIG. 46, the Na+/H+ exchangers (NHE1-6) are integral plasma membrane proteins that catalyze the exchange of extracellular Na+ for intracellular H+. In rat myocardium NHE1 is localized predominantly at the intercalated disk regions in close proximity to the gap junction protein connexin 43 of atrial and ventricular muscle cells. NHE-1 MRNA levels are increased in the injured arteries, NHE-1 expression in the diseased myocardium is increased in the injured arteries, NHE-1 expression in the diseased myocardium is increased. There is convincing evidence that it also plays a pivotal role in mediating tissue injury during ischemia and reperfusion. Ferrochelatase is the terminal enzyme of the heme biosynthetic pathway. Ferrochelatase is upregulated during erythropoiesis. Ferrochelatase may play a critical role in the regulation of here biosynthesis in differentiating erythrocytes. Reduced activity of the enzyme ferrochelatase leads to accumulation of protoporphyrins in erythrocytes. Accumulation of protoporphyrin IX results in toxicity chiefly of the marrow, skin, nervous system and liver.

As shown in FIG. 47, Phosphoinositide 3-kinase (PI 3-kinase) is a key signaling exzyme implicated in a variety of receptor-stimulated cell responses. Stimulation of receptors possessing (or coupling to) protein-tyrosine kinase activates herodimeric PI 3-K. P85 participates in the cell death process that is induced in response to oxidateive stress. P85 acts as a signal transducer in the cellular response to oxidative stress, mediating cell death regulated byp53. JAK2 is a non-receptor tyrosine kinase and is involved in the signal transduction by various cytokines, GMCSF and SCF. Levels of JAK2 protein expression increased significantly in mitogen- and anti IgM- stimulated B cells.

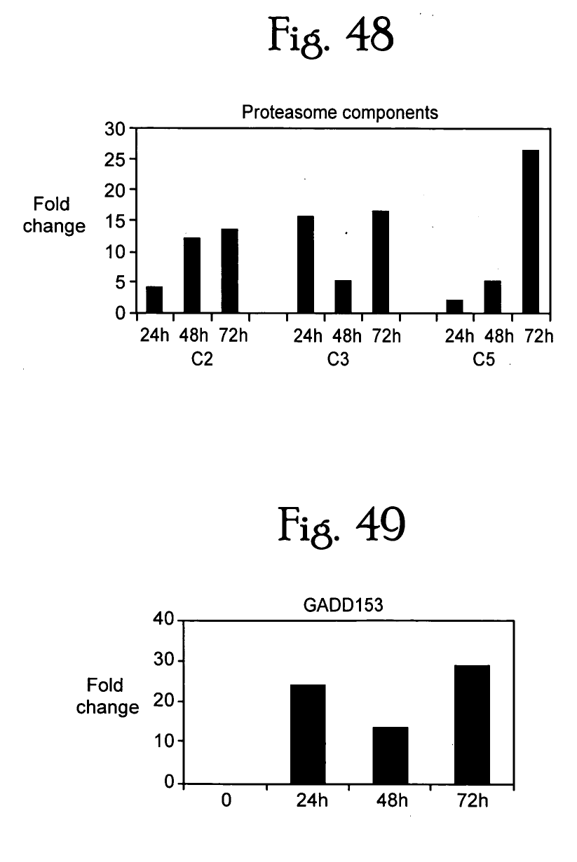

As shown in FIG. 48, the proteasome is multisubunit protease responsible for the generation of peptides loaded onto MHC class I molecular. C3 is the alph-type subunit of proteasome which is increased by dexamethasone or by cytokines. Chronic renal failure stimulates muscle proteolysis by activating the ATP-ubiquitin-proteasome-dependent pathway. In this case the level of MRNA encoding proteasome subunits C3 is increased. HC5 is a beta-type subunit of proteasome. Proteasome subunit C5 contains phosphoserine. Double labeling of human 20S proteasomes with antibodies to subunits C2 and CS has shown that these subunits are nearest neighbors. The 26S proteasome is the central protease of the ubizuitin-depentdent pathway of protein degradation. Metabolic acidosis and glucocorticoids are both required to stimulate protein degradation in muscles and increase the mRNAs for ubiquitin and the C2 proteasome subunit.

As shown in FIG. 49, growth arrest and DNA-damage inducible protein GADD153 whose expression is induced in response to growth arrest and DNA damage. Fas receptor ligation or cellular treatment with synthetic C-6 ceramide results in activation of transcription factor GADD153. Free-radical generation and thiol modification can transcriptionally activate GADD153, Ca2+ likely plays a role in the induction of GADD153 mRNA following DNA damage.

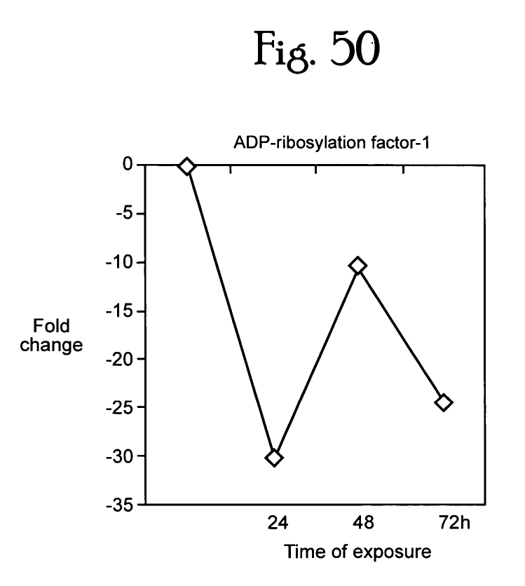

As shown in FIG. 50, ADP-ribosylation factor (Arf) is a member of the Ras super family of small molecular mass GTP-binding proteins. ARF1 is an ubiquitous molecular switch that acts as a transducer of diverse signals influencing coat assembly. In its active GTP-bound form, ARF1 is associated with Golgi membranes and promotes the recruitment of the cytosolic coat protein complex, named COPI, which results in membrane budding and vesicle formation. ARFI/COPI complex is involved in the formatin and maintenance of the Golgi complex. AFR1-GTP, through assembly of COPI to membranes and, possibly, through activation of phospholipase D (PLD), is likely to promote the formation and maturation of pre-Golgi intermediates into Golgi elements, whereas ARF-GDP causes COPI dissociation and stimulates the formation of retrograde transport structures that recycle Golgoi membrane back to the ER. ARF1 activation is promoted by guanine nucleotide exchange factors (GEFs), which catalyze the transition of GTP-bound ARF 1.

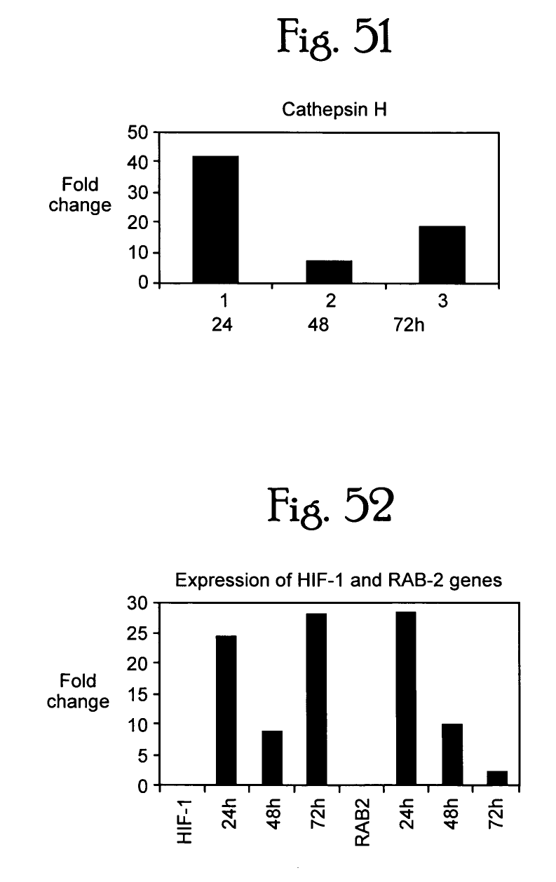

As shown in FIG. 51, Cysteine proteinases are located within lysosomes. Cathepsin H is an amino peptidase that is predominantly synthesized in kidney. Elevated activities of cysteine proteinases, the cathepsins B, H, L have been demonstrated in a variety of tumors and have been suggested to contribute to invasion and metastasis. Levels of cathepsin H antigen were found to be significantly higher in glioblastomas and anaplastic astrocytoma when compared with normal brain tissue and low grade gliomas.

As shown in FIG. 52, expression of HIF-1 alpha subunit increases exponentially as O2 concentration is decreased. HIF-1 activates transcription of hypoxia-inducible genes, including those encoding EPO, VEGF, heme oxygenase-1, INOS, and other glycolytic enzymes: HIF-1 alph is associated with the molecular chaperone hsp90. Interferons stimulate the expression of HIF-1alpha gene. Ras-related GTP-binding protein family, the Rab proteins, are implicated in intracellular vesicle trafficking. Several Rab GRPases have been localized to distinct compartments of theendocytic pathway. The Rab2 protein is over expressed in peripheral blood mononuclear cells from patients exhibiting Sezare syndromes and otherlymphoid and myeloid malignancies.

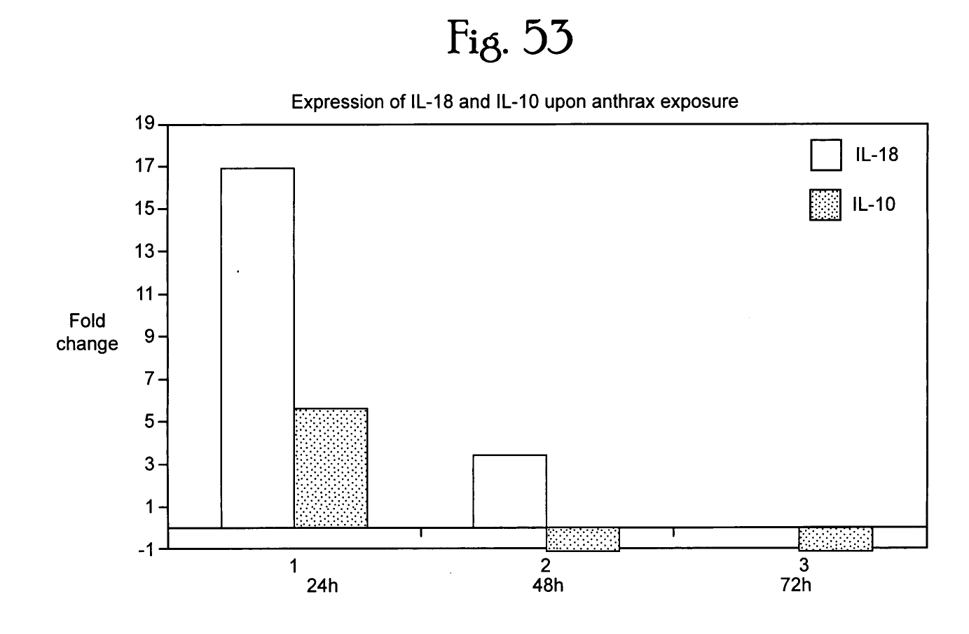

FIGS. 53-55 show further gene changes resulting from exposure to anthrax in monkey.

These peripheral blood human lymphoid cells can be obtained readily from patients and provide a reservoir of information due to their responses to toxins, infectious agents, etc.

We have catalogued patterns of responses for several toxins; the objective was to relate genes expressed in response to a biological warfare insult, to a map of responses predictive of physiological responses. Examples of maps of responses are shown in FIG. 37-40b. Each gene on the map appears as a band. The band pattern that shows SEB exposure is different than the band pattern for anthrax, cholera, etc. Since each band contains a particular gene, the gene pattern for SEB for example, can be placed on a DNA chip for use in field diagnosis of toxin exposure.

One need not know the identity of the toxic agent to determine the likely progression of symptoms, based on markers/mediators induced. The advantages in screening for specific mRNA for diagnostic markers induced by BW agents is that it will provide a target for early detection of surrogate markers of impending illness. Having identified what genes are affected by the toxic agent, we are able to design strategies for treatment approaches to block their function and thus prevent the lethal shock or any other symptoms manifested by the agent.

Advantages of the Invention Over Current Processes:

Structural based probes may not identify biologically altered toxic agents and most certainly will not detect trace levels of potentiating agents which have the ability to dramatically enhance toxicity. Use of the present system in which host response to exposure is examined, not only takes into account bioengineered agents or contaminants, but also assists in designing appropriate treatment based on factors such as degree of exposure and the individual response to the toxic agent.

Problems that the Invention is Designed to Solve:

Identification of toxic agents that have the potential to be used in terrorist attacks or accidental exposures, have previously been based on structural characteristics of the known toxic agents. Because of the threat of biologically altered toxic agents or undetectable levels of trace potentiating contaminants, we have proceeded to develop alternate approaches which rely on an individual host's response and is independent of the need to determine which toxic agent is present. Instead, the type of impending illness (shock, neurological toxicity, etc) can be determined by analyzing gene expression patterns of the peripheral blood lymphoid cells from exposed individuals. In vivo, we have seen gene expression patterns that are indicative of shock as early as 30 min post-SEB exposure. For in vitro studies, we chose 2 hr post exposure as the first time period; we also examined 16hr, 24h and later time periods as well.

Predicting exposure of a person to these agents before the symptoms appear will be of great advantage for timely treatment which can decrease morbidity and mortality from exposure to toxic agents. As stated above, these genes can be places on a blot or a small DNA chip that can be used for screening blood cell samples for rapid detection.

Other Uses for the Invention:

In the studies carried out so far, SEB and LPS induced gene alterations were compared since both agents can lead to lethal shock. Exposures to SEB can be detected based on host response and tailored treatment designed. Septic shock, induced by LPS from gram negative bacteria, is a usual emergency room occurrence daily; perhaps >20% of all emergency room cases are related to septic shock. Over at least the past 30 years, the finest pharmaceutical companies in the world have vigorously pursued studies to identify intervention tactics for septic shock; successes have occurred mainly. for early stages of shock. We have now identified genes, never before associated with lethal shock, that directly influence vascular tone (possibly the most critical element of lethal shock). Targeting these genes provide new approaches to combat this deadly illness.

Novel Aspects of the Invention:

We have identified a panel of host genes altered in response to BW agents that can be used as diagnostic markers. This has not been previously described. The advantages in screening for specific mRNA markers induced by toxic agents is that it provides a target for early detection of surrogate markers of impending illness. Having identified what genes are affected by the toxins, we have designed strategies for treatment approaches to block their function and thus prevent the lethal shock.

Patterns of Mediator Production Reflect Exposure to a Specific Toxic Agent:

We had previously observed that various toxins produced a distinctive pattern in production of mediators of illness when using either cultures of human lymphoid cells or when using plasma and/or lymphoid cells from animal experiments. It is impractical to try to measure mediators produced because a) they appear, usually transiently, from minutes to hours or days and b) they are usually unstable. Therefore, we decided to create a library of responses to toxins using mRNA, which has none of the problems associated with the mediators, themselves.

Patterns of Gene Expression Reflect Exposure to a Specific Toxic Agent:

We found that each toxic agent alters gene expression in the host in a unique pattern. Lymphoid cells provide a readily accessible reservoir of information that can reveal direct or indirect responses to toxic agents. As prototype toxic agents in our initial studies, we assessed the biologic effects on lymphoid cells by certain toxins that induce lethal systemic shock in primates. Though different mechanisms staphylococcal enterotoxin B (SEB) induce production of a cascade mediators whose activities lead to shock. The release of endotoxin, of which lipopolysaccharide (LPS) is its smallest active unit, from the cell wall of gram-negative bacteria, and subsequent production of numerous host mediators, is the initiating event of septic shock (Pugin, J., C. C. Schurer-Maly, D. Leturcq, and et. al. 1993. Lipopolysaccharide activation of human endothelial and epithelial cells is mediated by lipopolysaccharide-binding protein and soluble CD14. Proc Natl Acad Sci USA. 90:2744-2748; Wright, S. D., R. A. Ramos, P. S. Tobias, and et. al. 1990. CD14, a receptor for complexes of lipopolysaccharide (LPS) and LPS binding protein. Science. 249:1431-1433.1990). In contrast, SEB acts as a super antigen, stimulating T cell proliferation (Jett, M., R. Neill, C. Welch, T. Boyle, E. Bernton, D. Hoover, G. Lowell, R. E. Hunt, S. Chatterjee, and P. Gemski. 1994. Identification of staphylococcal enterotoxin B sequences important for induction of lymphocyte proliferation by using synthetic peptide fragments of the toxin. Infect Immun. 62(8):3408-15.1994; Neill, R. J., M. Jett, R. Crane, J. Wootres, C. Welch, D. Hoover, and P. Gemski. 1996. Mitogenic activities of amino acid substitution mutants of staphylococcal enterotoxin B in human and mouse lymphocyte cultures. Infect Immun. 64(8):3007-15. 1996), inducing a number of cytokine genes and other mediators in lymphocytes and monocytes (Yan, A., G. Yang, and M. Jett. 1997, Cholera toxin induces TNF-a production by human monocytes via cAMP independent pathways. FASEB J. 10:2746.). In our laboratory we have shown that SEB induces high levels of CD69 (Yan,, 1997. Protein kinase C is involved in SEB induced TNF-α production. FASEB J. 10:1922) while LPS produces a minor change in this surface marker. In contrast, TNF-α production is rapidly elevated by LPS whereas SEB produces modest changes in its production (Yan). These changes which we have characterized are just a few of a battery of potential biomarkers indicative of patterns of impending illness. Production of a unique pattern of mediators of serious illness in response to toxic agents, is indicative of the type of illness or toxicity that will follow.

We have now proceeded to identify a spectrum of genes altered in response to toxic agents using the technique of differential display. Briefly, we have identified 829 altered genes in response to SEB; many of these genes differ from the genes activated by LPS. Furthermore, our studies with SEB have revealed completely new responses to the toxin that have never before been characterized and present new therapeutic approaches. We have further verified in monkeys challenged with SEB (compared with using each monkey as its own control in a saline sham), that the selected genes were altered as predicted in response to the toxin. These genes not only provide diagnostic capabilities for toxic agents, they indicate exposure dose, and also they also provide potential new targets for events that ultimately lead to SEB induced lethal shock. We have further characterized gene responses induced by several other biothreat agents and they also provide new targets for therapy.

Lymphoid Cells:

This approach centers on the fact that peripheral blood lymphoid cells can serve as a reservoir of historical information and can be readily obtained from an exposed individual. Furthermore, even though lymphocytes may not be the cells most affected by a biological, infectious or chemical agent, they tend to respond to BW agents by either direct or secondary stimulations. Indeed, certain tissues most affected by many toxic agents are inaccessible.

Our approach relies on determination of a battery of unique genes altered in response to each of the toxic agents. We have used staphylococcal enterotoxin B (SEB) as a prototype and have found 829 genes with significant alterations in expression upon exposure, in vitro, of human peripheral blood lymphocytes to the toxin (See FIG. 37). At this time, we have isolated, amplified, sequenced and identified from databases about 120 of these genes. One codes for the cytokine, IL-6, which has been extensively characterized as being increased in response to many shock-inducing toxins. The other identified genes have not been associated previously with staphylococcal illnesses or lethal shock and represent potentially new therapeutic targets as well as unique markers of SEB exposure for diagnostic purposes. We have verified these findings in lymphocytes of monkeys challenged with SEB; using PCR primers designed for the selected genes, we have found unique patterns in alteration of gene expression as early as 30 min post-aerosol challenge. The animals were placed in the aerosol chamber after being anesthetized with ketamine. They were exposed for 20 min at a cumulative level between 12-25 ug/kg SEB.

Global Library:

This invention provides for a library of gene responses to BW agents. These genes can be targeted for treatment regimes for these agents. We have provided a list of genes that are induced by Brucella, Plague, SEB, SE's Anthrax, VEE and Dengue. These agents fall into groups causing similar gene alterations for some agents, yet pinpointing unique responses with a battery of other genes. With SEB and LPS, IL-6, TNF-alpha and a few other mRNA changes, may not distinguish between the two shock-inducing toxins. In contrast 6 of the numerous genes exhaustively examined to date show unique alteration in response to SEB and not to LPS. Selected genes act as markers, in a time-dependent manner, predicting the pattern of illnesses before the actual symptoms appear. Identification of specific genes that are differentially expressed in response to BW agents has revealed molecular pathogenesis that will enable us to design intervention to prevent or ameliorate impending severe illness.

The host gene expression patterns act as diagnostic markers. We have generated a library of genes altered by each toxic agent. These libraries consist of hundreds of genes altered upon exposure to each agent. See Tables 2-9.

We have shown changes in gene expression in lymphoid cells induced by Brucella, Plague, SEB, Anthrax, VEE and Dengue. We have shown gene changes in monkeys exposed to Anthrax and SEB. We have shown changes in gene expression in kidney cells induced by SEB, and have confirmed the changes in monkey samples. We have compared the pattern in SEB with LPS induced changes in both the cell systems. We have also shown the effect of drugs to block the SEB induced effects in lymphoid cells.

Changes in Gene Expression Induced by SEB.

We decided to examine the changes in levels of gene expression induced by these toxins in order to move away from the inherent difficulties in quantitating cytokine changes and to try to identify new therapeutic targets. Using SEB as a prototype, we studied changes in expression of mRNA using selected RT-PCR primers and subsequently performed the technique, differential display (DD). Table 1 shows changes in expression patterns of numerous genes both up- and down-regulated. These genes have been isolated, cloned, sequenced and characterized.

Genes 1, 2 and 5, that have been positively identified by database comparisons, are genes coding for proteins, not previously implicated in SEB action on lymphoid cells. They have varying activities and functions; there is a common theme of association with adhesion molecule function. These proteins may provide clues for new approaches in the treatment of lethal shock.

Although some gene sequences are not identified, the diagnosis of toxin can be made based on the location of the gene on the gel as shown in FIG. 37.

Discussion of the genes in Table 1b.

Gene #1—Connective Tissue Activating Protein III (CTAP-III)

A cDNA which codes for a protein released from activated platelets and represents an inactive precursor connective tissue activating protein III (CTAP-III) (85 amino acids) was down regulated. This inactive precursor chemokine has shown to be proteolitically cleaved by leukocytes and leukocyte derived proteases at the N-terminus (Harter et al., 1994). These proteases have been shown to proteolitically process the above inactive chemokine to a neutrophil activating chemokine near sites of inflammation and vascular lesions (Harter, et al., 1994). The activation of the neutrophil activating chemokine has shown to aggravate the course of thrombotic diseases and their sequelae, as in atherosclerosis, by inducing inflammation and tissue damage (Walz, et al., J. Exp. Med. 170(5), 1745-1750, 1989). Inflammation and tissue damage are two conditions that are widely associated with SEB exposure. Here we show a cDNA, which had a high identity to CTAP-III, which was down-regulated through DD-PCR, and the down regulation was confirmed through RT-PCR and northern hybridization (FIG. 1). This cDNA has never been implicated with SEB activation and explains some of the conditions exposed by SEB exposure.

Gene #2—Chondroitin Sulphate Proteoglycan Versican 1

A cDNA that was down regulated is known to code for a chondroitin sulphate proteoglycan versican V1 that belongs to a growing family of large aggregating proteoglycans (Doege, et al., J. Biol. Chem, 266, 894-902, 1991; Doege, et al., J. Biol. Chem, 262, 17757-17767, 1987). The side chains containing a few chondroitin sulphate chains of these proteins protects the endothelium from oxidant injury and direct cytotoxycity (Nakazona, et al., Proc. Natl. Acad. Sci. USA, 88, 10045-10048, 1991; Abrahamsson, et al., Circ. Res., 70, 264-271 1992; Redni, et al., biochem. J., 252, 515-519, 1988). It is known that the changes in heparan sulfate metabolism might lead to profound changes in the physiology of blood vessels and removed from the endothelium in the course of inflammation. This was present in all types of blood vessels, ranging from the large caliber aorta to smallest capillaries. A decrease in proteoglycan may contribute to the loss of barrier properties therefore reducing in the thickness of the blood vessels, which may contribute to low blood pressure conditions, which is common in patients exposed to SEB and are symptoms associated with SEB induced shock. It is the first time such a gene has been identified to explain the low blood pressure conditions associated with SEB.

Gene #3

A novel gene that appeared on the gel but did not match with any of the available sequences of GenBank.

Gene #4—Interleukin-6 (IL-6)

Expressing of high levels of interleukin-6 by SEB is well documented. Experiments done on peripheral blood mononuclear cells (PBMC), with SEB have indicated the detection of elevated levels of IL-6 within 48 hours (Sperber, et al., Clin Degn Lab Immunol., 4, 473-477, 1995). Other experiments done using nonlethal dose SEB studies on human primates have indicated significant increased levels of IL-2 and IL-6 after four hours of receiving non lethal doses of SEB (Kerakaumer, et al., Mil. Med., 9, 612-615, 1997). Our results agreed with the above results, as we also observed high levels of IL-6 production within two hours of SEB induced human lymphoid cells first by DD-PCR and second by RT-PCR (Fig.5). As IL-6 is a common cytokine induced by many toxins, it cannot be used to differentiate the effect of SEB from other toxins.

Gene #5—Myosin Class 1 (Myc-1)

A cDNA, which coded for myosin class 1 was clearly up-regulated through DD-PCR. This motor domain containing proteins have shown to lead to significant cardiac dysfunction (Colbert, et al., J. Clin. Invest., 100, 1958-1968, 1997) showed a two fold up-regulation through RT-PCR and may explain the cardiac discomfort observed in subjects who are already suffering from other diseases and elderly who have been exposed to SEB.

Gene #6—Hypoxia Inducible Factor 1 (HIF-1)

Upon stimulation by SEB a set of genes that are observed under reduced oxygen content were differentially expressed. A key step to hypoxia inducible activation is the formation of a heterodimeric complex of two helix loop helix PAS proteins (Wang, et al., Proc. Natl. Acad. Sci.USA, 92, 5510-5514, 1995). The helix loop helix transcriptional factor consists of a 120 kDa subunit complexed with a 90-94 kDa subunit induces respiratory distress. The up regulation of this cDNA, which codes for hypoxia inducible factor-I (HIF-1) detected through DD-PCR was confirmed by RT-PCR (FIG. 4). The increase in cDNA expression of the helix loop helix transcriptional factor which encodes glycolytic enzymes and responsible for respiratory distress has never been implicated with SEB and clearly could directly be involved in respiratory problems due to it's up regulation.

Gene #7, #9 and #10

Novel genes that appeared on the gels but did not match with any of the available sequences in Gen Bank.

Gene #8—Guanylate Binding Protein (GBP)

An up-regulated cDNA detected through DD-PCR is known to code for an interferon (IFN) induced 67 kDa guanylate binding protein-2, which has a wide variety of basic cellular functions such as protein synthesis, signal transduction, and intracellular protein transcription (Bourne, et al., Cell, 53, 669-671, 1988). Its ability to increase cyclase activity results in the production of high levels of NO, vasodilation and a threat to the endothelium. SEB induction of this gene suggests (FIG. 3) its role in producing high levels of cAMP by increasing cyclase activity as well vasodilation, which might in turn lead to lethal shock. This is a gene that not only has never been implicated with SEB but also is specific for this toxin.

Confirmation of Gene Changes in Monkey Samples Exposed to SEB

### EXAMPLE 1

We exposed several rhesus monkeys with a sublethal dose of SEB (12-24 ug/kg cumulative via aerosol) and the controls with a saline challenge, isolated blood cells and prepared RNA from them. RT-PCR was performed for three separate genes that were altered in response to SEB in human lymphocytes. IL6 showed an increase over the control monkey samples suggesting that this cytokine does play a crucial role in SEB induced toxicity (FIG. 7). We further analyzed the levels of CTAP and GBP and found both the genes to be up regulated in 30 min after exposure to SEB (FIG. 8, 9). This confirms the data we observed in vitro with human lymphoid cells. These genes can be thus be used as markers for exposure to SEB in a time dependent manner.

### Differences in Responses in SEB and LPS Exposed Cells

Comparison of Changes in Gene Expression in SEB and LPS Induced Lymphoid Cells:

When genes identified by DDPCR were analyzed and compared in two different toxins, we found there were some differences in their expression patterns. As shown in FIG. 10-13, four genes showed different expression patterns induced by SEB or LPS. This suggests that each toxin or BW agent will have a unique pattern of gene expression that is induced in the host. Table A further summarizes the total changes observed in lymphoid cells in response to these two toxins.

In an attempt to determine how the kidneys may be contributing to SEB-induced lethal shock, Gene changes observed in human kidney cells (renal proximal tubule epithelial cells-RPETC):

Expression pattern of RhoE in Human Lymphoid Cells

RhoE is a small G protein that lacks intrinsic GTPase activity (Foster, et al., 1996). This protein is involved in cell adhesion. As shown in FIG. 14 there is a distinct down regulation of this protein in kidney cells. This protein has been shown to block actin stress fiber formation that ultimately is known to induce apoptosis. The down regulation of this gene suggests that cell adhesion is lost in kidney cells, ultimately inducing cell death.

Comparison of Gene Changes Induced by SEB and LPS in Kidney Cells:

Genes such as GBP, IL6 and Ferritin were induced by LPS in the kidney cells (FIG. 15-18). Ferritin showed a time dependent decrease in expression in response to LPS (FIG. 17a,b). Changes in IL6 occurred much later after 24 hrs of exposure to the toxin however there was no change observed by SEB in these cells. In contrast SEB induced a dramatic change in lymphoid cells by 2 hrs of exposure whereas LPS showed no change in these cells.

Genes encoding HIF-1 and Myosin heavy chain were both up regulated in kidney cells but LPS did not show any change (FIG. 19, 20). We also observed that Superoxide dismutase was also stimulated in response to SEB.

### Methods or Procedures

Primary Cell Cultures: Cell Isolation/Purification from Plasma of Healthy Human Donors.

Human lymphocytes and monocytes were prepared from leukopacks from noimal donors according to Jett et al 1994 using lymphocyte separation medium histopaque 1077. Lymphocytes and monocytes were purified and separated further by counterflow centrifugation-elutriation with PBS as the eluant. Jett et al 1994.

Differential Display:

The differential display approach was introduced in the past few years and has become a potent tool for identifying genes that are differentially expressed in various eukaryotic cells and organs or under altered conditions. Differential Display was used to obtain the results shown in tables 1a, 1b.

The cells (12.5 E6 monocytes plus 50E6 lymphocytes in plastic tissue culture flasks containing 175 cm²) were exposed to these toxins for various appropriate time periods (1 hr-24hrs) andmRNA was isolated. The technique of differential display involves isolation of undegraded mRNA free of genomic DNA. Reverse transcriptase (RT) is necessary for conversion of mRNA to single stranded cDNA by using a two base-anchored oligo-dT primer T12MA, T12MC, T12MG and T12MT where M is a mixture of dA, dC and dG obtained from Beckman Coulter, Calif. A fraction of this reaction mixture of the cDNA was amplified by PCR using appropriate primers and radio labeled dNTP. The PCR products were separated on a 6% Sequencing polyacrylamide gel, after developing the gel we looked for differences in the treated vs untreated lanes for presence/absence/intensity of bands as described previously. Both positive and negative controls were included to avoid false positives. In addition to samples with and without toxin, controls include +/− RT product, +/−primer, etc. Once the different bands are identified, they were cut out of the gel, eluted by soaking in PCR buffer at 37 C for 30 min and reamplified by a repeated PCR using the same primers pairs of AP and ARP to confirm the changes. The final confirmation was carried out on a Northern blot, where the MRNA samples were run on a gel and each of these bands labeled and used as a probe to see if the changes are reproducible. Once this is confirmed then the cDNAs was cloned into a vector. Cloning was performed in a TA-TOPO vector from Invitrogen according to their protocol and sequenced to identify the nature of the gene. The sequence was compared to the gene bank database to look for homology with other already identified genes or find out if they are unique in any way. RT-PCR was also performed to confirm the changes in gene expression by each agent.

This technique is highly sensitive and reproducible, and is a rapid method for identifying unique genes, quantitatively, which are altered upon treatment of cells with the compound of interest. This information provides a library of genes that are activated by toxins/agents producing serious illness, it will aid in identification of new treatment modalities. Thus this technique has enormous potential; identifying the changes occurring at the molecular level in a system has radically changed concepts in biomedical research by opening new avenues for diagnosis and therapy. We have already used this technique and have identified many genes altered in expression in our prototype studies with SEB.

Other techniques that have been used are Gene Microarray technique to identify the changes induced by these toxic agents.

Gene array:

This technique allows us to screen thousands of genes for their expression pattern in one experiment. The gene array blots were purchased from Clontech laboratories or were slides custom printed in house, the RNA samples were labeled with 33P and hybridized to the blots according to the manufacturer's instructions. For slides RNA was labeled with fluorescent dyes, hybridized to the slide and scanned in Axon scanner. The image of the blots was scanned in a BIORAD Multiflor scanner and the data was analyzed using various softwares. ATLAS software 2.0, Gene pix, Gene Spring was used to get numbers for each spot for control and treated samples. The numbers were normalized and then the ratio obtained by dividing the adjusted numbers of treated sample over the control. The tables presented here represent the fold change induced by each agent at various time points.

Using these techniques, we screened 7,000 genes at a time to yield information in a time efficient manner and to quickly build a gene library for each toxic agent.

Measurement of Gene Changes by Using DNA Chips:

This is an innovative approach of analyzing changes in gene expression in a sample for a large number of genes simultaneously. The development of recent technologies allows us to immobilize DNA to a solid surface such as glass and exposed to a set of labeled probes. The array is then exposed to fluorescent labeled sample RNA, hybridized and the positive signals analyzed.

Biorobotics machine can spot thousands of genes on 48 slides at a time in duplicate on glass microscope slides in an area of 2.5 cm by 0.75 cm with the use of this high speed arraying robotic machine. Because allele-specific probes for each mRNA are specifically chosen and synthesized in known locations on the arrays, the hybridization patterns and intensities can be interpreted in terms of the identity and the concentrations of various mRNAs simultaneously. Multiple spots for each cDNA can be used to better quantify the concentration of mRNA. Probes specific for each symptoms will be used such as genes for lethal shock, or genes for neurotoxic agents that will determine which agent was involved in causing the gene changes in the blood sample.

The genes listed for each agent have been selected to construct gene chip specific for each agent, the inventors also have combined all the gene list and has created a gene chip with all the genes presented here. These chips can be used routinely to screen several samples in a cost effective manner.

### EXAMPLE 2

In this example, lymphoid cells are treated with pathogens/toxins: 2, 6, 16 hr exposure; RNA is isolated. Lymphoid cells are exposed to various BW agents for defined time periods and RNA free of genomic DNA is isolated using trizol method. Enough human lymphoid cells are started to isolate RNA at all the time points for each BW agent. This RNA is used for screening of changes in gene expression pattern by several methods.

### EXAMPLE 3

In this example, DD-PCR, +/− SAGE or Gene Array is used to isolate altered genes, purify, and amplify. DD-PCR is performed using various combinations of anchored and arbitary primers to cover the entire cDNA population. The DD-PCR products are resolved on a sequencing gel and changes for each agent analyzed. An example of this is shown in Table 1a. (Table 1a is a table describing the number of genes altered with each primer combination using DD-PCR with SEB treated cells.) At each step proper negative (reaction minus RT products, etc) and positive controls (supplied RNA from manufacturer) are used and samples are handled in duplicates to avoid false signals. Genes are up- or down-regulated by each BW agent. Gene arrays from Genome Systems Inc. St. Louis, Mo., can be used to screen a whole library of 18,000 genes at a given time. To obtain more global changes SAGE can be used, a new technique for analyzing the whole cDNA more rapidly.

The techniques outlined in the Examples above are used to identify specific genes altered in response to the 6 listed BW agents. We have also verified the changes using dose and time course variations in direct analysis using standard PCR primers. Changes identified from all these techniques can be verified by northern blots to avoid false positives. Some of the BW agents used may require the longer (24 h) incubation times for gene changes to appear; also, secondary effects (because of other tissues being the BW target) may cause gene changes which would not be seen in the in vitro system. Potentially, some of those changes will still be picked up upon in vivo exposure to the BW agent.

### EXAMPLE 4

Purify, sequence genes from Example 3, identify using GENBANK databases; catalogue the genes identified for each specific agent and select genes which will discriminate among a variety of B/W agents. Each gene is re-amplified and sequenced using either cycle sequencing kit (Amersham) or using the ABI kit. We have currently found that ⅔ of the genes give a positive match in the Genebank database. Any new genes that look important as a BW agent marker, are cloned into a bacterial plasmid; we can then screen a cDNA library and identify the gene. This will provide a selected a pattern or panel of genes for each BW agent.

### EXAMPLE 5

After confirming the changes identified by DDPCR, and Gene array, specific oligos can be designed or cDNAs that will be used to verify responses to various agents in vitro and in vivo. These genes can be attached to a matrix (membrane or on glass surface) for establishing a diagnostic tool for rapid detection. Since these are known genes whose sequence information is already available in the Gene Bank, antisense oligos to these genes can be also designed for specific treatment.

### EXAMPLE 6

RT-PCR and northern analyses to confirm these changes, and determine alterations at intermediate time periods. Develop a quantitative PCR for selected genes: Specific primers are designed for each gene identified and a northern blot analysis is performed for all the RNA samples. A standardize method is used to quantify our PCR results-using nonradioactive probes [biotin-labeled specific probes for a PCR ELISA]. All necessary controls are used for such a procedure.

### EXAMPLE 7

Expose animals/non-human primates to the BW agent in question: Blood samples are taken from various animals exposed to respective BW agents at 0, 2, 16 h; the blood samples are collected, lymphoid cell fraction are isolated, RNA is extracted, quantitative PCR measurements based on the unique genes altered in response to each specific agent are performed. The selected genes are confirmed by simple RT-PCR methods, then if appropriate these samples are tested on DNA array matrices.

TABLE 1a
Genes identified using DD-PCR primer combinations

| | | Genes identified using DD-PCR | | | | | | | | | | |
|---|---|---|---|---|---|---|---|---|---|---|---|---|
| | | primer combinations | | | | | | | | | | |
| | AP1 | AP2 | AP3 | AP4 | AP5 | AP6 | AP7 | AP8 | AP9 | AP10 | AP11 | AP12 |
| ARP1 | 1 | 1 | 1 | 0 | 4 | 7 | 2 | 5 | 4 | 3 | 1 | 0 |
| ARP2 | 3 | 0 | 1 | 5 | 1 | 5 | 5 | 6 | 3 | 1 | 1 | 1 |
| ARP3 | 0 | 2 | 3 | 2 | 0 | 0 | 5 | 9 | 0 | 7 | 3 | 1 |
| ARP4 | 0 | 0 | 1 | 4 | 5 | 6 | 4 | 3 | 0 | 2 | 0 | 2 |
| ARP5 | 0 | 6 | 0 | 9 | 0 | 0 | 0 | 0 | 1 | 0 | 6 | 1 |
| ARP6 | 2 | 3 | 3 | 3 | 0 | 0 | 0 | 20 | 2 | 0 | 4 | 3 |
| ARP7 | 2 | 7 | 6 | 2 | 0 | 9 | 0 | 9 | 1 | 0 | 5 | 2 |
| ARP8 | 1 | 5 | 1 | 3 | 0 | 0 | 0 | 6 | 1 | 0 | 1 | 2 |
| ARP9 | 13 | 0 | 0 | 0 | 7 | 3 | 9 | 0 | 0 | 3 | 2 | 3 |
| ARP10 | 15 | 5 | 0 | 0 | 0 | 4 | 1 | 0 | 1 | 16 | 0 | 4 |
| ARP11 | 11 | 1 | 0 | 0 | 1 | 2 | 2 | 0 | 2 | 0 | 3 | 7 |
| ARP12 | 14 | 1 | 0 | 0 | 1 | 1 | 1 | 0 | 2 | 2 | 5 | 8 |
| ARP13 | 5 | 6 | 7 | 0 | 2 | 0 | 8 | 12 | 0 | 0 | 1 | 0 |
| ARP14 | 12 | 6 | 8 | 0 | 5 | 6 | 10 | 20 | 0 | 0 | 12 | 5 |
| ARP15 | 13 | 1 | 16 | 0 | 5 | 10 | 12 | 8 | 0 | 0 | 6 | 1 |
| ARP16 | 18 | 3 | 11 | 0 | 2 | 5 | 14 | 20 | 0 | 0 | 5 | 9 |
| ARP17 | 3 | 3 | 6 | 5 | 0 | 2 | 0 | 0 | 0 | 0 | 3 | 6 |
| ARP18 | 6 | 2 | 14 | 5 | 11 | 6 | 0 | 0 | 0 | 0 | 2 | 6 |
| ARP19 | 5 | 7 | 12 | 10 | 12 | 5 | 0 | 0 | 0 | 0 | 15 | 0 |
| ARP20 | 0 | 8 | 3 | 0 | 0 | 4 | 6 | 0 | 0 | 0 | 8 | 4 |
| | 124 | 67 | 93 | 48 | 56 | 75 | 79 | 118 | 17 | 34 | 83 | 65 |

| | | |
|---|---|---|
| TOTAL Bands: 859 | Sequence matches: 141 | 475 clones being |
| | | sequenced |

Completed combinations 100%

TABLE 1b
GENES IDENTIFIED BY DD-PCR WITH SEB TREATED CELLS

| ANCHORED | ARBITRARY | | |
|---|---|---|---|
| PRIMER | PRIMER | Fold Altered | GENE NAME |
| AP1 | ARP2 | Up/8 | IL-6 |
| AP3 | ARP3 | Up/3 | Myosin 1 |
| AP4 | ARP3 | Up/9 | Hypoxia Inducible Factor-1 (HIF-1) |
| AP1 | ARP7 | Up/3 | Guanylate Binding Protein Isoform I (GBP-2) |
| AP1 | ARP10 | UP/3 | Aminolevulinate delta synthase 2 (ALAS2) |
| AP4 | ARP3 | Up/4 | AMP deaminase (AMPD3) |
| AP1 | ARP17 | Up/4 | IL17 |
| AP1 | ARP17 | Up/6 | DNAJ-like (homolog) 2 protein (HSJ2, DNAJ2, |
| | | | hDJ2) |
| AP1 | ARP17 | Up/3 | RNA helicase |
| AP1 | ARP18 | Up/20 | Cathepsin L |
| AP1 | ARP18 | Up/4 | Transcription factor-20 |
| AP8 | ARP2 | Up/4 | Protein Tyrosine Phosphatase from Rat; (M31724 |
| | | | for human) |
| AP3 | ARP7 | down/3 | MADD |
| AP1 | ARP12 | Up/4 | Phenylalkylaminebinding protein |
| AP1 | ARP12 | Up/4 | Highly expressed in cancer (HEC) |
| AP1 | ARP16 | Up/25 | Dendritic cell protein (GA17) |
| AP1 | ARP16 | Up/3 | Aryl sulfatase D & E genes; large transcript |
| AP1 | ARP10 | Up/3 | cyclin protein gene |
| AP1 | ARP10 | Up/5 | pro-platelet basic protein |
| AP1 | ARP9 | Up/5 | PDGFRA, platlet derived growth factor receptor |
| | | | DNA |
| AP1 | ARP9 | Down/5 | Erythropoetin |
| AP1 | ARP10 | up/3 | human STS WI-12000 |
| AP8 | ARP4 | Up/3 | did not match with available sequences |
| AP6 | ARP1 | Up/8 | mannosidase, beta A, lysosomal (MANBA) gene, |
| | | | and UBE2D3) genes |
| AP8 | ARP3 | Down/5 | interferon gamma receptor 1 (IFNGR1) mRNA |
| AP6 | ARP1 | Up/5 | Human DNA for Ig gamma heavy-chain, |
| AP6 | ARP1 | Up/7 | Sequence 34 from Patent EP0892047 |
| AP1 | ARP16 | Up/6 | Human G protein-coupled receptor (STRL22) |
| AP1 | ARP16 | Down/3 | promyelocytic leukemia zinc finger protein (PLZF) |
| | | | gene |
| AP1 | ARP16 | Up/3 | betaine-homocysteine S-methyltransferase |
| | | | (BHMT) mRNA |
| AP1 | ARP18 | Down/3 | SATB-1 binding sequence |
| AP3 | ARP7 | Down/3 | Monocyte chemotactic protein 3 (MCP-3) |
| AP1 | ARP6 | Down/4 | Ferritin Heavy chain (FTH!; FTHL6) |
| AP6 | ARP7 | Down/3 | cytochrome P-450 |
| AP1 | ARP15 | UP/3 | Homo sapiens Down Syndrome critical region, |
| | | | partial sequenc |
| AP1 | ARP16 | UP/7 | F15613 containing ZNF gene family member, |
| AP1 | ARP15 | UP/4 | Human interleukin 8 (IL8) gene |
| AP1 | ARP14 | UP/4 | Homo sapiens elf-1 related protein (ELFR) mRNA, |
| | | | complete cds |
| AP1 | ARP13 | UP/3 | Homo sapiens mRNA for dual specificity |
| | | | phosphatase MKP-5 |
| AP1 | ARP15 | UP/4 | Homo sapiens regulator of G protein signaling 10 |
| | | | mRNA, complete |
| AP1 | ARP14 | DOWN/3 | Human G protein-coupled receptor (GPR2) gene, |
| | | | partial cds |
| AP10 | ARP3 | DOWN | Human mRNA for TI-227H |
| AP12 | ARP4 | UP | H. sapiens Wnt-13 Mrna |
| AP10 | ARP1 | UP | Sequence 38 from Patent EP0892047 |
| AP1 | ARP16 | UP/8 | Homo sapiens N-terminal acetyltransferase complex |
| | | | ard 1 subunit |
| AP1 | ARP13 | Down/3 | KIAA0020 gene |
| AP2 | ARP10 | UP | Sequence 27 from Patent WO9957151 |
| AP2 | ARP10 | UP | Sequence 42 from Patent WO9957151 |
| AP1 | ARP14 | UP | Sequence 5 from Patent WO0040752 |
| AP8 | ARP3 | DOWN | alpha-platelet-derived growth factor receptor, exon |
| AP1 | ARP11 | UP | ribosomal protein L15 (RPL15) mRNA |
| AP1 | ARP10 | UP | proliferating cell nuclear antigen (PCNA) mRNA |
| AP1 | ARP14 | UP | attractin precursor (ATRN) gene, exon 21 |
| AP2 | ARP10 | UP | HR gene for hairless protein, exon 2 |
| AP1 | ARP16 | UP | N-terminal acetyltransferase complex ard 1 subunit |
| AP1 | ARP15 | UP | Down Syndrome critical region, partial sequence |
| AP1 | ARP11 | UP | HSM801431 Homo sapiens mRNA; cDNA |
| | | | DKFZp434N2072 (from clone DKFZp434N2072) |
| AP2 | ARP19 | DOWN | mRNA for putative cell cycle control protein |
| | | | (SDP35 |
| AP2 | ARP17 | DOWN | ITGB4 gene for integrin beta 4 subunit, exons 3-41 |
| AP12 | ARP6 | DOWN | Human transcriptional repressor (GCF2) |
| AP12 | ARP8 | UP | Homo sapiens ribosomal protein L26 (RPL26 |
| AP3 | ARP17 | UP | Sequence 27 from Patent WO9957151 |
| AP3 | ARP17 | UP | HR gene for hairless Protein |
| AP3 | ARP4 | DOWN | chondroitin sulfate proteoglycan versican |
| AP8 | ARP3 | UP/5 | regulator of G protein signaling 10 |
| AP1 | ARP14 | UP/3 | Sequence 5 from Patent WO0040752 |
| NOVEL | | | |
| DNA | | | |
| SEQUENCES | | | |
| AP1 | ARP2 | Up/51 | Novel |
| AP3 | ARP3 | Down/3 | Novel |
| AP1 | ARP2 | Up/13 | Novel |
| AP1 | ARP18 | Down/5 | Novel |

TABLE A
COMPARISON OF EFFECTS OF SEB AND LPS ON A SET OF
DIFFERENTIALLY EXPRESSED GENES.

| | SEB (100 ng/ml) | | LPS (100 ng/ml) | |
|---|---|---|---|---|
| IDENTITY | 4 hrs/ | 24 hrs/ | 4 hrs/ | 24 hrs/ |
| OF GENE | change fold | change fold | change fold | change fold |
| 5-LO | UP/1.5 | UP/3 | X | X |
| IL-6 | UP/32 | UP/30 | UP/11 | UP/10 |
| PROTEOGLYCAN | DOWN/0.8-0.5 | DOWN/0.55 | N.D | N.D |
| V₁ | | | | |
| CTAP-III | DOWN/0.40 | DOWN/0.50 | DOWN/0.3-0.4 | DOWN/0.3 |
| GBP-2 | UP/7-3.5 | UP/3.2 | X | X |
| FERRITIN | UP/1.4-0.8 | DOWN/0.8 | N.D | N.D |
| HEAVY | | | | |
| CHAIN | | | | |
| HIF-1 | UP/2.2-2.7 | UP/2.7 | DOWN/0.4 TO | UP/1.3 |
| | | | +1.3 | |

Excised cDNA of differentially expressed genes by SEB were subjected to RT-PCR using custom designed primers. Equal quantities of expressed DNA were resolved on an agarose gel, quantified, normalized with actin and the expression was compared to control levels.
X represents no effect,
‘up’ and ‘down’ represents an up and down regulation of the gene by the respective toxin respectively and
N.D. represents the values not obtained at the respective time point.

TABLE B
SEB-INDUCED DIFFERENTIAL GENE EXPRESSION IN RPTEC

| | AP* 1 | AP 2 | AP 3 | AP 4 |
|---|---|---|---|---|
| ARP* 1 | 1 upregulated | | 2 upregulated | 3 downregulated |
| ARP 2 | 1 upregulated | | 3 downregulated | |
| | 1 downregulated | | | |
| ARP 3 | 2 upregulated | 2 downregulated | | 4 upregulated |
| | 1 identified | | | |
| ARP 4 | 1 upregulated | | 4 downregulated | 2 downregulated |
| | | | 1 identified | |
| ARP 5 | 3 upregulated | | 2 downregulated | |
| | 1 downregulated | | | |
| ARP 6 | | | | |

Renal proximal tubule epithelial cells were incubated with or without 50 ng/ml SEB for 12 hours. Total mRNA was isolated and DD-PCR performed as described. The 32 differentially expressed genes are currently at various stages of isolation, purification, sequencing, and identification.
*AP - anchored primer
**ARP - arbitrary primer
14 up regulated
18 down regulated

TABLE C
Changes in Gene Expression Identified by DD-PCR in Lymphoid Cells
Treated with Anthrax*

| | PRIMERS | | CHANGES IN | |
|---|---|---|---|---|
| GENE | Anchored | Arbitrary | EXPRESSION | FUNCTION |
| #1 | AP2 | ARP1 | DOWN | HCI-Human Collagenase Inhibitor |
| | | | REGULATED | Involved in tissue remodeling, blocks the activities |
| | | | | of metalloproteinases |
| #2 | AP1 | ARP3 | DOWN | ETF-3 Eukaryotic translation initiation factor-3 |
| | | | REGULATED | |
| #3 | AP2 | ARP1 | UP REGULATED | A NOVEL GENE. No matching sequence have |
| | | | | been found in either GENBANK and EMBL |
| | | | | databases. |
| #4 | AP2 | ARP1 | UP REGULATED | ILT-6 immunoglobulin like transcripts |
| | | | | Expressed in immune cells, acts as cell surface |
| | | | | receptors similar to NK cell receptors |
| #5 | AP1 | ARP18 | UP REGULATED | Cathepsin-L, a lysosomal enzyme involved in |
| #6 | AP1 | ARP18 | UP REGULATED | Long chain acyl CoA synthetase |
| #7 | AP2 | ARP18 | DOWN | Currently no positive match with gene database |
| | | | REGULATED | |
| #8 | AP1 | ARP18 | DOWN | FGF-13 |
| | | | REGULATED | |
| #9 | AP1 | ARP18 | UP REGULATED | Currently no positive match with gene database |
| #10 | AP1 | ARP18 | UP REGULATED | Currently no positive match with gene database |

Total of 85 bands have been identified to be altered by Anthrax in human lymphocytes using differential display. So far 10 bands have been sequenced, the rest are being sequenced currently.

Description of gene changes induced by each threat agent that can be used for diagnostic as well as therapeutic strategies:

Gene lists were obtained after screening of several gene arrays. Each agent was exposed to the cells and RNA isolated for gene array experiments. The untreated and treated samples were then labeled with 33P and hybridized to the arrays. The signals were obtained by scanning in a BIORAD scanner and the intensities of each spot was normalized with the housekeeping genes.

### Therapy For Lethal Shock

Gene Based Solution for Therapy:

The present invention uses gene expression patterns to identify genes that are turned on or off in response to exposure to a toxin agent. Some of the early genes have been used as diagnostic markers. With this understanding of the pathways involved in signaling of various biothreat agents, we have identified targets for therapeutic agents. The present invention is directed towards treatment of patients when exposed to various biological threat agents based on gene targets identified.

a. Major Gene Changes Induced by SEB Toxication:

Genes involved in various functions have been identified. These genes are regulated by exposure to a toxic agent and provide therapeutic potential for treatment of the disease caused by these agents by an understanding of the time of appearance of these gene changes and their function. For SEB, genes whose expression was downregulated after 24 hr of SEB lethal challenge are ABP (angiotesin-binding protein), AVRlA (arginine vasopressin receptor 1A), and VAP (vasopressin). Genes whose expression was upregulated after 24 hr of SEB lethal challenge are ANG2 (angiopoietin 2), Tie2 (it is receptor for ANG2), VEGF, (vascular endothelial growth factor), FLT1 (VEGF receptor), iNOS (its product is nitric oxide (NO), NO is a potent vascular dilator)). Several cytokines and cytokine regulated genes such as Interleukin-2, TNF-alpha, Interleukin-6, Guanylate binding protein, Interferon-gamma were also upregulated compared to saline treated pigs. It is important to know time zero of exposure to a toxic agent that induces cytokine release to calculate the appropriate anti-cytokine therapy.

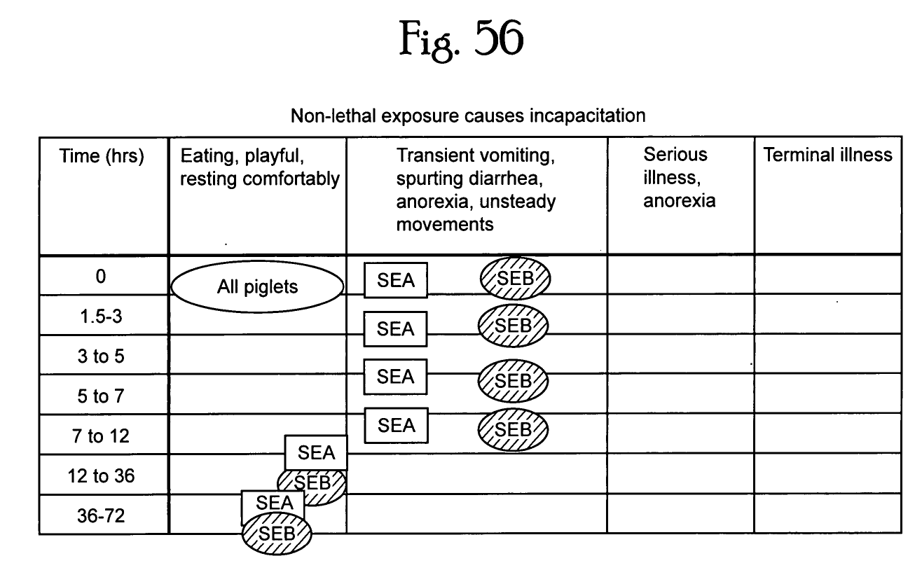

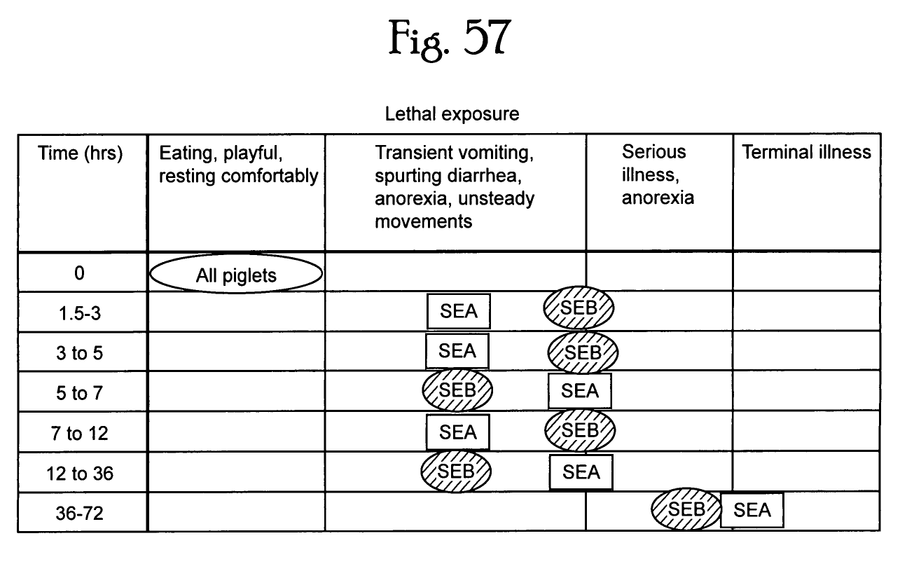

In FIGS. 56 and 57, a graphic representation of some of the symptoms from exposure of piglets to incapacitating vs lethal doses of SEB are shown, respectively. In general, the initial symptoms displayed by the animals include brief episodes (30 min) of intermittent vomiting, but spurting diarrhea occurred for ˜8 h and general diarrhea lasted for ˜5 days. Humans accidentally exposed display a very similar progression of illness as was seen the piglet; in addition, people report experiencing dreadful dizziness. The piglets must experience some similar response, since there is occasional staggering. However, the main action is for the animals to lie quietly in groups under their heat lamps. The animals displayed anorexia, the duration of which is related to the challenge dose. Gene expression profiles were determined in this animal model and listed below are some of the genes that play a role in the progression of the disease.

### I. DOWN REGULATED GENES IN SEB

For genes that are downregulated, increasing the proteins or their products helps in treatment of the disease.

1. ABP (angiotesin-binding protein): Involved in contractile responses of arteries and muscle cells to angiotensin II. Tissue angiotensin II is known in the regulation of inflammatory and fibrogenic components of repair in vascular and nonvascular sites of cardiac injury, the rat heart. This protein is involved in healing and downregulation of this gene is bad for the body (Sun et al, J Lab Clin Med. 2004 January;143(1):41-51).

2. Vasopressin: Vasopressin is a protein secreted by the kidney and can induce vasoconstriction. Vasopressin is emerging as a rational therapy for vasodilatory shock states. Unlike other vasoconstrictor agents, vasopressin also has vasodilatory properties. There are now multiple agents being developed for the treatment of heart failure designed to block many of the neurohormones that are increased in these patients. One of the hormones that is increased in chronic heart failure is vasopressin. Vasopressin reduces free water secretion and at high concentrations, causes vasoconstriction in the peripheral vasculature. Administering vasopressin to a patient that shows the symptom of down regulation of the gene for vasopressin is an effective treatment.

### II. Upregulated genes in SEB

For genes that are upregulated, blocking these genes or gene products with antisense to these genes is beneficial for the treatment of the disease.

1. INOS: INOS's product is NO. NO is a potent vascular dilator.

Nitric oxide (NO), a potent vasodilator, plays a significant role in the vascular hyposensitivity to vasoconstrictors related to portal hypertension. Chronic NO inhibition ameliorates portal-systemic collaterals in portal hypertensive rats.

2. Angiogenic growth factors such as Vascular Endothelial Growth Factor (VEGF) and Fibroblast Growth Factor (FGF) induce NO and require NO to elicit an effect.

3. 5HT2A: 5HT2A is also a potent vascular constrictor. 5HT2A can lead to the smooth muscle in the veins to constrict and thus lead to even further vascular and capillary damage

4. VEGF and Flt and their related genes are responsible for the vascular leakage by damaging endothelial cells.

### Animal Experiments to Test Various Drugs Using the Piglet Model for SEB Intoxication

1. Effect of antithrombin for treatment of lethal shock induced by SEB:

We have identified genes that are involved in coagulation and therefore antithrombin was tested for its effect to block lethal shock in our piglet animal model. Lethal shock is triggered by inflammatory mediators, vascular leakage and ischemia. We believe that antiihrombin can block these effects.

Antithrombin HI (AT III) is a serine protease inhibitor, which acts as a major inhibitor of thrombin. Apart from its role in homeostasis, AT III exerts anti-inflammatory properties and improves survival in animal sepsis models and disseminated intravascular coagulation (DIC). AT III reduces leukocyte-endothelial cell interaction, prevents microvascular leakage and ameliorate ischemia/reperfusion injury.

When antithrombin was administered after the symptoms (2 hrs after exposure to the toxin) appeared after exposure to lethal dose of SEB (a biological threat agent), the animals showed improved pathology when compared to the untreated controls. When antithrombin was given long after the symptoms appeared, that is 6 hrs after exposure and 24 hrs after exposure, the pigs still survived the lethal dose of the toxin suggesting therapeutic potential as a treatment regimen long after exposure. Antithrombin can be administered 2-24 hours after exposure and it is preferred to administer 2-12 hours after exposure.

Anti-Thrombin (lmg / animal-250-300 ug/ Kg) was administered in two ways:
A) At symptoms and at 24 hrs—minimal pathology (very effective compared to untreated controls); 2/2 animals
B) At 6 hrs and at 24 hrs—Minimal to mild/moderate pathology (much better than untreated controls); 2/2 animals
   This treatment results in 100% improvement in survivability.
   2. Effect of Pentoxifylin on Lethal Shock:

This drug blocks the effects of a cytokine called TNF-a, tumor necrosis factor-alpha. Pentoxiflyline is a methylxanthine derivative that inhibits the production of TNF-a by endotoxin-stimulated monocytes/macrophages at the transcriptional level. It is effective in reducing TNF-a levels in mice with endotoxic shock. Pentoxifylin is an anticytokine.

Pentoxifylline (50 mg/animal, 12.5-16.5 mg/Kg body weight) was administered in two ways:
A) At 2 hr and at 24 hrs—Mild pathology; 3/3 animals—3 out of 3 animals survived after the treatment.
B) At symptoms and at 24 hrs—Moderate pathology 2/3 animals; 1/3 animal worse-off—similar to SEB control.

It is preferred to administer Pentoxifylin within 4 hours of exposure to a lethal shock inducing agent. When administered at 24h after SEB challenge, it had no effect. So early administration is the key for effective therapeutic window.

3. Tyrosine Kinase Inhibitors for Treatments of Lethal Shock:

There were several tyrosine kinases that were activated upon exposure to these toxic agents. We tested to see if inhibiting these kinases would have any effect on the symptoms induced by the toxin in the animals. These inhibitors (Herbimycin, Genistin) did not show any significant changes upon treatment compared to the untreated controls.

Herbimycin (250 ug/ animal—Herbimycin 62.5 ug/Kg, Genistin 50 ug/Kg) was administered:
At symptoms and at 24 hrs—2/2 animals; No discernable change in pathology from the SEB controls.

Genistin (200 ug/animal) was administered:
At symptoms and at 24 hrs—2/2 animals; No discernable change in pathology from the SEB controls.
   4. Hetastarch:

Hetastarch 6% in 0.9% saline administered at 72 hours during stage of lethal shock failed to revive an SEB intoxicated Piglet.

5. Effect of Zofran for Treatment of Incapacitation:

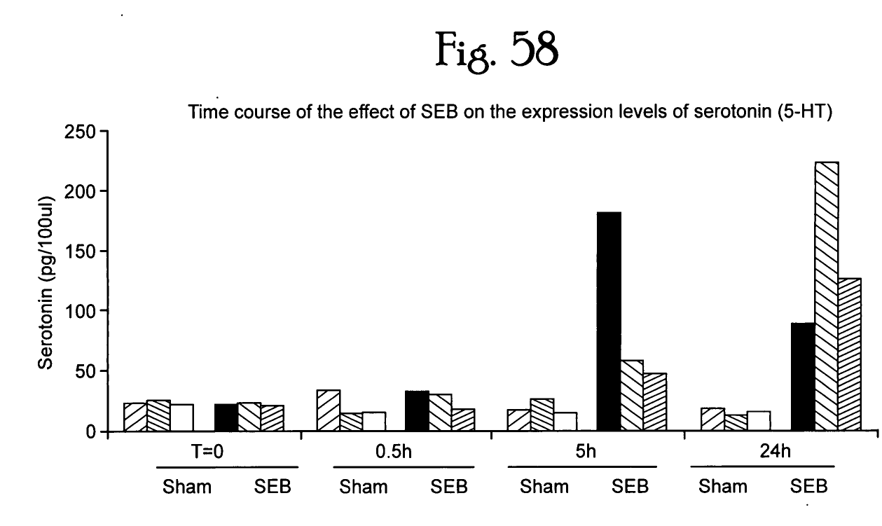

Treatment for SEB-induced incapacitation: In a prior Non human primate (NHP) incapacitation study in which we examined the appearance of various inflammatory mediators in plasma, we observed elevated plasma serotonin (5-HT) levels, and we realized that many of the clinical signs could result from the elevated levels of that mediator (FIG. 58). FIG. 58 shows a time course of the effect of SEB on the expression levels of serotonin (5-HT).

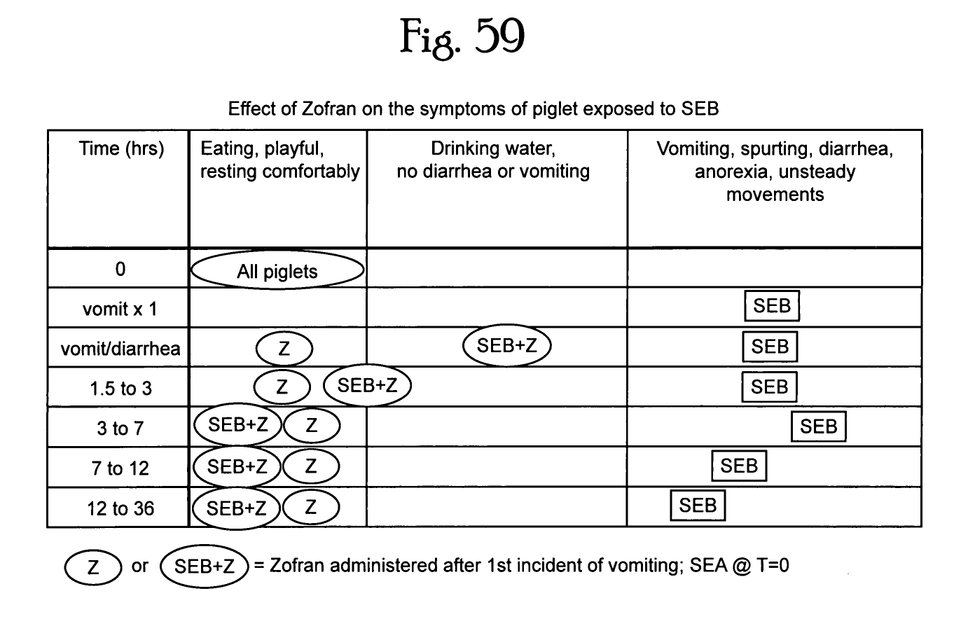

We developed the piglet model to test 5-HT receptor blockers, because NHP are difficult to use for incapacitation studies, since they cannot be handled without anesthesia, and NHP hide signs of illness. Both Kytril and Zofran were effective as the 5-HT receptor blockers (Zofran was easier to use). We did not administer the drug until after the onset of vomiting and diarrhea. There were usually one or two more incidents of retching or diarrhea after administration of Zofran, then the animal would usually go to the food dish and begin to eat. (FIG. 59). Very shortly after that, the Zofran-rescued animal would nudge littermates and playfuilly nip at them. At that point, it was necessary to transfer the Zofran-treated animals to the “control” pig run in order to keep them away from their non-rescued littermates. Although Zofran and Kytril are most effectively used to ameliorate the vomiting and nausea induced by chemotherapy, we found that the diarrhea was stopped as well.

When Zofran was administered at symptoms the animals recovered from emesis and there was a slight improvement in lowering the temperature at 72h post treatment. However there was slight improvement in lowering the temperature at 72 hours post treatment. However, there was no change in the blood pressure levels in treated and untreated animals.

Zophran® (1,2,3,9-tetrahydro-9-methyl-3-[(2-methyl-1H-imidazol-1-yl)]4H-carbazol-4-one,monohychloride, dehydrate) is manufactured by Glaxo Wellcome, Inc., Research Triangle, North Carolina. We have shown that Zofran blocks the cytokine surge in these animals, no one before has shown effect of Zofran on SEB induced symptoms or on cytokine responses.

It is preferred to administer Zofran within 2 to 3 hours of exposure to a lethal shock inducing agent. It is preferred to administer Kytril within 2 hours of exposure to a lethal shock inducing agent.

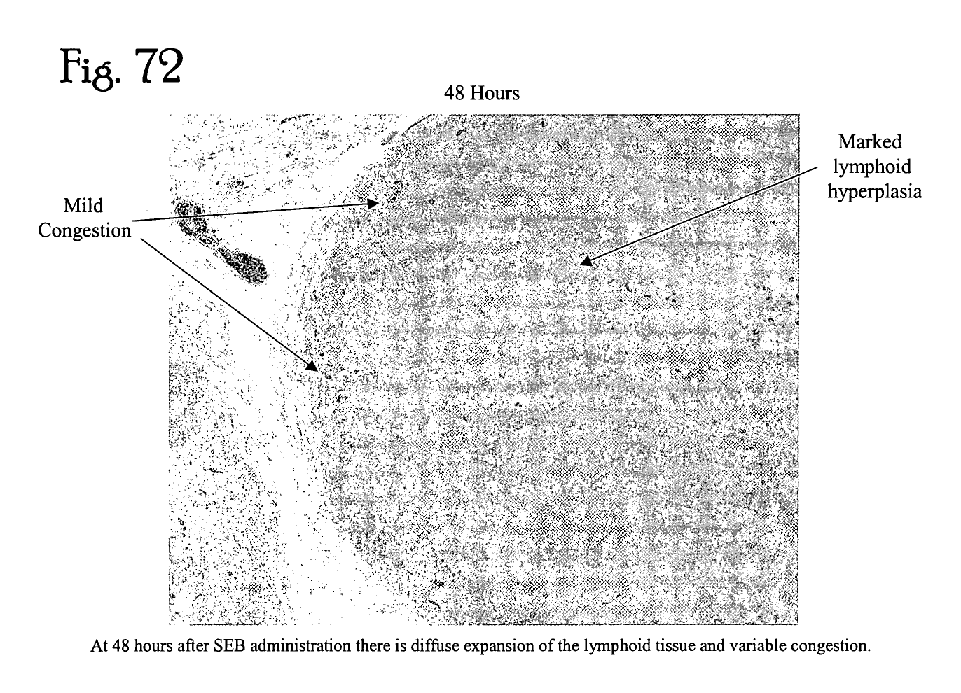

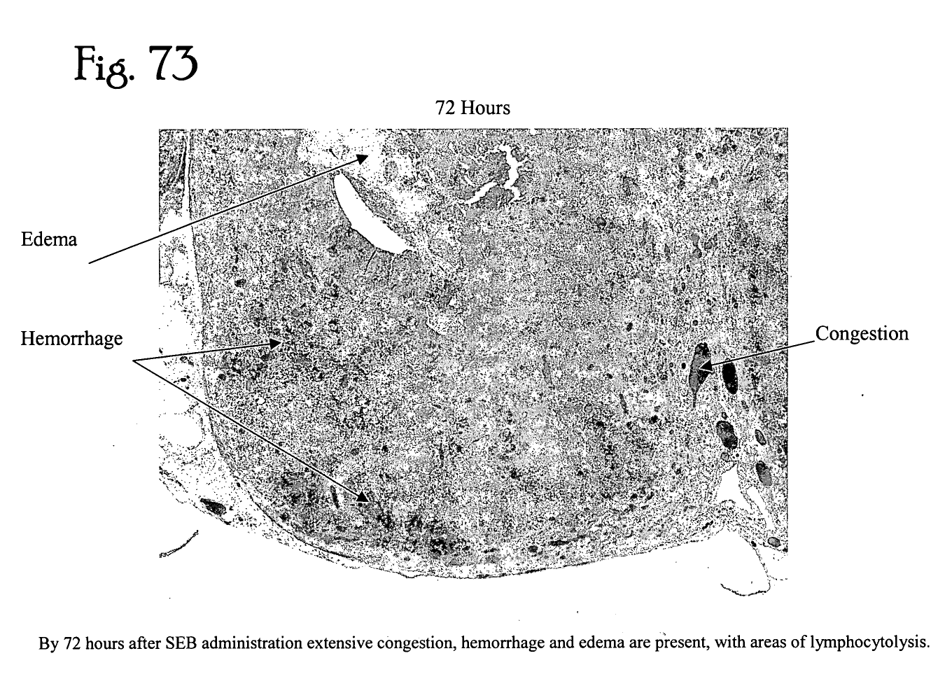

As shown in FIG. 72 and 73 are histopathological sections of lymph nodes showing the progression of the lethal shock in piglet model. At 48hrs mild congestion and lymphoid hyperplasia is observed. At 72 h you can see massive hemorrhage and edema in these lymph glands.

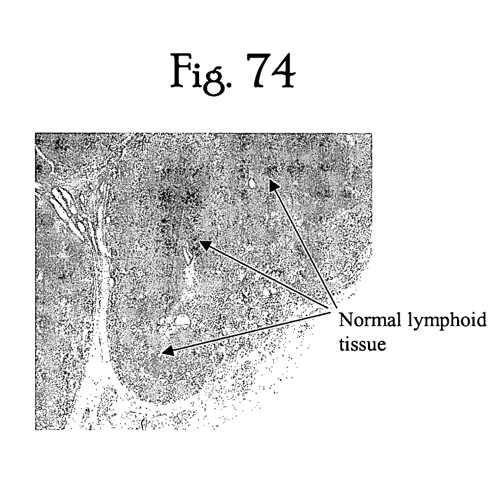

FIG. 74 shows a comprehensive picture of the different stages of damage seen in the lymphoid tissues during lethal shock. These are some of the symptoms that are caused during lethal shock. Blocking these steps is important to the success of treating lethal shock. Use of antithrombin was able to block these effects.

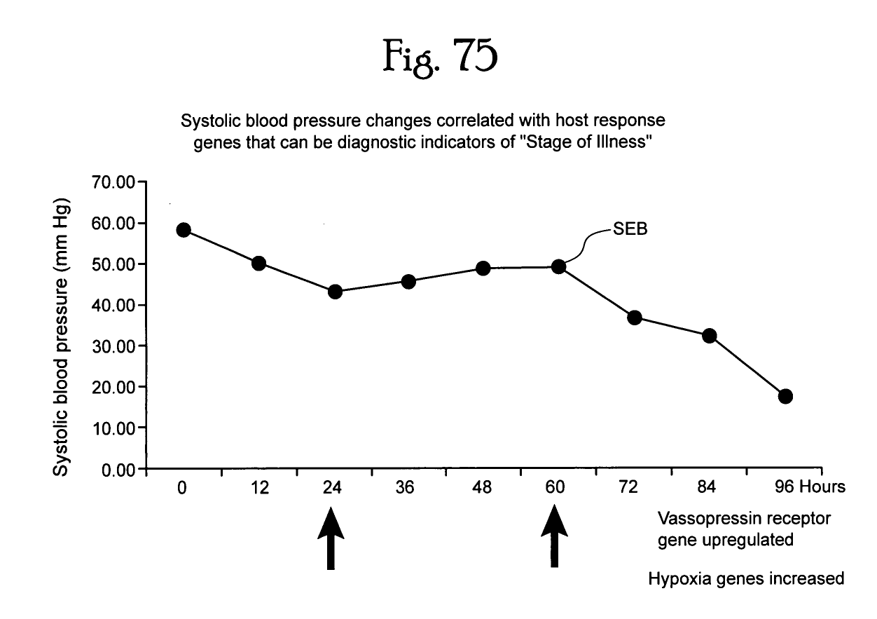

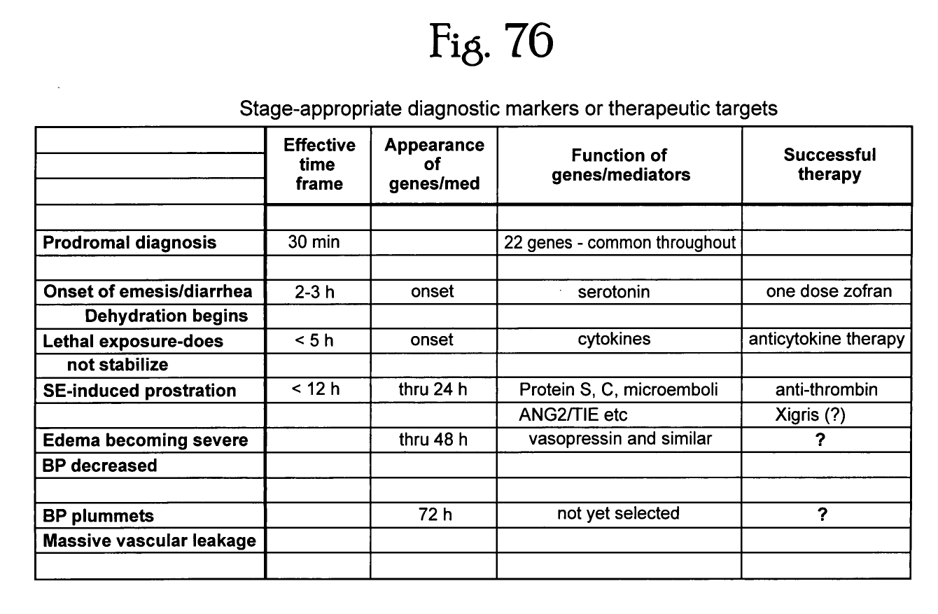

FIG. 75 describes the trend of the blood pressure that shows an initial drop at 24 h followed by a severe drop at 96h post exposure to SEB in the piglets. At each of these stages of illness we have identified genes that could help recover the animal from death. Genes or proteins that have been targeted early and have shown effectiveness are H-2 blockers, anticytokines, Zofran, antithrombin. Genes involved in inducing hypoxia and vasopressin receptors are some of the genes that can be targeted at later stages to rescue the animal.

FIG. 76 summarizes some of the stage appropriate markers that are good therapeutic targets. We have shown that even before the symptoms appear we have identified genes that are turned on by 30 min of exposure to the toxic agent. As time progresses and the symptoms get worse we have identified genes that have shown success in our animal model experiments.

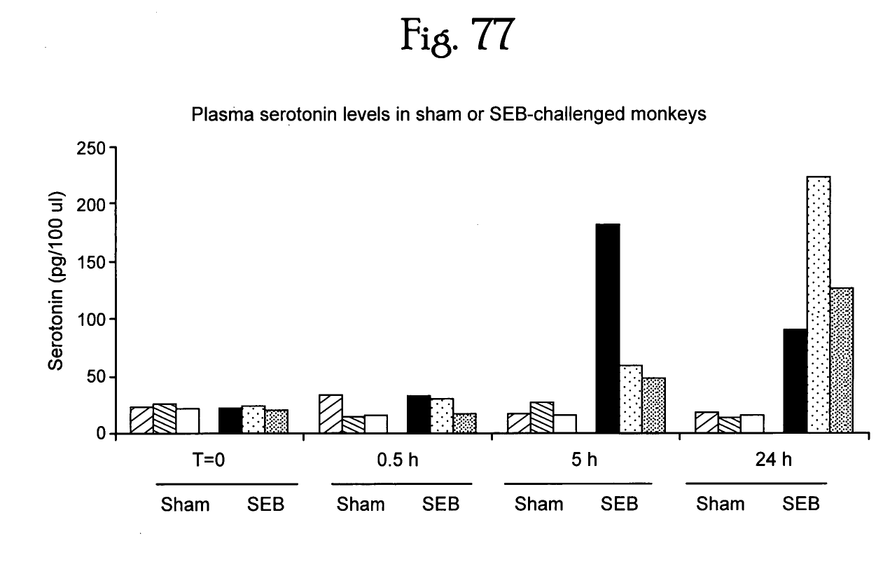

FIG. 77 shows the serotonin levels in plasma samples of monkeys that were challenged with SEB. We have levels of unexposed Sham controls compared to levels of SEB treated animals at various time periods after exposure. At 5 h post exposure there is a significant increase in the levels of serotonin in only SEB treated animals that persisted till 24 h. In our studies we have used serotonin receptor blockers such as Zofran, Kytril to see if we can block some of the symptoms caused by such increase in serotonin levels. We have shown that these drugs indeed can block the symptoms when the toxin is given at a non lethal dose.

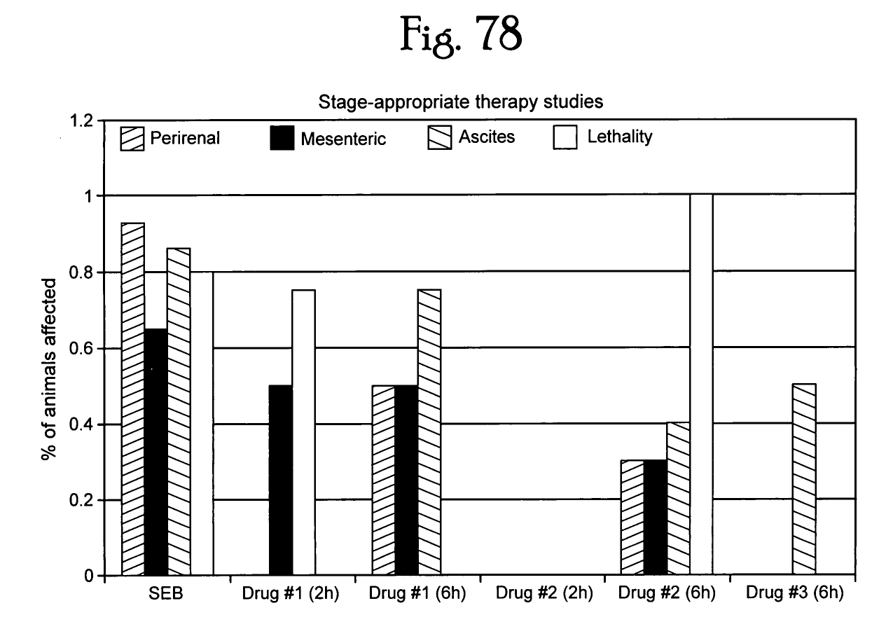

FIG. 78 compares the results of 3 drugs on the ability to rescue the animals from various endpoints in the disease progression. Drug #1 was antithrombin, drug # 2 was Pentoxyfilin and drug #3 was Herbimycin. Most of the drugs were able to reduce the perirenal, mesenteric adema, ascites and 2 out of three drugs were able to block lethality. The anticytokine therapy is time dependant, when given early it was able to block most of the effects however when given 6 h post challenge, it could not block lethality.

It has been found that different drugs administered at different times block edemas (FIG. 78). With edema, it takes about 6 hours to know what you have been exposed to. Drug 1 is antithrombin, Drug 2 is pentoxifyllin and Drug 3 is herbimycin. Pentoxifylin is an anticytokine and works well up to 4 hours post exposure to SEB. Drug 1 is antithrombin and blocks microemboli formation and prevents hemorrhage. Drug 3 is a tyrosine kinase inhibitor, these kinases have been shown to be involved in signaling cascade of SEB. Using this drug at 6 h was partially effective in rescuing the animals.

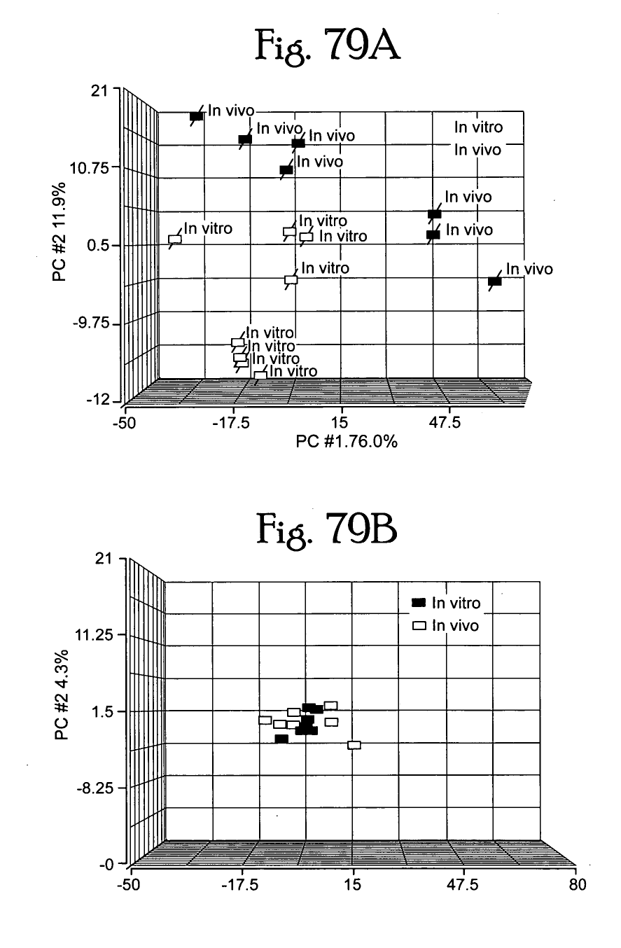

FIG. 79 show the gene expression profiles induced by SEB in vitro and a comparison of those genes with the in vivo gene profiles. Genes that were identified in vitro can be used to predict the in vivo outcome of the disease. It is a Principal component analysis of genes from each experiment. FIG. 79A shows genes that are different in the two system, however one can identify genes that are common as shown in FIG. 79B, which can be used for predictive modeling. This figure shows that the genes that we have discovered in the in vitro system can be used to target genes during the course of illness in vivo and therefore gives us a powerful tool for effective therapy.

TABLE AAA
Physical responses of piglets for incapacitation and lethal shock with
and without treatment with Zofran. (for the incapacitation study) or
with/without treatment with regulators of endothelin production in studies
of lethal shock. Table AA shows measurements of vital signs for
incapacitation studies (colunm 2) or for lethal shock (column 4). The
systolic blood pressure patterns in the lethal model at later time periods
can become unmeasurable, even using the Doppler device.
For the incapacitation model, blood pressure decreased initially.

| | | | | lethal | |
|---|---|---|---|---|---|
| | normal | Incap | Incap + Z | shock | lethal + T |
| Temp-24 h | 100-102° | 104-107° | 101-103° | 98-103° | 102-104° |
| Temp-48 h | | 102-104° | 100-103° | 94-97° | 102-104° |
| BP-24 h | 50-65 | 30-40 | 50-60 | 15-30 | 35-50 |
| BP-48 h | | 35-50 | 50-65 | <10 | 40-55 |
| Blood Gases | norm | norm | norm | pulm distr | near norm |

| | |
|---|---|
| host gene | Common responses relate to receptor mediat [symbol] |
| expression | /signal cascades; lethal exposures show |
| profiles | loss of vascular tone & pulmonary disress |

| | |
|---|---|
| Pathology/ | Lethal model has massive vascular leakage |
| histology | |

6. Use of EPO as a Treatment for Lethal Shock:

Erythropoietin, the principal growth factor of erythropoiesis, stimulates proliferation and differentiation of erythropoietic cells (Erslev, 1987) and amplifies the production of red blood cells by inhibiting the premature death (apoptosis) of their precursor cells (Koury and Bondurant, 1988).

Erythropoietin is the only know hematopoietic growth factor that acts like a hormone (Spivak, 1995). It is predominantly produced by the pertubular cortical fibroblast-like cells of the kidney. The site of its action is hematopoietic cells in the bone marrow. Expression of EPO is strictly tissue specific and in fact tissue hypoxia is the only physiological stimulus for EPO production (Spivak, 1995). A key element in this stimulation is a heterodimeric transcription factor called hypoxia inducible factor I (HIF-I), which upon activation binds to an enhancer element 3′ to the EPO gene (Wang and Semenza, 1995). For over a decade, treatment with recombinant erythropoietin was part of the therapy of renal diseases and chemotherapy-induced anemia (Krantz, 1995). We have examined the role of erynthropoietin in controlling the blood pressure in SEB induced cells in vivo.

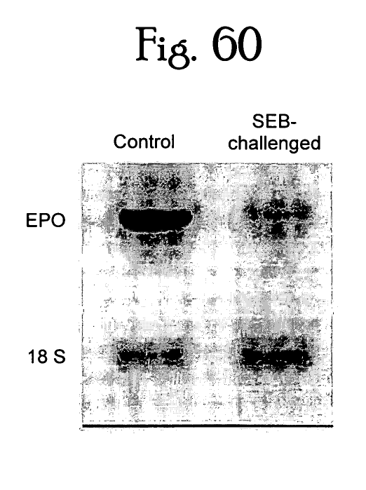

No one has examined regulating the blood pressure in SEB induced lethal shock in vivo using erythropoetin or other proteins in its regulatory pathway. Our results suggested that kidney cells play a very important role in SEB induced lethal shock. A very preliminary finding is that the kidney from a piglet treated with SEB did not show detectable EPO gene expression while a control animal kidney expressed the EPO gene in abundance. We hypothesize that giving EPO to patients who have been exposed to SE toxins will be able to regulate the blood pressure. See FIG. 60. FIG. 60 shows gene expression for EPO vs 18S in kidneys from piglets lethally challenged with SEB 48 h post exposure. Down regulation of EPO gene in SEB challenged piglets. EPO can be used to treat lethal shock.

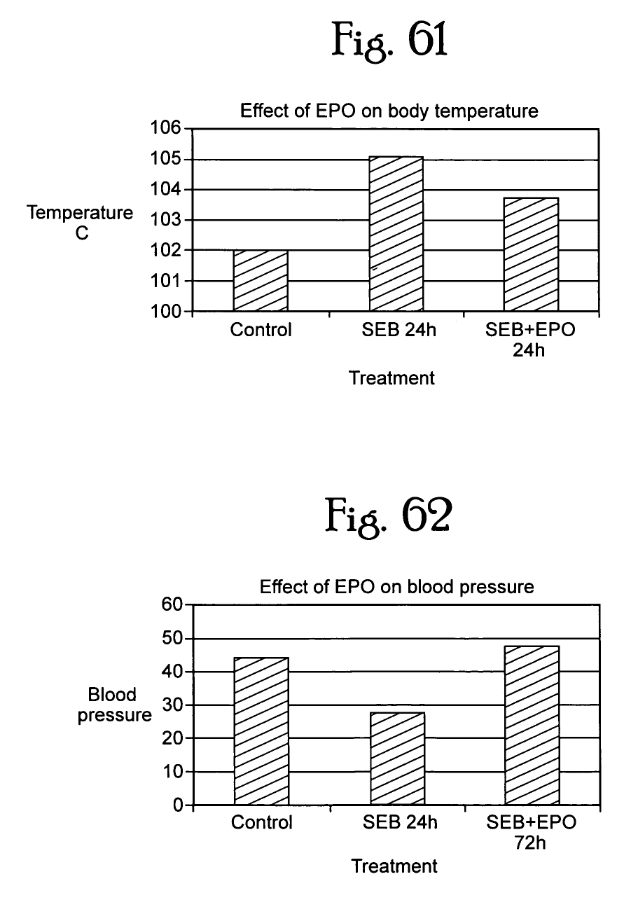

As shown in FIG. 61, The body temperature rises during SEB toxication and EPO treatment was able to bring the temperature down significantly. As sown in FIG. 62, the blood pressure drops during SEB induced lethal shock; EPO treatment was able to restore the blood pressure to the control values. Therefore, EPO can be used for the treatment of lethal shock. For FIGS. 61 and 62, Erythropoietin (500 U /Kg body weight) was administered at 2 hr/ 12 hr/ 24 hr.

Erythropoitin (500 U /Kg body weight) was administered in the following ways:

Gross Pathology:

A) Administered at 2 hr/ 12 hr/ 24hr: 2/3 piglets had moderate gross pathology, while 1/3 had similar pathology compared to SEB.

B) At 2 hrs post SEB—1/1 pig had moderate pathology—probably 10-20% improvement in pathology over SEB controls

C) at 12 hours post SEB—4/5 pigs had similar pathology to SEB control, while 1/5 pig showed a slight reduction in pathological symptoms

D) At symptoms (i.e., 3-4 hrs post SEB)—2/2 pigs—probably 10% improvement in pathology over SEB controls, but the animals died at 96 hours (Lethal shock).

It is preferred to administer EPO at 2-12 hours after exposure to a lethal shock inducing agent.

Some of the Promising Treatments/Prophylaxes Based on Gross Pathology Are:

Pentoxifylline (best therapeutic up to 4 hours)—No perirenal or mesenteric edema, though there is mesenteric lymphadenopathy (FIG. 78).

Anti-thrombin—No generalized lymphadenopathy, but some perirenal and mesenteric edema observed (FIG. 78).

Anti-translocating Peptide—definitely appears to be the best of the lot. Peptide was administered 2-5 mins prior to SEB intoxication.

### EAMPLE 8

### Functional Piglet Model for the Clinical Syndrome and Post Mortem Findings Induced by Staphyloccal Entertoxin B

Staphylococcal enterotoxin B (SEB) causes serious gastrointestinal illness, and intoxication with this superantigen can lead to lethal toxic shock. In order to overcome significant shortcomings of current rodent and non-human primate models, we developed a piglet model of lethal SEB intoxication. Fourteen-day-old Yorkshire piglets were given intravenous SEB, observed clinically and euthanized at 4, 6, 24, 48, 72 or 96 hours post treatment. Clinical signs were biphasic with pyrexia, vomiting and diarrhea within 4 hours, followed by terminal hypotension and shock by 96 hours. Widespread T-lymphocyte proliferation was apparent in most piglets by 24 hours and all piglets by 48 hours. By 72 hours lymphadenopathy had progressed to markedly enlarged, dark red lymph nodes characterized histologically by hemorrhage, edema, perivascular fibrin accumulation and widespread lympholysis. At 72 hours there was severe widespread edema, most prominent in the mesentery, between loops of spiral colon, and in retroperitoneal connective tissue. Additional histologic changes included perivascular aggregates of large lymphocytes variably present in the lung and brain, circulating lymphoblasts and lymphocytic portal hepatitis. Study of this piglet model will further elucidate the pathogenesis of SEB intoxication and enable us to test new therapeutic regimes.

The Staphylococcal enterotoxins (SE) are a group of pyrogenic exoproteins produced by gram-positive Staphylococcus aureus. Exposure to SE has been shown to initiate a range of clinical abnormalities from gastrointestinal upset to lethal toxic shock syndrome (TSS). Once introduced into host tissues these proteins have the ability to elicit pathology in many different systems. Within 4 hours of ingestion SE symptoms can be documented and these include: vomiting, diarrhea, nausea, and abdominal pain (Jett M, Brinkley W, Neill R, Gemski P, Hunt R: Infect Immun 1990, 58:3494-3499). Normally enterotoxicosis abates within 24 hours with mild anorexia that persists for up to five days. Currently there are twelve serotypes of SE described, named sequentially by letter (Jarraud S, Peyrat M A, Lim A, Tristan A, Bes M, Mougel C, Etienne J, Vandenesch F, Bonneville M, Lina G: J Immunol 2001, 166:669-677). Staphylococcal enterotoxin B (SEB) is one of the most clinically significant and well-studied members of this family. SEB is known to induce typical food poisoning symptoms, such as fever, vomiting and diarrhea, is implicated as a potent inducer of TSS, and is a potential biological threat agent (Marrack P, Kappler J: Science 1990 Jun 1;248(4959):1066). Much of the lethal effects of SEB have been attributed to superantigenicity and subsequent T-cell proliferation with massive inflammatory cytokine release (Miethke T, Wahl, C.,et al.: Journal of Experimental Medicine 1992, 175:91-98; Johnson H M, Torres B A, Soos J M: Proc Soc Exp Biol Med 1996, 212:99-109).

Unlike traditional antigens, superantigens (SAgs) can stimulate up to 20% of the host's T-cell repertoire. This is accomplished by their unique ability to bypass conventional antigen processing and presentation. Extracellular SE successfully binds both MHC II on antigen presenting cells and the T-cell receptor; creating a functional immunological synapse Jardetzky T S, et al: Nature 1994, 368:711-718). Specifically, it has been shown that interactions with SAgs primarily involves the variable region of the TCR beta chain (Johnson H M, Torres B A, Soos J M: Proc Soc Exp Biol Med 1996, 212:99-109). Subsequent to proliferation, most T cells whose cognate antigen is not present will undergo clonal deletion, resulting in immunosupression. By contrast, in susceptible individuals activated T cells may continue to be stimulated and exacerbate autoimmune disease (Johnson H M, Russell J K, Pontzer C H: Faseb J. 1991, 5:2706-2712).

Of great interest is SEB's ability to interact with non-immunological tissue. In the gastrointestinal tract it has been shown that SEB posses the ability to bind and traverse protective intestinal epithelia (Hamad A R, Marrack P, Kappler J W: J Exp Med 1997, 185:1447-1454; McKay D M, Singh P K: J Immunol 1997, 159:2382-2390). After this process of transcytosis, SEB gains access to circulation and systemic tissue. In the kidney proximal tubule SEB has been shown to bind galactosylceramide. This binding has potential implication in the etiology of SEB-induced hypotension and renal failure (Chatterjee S, Khullar, M., and Shi, W. Y.: Glycobiology 1995, 5:327-333; Chatterjee S, Jett M: Mol Cell Biochem 1992, 113:25-31; Normann S J: Lab Invest 1971, 25:126-132). In in vitro systems SEB demonstrated marked effects on pulmonary arterial cells. Toxin exposure elicited barrier dysfunction which occurred in the absence of effector cells or their intermediate products (Campbell W N, Fitzpatrick M, Ding X, Jett M, Gemski P, Goldblum S E: Am J Physiol 1997, 273:L31-39).

Many in vivo systems for studying SEB have been and are currently being employed. However this area is deficient in an effective and economic animal model, which closely parallels human staphylococcal enterotoxicosis. The non-human primate model (Macaca mulatta) (Normann S J, Jaeger R F, Johnsey R T: Lab Invest 1969, 20:17-25; Stiles J W, Denniston J C: Lab Invest 1971, 25:617-625) has proven to diagram SEB disease progression, but is limited because of high cost, short supply, and complexity of animal care. Rabbit models have been developed to specifically map the lesion progression of toxic shock syndrome toxin-1 (TSST-1, another exotoxin produced by S. aureus) however high doses are required and they need to be introduced via continual peritoneal infusion. Multiple strains of the murine species have also been used as in vivo models for SEB. Results are often skewed and hard to interpret because mice are insensitive to the effects of SEB and traditional mouse models of SEB intoxication require either genetic manipulation (Anderson M R, Tary-Lehmann M: Clin Immunol 2001, 98:85-94; Yeung R S, et al. :Eur J Immunol 1996, 26:1074-1082; Chen J Y, Qiao Y, Komisar J L, Baze W B, Hsu I C, Tseng J: Infect Immun 1994, 62:4626-4631) or prior sensitization, with D-galatosamine, or endotoxin (Miethke T, Wahl, C., Heeg, K., Echtenacher, B., Krammer, P., and Wagner, H.: Journal of Experimental Medicine 1992, 175:91-98). Even, with co-administered D-gal, the clinical syndrome in mice does not mimic that seen in higher order mammals.

In the present study a lethal SEB model using 14-day-old Yorkshire piglets was assessed for diagnostic parameters and relevance to human disease progression. This model could provide a promising alternative to traditional in vivo models for SEB. Piglets are easy to obtain, cost efficient, and require minimal care compared to those of primates. This paper characterizes the clinical syndrome, histological lesions and post mortem findings of intravenous SEB-exposed (lethal dose) piglets at varying time points.

Materials and Methods

Animals:

All animal use was carried out in accordance with AR 70-18, paragraph 12.d., in compliance with the Animal Welfare Act, adhering to the principles enunciated in The Guide for the Care and Use of Laboratory Animals. Litters of ˜8, 12-day-old, male and female Yorkshire piglets were obtained from Archer Farms (Darlington, Md.) and housed in groups of ˜3 piglets (assigned by treatment) in metal runs lined by rubber mats. Piglets were maintained under controlled lighting (12-hour light-dark cycle), at a temperature of 85° F. and humidity of ˜60%. Animals were fed swine pre-starter complete feed (Hubbard Feeds, Mankato, Minn.). Piglets had continual access to feed, water and a 2-3 heat lamp sources at one end of the run. At ˜18-days of age, anesethetized piglets (isofluorane (3% initially, achieving maintenance at ˜1.5-2%) (Abbott Labs, North Chicago, Ill.) received a lethal dose of SEB (150 μg/kg) or an equivalent volume of saline, administered into the ear vein using a 22 g 3/4 inch catheter. At 4, 6, 24, 48, 72 or 96 hours post treatment, animals were anesthetized with isofluorane, terminal measurements and blood were obtained and the piglets were euthanized using Buthanasia-D (Bums Biotech, Omaha, Nebr.) administered via intracardiac injection.

Toxin Preparation:

SEB, lot 14-30, purified by the method of Schantz et al (Schantz EJ, et al.: Biochemistry 1965, 4:1011-1016), was stored as a dry powder in pre-measured vacuum ampules. A working stock solution was made by dissolving the SEB in sterile pyrogen-free water to achieve a concentration of 5 mg/ml and that solution was aliquoted and stored frozen. At the time of use, an appropriate aliquot was thawed and diluted with i.v. injectable saline to 300 μg/ml. LD_{˜95} was achieved using 150 μg/kg. Lethality was also observed at 50 μg/kg but not at 30 μg/kg.

Clinical Observations and Measurements:

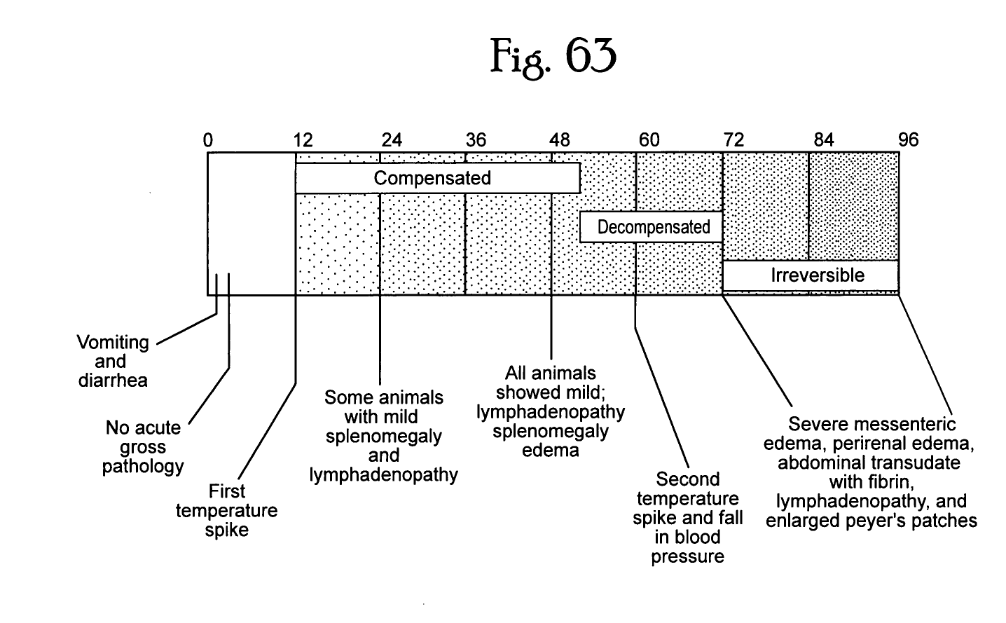

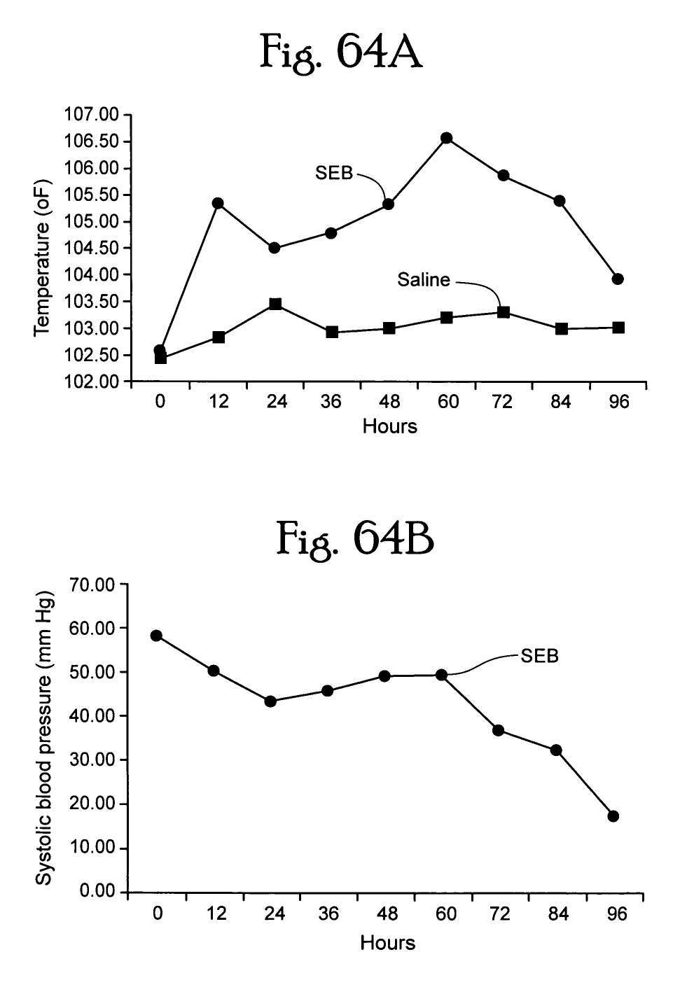

Animals were monitored continuously for clinical signs for the first 18 hours post treatment and every 6 hours until euthanasia. Recorded clinical observations included clinical sign results for at least 3 piglets per time period and for 3 different experiments (FIG. 63). Rectal body temperature was measured at least hourly 0-12 h and 1-2× daily thereafter (FIG. 64a). Systolic blood pressure was measured by Ultrasonic Doppler Flow Detector (Model 811BL; Parks, Medical Electronics; Aloha, Oreg.). (see FIG. 64b)

Gross and Microscopic Pathology:

After euthanasia a complete necropsy was performed as follows: 4 hours (1 piglet), 6 hours (1 piglet), 24 hours (5 piglets), 48 hours (5 piglets), 72 hours (7 piglets) and 96 hours (4 piglets). At least one saline control piglet was examined per litter, with a total of 7 saline controls. A full set of tissues from each animal was fixed in 10% neutral buffered formalin. Fixed tissues were routinely trimmed, embedded in paraffin, sectioned at 5-7 μm and stained with hematoxylin and eosin for microscopic examination. Tissues examined microscopically for this report were: thymus, stomach, jejunum, spiral colon, descending colon, liver, spleen, pancreas, kidney, adrenal gland, urinary bladder, multiple lymph nodes, lung, heart, and brain.

Gene Studies

Whole blood samples were collected into CPT™ Vacutainer™ tubes (BD, Franklin Lakes, N.J.) at various time points and processed in accordance with the manufacturer's specifications which allow for the enrichment of peripheral blood mononuclear cells (PBMC). Total RNA was subsequently isolated from PBMCs using TRIzol reagent (Life Technologies, Grand Island, N.Y.) following the manufacturer's protocol.

Preliminary gene array yielded data that implicated several gene profile changes post-SEB treatment (data not presented). Five representative genes were chosen and primer pairs to be used for PCR were designed based on known mRNA sequences (Genbank, PubMed) using Primer software³ or Genelooper 2.0 from Geneharbor.

Equal amounts of total RNA were reverse transcribed to cDNA using oligo (dT) and Superscript reverse transcriptase II (Invitrogen, Carlsbad, Calif.). The obtained cDNA was used as a template for PCR reactions using PCR master mixture (Roche, Indianapolis, Ind.). Each cDNA was subjected to 25-30 PCR cycles using a GeneAmp 9600 thermal cycler (Perkin Elmer, Norwalk, Conn.) with conditions that resulted in a single specific amplification product of the correct size. Amplification was empirically determined to be in the linear range. mRNA amounts were normalized relative to 18S rRNA. Reaction products (10 μl ) were visualized after electrophoresis on a 1% agarose gel using SYBR Green I (Kemtek, Rockville, Md.). Gels were digitized using a BioRad Molecular Imager FX (BioRad, Hercules, Calif.) and band intensities were used to calculate mRNA abundance.

Results

Clinical Signs

Administration of SEB either IV or intratracheal at 150 μg/kg was lethal (or deemed non-survivable by the attendant veterinarian) in 31/31 piglets. An IV dose of 50 μg/kg resulted in lethality while 30 μg/kg was not lethal. After administration of the SEB, pre-established behavioral characteristics were recorded for each animal as a function of time post exposure during the course of the experiment (continually for the first 6 h and intermittently during the rest of the experiment). Five descriptions of piglet behavior for each of 3 categories (healthy, incapacitation, prostration) were established based on observed behavior from other studies with piglets. The animals showed onset of typical incapacitation signs (transient vomiting [˜3-6 episodes], severe diarrhea, anorexia) at 0.8-1.5 h post exposure (FIG. 63). The diarrhea, anorexia persisted during the remainder of the experiment. From 3-7 h, the animals seldom left the lamp-heated areas of the cage, in showing continually increasing signs of prostration. Euthanasia was carried out as early as was possible, for the experimental objective, in order to minimize distress. Plotted rectal temperatures showed two febrile peaks at 12 and 60 hours with the 60 h time point being most extreme. Around day 3 temperatures began to fall and showed no evidence of homeostatic recovery (FIG. 64A). Systolic blood pressures were variable throughout most of the time course however a distinct hypotensive trend was observed at or around the third day of observation (FIG. 64B).

Gross Findings

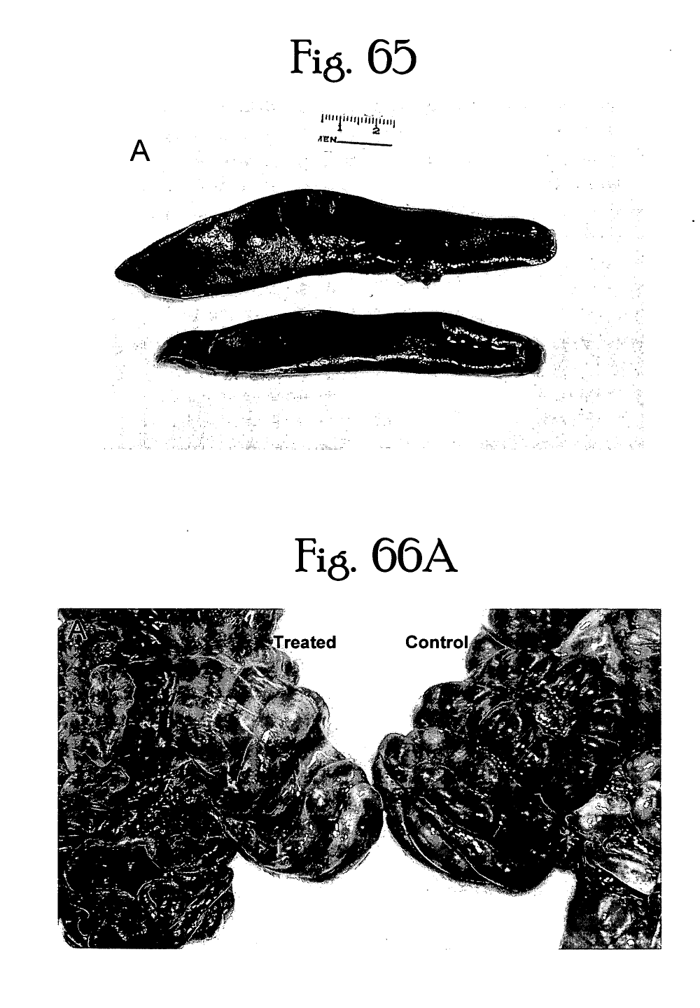

Gross changes were progressive over time. No significant gross changes were present in the both piglets necropsied at 4 and 6 hours post SEB treatment or in any saline control animals. By 24 hours mildly enlarged mesenteric lymph nodes and mild splenomegaly were present in 2 of 5 animals. By 48 hours post SEB treatment in all animals there was consistent mild splenomegaly when compared to control animals (FIG. 65) and diffuse mild to moderate enlargement of the mesenteric lymph nodes that were often bright to dark red. Six of seven animals at this time point had mild to moderate perirenal, mesenteric, gall bladder and gastric wall edema and mildly enlarged and congested peripheral lymph nodes. Two of the seven animals had prominent red peyer's patches and a marked abdominal transudate with strings of fibrin.

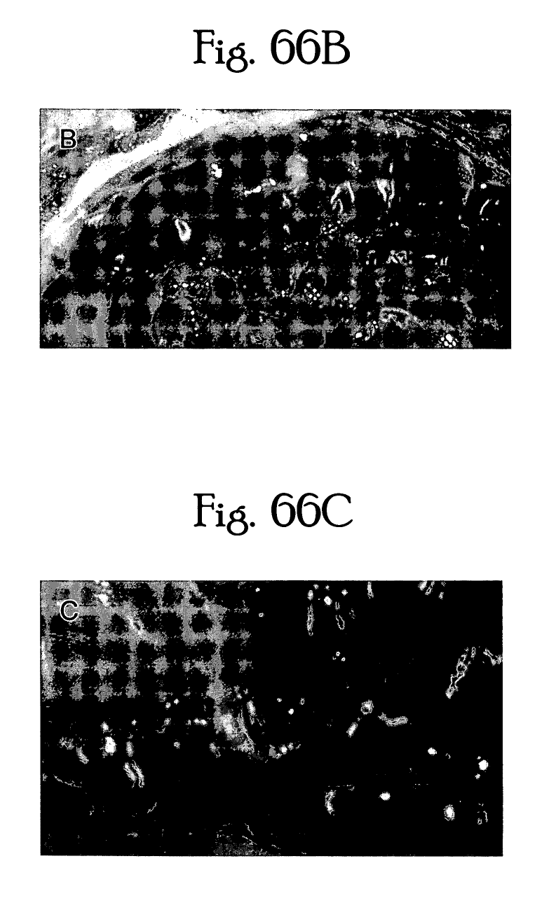

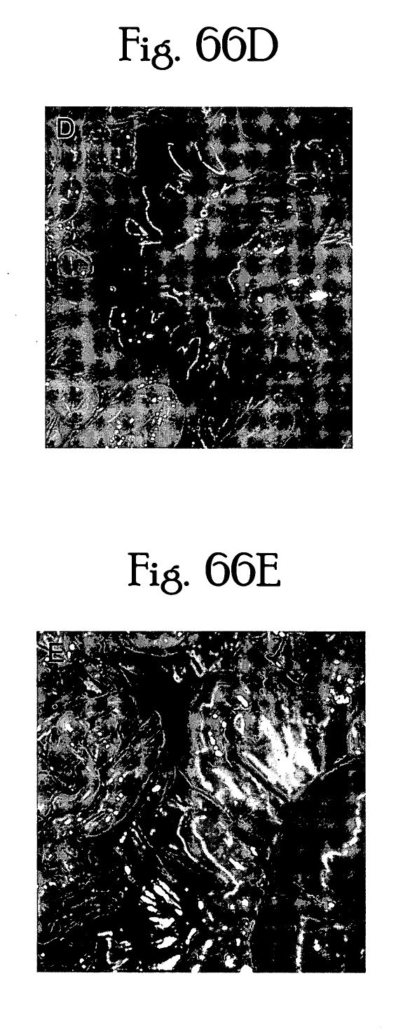

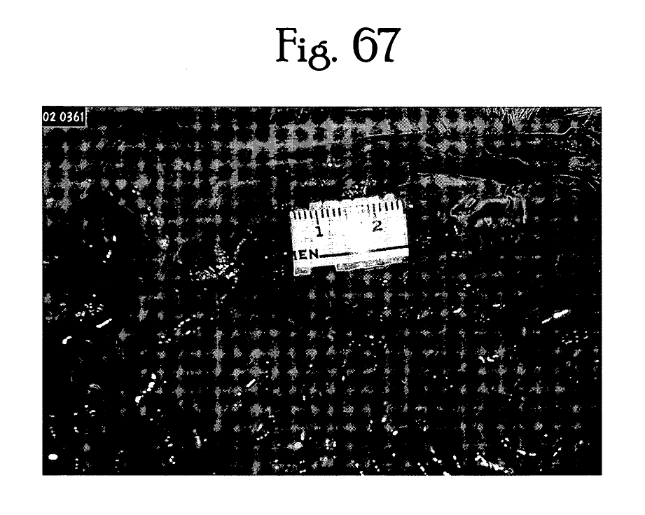

Gross lesions were most remarkable at 72 and 96 hours post exposure. All animals necropsied at these time periods had severe mesenteric edema that was most prominent between loops of spiral colon, (FIG. 66A), as well as perirenal edema (FIG. 66A), variable edema of the gall bladder and gastric wall and mild diffuse subcutaneous edema. This was accompanied by a marked abdominal transudate (protein, 2.5 g /dL, with few cells) and with strands of fibrin (FIG. 66C). Mesenteric lymph nodes were greatly enlarged, dark red (FIG. 66D-E) and often contained multifocal white areas of necrosis. Peripheral lymph node involvement was similar and varied from minimal to severe. Peyer's patches were often prominent and red (congested) (FIG. 67).

Microscopic Findings

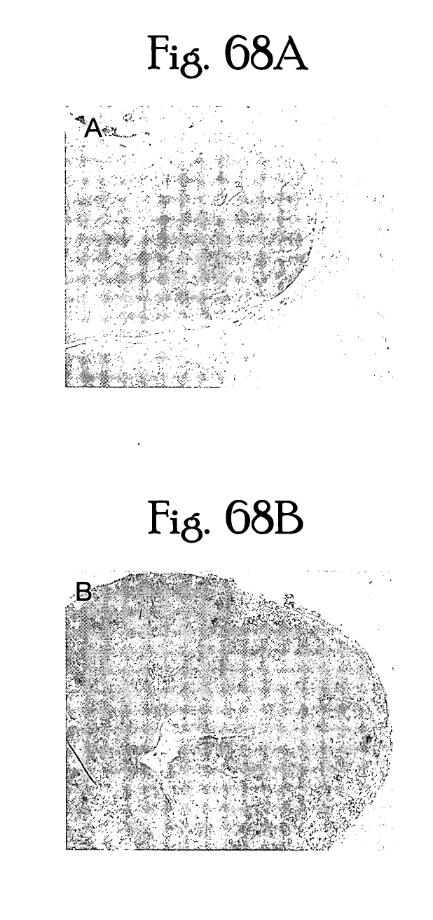

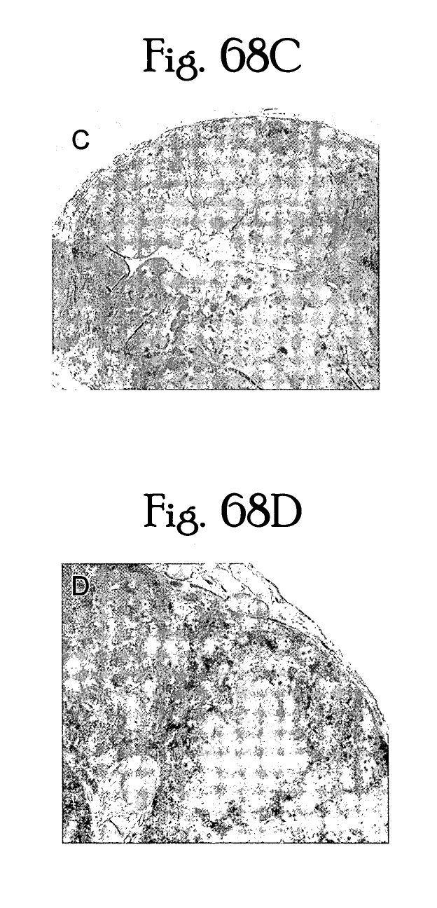

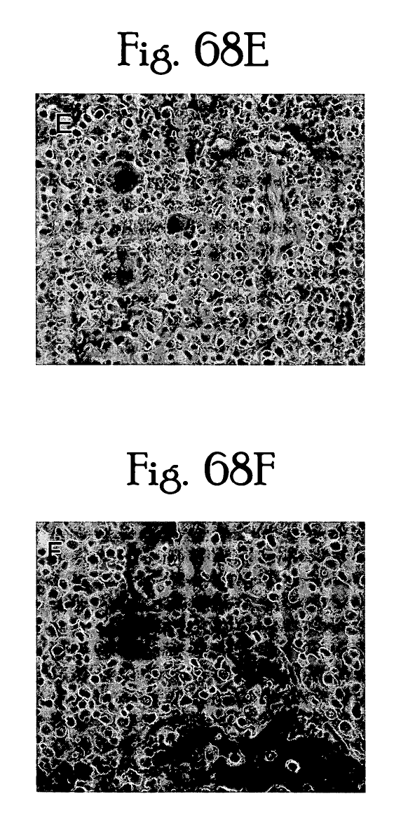

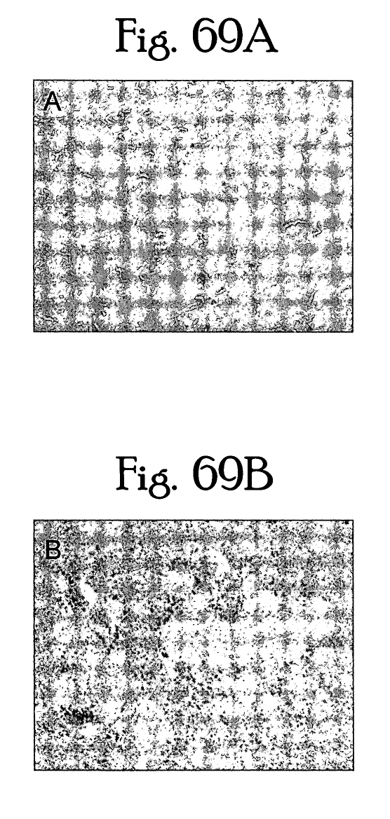

Histologic examination of selected tissues confirmed gross observations and helped to further characterize changes. The general progression of histologic changes in the mesenteric lymph nodes was: mild lymphoid hyperplasia by 24 hours, progression to moderate lymphoid hyperplasia and congestion by 48 hours, and marked lymphoid necrosis with hemorrhage, edema and fibrin accumulation by 72 to 96 hours (FIG. 68A-D). Mild to moderate diffuse lymphoid hyperplasia was present in mesenteric lymph nodes in all animals examined at 24 hours post exposure. At 48 hours, all mesenteric lymph nodes examined had moderate to severe diffuse lymphoid hyperplasia. Many blood vessels in these nodes were congested and the loose peripheral tissue analogous to medullary sinuses contained many free erythrocytes. In addition, there were a few small scattered areas of hemorrhage and lymphoid necrosis. Lymphoid necrosis was much more extensive in 6 of 7 and 3 of 3 mesenteric lymph nodes examined at 72 and 96 hours respectively. At these time points extensive lymphoid necrosis characterized by abundant karyorrhectic debris was accompanied by marked hemorrhage and edema often with fibrin lining small caliber vessels and prominent fibrin thrombi (FIGS. 68E-F). Changes in the peripheral lymph nodes were similar but much less severe and tended to occur at the later time periods.

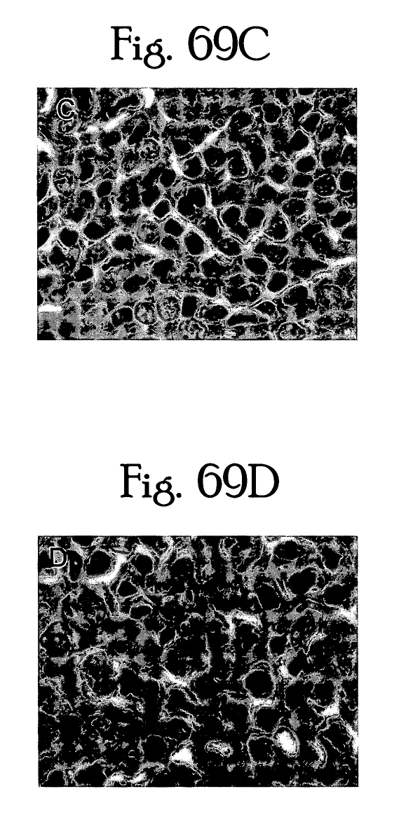

Lymphoid hyperplasia was also present in all spleens examined at 24 hours post treatment and later. This change was characterized by mild diffuse expansion of the periarteriolar lymphoid sheaths (PALS) (FIGS. 69A-B). The lymphocytes in the affected PALS were larger, with increased cytoplasm and a large irregularly round stippled nucleus and there were increased numbers of mitotic figures in these areas (FIGS. 69C-D).

Severe mesenteric edema between loops of spiral colon seen grossly at 48 and 96 hours (FIG. 66A) was verified histologically. Microscopically mesenteric connective was loosely arranged and widely separated by a lightly eosinophilic to clear material and delicate eosinophilic fibrillar material (edema) and many extravasated red blood cells. Mesenteric lymphatics were consistently ectatic.

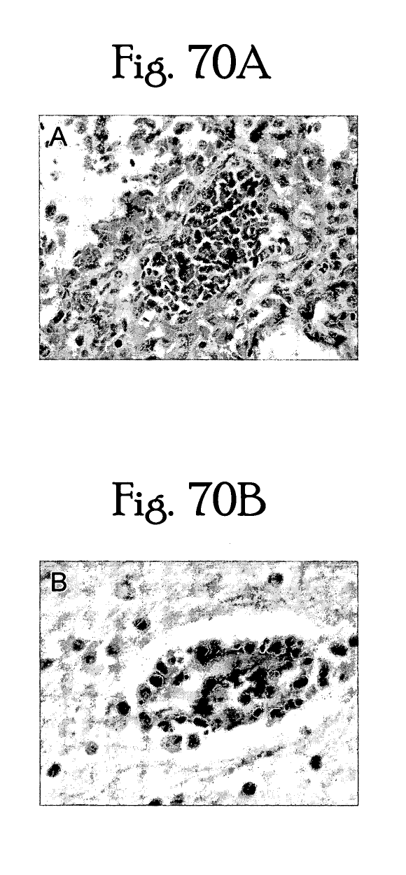

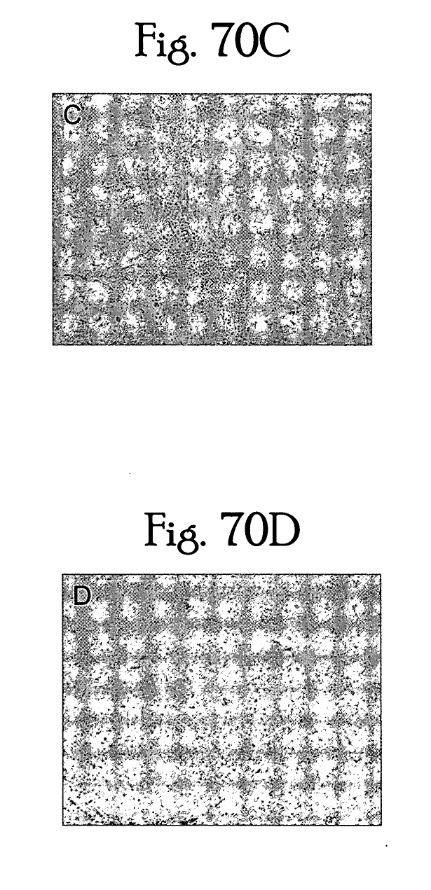

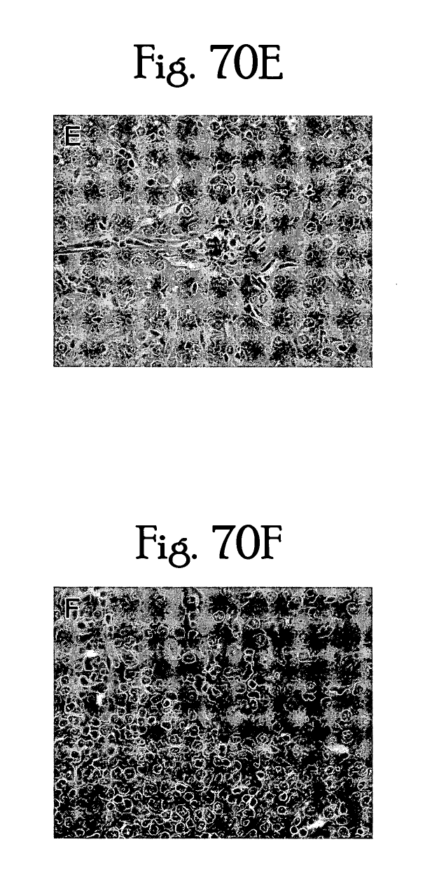

Additional histologic findings included lymphoblastic perivascular infiltrates and mild portal lymphoplasmacytic hepatitis. Small perivascular lymphocytic cuffs were present in the lungs of most animals examined at 48 hours and later (5 of 6 and 48 hours, 7 of 7 at 72 hours and 3 of 4 at 96 hours) (FIG. 70A) and in the brain of two animals examined at 96 hours (FIG. 70B). Cuffs often contained evidence of lymphoid necrosis with accumulation of karyorrhectic debris. Mild lymphoplasmacytic portal hepatitis (FIGS. 70C-F) was variably present at 24 hours and later: 3 of 5 piglets at 24 hours, 3 of 5 piglets at 48 hours, 6 of 7 piglets at 72 hours and 1 of 4 piglets at 96 hours.

SEB-Induced Gene Changes

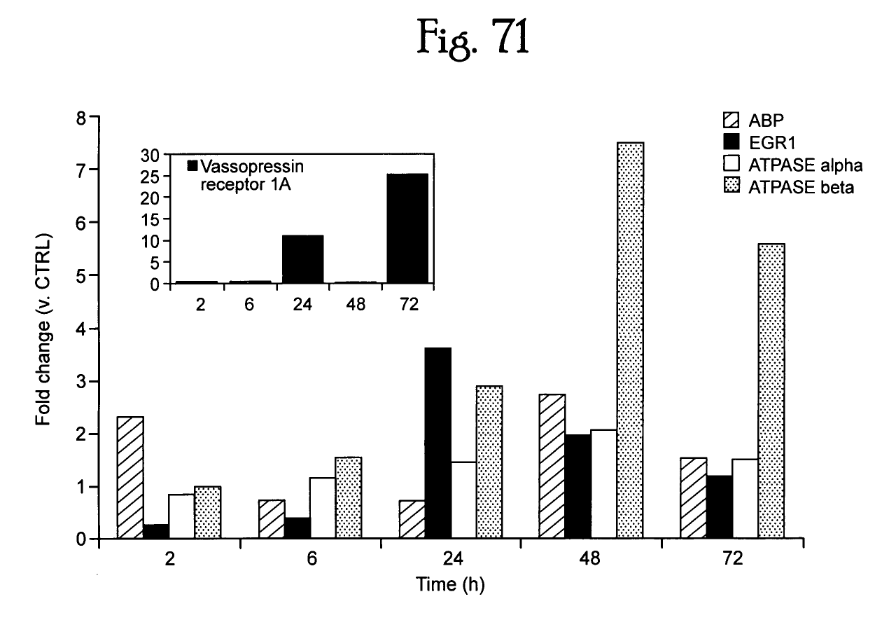

After initial survey using custom gene microarrays, five genes were selected for study at 2, 6, 24, 48, and 72 hours post SEB exposure using RT-PCR (FIG. 71). mRNA levels for vasopressin receptor 1a (V1a),; a peripheral receptor associated with vasoconstriction, were markedly increased at 24 and 72 hours (˜10-fold and ˜25-fold respectively). Interestingly the timing of the V1a gene changes coincide with observed systolic blood pressure changes graphed in FIG. 64A. Na, K-ATPase subunits α and β gene profiles showed a time dependent increase which were greatest at 48 hours. Although both subunits followed a similar trend, the βisoform proved to have a larger increase as compared to that of the α isoform (˜8-fold, v. ˜2-fold at 48 hours). Early growth response gene 1 (Egr1), a key transcription factor implicated in many disease processes including hypoxia, showed an increase at all time points. Most remarkably was an increase in mRNA levels at the 24-hour time point. Finally, the gene profile for the soluble angiotensin binding protein (sABP) was also increased at all time points with highest levels found at 48 hours.

Discussion

We have developed a clinically relevant piglet model of lethal SEB intoxication that we propose is superior to the current monkey and rodent models. This model more realistically parallels SEB intoxication in people than described mouse models and piglets are easier to obtain, maintain and handle than the non-human primate model.

This piglet model exhibits a biphasic clinical response to SEB intoxication that is virtually identical to that described in people but is not described in mouse models. Although lethal SEB intoxication has been achieved in previously manipulated mouse models, none of these models exhibit the typical initial gastrointestinal signs described in humans. In addition, the small size of these animals (Mice) makes obtaining many clinical measurements such as repeat routine hematology, serum chemistries, blood pressure and body temperature difficult.

The monkey model of lethal SEB intoxication is more clinically relevant than mouse models. However, although rhesus monkeys show a subtle clinical biphasic response to SEB intoxication it is not as exuberant or easily detected and monitored as that seen in the piglet model (One author's personal observation, MJ). This is likely a result of the fact that the laboratory Rhesus monkey retains many behavioral characteristics of its wild counterpart, including remarkable masking of clinical disease, which increases survival under natural adverse conditions; this is in marked contrast to the domestic pig whose disposition has been markedly altered by selective breeding. In addition, working with non-human primates, especially rhesus macaques, comes with a unique set of limitations, most notably high expense, limited supply and biosafety concerns. The aggressive nature of these monkeys and complications associated with Herpes B positive colonies make heavy sedation or anesthesia necessary for many routine procedures. In contrast, the piglets used in this model are easy to obtain and relatively inexpensive. The social nature of these animals allows routine procedures to be preformed without anesthesia or sedation and with minimal stress to the animal and handler.

In addition, study of other porcine models of human disease indicate that this species shows strong similarities to humans with respect to vascular responsiveness (Feletou M, Teisseire B: Edited by Swindle M M, Moody D C, Phillips L D. Ames, Iowa State Universtiy Press, 1992, pp 74-95) and is a good model in which to study cardiovascular disease. In fact, Lee et al (Lee P K, Vercellotti G M, Deringer J R, Schlievert P M: J Infect Dis 1991, 164:711-719) used porcine aortic endothelial cells to demonstrate that TSST-1), has a direct toxic effect on endothelium. There is also a described swine model of septic shock that culminates in a hypotensive crisis (Hoban L D, et al.: Awake porcine model of interperitoneal sepsis. Edited by Swindle M M, Moody D C, Phillips L D. Ames, Iowa State Universtiy Press, 1992, pp 246-264) that is similar to that observed in this model.

We have shown that administration of intravenous SEB to piglets results in terminal hypotension and shock similar to that seen in toxic shock syndrome in people and SEB intoxication in the rhesus macaque. Postmortem findings in people, monkeys and piglets indicate that hypotension and shock in SEB intoxication is a result of leakage of fluid from vessels into extravascular spaces. Pulmonary edema is the most consistent and remarkable gross lesion associated with death in the primate model of intravascular SEB intoxication (Finegold M J: Lab Invest 1967, 16:912-924) and in people with toxic shock syndrome (Larkin S M,et al: Ann Intern Med 1982, 96:858-864). One major difference in this piglet model compared to the disease in humans is that terminal edema is predominantly focused on the abdomen rather than the thorax resulting in severe mesenteric and perirenal edema with comparatively minor edema at other sites. It is interesting to note, that other natural and experimental angiotoxic diseases in the pig result in vascular leakage with edema predominantly in the abdominal region. In edema disease, a well characterized porcine disease, direct endothelial binding of Shiga-like toxin type Hie (SLT-IIe) secreted by E. coli, results in marked spiral colon mesenteric edema similar to that seen in this SEB piglet model (Gelberg H B: Alimentary system. Thomson's Special Veterinary Pathology. Edited by McGavin M D, Carltom W W, Zachary J F. St. Louis, Mosby, 2001, pp 42-43). In another porcine model that displays classical signs of circulatory shock, edema of the gastric wall and gall bladder is a result of experimental intravenous administration of T-2 toxin, a mycotoxin secreted by Fusarium species thought to cause moldy corn disease in swine (Pang V F, Lorenzana RM, Beasley VR, Buck WB, Haschek WM: Fundam Appl Toxicol 1987, 8:298-309). The abdominally focused edema in pigs may constitute a species difference that should be considered, especially in research aimed at treating late stage hypotensive shock and pulmonary edema. However, we feel strongly that this model is still a valid model for pathogenesis studies and lethal SEB intoxication prophylactic, early and mid-stage treatment trials.

Another characteristic unique to swine is the unique porcine lymph node architecture. Porcine lymph nodes are essentially reversed from other mammalian lymph nodes in that lymphoid tissue is centrally located and surrounded by loose peripheral lymphoreticular tissue resembling the medullary sinuses in other species. Although porcine lymph nodes are morphologically different, the functional flow of lymph is essentially identical to other species (Landsverk T: Immune system. Textbook of Veterinary Histology. Edited by Dellmann D, Eurell J A. Baltimore, Williams & Wilkins, 1998, pp 137-142) and in the author's (YAV) opinion does not represent a significant species difference, except perhaps in interpretation of lesions by a swine-naive histopathologist.

Histological lesions in this piglet model are similar to those described in other animal models of SEB intoxication. Ulrich et al (Ulrich R G, et al.: Textbook of Military medicine. Part I Warfare, Weaponry, and the Casualty. Ed. by Sidell F R, Takafuji E T, Franz D R. Washington, Office of the Surgeon General, 1997, pp 621-630 )provides a detailed description of both pulmonary and non-pulmonary lesions associated with lethal aerosol SEB exposure in the rhesus macaque. This model also had wide spread T-lymphocyte hyperplasia with enlarged lymph nodes, expanded PALS and circulating lymphoblasts. In addition, lymphocytic portal infiltrates similar to those seen in this model where also reported in the exposed monkeys. Another report of lethal aerosol SEB exposed monkeys described pulmonary perivascular lymphocytic infiltrates similar to those seen in this study (Mattix M E, Hunt R E, Wilhelmsen C L, Johnson A J, Baze W B: Toxicol Pathol 1995, 23:262-268). Lymphoid hyperplasia followed by lympholysis in the spleen is described in an Actinomycin-D primed mouse model (Chen J Y, Qiao Y, Komisar J L, Baze W B, Hsu I C, Tseng J: Infect Immun 1994, 62:4626-4631.). A similar change was noted in a mouse model of aerosol SEB exposure (Vogel, Pa., personal communication). These findings are consistent with the immunological manesfestations of SAg exposure.

As in the mouse models marked lympholysis was apparent in most piglets at 72 and 96 hours post SEB administration. However, this change was limited to severely affected lymph nodes and was not apparent in the thymus or spleen. It is possible that the severe lymphoid depletion noted at autopsy of several lethal cases of human toxic shock syndrome (Larkin S M,et al: Ann Intern Med 1982, 96:858-864) was a sequela of massive lympholysis. As TSS is lethal only in a small percentage of cases it is interesting to hypothesize that this change may be associated with lethality.

In summary we have characterized the clinical syndrome and post mortem findings of a 14-day-old Yorkshire piglet model of lethal SEB intoxication. We propose that this model is superior to previously described models. It is our hope that study of this piglet model will further elucidate the pathogenesis of SEB intoxication and enable us to test new therapeutic regimes.

The febrile state of treated animals is of particular interest and raises many questions. Studies using SEA mutants suggest that the emetic and superantigenic activity of SEs may be separate³². Immediately following exposure, piglets presented with an emetic phase that was not associated with temperature increase. Marked temperature elevation was not recorded in animals until after the last emetic event. If superantigenic T cell stimulation and subsequent cytokine production was solely responsible, one would suspect that the timing of emesis and fever would closely overlap. These data support the previously discerned hypothesis that the gastrointestinal and pyrogenic effects of SE may in fact be of different mechanism.

The timing of clinical symptoms, vital measurements, and pathologic lesions appears to be in direct concert (FIG. 63). The initial phases of intoxication caused severe incapacitation, and occurred in the absence of gross or histological lesions. Animals appeared to recover after initial onset, left only with residual diarrhea and fever. Gross lesions appear to develop around 24 hours corresponding with a further increase in body temperature. At hour 60 animal temperatures began to fall, corresponding with incremental reductions in systolic blood pressure and marked progression of pathologic lesions.
In order to elucidate pathways responsible for SEB lesions we have begun to profile gene changes. In this study we present data on five genes that were flagged subsequent to preliminary gene array surveying (FIG. 71). The transcription factor Egr1, is shown to have altered expression in the face of hypoxia (Streefkerk J O, et al. Fundam Clin Pharmacol. 2004 Feb;18(1):45-50.; Russell S D, DeWald T. Am J Cardiovasc Drugs. 2003;3(1):13-20). Also, Egr family members have been implicated in the non-lymphoid expression of FasL and TNF(Huang H C, Wang S S, Chan C C, Lee F Y, Chang F Y, Lin H C, Hou M C, Tai C C, Lai I N, Lee S D. J Hepatol. 2004 Feb;40(2):234-8). Interestingly, mRNA levels of Egr1 were highest at 24 h when the first signs of pathological lesions became evident. sABP, a binding protein for angiotensin is found widely distributed in peripheral tissues and in the brain(Fourrier F, Chopin C, Huart J J, Runge I, Caron C, Goudemand J. Chest. 1993 September; 104(3): 882-8). Although its physiologic relevance is uncertain, sABP may play a role in the balance of smooth muscle contraction (Warren B L, et al. JAMA. 2001 Oct 17; 286(15): 1869-78). V1a, unlike V2a, is recognized to initiate vasoconstriction upon binding of its ligand vasopressin (anti-diuretic hormone, ADH). This vasoconstriction is part of a marked compensatory response to hypotension.

By increasing peripheral vasculature resistance, blood pressure can be returned to a level that ensures adequate tissue perfusion. In this study, V1a mRNA levels are increased notably at 24 h, a time when systolic blood pressure re-equilibrates, and these levels are further increased at 72 h at the onset of the hypotensive crisis.

The complex nature of SE pathophysiology has posed many questions and much of the host's response to these toxins has been explained in terms of their effect on the body's immune system. As we progress further in understanding the chronology and severity of lesions induced by SEB, it will be necessary to further investigate SEs interaction with non-immunological tissue. Most notably would be the correlation of SEs effect on endothelium and on epithelial tissues with the presence of irreversible shock.

In summary we have characterized the clinical syndrome and post mortem findings of a 14-day-old Yorkshire piglet model of lethal SEB intoxication and propose that this model is superior to previously described models. It is our hope that study of this piglet model-will further elucidate the pathogenesis of SEB intoxication and enable us to test new therapeutic regimes.

References

1. Jett M, Brinkley W, Neill R, Gemski P, Hunt R: Infect Immun 1990, 58:3494-3499.
2. Jarraud S, Peyrat M A, Lim A, Tristan A, Bes M, Mougel C, Etienne J, Vandenesch F, Bonneville M, Lina G: J Immunol 2001, 166:669-677
3. Marrack P, Kappler J: Science 1990 Jun 1;248(4959):1066] [see comments]. Science 1990, 248:705-711
4. Miethke T, Wahl, C., Heeg, K., Echtenacher, B., Krammer, P., and Wagner, H.: Journal of Experimental Medicine 1992, 175:91-98
5. Johnson H M, Torres B A, Soos J M: Superantigens: structure and relevance to human disease. Proc Soc Exp Biol Med 1996, 212:99-109
6. Jardetzky T S, Brown J H, Gorga J C, Stern L J, Urban R G, Chi Y I, Stauffacher C, Strominger J L, Wiley D C: Three-dimensional structure of a human class II histocompatibility molecule complexed with superantigen. Nature 1994, 368:711-718
7. Johnson H M, Russell J K, Pontzer C H: Staphylococcal enterotoxin microbial superantigens. Faseb J 1991, 5:2706-2712
8. Hamad A R, Marrack P, Kappler J W: Transcytosis of staphylococcal superantigen toxins.

J Exp Med 1997, 185:1447-1454
9. Shupp J W, Jett M, Pontzer C H: Identification of a transcytosis epitope on staphylococcal enterotoxins. Infect Immun 2002, 70:2178-2186
10. McKay D M, Singh P K: Superantigen activation of immune cells evokes epithelial (T84) transport and barrier abnormalities via IFN-gamma and TNF alpha: inhibition of increased permeability, but not diminished secretory responses by TGF-beta2. J Inmunol 1997, 159:2382-2390
11. Chatterjee S, Khullar, M., and Shi, W. Y.: Digalactosylceramide is the receptor for staphylococcal enterotoxin-B in human kidney proximal tubular cells. Glycobiology 1995, 5:327-333
12. Chatteijee S, Jett M: Glycosphingolipids: the putative receptor for Staphylococcus aureus enterotoxin-B in human kidney proximal tubular cells. Mol Cell Biochem 1992, 113:25-31
13. Normann S J: Renal fate of staphylococcal enterotoxin B. Lab Invest 1971, 25:126-132
14. Campbell W N, Fitzpatrick M, Ding X, Jett M, Gemski P, Goldblum S E: SEB is cytotoxic and alters EC barrier finction through protein tyrosine phosphorylation in vitro. Am J Physiol 1997, 273:L31-39
15. Normann S J, Jaeger R F, Johnsey R T: Pathology of experimental enterotoxemia. The in vivo localization of staphylococcal enterotoxin B. Lab Invest 1969, 20:17-25.
16. Stiles J W, Denniston J C: Response of the Rhesus monkey, Mucaca mulatta, to continuously infuded Staphylococcal Entertoxin B. Lab Invest 1971, 25:617-625
17. Ulrich R G, Sidell S, Taylor T J, Wilhelmsen C L, Franz D R: Staphylococcal enterotoxin B and related pyrogenic toxins. Textbook of Military medicine. Part I Warfare, Weaponry, and the Casualty. Medical Aspects of Chemical and Biological Warefare. Edited by Sidell F R, Takafuji E T, Franz D R. Washington, Office of the Surgeon General, 1997, pp 621-630
18. Anderson M R, Tary-Lehmann M: Staphylococcal enterotoxin-B-induced lethal shock in mice is T-cell-dependent, but disease susceptibility is defined by the non-T-cell compartment. Clin Immunol 2001, 98:85-94.
19. Yeung R S, Penninger J M, Kundig T, Khoo W, Ohashi P S, Kroemer G, Mak T W: Human CD4 and human major histocompatibility complex class II (DQ6) transgenic mice: supersensitivity to superantigen-induced septic shock. Eur J Immunol 1996; 26:1074-1082.
20. Chen J Y, Qiao Y, Komisar J L, Baze W B, Hsu I C, Tseng J: Increased susceptibility to staphylococcal enterotoxin B intoxication in mice primed with actinomycin D. Infect Immun 1994, 62:4626-4631.
21. Schantz E J, Roessler W G, Wagman J, Spero L, Dunnery D A, Bergdoll MS: Purification of staphylococcal enterotoxin B. Biochemistry 1965, 4:1011-1016
22. Felfetou M, Teisseire B: Vascular pharmacology of the micropig: Importance of the endothelium. Swine as Models in Biomedical Research. Edited by Swindle M M, Moody DC, Phillips L D. Ames, Iowa State Universtiy Press, 1992, pp 74-95
23. Lee P K, Vercellotti G M, Deringer J R, Schlievert P M: Effects of staphylococcal toxic shock syndrome toxin 1 on aortic endothelial cells. J Infect Dis 1991, 164:711-719.
24. Hoban L D, Paschall J A, Eckstein J, Nadkari V, Lee C R, Williams T J, Reusch D, Nevola J J, Carcillo J A: Awake porcine model of intrperitoneal sepsis. Swine as Models in Biomedical Research. Edited by Swindle M M, Moody D C, Phillips L D. Ames, Iowa State Universtiy Press, 1992, pp 246-264
25. Finegold M J: Interstitial pulmonary edema. An electron microscopic study of the pathology of Staphylococcal enterotoxemia in Rhesus monkeys. Lab Invest 1967, 16:912-924
26. Larkin S M, Williams D N, Osterholm M T, Tofte R W, Posalaky Z: Toxic shock syndrome: clinical, laboratory, and pathologic findings in nine fatal cases. Ann Intern Med 1982, 96:858-864.
27. Gelberg H B: Alimentary system. Thomson's Special Veterinary Pathology. Edited by McGavin M D, Carltom W W, Zachary J F. St. Louis, Mosby, 2001, pp 42-43
28. Pang V F, Lorenzana R M, Beasley V R, Buck W B, Haschek W M: Experimental T-2 toxicosis in swine. III. Morphologic changes following intravascular administration of T-2 toxin. Fundam Appl Toxicol 1987, 8:298-309.
29. Landsverk T: Immune system. Textbook of Veterinary Histology. Edited by Dellmann D, Eurell J A. Baltimore, Williams & Wilkins, 1998, pp 137-142
30. Mattix M E, Hunt R E, Wilhelmsen C L, Johnson A J, Baze W B: Aerosolized staphylococcal enterotoxin B-induced pulmonary lesions in rhesus monkeys (Macaca mulatta). Toxicol Pathol 1995, 23:262-268.
31. R. C. Webb, M. A. Kiron, E. Rosenberg and R. L. Soffer Antiserum to angiotensin-binding protein inhibits vascular responses to angiotensin II. Am J Physiol Heart Circ Physiol 255: H1542-H1544, 1988
32. Sun Y, Zhang J, Lu L, Bedigian M P, Robinson A D, Weber K T.Tissue angiotensin II in the regulation of inflammatory and fibrogenic components of repair in the rat heart. J Lab Clin Med. 2004 January;143(1):41-51.
33. Streefkerk J O, Hoogaars W M, Christoffels V M, Sand C, Pfaffendorf M, Peters S L, Van Zwieten P A. Vasopressin-induced vasoconstriction is dependent on MAPKerk 1/2 phosphorylation. Fundam Clin Pharmacol. 2004 February;18(1):45-50.
34. Russell S D, DeWald T.Vasopressin receptor antagonists: therapeutic potential in the management of acute and chronic heart failure. Am J Cardiovasc Drugs. 2003;3(1):13-20.
35. Huang H C, Wang S S, Chan C C, Lee F Y, Chang F Y, Lin H C, Hou M C, Tai C C, Li I N, Lee S D. Chronic inhibition of nitric oxide increases the collateral vascular responsiveness to vasopressin in portal hypertensive rats. J Hepatol. 2004 Feb;40(2):234-8.
36. Fourrier F, Chopin C, Huart J J, Runge I, Caron C, Goudemand J. Chest. Double-blind, placebo-controlled trial of antithrombin m concentrates in septic shock with-disseminated intravascular coagulation. 1993 September; 104(3): 882-8.
37. Warren B L, Eid A, Singer P, Pillay S S, Carl P, Novak I, Chalupa P, Atherstone A, Penzes I, Kubler A, Knaub S, Keinecke H O, Heinrichs H, Schindel F, Juers M, Bone R C, Opal S M; Caring for the critically ill patient. High-dose antithrombin III in severe sepsis: a randomized controlled trial. JAMA. 2001 Oct 17; 286(15): 1869-78
38. T Krakauer Induction of C C chemokines in human peripheral blood mononuclear cells by staphylococcal exotoxins and its prevention by pentoxifylline J Leukoc Biol. 1999 July;

66(1): 158-64

39. Teresa Krakauer* and Bradley G. Stiles Pentoxifylline Inhibits Superantigen-Induced Toxic Shock and Cytokine Release Clin Diagn Lab Immunol. 1999 July; 6(4): 594-8.

TABLE 2
GENE CHANGES INDUCED BY BRUCELLA IN VITRO
IN HUMAN LYMPHOID CELLS

| Time chat | Function | Gene Name | 6 h | 24 h |
|---|---|---|---|---|
| A U | INTERLEUKINS | interferon gamma precursor (IFN-gamma; IFNG); immun | 11.681 | 44.32 |
| A U | INTERLEUKINS | interleukin-6 precursor (IL-6); B-cell stimulatory factor 2 (l | 5.372 | 6.72 |
| A U | GROWTH FACTORS, CYTO | macrophage inflammatory protein 1 alpha precursor (MIP | 3.998 | 4.39 |
| A U | GROWTH FACTORS, CYTO | placenta growth factors 1 + 2 (PLGF1 + PLGF2) | 3.983 | 3.85 |
| A U | GROWTH FACTOR & CHEM | corticotropin releasing factor receptor 1 precursor (CRF-F | 2.149 | 2.8 |
| A D | TRANSCRIPTION FACTORS | signal transducer and activator of transcription 6 (STAT6) | −1.389 | −2.5 |
| A D | PROTEIN TURNOVER | cathepsin D precursor (CTSD) | −3.769 | −3.125 |
| A D | HOMEOSTASIS & DETOXIFI27-kDa | heat-shock protein (HSP27); stress-responsive pr | −1.618 | −1.563 |
| 6 h U | TYROSINE KINASE RECEPTERBB-3 | receptor protein-tyrosine kinase precursor; epide | 3.119 | 1.74 |
| 6 h U | TRANSCRIPTION FACTOR-I | c-rel proto-oncogene protein | 2.990 | 0 |
| 6 h U | PROTEIN TURNOVER | leukocyte elastase inhibitor (LEI); monocyte/neutrophil el [symbol] | 2.355 | 0 |
| 6 h U | PROTEIN TURNOVER | placental plasminogen activator inhibitor 2 (PAI-2; PLANT | 2.170 | 0 |
| 6 h U | OTHER ONCOGENES | T-lymphoma invasion and metastasis inducing TIAM1 | 17.31 | 0 |
| 6 h U | NEUROTRANSMITTER SYN | tryptophan 5-hydroxytase (TRPH); tryptophan 5-monooxy | 14.59 | 0 |
| 6 h U | NERVOUS SYSTEM-RELATE | transcription factor AREB6 | 11.78 | 0 |
| 6 h U | NERVOUS SYSTEM-RELATE | basic transcription element-binding protein 2 (BTEB2); G [symbol] | 5.542 | 0 |
| 6 h U | NERVOUS SYSTEM-RELATE | interferon regulatory factor 7 (IRF-7) | 3.217 | 0 |
| 6 h U | NERVOUS SYSTEM-RELATE | hypoxia-inducible factor 1 alpha (HIF1 alpha): ARNT-inter | 2.801 | 0 |
| 6 h U | NERVOUS SYSTEM-RELATE | nuclear factor kappa-B DNA binding subunit (NF-kappaB; | 2.524 | 0 |
| 6 h U | NERVOUS SYSTEM-RELATE | transcription initiation factor TFIID 31-kDa subunit; TAFII; | 2.453 | 0 |
| 6 h U | KINASE SUBSTRATES & INE | 14-3-3 protein sigma: stratifin: epithelial cell marker prote | 10.246 | 0 |
| 6 h U | INTERLEUKINS | interleukin-1 alpha precursor (IL-1 alpha: IL1A); hematop [symbol] | 45.08 | 0 |
| 6 h U | INTERLEUKINS | interleukin-12 beta subunit precursor (IL-12B); cytotoxic I [symbol] | 14.9 | 0 |
| 6 h U | INTERLEUKINS | interleukin-10 precursor (IL-10): cytokine synthesis inhibit | 2.363 | 0 |
| 6 h U | INTERLEUKIN & INTERFER [symbol] | interleukin-5 receptor alpha subunit precursor (IL-5R-alph | 3.278 | 0 |
| 6 h U | HORMONES | erythroid differentiation protein (EDF); inhibin beta A sub | 30.18 | 0 |
| 6 h U | GROWTH FACTOR & CHEM | tumor necrosis factor-inducible protein TSG-6: hyaluronal | 10.086 | 0 |
| 6 h U | GROWTH FACTOR & CHEM | CDW40 antigen: CD40L receptor precursor: nerve growth | 2.954 | 0 |
| 6 h U | DNA POLYMERASES, REPL | DNA topoisomerase I (TOP 1) | 3.297 | 0 |
| 6 h U | DNA DAMAGE REPAIR PRO | nibrin (NBS1) | 2.839 | 0 |
| 6 h U | DEATH RECEPTORS | fasL receptor, apoptosis-mediating surface antigen fas; A | 5.710 | 0 |
| 6 h U | DEATH LIGANDS | TNF-related apoptosis inducing ligand (TRAIL): APO-2 lig | 3.797 | 0 |
| 6 h U | CYCLINS | G1/S-specific cyclin D2 (CCND2) + KIAK0002 | 7.645 | 0 |
| 6 h U | CELL SURFACE ANTIGENS | integrin beta 8 precursor (ITGB8) | 4.591 | 0 |
| 6 h U | BCL FAMILY | BCL-2-related protein A1 (BCL2A1); BFL1 protein: hemop | 24.361 | 0 |
| 6 h U | APOPTOSIS-ASSOCIATED I | IEX-1L anti-death protein: PRG-1; DIF-2 | 9.854 | 0 |
| 6 h U | APOPTOSIS-ASSOCIATED I | cytoplasmic antiproteinase 3 (CAP3): protease inhibitor 1 | 2.222 | 0 |
| 6 h M | TYROSINE PHOSPHATASES | protein-tyrosine phosphatase 1B (PTP-1B) | 1.736 | 0 |
| 6 h M | SIGNAL TRANSDUCTION RI | urokinase-type plasminogen activator receptor GPI-anch [symbol] | 1.954 | 0 |
| 6 h M | NON-RCEPTOR TYROSINE | C-fgr proto-oncogene (p55-FGR); SRC2 | 1.595 | 0 |
| 6 h M | NERVOUS SYSTEM-RELATE | CYCLIC-AMP-DEPENDENT TRANSCRIPTION FACTOR | −1.844 | 0 |
| 6 h M | NERVOUS SYSTEM-RELATE | BRCA1-associated ring domain protein | −1.809 | 0 |
| 6 h M | NERVOUS SYSTEM-RELATE | homeobox A1 protein (HOXA1): HOX1F | 1.551 | 0 |
| 6 h M | GROWTH FACTOR & CHEM | neuromedin B receptor (NMBR): neuromedin-B-preferring | 1.589 | 1.43 |
| 6 h M | G PROTEINS | ras-related protein RAB-7 | 1.924 | 0 |
| 6 h M | DNA DAMAGE REPAIR PRO | ubiquitin-conjugating enzyme E2 17-kDa (UBE2A): ubiqui | 1.696 | 1.020 |
| 6 h M | DISEASE-RELATED PROTE | major prion protein precursor (PRP); PRP27-30; PRP33- [symbol] | 1.569 | 0 |
| 6 h M | CELL SURFACE ANTIGENS | cadherin 3 (CDH3); placental cadherin precursor (P-cadh | 1.826 | 0 |
| 6 h D | TUMOR SUPPRESSORS & F | mothers against dpp homolog 4 (SMAD4): MADR4: pancl | −2.640 | 0 |
| 6 h D | RECEPTOR-ASSOCIATED F | ink adaptor protein | −2.146 | 0 |
| 6 h D | RECEPTOR-ASSOCIATED F | proto-oncogene tyrosine-protein kinase Ick; p56-Ick; lymp | −2.160 | 0 |
| 6 h D | PROTEIN TURNOVER | cathepsin L precursor, major excreted protein (MEP) | −1.313 | 0 |
| 6 h D | PROTEIN TURNOVER | proteasome inhibitor HPI31 subunit | −1.962 | 0 |
| 6 h D | PROTEIN TURNOVER | metalloproteinase inhibitor 1 precursor (TIMP1); erythroid | −2.386 | 0 |
| 6 h D | PROTEIN TURNOVER | proteasome component C5; macropain subunit C5; prote | −2.513 | 1.96 |
| 6 h D | OTHER ONCOGENES | matrix metalloproteinase 11 (MMP11); stromelysin 3 | −1.878 | 0 |
| 6 h D | OTHER CELL CYCLE REGU | CDC25B; CDC25HU2; M-phase inducer phosphatase 2 | −1.731 | 0 |
| 6 h D | NERVOUS SYSTEM-RELATEZFM1 | protein alternatively spliced product | −2.086 | 0 |
| 6 h D | NERVOUS SYSTEM-RELATE | early growth response protein 1 (hEGR1); transcription fa | −2.393 | 0 |
| 6 h D | NERVOUS SYSTEM-RELATE | GA-binding protein beta-2 subunit (GABP-beta2); transcri | −12 | 0 |
| 6 h D | INTRACELLULAR KINASE N | ribosomal protein S6 kinase II alpha 1 (S6KII-alpha 1); rit | −1.909 | 0 |
| 6 h D | HOMEOSTASIS & DETOXIFI | glutathione peroxidase (GSHPX1; GPX1) | −3.063 | 0 |
| 6 h D | HOMEOSTASIS & DETOXIFI | cytosolic superoxide dismutase 1 (SOD1) | −16.02 | 0 |
| 6 h D | GROWTH FACTORS, CYTO | teratocarcinoma-derived growth factor 1 (TDGF1): epiden | −1.664 | 0 |
| 6 h D | GROWTH FACTORS, CYTO | endothelin 2 (ET2) | −15.25 | 0 |
| 6 h D | GROWTH FACTOR & CHEM | C5a anaphylatoxin receptor (C5AR); CD88 antigen | −2.137 | 0 |
| 6 h D | G PROTEINS | ras-related protein RAB2 | −1.572 | 0 |
| 6 h D | G PROTEINS | ras-related protein RAP-1B; GTP-binding protein SMG p2 | −1.642 | 0 |
| 6 h D | G PROTEINS | ras-related C3 botulinum toxin substrate 1; p21-rac1; ras- | −2.060 | 0 |
| 6 h D | G PROTEINS | guanine nucleotide regulatory protein alpha-13 subunit; G | −2.411 | 0 |
| 6 h D | DNA-BINDING & CHROMATI | high mobility group protein (HMG-l) | −2.517 | 0 |
| 6 h D | DEATH RECEPTORS | CD27L antigen receptor precursor, T-cell activation CD27 | −4.837 | 0 |
| 6 h D | DEATH RECEPTOR-ASSOC | CD4O receptor-associated factor 1 (CRAF1) | −3.291 | 0 |
| 6 h D | APOPTOSIS-ASSOCIATED I | ALG-2 calcium-binding protein | −2.671 | 0 |
| 6 h D | APOPTOSIS-ASSOCIATED I | SL cytokine precursor, FLT3 ligand (FLT3LG) | −3.144 | 0 |
| 24 h U | INTERLEUKINS | interleukin-1 beta precursor (IL-1; IL1B); catabolin | 1.554 | 6.95 |
| 24 h U | INTERLEUKIN & INTERFER [symbol] | interleukin-2 receptor alpha subunit precursor (IL-2 recept | 1.240 | 2.13 |
| 24 h U | GROWTH FACTORS, CYTO | macrophage inflammatory protein 2 alpha (MIP2-alpha); [symbol] | 6.183 | 27.9 |
| 24 h U | GROWTH FACTORS, CYTO | macrophage inflammatory protein 1 beta precursor (MIP1 | 2.128 | 11.22 |
| 24 h U | GROWTH FACTORS, CYTO | interleukin-8 precursor (IL-8); monocyte-derived neutroph | 1.762 | 8.26 |
| 24 h U | GROWTH FACTORS, CYTO | cytokine humig; interferon-gamma-induced monokine (MI | 1.448 | 17.72 |
| 24 h U | GROWTH FACTORS, CYTO | interferon gamma-induced protein precursor (gamma-IP1 | 1.325 | 6655 |
| 24 h U | G PROTEINS | guanine nucleotide-binding protein G(I)/G(S)/G(T) beta s [symbol] | 1.976 | 6.71 |
| 24 h U | DEATH LIGANDS | tumor necrosis factor precursor (TNF-alpha: TNFA); cach | 1.440 | 2.89 |
| 24 h U | APOPTOSIS-ASSOCIATED I | interferon-inducible RNA-dependent protein kinase (P68 k | 2.167 | 9.32 |
| 24 h D | TYROSINE PHOSPHATASE [symbol] | serine/threonine protein phosphatase PP1-alpha 1 catalyt | 1.049 | −3.571 |
| 24 h D | TYROSINE KINASE RECEPT | macrophage colony stimulating factor 1 receptor precurso | −3.323 | −25 |
| 24 h D | TUMOR SUPPRESSORS & F | c-myc purine-binding transcription factor puf; nucleoside | 1.080 | −5.263 |
| 24 h D | TRANSCRIPTION FACTORS | cAMP-response element binding protein (CREB) | 1.126 | −8.333 |
| 24 h D | TRANSCRIPTION FACTORS | interferon regulatory factor 1 (IRF1) | −1.085 | −2.041 |
| 24 h D | TRANSCRIPTION FACTOR-I | c-jun proto-oncogene; transcription factor AP-1 | 1.611 | −2.083 |
| 24 h D | TRANSCRIPTION FACTOR-I | ets-related protein tel; ets translocation variant 6 (ETV6) | 1.421 | −3.704 |
| 24 h D | SIGNAL TRANSDUCTION RI | thrombin receptor (TR); F2R; PAR1 | 1.025 | −3.846 |
| 24 h D | SIGNAL TRANSDUCTION RI | stromal cell derived factor 1 receptor (SDF1 receptor); fus | −1.062 | −2.564 |
| 24 h D | SIGNAL TRANSDUCTION RI | ephrin type-A receptor 1 precursor, tyrosine-protein kinas [symbol] | −1.296 | −2.381 |
| 24 h D | RECEPTOR-ASSOCIATED F | growth factor receptor-bound protein 2 (GRB2) isoform; C [symbol] | 1.175 | −2.273 |
| 24 h D | NERVOUS SYSTEM-RELATE | putative transcription activator DB1 | 1.043 | −2.174 |
| 24 h D | NERVOUS SYSTEM-RELATE | guanine nucleotide-binding protein G-s alpha subunit (G [symbol] | −1.227 | −2.941 |
| 24 h D | KINASE SUBSTRATES & INI | hint protein; protein kinase C Inhibitor 1 (PKCI1) | 1.246 | −3.571 |
| 24 h D | KINASE SUBSTRATES & INI | protein kinase C substrate 80-kDa-protein heavy chain (P | 1.089 | −2.941 |
| 24 h D | KINASE SUBSTRATES & INI | 14-3-3 protein beta/alpha; protein kinase C Inhibitor prote | −1.037 | −2.857 |
| 24 h D | HOMEOSTASIS & DETOXIFI | dioxin-inducible cytochrome P450 1B1 (CYP 1B1) | −1.221 | −4.348 |
| 24 h D | GROWTH FACTORS, CYTO | migration inhibitory factor-related protein 14 (MRP14-); ca | −1.165 | −1.031 |
| 24 h D | GROWTH FACTORS, CYTO | migration inhibitory factor-related protein 8 (MRP8); calgr | −1.289 | −1.667 |
| 24 h D | GDP/GTP EXCHANGERS & | rho GDP dissociation inhibitor 1 (RHO-GDI 1): RHO-GDI | 1.150 | −2.083 |
| 24 h D | DNA DAMAGE REPAIR PRO | mutL protein homolog; DNA mismatch repair protein ML [symbol] | −1.235 | −1.563 |
| 24 h D | CELL CYLCLE REGULATIN [symbol] | CDC-like kinase 1 (CLK1) | 1.130 | −2.5 |
| 24 h D | CALPAINS | calcium-dependent protease small (regulatory) subunit; c: | −1.253 | −1.613 |
| 24 h D | ADENYLYL/GUANYLYL CYC | cGMP-inhibited 3′,5′-cyclic phosphodiesterase B (CGI-PD | −1.139 | −3.333 |

AU = upregulated at all time points;
AD = downregulated at all time points:
U = upregulated;
M = moderately upregulated
D = downregulated

TABLE 3
GENE CHANGES INDUCED BY PLAGUE IN
VITRO IN HUMAN LYMHPOID CELLS

| | | FOLD CHANGE | | | |
|---|---|---|---|---|---|
| Function | Protein/gene | 1 hr | 2 hr | 4 hr | 8 hr |
| Suppressors | vascular endothelial growth factor receptor 1 (VEGF [symbol] | 5.50 | 1.05 | 4.52 | 1.79 |
| Suppressors | tyrosine-protein kinase ABL2; tyrosine kinase ARG (A [symbol] | 9.72 | 1.29 | 19.83 | 1.42 |
| Suppressors | c-jun proto-oncogene; transcription factor AP-1 | 1.37 | 1.04 | 1.32 | 2.25 |
| Suppressors | myb proto-oncogene; c-myb | 9.02 | 13.57 | 38.08 | 1.80 |
| Suppressors | ERBB-3 receptor protein-tyrosine kinase precursor, e [symbol] | 1.57 | 1.54 | 3.02 | 1.75 |
| G Proteins | ras-related protein RAP-1B; GTP-binding protein SM [symbol] | 1.19 | 3.86 | 1.06 | 1.38 |
| Death Receptor Ligands | tumor necrosis factor precursor (TNF-alpha; TNFA); [symbol] | 2.16 | 7.69 | 3.64 | 6.06 |
| Repressors | helix-loop-helix protein; DNA-binding protein inhibitor | 2.00 | 1.54 | 4.43 | 2.03 |
| Cell Surface Antigens | tumor necrosis factor-inducible protein TSG-6; hyalur [symbol] | 31.63 | 1.92 | 5.96 | 1.10 |
| Cytokines & Chemokines | macrophage inflammatory protein 1 alpha precursor [symbol] | 2.99 | 2.46 | 1.57 | 2.54 |
| Interleukins & Interferons | Interleukin-6 precursor (IL-6); B-cell stimulatory facto [symbol] | 5.31 | 62.12 | 9.19 | 4.07 |
| Hormones | corticotropin-releasing factor-binding protein | 3.72 | 3.71 | 3.77 | 1.00 |
| Suppressors | ezrin; cytovillin 2; villin 2 (VIL2) | −2.20 | −1.33 | −2.32 | −1.05 |
| Cyclins | cyclin H (CCNH); MO15-associated protein | −2.08 | −7.81 | −2.02 | −1.11 |
| Ribosomal Proteins | fte-1; yeast mitochondrial protein import homolog; 40 | −1.34 | −1.89 | −4.17 | −1.16 |
| ATPase Transporters | sodium/potassium-transporting ATPase alpha 1 subu [symbol] | −1.37 | −3.11 | −10.36 | −1.06 |
| Effectors & Modulators | stromal cell derived factor 1 receptor (SDF1 receptor [symbol] | −13.56 | −6.65 | −3.22 | −1.24 |
| Network Members | cAMP-dependent protein kinase I alpha regulatory su [symbol] | −1.42 | −2.89 | −19.77 | −1.39 |
| Inhibitors | 14-3-3 protein beta/alpha; protein kinase C inhibitor p [symbol] | −1.34 | −1.98 | −5.23 | −1.38 |
| Proteins & Ligases | DNA excision repair protein ERCC1 | −9.50 | −3.90 | −8.30 | −1.10 |
| Inhibitors | protein kinase C substrate 80-kDa protein heavy chai [symbol] | −1.69 | −6.89 | −14.74 | −1.29 |
| Calpains | calcium-dependent protease small (regulatory) subun [symbol] | −1.06 | −2.63 | −19.78 | −1.68 |
| Associated Proteins | cytoplasmic dynein light chain 1 (HDLC1); protein init [symbol] | −1.08 | −9.45 | −12.22 | −1.41 |
| Caspases | caspase-4 precursor (CASP4); ICH-2 protease; TX pr [symbol] | −1.10 | −1.79 | −35.00 | −1.28 |
| Proteins & Ligases | mutL protein homolog; DNA mismatch repair protein | −1.77 | −1.96 | −11.42 | −1.71 |
| Phosphatases | serine/threonine protein phosphatase 2B catalytic sut [symbol] | −5.35 | −4.20 | −10.65 | −1.84 |
| Associated Proteins | ALG-2 calcium-binding protein | −7.13 | −1.71 | −1.73 | −1.61 |
| Calpains | calpain 2 large (catalytic) subunit; M-type calcium-act [symbol] | −19.57 | −1.67 | −2.61 | −1.10 |
| Repressors | ets domain protein elk-3; NET; SRF accessory protei [symbol] | −19.80 | 3.58 | −4.17 | −1.86 |
| Repressors | putative transcription activator DB1 | −3.66 | −1.03 | −4.21 | −1.19 |
| Receptors | leukocyte adhesion glycoprotein p150, 95 alpha subu [symbol] | −1.56 | −6.49 | −7.53 | −1.35 |
| Chromatin Proteins | high mobility group protein (HMG-I) | −1.03 | −5.60 | −26.06 | −1.06 |
| Repressors | 26S protease regulatory subunit 6A; TAT-binding pro [symbol] | −2.01 | −5.72 | −5.09 | −1.28 |
| Repressors | nucleobindin precursor (NUC) | −14.07 | −5.95 | −3.20 | −1.07 |
| Receptors | alpha1 catenin (CTNNA1); cadherin-associated protei [symbol] | −1.85 | −2.23 | −2.40 | −1.37 |
| Repressors | transcription factor LSF | −6.20 | −3.20 | −8.02 | −1.47 |
| Cytokines & Chemokines | thrombomodulin precursor (THBD; ThRM); fetomodu [symbol] | −13.75 | −22.44 | −5.30 | −1.17 |
| Cytokines & Chemokines | vascular endothelial growth factor precursor (VEGF); | −1.24 | −1.03 | −2.26 | −1.11 |
| Cytokines & Chemokines | migration inhibitory factor-related protein 14 (MRP14) | −1.16 | −1.44 | −9.04 | −2.54 |
| Protease Inhibitors | metalloproteinase inhibitor 1 precursor (TIMP1); eryt [symbol] | −1.03 | −1.68 | −14.50 | −1.26 |
| Cytokines & Chemokines | migration inhibitory factor-related protein 8 (MRP8); [symbol] | −1.12 | −1.50 | −3.05 | −2.80 |
| Xenobiotic Metabolism | dioxin-inducible cytochrome P450 1B1 (CYP1B1) | −1.30 | −2.63 | −26.39 | −3.66 |
| Cysteine Proteases | cathepsin L precursor; major excreted protein (MEP) | −18.50 | −7.01 | −6.00 | −3.11 |
| Suppressors | transmembrane 4 superfamily protein; SAS | −6.14 | 1.06 | 12.63 | 1.84 |
| Cyclases & Diesterases | bane marrow stromal antigen 1 (BST-1); ADP-ribosyl [symbol] | −4.91 | 6.33 | 4.77 | 1.23 |
| Extracellular | acyl-CoA-binding protein (ACBP); diazepam binding i [symbol] | 1.31 | 1.50 | 3.37 | −1.04 |
| Factors | ZFM1 protein alternatively spliced product | −7.66 | 1.23 | 4.63 | 1.31 |
| Chemokine Receptors | neuromedin B receptor (NMBR); neuromedin-B-prefe [symbol] | 1.31 | 2.16 | 4.23 | −1.11 |
| Receptors | CD44 antigen hematopoietic form precursor (CD44H) | 1.29 | 1.14 | 1.49 | 2.21 |
| Receptors | Integrin beta 8 precursor (ITGB8) | 1.47 | 1.65 | 5.52 | 1.28 |
| Interleukins & Interferons | interleukin-10 precursor (IL-10); cytokine synthesis in [symbol] | −5.39 | 2.27 | 7.18 | 1.17 |
| Communication Proteins | B94 protein | 2.74 | 2.37 | 58.06 | 1.32 |
| Heat Shock Proteins | 70-kDa heat shock protein 1 (HSP70.1; HSPA1) | 2.41 | 1.13 | 1.01 | −4.17 |
| Cytokines & Chemokines | macrophage inflammatory protein 2 alpha (MIP2-alp [symbol] | 1.68 | 1.21 | 1.61 | 8.04 |
| Interleukins & Interferons | interleukin-1 beta precursor (IL-1; IL1B); catabolin | 1.61 | 1.44 | −2.58 | 1.00 |
| Cytokines & Chemokines | placenta growth factors 1 + 2 (PLGF1 + PLGF2) | 1.34 | 1.08 | 1.04 | 10.02 |
| Kinases | stem cell tyrosine kinase 1 (STK1); FL cytokine recep [symbol] | 3.20 | 0.00 | 2.91 | 2.54 |
| CDK Inhibitors | cyclin-dependent kinase 4 inhibitor (CDK4I; CDKN2); | 0.00 | 1.40 | 12.46 | 1.98 |
| Suppressors | C-src proto-oncogene (SRC1) | 2.55 | 0.00 | 2.43 | 1.12 |
| Suppressors | c-rel proto-oncogene protein | 4.64 | 1.02 | 0.00 | 2.84 |
| Channels & Transporters | T-lymphocyte maturation-associated protein MAL | 1.30 | 0.00 | 9.19 | 3.74 |
| Death Receptor Ligands | lymphotoxin-alpha precursor (LT-alpha); tumor necro [symbol] | 0.00 | 6.50 | 6.57 | 1.53 |
| Bcl Family Proteins | BCL-2-related protein A1 (BCL2A1); BFL1 protein; h [symbol] | 1.06 | 8.61 | 0.00 | 1.57 |
| Repressors | microphthalmia-associated transcription factor (MITF [symbol] | 16.47 | 2.30 | 0.00 | 3.15 |
| Xenobiotic Transporters | glutathione reductase (GRase; GSR; GR) | 0.00 | 9.50 | 14.52 | 1.21 |
| Cytokines & Chemokines | macrophage inflammatory protein 1 beta precursor ( [symbol] | 5.86 | 27.09 | 0.00 | 2.82 |
| Interleukins & Interferons | interleukin-12 beta subunit precursor (IL-12B); cytoto [symbol] | 0.00 | 2.72 | 3.66 | 1.50 |
| Cytokines & Chemokines | granulocyte-macrophage colony stimulating factor (G | 0.00 | 12.20 | 3.75 | 1.59 |
| Interleukins & Interferons | interleukin-1 alpha precursor (IL-1 alpha; IL1A); hem [symbol] | 3.06 | 4.22 | 0.00 | 4.99 |
| Suppressors | ets-related protein tel; ets translocation variant 6 (ET [symbol] | 1.86 | −1.80 | −2.60 | −1.16 |
| Network Members | protein kinase C delta (NPKC-delta) | 1.15 | 1.03 | −22.27 | −1.26 |
| Receptor-Associated | Ink adaptor protein | −1.37 | −3.04 | −16.97 | 1.44 |
| Network Members | MAP kinase-activated protein kinase 2 (MAPKAP kin [symbol] | 1.17 | −1.08 | −29.09 | −1.18 |
| Network Members | dual specificity mitogen-activated protein kinase kina [symbol] | 1.67 | −1.12 | −17.36 | −1.36 |
| Effectors & Modulators | ephrin type-A receptor 1 precursor; tyrosine-protein ki [symbol] | −1.29 | −2.64 | −14.01 | 1.13 |
| Receptor-Associated | growth factor receptor-bound protein 2 (GRB2) isofon [symbol] | 1.16 | −1.27 | −50.48 | −1.74 |
| Phosphatases | PTPCAAX1 nuclear tyrosine phosphatase (PRL-1) | −1.24 | −1.46 | −11.60 | 1.04 |
| Associated Proteins | defender against cell death 1 (DAD1) | −1.08 | −2.25 | −5.06 | 1.02 |
| Death Receptors | tumor necrosis factor receptor 1 (TNFR1); tumor nec [symbol] | −5.38 | −5.23 | −6.75 | 1.46 |
| Death Receptors | tumor necrosis factor receptor (TNFR) + tumor necr [symbol] | 1.17 | −1.62 | −7.45 | −2.02 |
| GTPase Activity | rho GDP dissociation inihibitor 1 (RHO-GDI 1); RHO- [symbol] | 1.10 | −1.05 | −4.13 | −2.79 |
| Bcl Family Proteins | apoptosis regulator bcl-x | −15.05 | −1.93 | −3.34 | 1.01 |
| Bcl Family Proteins | induced myeloid leukemia cell differentiation protein [symbol] | −1.53 | −1.90 | −37.83 | 1.02 |
| Phosphatases | serine/threonine protein phosphatase PP1-alpha 1 ca [symbol] | −1.45 | −4.75 | −3.87 | 1.77 |
| Repressors | TIS11B protein; EGF response factor 1 (ERF1) | −1.08 | −1.13 | −28.57 | 4.02 |
| Factors | hypoxia-inducible factor 1 alpha (HIF1 alpha); ARNT- [symbol] | −27.83 | −2.54 | −1.53 | −1.35 |
| Receptors | beta catenin (CTNNB) | −3.90 | −4.94 | 1.43 | 1.28 |
| Receptors | cadherin 3 (CDH3); placental cadherin precursor (P-c [symbol] | 0.00 | −4.12 | −2.31 | −1.75 |
| Chemokine Receptors | C5a anaphylatoxin receptor (C5AR); CD88 antigen | −3.89 | −6.91 | −4.41 | 1.54 |
| RNA Polymerase | transcription Initiation factor TFIID 31-kDa subunit; T [symbol] | −5.23 | 1.09 | −1.70 | 1.25 |
| Repressors | heat shock factor protein 1 (HSF1); heat shock trans [symbol] | −3.38 | −5.09 | −5.83 | 1.04 |
| Activities) | DNA-binding protein A | −1.14 | −1.91 | 2.97 | −1.59 |
| Factors | transcriptional regulator interferon-stimulated gene fa [symbol] | −2.64 | −1.12 | 1.99 | −2.10 |
| Repressors | zinc finger protein 91 (ZNF92); HPF7; HTF10 | −3.61 | −1.07 | 2.23 | 1.19 |
| CDK Inhibitors | special AT-rich sequence binding protein 1 (SATB1); | −10.47 | −2.07 | −2.38 | 2.38 |
| Repressors | transcription factor Sp1 (TSFP1) | −5.68 | −1.32 | −13.27 | 1.48 |
| Receptors | fibronectin receptor beta subunit (FNRB); integrin bet [symbol] | −1.31 | −3.14 | −8.50 | 1.13 |
| Factors | transcription factor ETR101 | −1.35 | −7.77 | −6.03 | 1.16 |
| Repressors | tristetraproline (TTP); TIS11; ZFP36; growth factor-in | 1.26 | −1.25 | −2.45 | −1.42 |
| Xenobiotic Transporters | microsomal glutathione S-transferase 12 (GST12; M [symbol] | −1.64 | −1.08 | −1.04 | −1.07 |
| Receptors | vitronectin receptor alpha subunit (VNRA); integrin al [symbol] | −2.12 | −1.42 | −5.36 | −2.27 |
| Xenobiotic Transporters | glutathione S-transferase pi (GSTP1; GST3) | −1.36 | −1.24 | 1.02 | −1.41 |
| Factors | CCAAT-binding transcription factor subunit B (CBF-B | −2.02 | −1.50 | −22.65 | −1.34 |
| Receptors | leukocyte adhesion glycoprotein LFA-1 alpha subunit | −1.44 | −1.72 | −1.66 | 1.17 |
| Receptors | interleukin-7 receptor alpha subunit precursor (IL-7R- [symbol] | −1.61 | −1.95 | 11.21 | −2.35 |
| Cytokines & Chemokines | endothelial-monocyte activating polypeptide II (EMA [symbol] | −7.55 | −1.22 | −5.53 | −1.31 |
| Protease Inhibitors | alpha-1-antitrypsin precursor; alpha-1 protease inhibi [symbol] | 1.14 | −1.53 | −10.10 | −2.13 |
| Cytokines & Chemokines | heparin-binding EGF-like growth factor (HBEGF); dip | −25.21 | −9.29 | −3.80 | 1.05 |
| Cytokines & Chemokines | T-cell-specific rantes protein precursor; sis delta; sm [symbol] | −1.23 | −2.20 | −41.42 | 1.06 |
| Cytokines & Chemokines | thymosin beta-10 (TMSB10; THYB10); PTMB10 | 1.21 | −1.64 | −2.25 | −3.17 |
| Communication Proteins | thymosin beta 4; FX | 1.34 | −1.73 | −36.03 | −1.73 |
| Protease Inhibitors | cytoplasmic antiproteinase 2 (CAP2); protease inhibit [symbol] | −2.21 | −4.47 | −7.47 | 1.31 |
| Proteins | glutathione S-transferase A1 (GTH1; GSTA1); HA su [symbol] | −3.40 | −1.02 | −7.33 | 2.20 |
| Suppressors | MAD protein; MAX dimerizer | −2.92 | −1.98 | 0.00 | −1.26 |
| Suppressors | macrophage colony stimulating factor I receptor prec [symbol] | −8.81 | −10.95 | 0.00 | −1.13 |
| G Proteins | ras-related protein RAB2 | −3.76 | −1.11 | −4.60 | −1.27 |
| Effectors & Modulators | ephrin A receptor 4 precursor; tyrosine-protein kinase | −16.93 | −2.55 | 0.00 | −1.83 |
| Inhibitors | macMARCKS; MARCKS-related protein (MRP); MLP | −9.56 | −5.64 | 0.00 | −1.28 |
| Proteins & Ligases | xeroderma pigmentosum group C repair complement | −2.85 | −2.85 | 0.00 | −1.14 |
| Transducers, Effectors & | leucine-rich repeat protein SHOC-2; ras-binding prot [symbol] | −2.87 | 0.00 | −11.66 | −1.30 |
| Phosphatases | protein phosphatase 2C alpha isoform (PP2C-alpha) | −5.26 | 0.00 | −1.17 | 1.12 |
| Receptors | cell surface adhesion glycoproteins LFA-1/CR3/p150, | −13.23 | −2.86 | 0.00 | −1.24 |
| Receptors | interleukin-4 receptor alpha subunit precursor (IL-4R- [symbol] | −1.86 | −2.60 | 0.00 | −1.88 |
| Receptors | fibronectin receptor alpha subunit (FNRA); integrin al [symbol] | −6.59 | −1.93 | 0.00 | −1.51 |
| Chemokine Receptors | N-sam; fibroblast growth factor receptor1 precursor ( [symbol] | −9.14 | −3.13 | 0.00 | −2.05 |
| Metalloproteinases | matrix metalloproteinase 9 (MMP9); gelatinase B; 92 [symbol] | 0.00 | −2.29 | −3.19 | −1.22 |
| Aspartic Proteases | cathepsin D precursor (CTSD) | −15.96 | −2.86 | 0.00 | −1.86 |
| CDK Inhibitors | cyclin-dependent kinase inhibitor 1 (CDKN1A); melan [symbol] | 1.21 | −1.61 | 1.16 | −2.26 |
| Suppressors | c-myc purine-binding transcription factor puf; nucleos [symbol] | 1.24 | −5.92 | −5.14 | 1.08 |
| Suppressors | C-fgr proto-oncogene (p55-FGR); SRC2 | 1.44 | −1.09 | 1.17 | −22.46 |
| Cyclins | cyclin K | 1.43 | −1.40 | −6.70 | 1.99 |
| Effectors & Modulators | urokinase-type plasminogen activator receptor GPI-a [symbol] | 1.68 | −1.51 | −10.75 | 2.21 |
| Effectors & Modulators | CC chemokine receptor type 1 (CC CKR1; CCR 1): [symbol] | −2.26 | −1.76 | 5.07 | 1.49 |
| G Proteins | Gem; Induced Immediate early protein; ras family me [symbol] | −6.17 | 1.89 | 4.28 | −1.22 |
| G Proteins | ras-related protein RAB5A | −11.45 | 3.13 | −1.32 | 1.87 |
| G Proteins | ras-related protein RAB-7 | −1.01 | 1.16 | −4.62 | 1.19 |
| Repressors | signal transducer and activator of transcription 6 (ST [symbol] | −2.07 | 1.13 | −4.47 | 1.16 |
| Associated Proteins & | FAN protein | 2.38 | 2.11 | −4.24 | −1.41 |
| Associated Proteins | cytoplasmic antiproteinase 3 (CAP3); protease inhibit [symbol] | 1.39 | −1.62 | −4.26 | 1.04 |
| Associated Proteins | IEX-1L anti-death protein; PRG-1; DIF-2 | 1.66 | −1.29 | −19.07 | 1.76 |
| Associated Proteins | growth arrest & DNA-damage-Inducible protein 153 ( [symbol] | −1.40 | 1.10 | −14.51 | 1.63 |
| Factors | 60S ribosomal protein L6 (RPL6); TAX-responsive en [symbol] | 1.06 | −1.24 | 2.63 | −1.25 |
| Factors | CCAAT transcription binding factor gamma subunit | −15.95 | 1.13 | −1.63 | 1.97 |
| Proteins & Ligases | HHR23A; UV excision repair protein protein RAD23A | 8.88 | −1.61 | −7.04 | 1.80 |
| Proteins & Ligases | ubiquitin-conjugating enzyme E2 17-kDa (UBE2A); u [symbol] | 1.09 | −1.61 | −1.42 | 1.54 |
| Repressors | nuclear factor NF-kappa-B p100 subunit; nuclear fact [symbol] | 1.32 | −1.47 | 49.94 | −1.05 |
| Extracellular | glia maturation factor beta (GMF-beta) | −18.82 | 1.38 | −10.79 | 1.53 |
| Repressors | Ini1 | −2.59 | −4.91 | 1.51 | 2.07 |
| Repressors | C-ets-2 | −1.37 | −2.19 | 1.79 | 1.04 |
| Factors | paired box protein PAX-5; B-cell specific transcription | −6.10 | −1.38 | 1.30 | 2.07 |
| Repressors | early growth response protein 1 (hEGR1); transcriptio [symbol] | −1.33 | 1.30 | −3.20 | 3.53 |
| CDK Inhibitors | Sp2 protein | −11.06 | 1.23 | −1.66 | 1.03 |
| Factors | transcriptional repressor protein yin & yang 1 (YY1); [symbol] | −3.36 | −1.04 | −16.36 | 1.01 |
| Heat Shock Proteins | mitochondrial matrix protein P1 precursor; p60 lymph [symbol] | −3.85 | −1.20 | −1.23 | 1.33 |
| Protease Inhibitors | leukocyte elastase inhibitor (LEI); monocyte/neutroph [symbol] | 1.07 | 2.53 | −5.50 | −2.24 |
| Nucleotide Metabolism | thioredoxin reductase | 4.54 | −2.51 | −6.46 | 1.18 |
| Metalloproteinases | matrix metalloproteinase 14 precursor (MMP14); me [symbol] | 9.81 | −1.23 | −12.74 | 1.09 |
| Cytokines & Chemokines | granulocyte chemotactic protein 2 (GCP 2); neutrophi [symbol] | 1.01 | −1.56 | −14.53 | 3.71 |
| Effectors & Modulators | interferon-gamma (IFN-gamma) receptor beta subuni [symbol] | −3.66 | 1.00 | 0.00 | 1.39 |
| Associated Proteins & | CD40 receptor-associated factor 1 (CRAF1) | 0.00 | 2.39 | −8.49 | 1.01 |
| Repressors | cAMP-response element binding protein (CREB) | 0.00 | 2.31 | 1.74 | −1.12 |
| Associated Proteins | cytochrome P450 reductase | −3.95 | 2.38 | 0.00 | 1.00 |
| Death Receptors | adenosine A1 receptor (ADORA1) | 0.00 | 10.21 | 46.16 | −1.45 |
| Chemokine Receptors | CDW40 antigen; CD40L receptor precursor; nerve g [symbol] | 0.00 | 3.29 | 24.52 | −2.74 |
| Chemokine Receptors | corticotropin releasing factor receptor 1 precursor (C [symbol] | 0.00 | 1.11 | 1.68 | −1.20 |
| Factors | DNA-binding protein HIP116; ATPase; SNF2/SWI2-r [symbol] | 0.00 | 2.28 | −1.58 | 1.83 |
| Cytokines & Chemokines | insulin-like growth factor binding protein 1 (IGFBP1); | 0.00 | 1.30 | 4.08 | −1.17 |
| Xenobiotic Transporters | glutathione peroxidase (GSHPX1; GPX1) | 1.45 | −12.93 | 0.00 | 1.10 |
| Hormones | erythroid differentiation protein (EDF); inhibin beta A | −3.60 | 1.08 | 0.00 | 1.82 |
| Hormones | renin-binding protein (RENBP; RNBP) | −12.36 | 1.60 | 0.00 | 1.17 |
| Cytokines & Chemokines | endothelin 2 (ET2) | 3.22 | 1.23 | 0.00 | −3.88 |
| Carboxypeptidases | methionine aminopeptidase 2 (METAP2); peptidase [symbol] | −5.22 | 5.38 | 0.00 | 1.15 |
| Activities) | zinc finger X-chromosomal protein (ZFX) | 2.09 | 4.22 | 0.00 | −1.17 |
| Network Members | calcium/calmodulin-dependent protein kinase I (CAM | 0.00 | 4.48 | 0.00 | 1.63 |
| Effectors & Modulators | serine/threonine-protein kinase receptor R4 precurso [symbol] | 20.70 | 0.00 | 0.00 | 2.34 |
| Death Receptor Ligands | fas antigen ligand (FASL); apoptosis antigen ligand ( [symbol] | 0.00 | 2.44 | 0.00 | 1.12 |
| Proteins & Ligases | nibrin (NBS1) | 0.00 | 4.44 | 0.00 | 1.04 |
| Death Receptors | adenosine A2A receptor (ADORA2A) | 0.00 | 4.42 | 0.00 | 1.14 |
| Extracellular | neurotrophin-4 (NT-4) | 0.00 | 4.18 | 0.00 | 1.75 |
| Extracellular | P2X purinoceptor 5 (P2X5) | 0.00 | 0.00 | 3.13 | 1.13 |
| Repressors | B-cell lymphoma 6 protein (bcl-6); zinc finger protein | 0.00 | 1.43 | 1.19 | 2.24 |
| Repressors | nuclear factor kappa-B DNA binding subunit (NF-kap [symbol] | 0.00 | 1.41 | 0.00 | 2.58 |
| Xenobiotic Transporters | selenium-binding protein | 0.00 | 2.44 | 0.00 | 1.52 |
| Receptors | Integrin alpha 7B precursor (IGA7B) | 1.41 | 0.00 | 0.00 | 2.66 |
| Cytokines & Chemokines | eosinophil granule major basic protein precursor (MB [symbol] | 0.00 | 5.35 | 0.00 | 1.52 |
| Cytokines & Chemokines | hepatocyte growth factor (HGF); scatter factor (SF); [symbol] | 0.00 | 4.03 | 0.00 | 1.11 |
| Interleukins & Interferons | interleukin-2 precursor (IL-2); T-cell growth factor (TC [symbol] | 0.00 | 5.83 | 0.00 | 1.11 |
| Hormones | inhibin alpha subunit precursor (INHA) | 0.00 | 5.75 | 0.00 | 1.00 |
| Interleukins & Interferons | interleukin-3 precursor (IL-3); multipotential colony-sti [symbol] | 0.00 | 5.84 | 0.00 | 1.00 |
| Cytokines & Chemokines | hepatocyte growth factor-like protein; macrophage-sti [symbol] | 4.88 | 0.00 | 0.00 | 1.00 |
| Communication Proteins | parathymosin | 0.00 | 2.95 | 0.00 | 1.07 |
| Suppressors | B-raf proto-oncogene (RAFB1) | −3.42 | −1.45 | 0.00 | −1.04 |
| Suppressors | p78 putative serine/threonine-protein kinase | −11.15 | 0.00 | −1.06 | 1.23 |
| Kinases | cyclin G-associated kinase (GAK) | −4.78 | −3.15 | 0.00 | 1.05 |
| Proteins | brain glucose transporter 3 (GTR3) | −6.70 | −9.77 | 0.00 | 1.05 |
| Proteins | E16 amino acid transporter | −1.26 | −2.20 | 0.00 | 1.51 |
| Network Members | ribosomal protein S6 kinase II alpha 1 (S6KII-alpha 1 [symbol] | 0.00 | −8.74 | −5.95 | 1.45 |
| Network Members | mitogen-activated protein kinase p38 (MAP kinase p [symbol] | 0.00 | −1.47 | −27.89 | 1.25 |
| G Proteins | Ral A; GTP-binding protein | −15.11 | −1.07 | 0.00 | −1.53 |
| Symporters & Antiporters | sodium- & chloride-dependent taurine transporter | −4.61 | −5.43 | 0.00 | 1.14 |
| Network Members | dual specificity mitogen-activated protein kinase kina [symbol] | 0.00 | −6.27 | −1.43 | 1.02 |
| Symporters & Antiporters | neutral amino acid transporter A (SATT); alanine/seri [symbol] | −3.45 | −1.21 | 0.00 | 2.27 |
| Phosphatases | protein-tyrosine phosphatase 1B (PTP-1B) | 1.31 | −2.21 | 0.00 | −1.20 |
| Death Kinases | interferon-inducible RNA-dependent protein kinase ( [symbol] | −4.46 | −1.76 | 0.00 | −7.33 |
| Repressors | interferon regulatory factor 1 (IRF1) | 0.00 | −6.44 | −2.88 | −4.05 |
| Replication Factors & | activator 1 40-kDa subunit; replication factor C 40-kD | 0.00 | −1.20 | 2.22 | −1.07 |
| Caspases | caspase-10 precursor (CASP10); ICE-LIKE apoptotic | −7.66 | −2.30 | 0.00 | 1.14 |
| Transducers Effectors & | zyxin + zyxin-2 | −12.38 | −3.57 | 0.00 | 1.82 |
| Extracellular | peripheral myelin protein 22 (PMP22); CD25 protein; | −2.65 | −0.00 | 1.32 | −1.83 |
| Extracellular | myelin-oligodendrocyte glycoprotein precursor (MOG [symbol] | −3.03 | −1.06 | 0.00 | 1.39 |
| Repressors | interferon regulatory factor 2 (IRF2) | 0.00 | −6.60 | 1.53 | −2.98 |
| Repressors | estrogen receptor hSNF2b; global transcription activa [symbol] | 0.00 | −1.29 | 7.58 | −1.17 |
| Extracellular | Alzheimer's disease amyloid A4 protein precursor; pr [symbol] | −8.17 | 0.00 | −2.43 | −1.38 |
| Receptors | interleukin-6 receptor alpha subunit precursor (IL-6R- [symbol] | −1.44 | −3.14 | 0.00 | 1.00 |
| Xenobiotic Transporters | glutathione S-transferase theta 1 (GSTT1) | −19.45 | −2.93 | 0.00 | 2.43 |
| Receptors | cell surface glycoprotein mac-1 alpha subunit precurs [symbol] | −13.73 | −1.91 | 0.00 | 1.09 |
| Cytokines & Chemokines | platelet-derived growth factor A subunit precursor (P [symbol] | −2.98 | 0.00 | −4.54 | 1.52 |
| Proteins | glutathione S-transferase mu1 (GSTM1; GST1); HB [symbol] | −7.00 | −2.17 | 0.00 | 1.60 |
| Suppressors | transforming protein p21/K-ras 2B | −5.49 | 0.00 | 0.00 | −1.06 |
| Suppressors | N-ras; transforming p21 protein | −3.28 | 0.00 | 0.00 | −1.29 |
| Suppressors | fos-related antigen 2 (FRA2) | 0.00 | −3.03 | 0.00 | −1.18 |
| Other Cell Cycle Proteins | myeloid cell nuclear differentiation antigen (MNDA) | −4.69 | 0.00 | 0.00 | −1.17 |
| Other Cell Cycle Proteins | diaphanous 1 (HDIA1) | −6.77 | 0.00 | 0.00 | −1.20 |
| Network Members | janus kinase 3 (JAK3); leukocyte janus kinase (L-JAK [symbol] | −5.26 | 0.00 | 0.00 | −1.22 |
| Phosphoinositol Kinases | 68-kDa type I phosphatidylinositol-4-phosphate 5-kin [symbol] | 0.00 | 0.00 | −3.81 | −1.14 |
| G Proteins | ras-related C3 botulinum toxin substrate 1; p21-rac1; | −12.38 | 0.00 | 0.00 | −1.54 |
| Phosphatases | leukocyte common antigen precursor (L-CA); CD45 a | 0.00 | −4.06 | 0.00 | −1.14 |
| Inhibitors | linker for activation of T-cells (LAT) | 0.00 | −1.67 | 0.00 | −1.22 |
| Cyclases & Diesterases | 3′5′-cAMP phosphodiesterase HPDE4A6 | 0.00 | −6.45 | −1.11 | −1.43 |
| Caspases | caspase-8 precursor (CASP8); ICE-like apoptotic pro [symbol] | −3.35 | 0.00 | 0.00 | −1.22 |
| Caspases | interleukin-1 beta convertase precursor (IL-1BC); IL-1 | 0.00 | −7.35 | 0.00 | −1.25 |
| Factors | cellular nucleic acid binding protein (CNBP); sterol re [symbol] | 0.00 | −2.10 | 0.00 | −1.55 |
| Repressors | metal-regulatory transcription factor | −6.45 | 0.00 | 0.00 | 1.57 |
| Repressors | transcription repressor protein PRDI-BF1; beta-interf [symbol] | −17.66 | 0.00 | 0.00 | −1.13 |
| Repressors | endothelial transcription factor GATA2 | −6.55 | 0.00 | 0.00 | −1.74 |
| Receptors | integrin alpha 4 precursor (ITGA4); VLA4; CD49D an [symbol] | −4.38 | 0.00 | 0.00 | 1.62 |
| Receptors | interleukin-1 receptor type II precursor (IL-1R2); IL-1 [symbol] | −6.25 | 0.00 | 0.00 | −1.26 |
| Chemokine Receptors | granulocyte-macrophage colony-stimulating factor re [symbol] | 0.00 | −2.72 | 0.00 | −1.18 |
| CDK Inhibitors | Sp3 protein | −5.90 | 0.00 | 0.00 | −1.29 |
| Receptors | interleukin-1 receptor type I precursor (IL-1R1); IL-1R [symbol] | −17.02 | 0.00 | 0.00 | −2.86 |
| Xenobiotic Transporters | heme oxygenase 1 (HO1); HSOXYGR | 0.00 | −3.89 | 0.00 | −1.31 |
| Proteosomal Proteins | proteasome component C2; macropain subunit C2; p [symbol] | −4.45 | 0.00 | 0.00 | −1.56 |
| Proteosomal Proteins | proteasome component C3; macropain subunit C3; [symbol] | −5.05 | 0.00 | 0.00 | −2.15 |
| Proteosomal Proteins | proteasome component C5; macropain subunit C5; p [symbol] | −13.45 | 0.00 | 0.00 | −1.37 |
| Hormones | ribonuclease/angiogenin inhibitor (RAI); placental rib [symbol] | 0.00 | −1.20 | 0.00 | −1.14 |
| Proteosomal Proteins | proteasome component C8; macropain subunit C8; [symbol] | −6.26 | 0.00 | 0.00 | −1.78 |
| Heat Shock Proteins | 27-kDa heat-shock protein (HSP27); stress-responsiv [symbol] | −3.96 | 0.00 | 0.00 | −1.49 |
| Xenoblotic Metabolism | cytosolic superoxide dismutase 1 (SOD1) | −3.25 | 0.00 | 0.00 | −1.01 |
| Cysteine Proteases | cathepsin H precursor | −11.12 | 0.00 | 0.00 | −2.14 |
| G Proteins | ADP-ribosylation factor 1 | 0.00 | −5.30 | 0.00 | −1.50 |
| G Proteins | transforming protein rhoB; ARHB; ARH6 | 13.75 | 0.00 | 0.00 | −1.15 |
| Network Members | dual-specificity mitogen-activated protein kinase kina [symbol] | 0.00 | 0.00 | −2.49 | 1.88 |
| G Proteins | guanine nucleotide-binding protein G-i/G-s/G-t beta s [symbol] | 0.00 | −4.45 | 0.00 | 1.43 |
| G Proteins | RaIB GTP-binding protein | −4.83 | 0.00 | 0.00 | 1.57 |
| Phosphatases | protein-tyrosine phosphatase 2C (PTP-2C); SH-PTP2 [symbol] | −2.63 | 0.00 | 0.00 | 1.19 |
| Death Kinases | death-associated protein kinase 1 (DAP kinase 1; DA [symbol] | 0.00 | −3.54 | 0.00 | 1.14 |
| Calcium-Binding Proteins | neurogranin (NRGN); RC3 | −3.23 | 0.00 | 0.00 | 1.01 |
| Phosphatases | protein phosphatase PP2A 55-kDa regulatory subunit | −6.14 | 0.00 | 0.00 | 1.06 |
| Proteins & Ligases | DNA-repair protein XRCC1 | 0.00 | −7.59 | 0.00 | 1.44 |
| Receptors | thrombospondin 2 precursor (THBS2; TSP2) | 0.00 | −13.37 | 0.00 | 1.03 |
| Cytokines & Chemokines | teratocarcinoma-derived growth factor 1 (TDGF1); e [symbol] | −10.71 | 0.00 | 0.00 | 1.13 |
| Cytokines & Chemokines | neuroleukin (NLK); glucose-6-phosphate isomerase ( [symbol] | −7.18 | 0.00 | 0.00 | 1.37 |
| Cytokines & Chemokines | OX40 ligand (OX40L); GP34; tax-transcriptionally act [symbol] | −3.16 | 0.00 | 0.00 | 1.00 |
| Cytokines & Chemokines | amphiregulin (AR); colorectum cell-derived growth fa [symbol] | 0.00 | −6.18 | 0.00 | 1.00 |
| Cytokines & Chemokines | connective tissue growth factor precursor (CTGF) | −3.01 | 0.00 | 0.00 | 1.00 |

TABLE 4
GENE CHANGES INDUCED BY SEB IN HUMAN LYMPHOID CELLS BY GENE ARRAY

| Protein/gene | 3 H | 6 H | 12 H | 18 H |
|---|---|---|---|---|
| interferon gamma-induced protein precursor (gamma-IP10) | 19.640 | 12.230 | 18.215 | 13.692 |
| cytokine humig; interferon-gamma-induced monokine (MIG) | 15.247 | 20.567 | 26.411 | 12.263 |
| interferon gamma precursor (IFN-gamma; IFNG); Immune ir | 13.460 | 20.600 | 17.807 | 22.888 |
| bone marrow stromal antigen 1 (BST-1); ADP-ribosyl cyclas | 8.680 | 1.393 | 0.000 | 1.788 |
| C-ets-2 | 8.013 | 3.134 | 0.000 | 0.000 |
| interleukin-5 receptor alpha subunit precursor (IL-5R-alpha; | 7.570 | 10.643 | 15.705 | 7.463 |
| cysteine protease ICE-LAP3 | 5.993 | 1.721 | 1.609 | 0.000 |
| activator 1 40-kDa subunit; replication factor C 40-kDa subu | 4.821 | 0.678 | 1.142 | 2.408 |
| interferon regulatory factor 7 (IRF-7) | 4.737 | 3.100 | 3.495 | 2.793 |
| insulin-like growth factor-binding protein 3 precursor (IGF-bi [symbol] | 4.167 | 0.000 | 0.000 | 0.000 |
| platelet-activating factor receptor (PAFR) | 4.110 | 2.015 | 4.393 | 0.000 |
| OX40 ligand (OX40L); GP34; tax-transcriptionally activated | 3.963 | 0.000 | 0.000 | 0.000 |
| T-lymphoma invasion and metastasis inducing TIAM1 | 3.940 | 0.000 | 0.293 | 0.000 |
| caspase-10 precursor (CASP10); ICE-LIKE apoptotic protea | 3.885 | 0.806 | 0.000 | 0.000 |
| proteasome component C3; macropain subunit C3; multicat [symbol] | 3.423 | 1.448 | 1.583 | 5.871 |
| granulocyte-macrophage colony stimulating factor (GM-CSF | 3.377 | 11.063 | 0.000 | 1.272 |
| Gem; induced immediate early protein; ras family member ( [symbol] | 3.200 | 0.000 | 0.000 | 0.000 |
| tumor necrosis factor receptor 1 (TNFR1); tumor necrosis fa | 3.198 | 0.253 | 0.373 | 0.000 |
| BCL-2 binding athanogene-1 (BAG-1); glucocorticoid recept [symbol] | 3.143 | 0.357 | 1.412 | 0.000 |
| placental plasminogen activator inhibitor 2 (PAI-2; PLANH2) | 3.061 | 1.011 | 1.015 | 1.372 |
| transcription factor NF-ATc | 3.047 | 0.000 | 1.860 | 0.000 |
| MAPKAP kinase (3pK) | 3.022 | 0.591 | 0.000 | 0.000 |
| endothelial-monocyte activating polypeptide II (EMAP II) | 2.993 | 0.447 | 1.327 | 2.577 |
| cadherin 3 (CDH3); placental cadherin precursor (P-cadheri [symbol] | 2.980 | 0.727 | 1.833 | 0.000 |
| proto-oncogene tyrosine-protein kinase lck; p56-lck; lympho [symbol] | 2.957 | 0.389 | 0.288 | 0.726 |
| protein kinase C alpha polypeptide (PKC-alpha; PKCA) | 2.843 | 0.728 | 0.539 | 0.000 |
| leucine-rich repeat protein SHOC-2; ras-binding protein SUR | 2.793 | 0.578 | 0.626 | 0.000 |
| transducin beta 5 subunit; GTP-binding protein G(i)/G(s)/G( [symbol] | 2.663 | 0.000 | 0.000 | 0.000 |
| BCL-2-related protein A1 (BCL2A1); BFL1 protein: hemopoi [symbol] | 2.650 | 0.000 | 0.000 | 0.000 |
| RaIB GTP-binding protein | 2.643 | 0.420 | 0.775 | 0.000 |
| caspase-2 precursor (CASP2); ICH-1L protease + ICH-1S p [symbol] | 2.563 | 0.464 | 0.538 | 0.969 |
| methionine aminopeptidase 2 (METAP2); peptidase M2; initi | 2.563 | 0.765 | 2.718 | 4.492 |
| corticotropin releasing factor receptor 1 precursor (CR [symbol] | 2.449 | 1.014 | 1.358 | 1.703 |
| DNA-binding protein HIP116; ATPase; SNF2/SWI2-related [symbol] | 2.404 | 3.028 | 2.157 | 0.479 |
| cell surface glycoprotein mac-1 alpha subunit precursor; CD | 2.347 | 4.743 | 5.360 | 3.028 |
| interleukin-1 receptor type I precursor (IL-1R1); IL-1R-alpha; | 2.330 | 0.349 | 0.000 | 0.000 |
| cdc2-related protein kinase PISSLRE | 2.306 | 0.160 | 1.149 | 0.673 |
| nuclear factor NF45 | 2.267 | 0.000 | 0.000 | 0.000 |
| B94 protein | 2.256 | 2.067 | 6.206 | 2.499 |
| estrogen sulfotransferase (STE; EST1) | 2.235 | 0.000 | 2.858 | 0.000 |
| putative sro-like adapter protein (SLAP) | 2.235 | 0.658 | 1.039 | 0.000 |
| calcium/calmodulin-dependent protein kinase I (CAMKI) | 2.183 | 0.000 | 0.229 | 0.754 |
| hepatocyte growth factor-like protein; macrophage-stimulatir | 2.183 | 1.649 | 0.000 | 0.923 |
| platelet-derived growth factor A subunit precursor (PDGFA; I | 2.180 | 0.453 | 0.526 | 0.000 |
| induced myeloid leukemia cell differentiation protein MCL-1 | 2.171 | 1.064 | 0.956 | 1.888 |
| ataxia telanglectasia (ATM) | 2.120 | 0.359 | 0.526 | 0.000 |
| cAMP-dependent 3′,5′-cyclic phosphodiesterase 4D (PDE43) | 2.107 | 0.405 | 0.722 | 0.000 |
| bone morphogenetic protein 4 (BMP4) + bone morphogenet | 2.097 | 0.000 | 0.000 | 0.000 |
| adenosine A1 receptor (ADORA1) | 1.980 | 0.000 | 0.000 | 0.000 |
| renin-binding protein (RENBP; RNBP) | 1.953 | 0.956 | 1.624 | 0.792 |
| lymphotoxin-beta (LT-beta; LTB); tumor necrosis factor C (T | 1.912 | 0.715 | 0.000 | 1.340 |
| translin; recombination hotspot binding protein | 1.902 | 0.481 | 0.828 | 2.843 |
| interferon regulatory factor 1 (IRF1) | 1.890 | 0.851 | 1.556 | 10.240 |
| C-jun N-terminal kinase kinase 2 (JNKK2); mitogen-activate [symbol] | 1.885 | 0.000 | 0.000 | 0.000 |
| inhibin alpha subunit precursor (INHA) | 1.883 | 0.000 | 0.000 | 0.000 |
| tumor necrosis factor precursor (TNF-alpha; TNFA); cachec [symbol] | 1.870 | 3.078 | 2.620 | 3.307 |
| natural killer cell enhancing factor (NKEFB) + thiol-specific a | 1.790 | 0.284 | 1.568 | 5.448 |
| hypoxia-inducible factor 1 alpha (HIF1 alpha); ARNT-interac | 1.743 | 1.293 | 1.902 | 0.000 |
| intercellular adhesion molecule-1 precursor (ICAM1); major [symbol] | 1.730 | 1.860 | 3.288 | 1.989 |
| macrophage inflammatory protein 1 beta precursor (MIP1-b [symbol] | 1.664 | 3.280 | 14.825 | 12.880 |
| cyclin-dependent kinase 4 inhibitor (CDK4I; CDKN2); p16-IN | 1.662 | 0.910 | 2.262 | 4.947 |
| T-lymphocyte maturation-associated protein MAL | 1.620 | 1.963 | 4.007 | 0.000 |
| inhibitor of apoptosis protein1 (HIAP1; API1) + IAP homolog | 1.565 | 0.970 | 3.320 | 0.000 |
| monocyte chemotactic protein 1 precursor (MCP1); monocyt | 1.552 | 1.150 | 1.215 | 1.137 |
| cyclin G-associated kinase (GAK) | 1.516 | 0.349 | 0.544 | 0.000 |
| protein-tyrosine phosphatase 1B (PTP-1B) | 1.501 | 1.183 | 2.075 | 6.212 |
| calpain 2 large (catalytic) subunit; M-type calcium-activated | 1.458 | 0.706 | 0.540 | 0.000 |
| interleukin-10 precursor (IL-10); cytokine synthesis inhibitory | 1.450 | 0.867 | 2.486 | 1.221 |
| cAMP-dependent protein kinase I alpha regulatory subunit; ti | 1.426 | 0.933 | 0.721 | 0.363 |
| heat shock cognate 71-kDa protein | 1.423 | 1.010 | 2.547 | 3.073 |
| NF-kappaB transcription factor p65 subunit; RELA; NFKB3 | 1.394 | 0.810 | 0.000 | 0.000 |
| granulocyte chemotactic protein 2 (GCP 2); neutrophil-activ [symbol] | 1.381 | 1.827 | 2.117 | 13.304 |
| transcriptional regulator interferon-stimulated gene factor 3 [symbol] | 1.341 | 1.964 | 1.751 | 3.634 |
| cyclin-dependent kinase inhibitor 1 (CDKN1A); melanoma di | 1.341 | 1.625 | 4.597 | 3.044 |
| calcium/calmodulin-dependent protein kinase type IV catalyt | 1.332 | 0.796 | 0.678 | 0.539 |
| apoptosis regulator bcl-x | 1.327 | 1.123 | 1.945 | 3.542 |
| nibrin (NBS1) | 1.320 | 1.760 | 5.157 | 0.000 |
| sodium/hydrogen exchanger 1 (Na+/H+ exchanger 1; NHE1) | 1.314 | 0.205 | 0.626 | 0.000 |
| ribosomal protein S6 kinase II alpha 3 (S6KII-alpha 3); ribos | 1.313 | 0.976 | 0.932 | 0.225 |
| G-protein-coupled receptor HM74 | 1.310 | 6.207 | 3.004 | 0.000 |
| interferon regulatory factor 2 (IRF2) | 1.298 | 0.905 | 1.780 | 3.252 |
| linker for activation of T-cells (LAT) | 1.261 | 0.660 | 0.933 | 0.843 |
| guanine nucleotide-binding protein G(I)/G(S)/G(T) beta subu | 1.251 | 1.046 | 2.923 | 17.465 |
| serine/threonine protein phosphatase 2B catalytic subunit al [symbol] | 1.251 | 0.598 | 0.475 | 0.679 |
| interleukin-1 beta precursor (IL-1; IL1B); catabolin | 1.242 | 1.621 | 3.284 | 2.565 |
| 26S protease regulatory subunit 6A; TAT-binding protein 1 [symbol] | 1.240 | 0.828 | 1.375 | 3.133 |
| macrophage inflammatory protein 1 alpha precursor (MIP1- [symbol] | 1.221 | 2.296 | 14.022 | 8.888 |
| heparin-binding EGF-like growth factor (HBEGF); diphtheria | 1.220 | 6.817 | 2.011 | 0.000 |
| dual specificity mitogen-activated protein kinase kinase 2 (M | 1.210 | 0.664 | 0.791 | 0.397 |
| cAMP-dependent protein kinase alpha-catalytic subunit (PK [symbol] | 1.207 | 0.249 | 0.186 | 0.000 |
| mitochondrial matrix protein P1 precursor, p60 lymphocyte [symbol] | 1.201 | 0.792 | 1.138 | 4.136 |
| TRRAP protein | 1.201 | 0.852 | 0.813 | 0.877 |
| early growth response protein 1 (hEGR1); transcription facto | 1.178 | 0.974 | 0.513 | 0.283 |
| ERBB-3 receptor protein-tyrosine kinase precursor; epiderm [symbol] | 1.176 | 1.508 | 2.822 | 1.956 |
| tuberin; tuberous sclerosis 2 protein (TSC2) | 1.165 | 0.830 | 0.512 | 0.295 |
| E16 amino acid transporter | 1.157 | 1.626 | 0.000 | 3.373 |
| death-associated protein kinase 1 (DAP kinase 1; DAPK1) | 1.153 | 0.548 | 0.126 | 0.364 |
| calcium-dependent protease small (regulatory) subunit; calp | 1.148 | 0.806 | 0.856 | 0.814 |
| protein kinase C substrate 80-kDa protein heavy chain (PKC | 1.135 | 0.868 | 0.840 | 0.670 |
| interferon-alpha/beta receptor beta subunit precursor (IFN-al | 1.133 | 0.220 | 0.490 | 0.000 |
| cAMP-dependent transcription factor ATF-4; DNA-binding pr | 1.131 | 0.833 | 1.250 | 1.149 |
| C-maf transcription factor | 1.106 | 0.294 | 0.000 | 0.898 |
| tumor necrosis factor receptor (TNFR) + tumor necrosis fa [symbol] | 1.099 | 0.653 | 0.120 | 1.707 |
| CDC-like kinase 1 (CLK1) | 1.098 | 0.759 | 0.814 | 0.549 |
| SL cytokine precursor FLT3 ligand (FLT3LG) | 1.097 | 1.097 | 0.000 | 2.734 |
| cyclin T CDK9-associated | 1.097 | 0.370 | 0.537 | 0.000 |
| 14-3-3 protein beta/alpha; protein kinase C inhibitor protein- | 1.093 | 0.810 | 0.928 | 0.878 |
| proliferating cell nucleolar antigen P120; NOL1 | 1.090 | 0.333 | 1.804 | 6.099 |
| thioredoxin reductase | 1.083 | 0.221 | 0.000 | 0.000 |
| cytoplasmic antiproteinase 3 (CAP3); protease inhibitor 19 (1 | 1.082 | 1.899 | 4.415 | 0.000 |
| proteasome component C2; macropain subunit C2; proteasc | 1.080 | 0.000 | 6.018 | 4.023 |
| protein kinase C delta (NPKC-delta) | 1.075 | 0.898 | 0.481 | 0.943 |
| nuclear factor NF-kappa-B p100 subunit; nuclear factor NF- [symbol] | 1.060 | 1.381 | 2.403 | 7.933 |
| ALG-2 calcium-binding protein | 1.056 | 0.431 | 0.217 | 0.981 |
| thrombin receptor (TR); F2R; PAR1 | 1.055 | 0.720 | 0.414 | 0.216 |
| granulocyte colony stimulating factor receptor precursor (GC | 1.036 | 0.303 | 0.169 | 0.200 |
| mitogen-activated protein kinase p38 (MAP kinase p38); cyt [symbol] | 1.036 | 0.971 | 0.733 | 0.236 |
| dioxin-inducible cytochrome P450 1B1 (CYP1B1) | 1.029 | 1.020 | 0.515 | 0.415 |
| growth factor receptor-bound protein 2 (GRB2) isoform; GR [symbol] | 1.029 | 0.796 | 0.771 | 0.640 |
| macrophage-specific colony-stimulating factor (CSF-1; MCS | 1.019 | 1.698 | 1.616 | 1.753 |
| cytosolic superoxide dismutase 1 (SOD1) | 1.005 | 1.811 | 2.376 | 2.182 |
| fibronectin receptor beta subunit (FNRB); integrin beta 1 (IT [symbol] | 1.003 | 0.604 | 0.747 | 0.294 |
| leukocyte common antigen precursor (L-CA); CD45 antigen; | 1.003 | 0.903 | 0.545 | 0.939 |
| leukocyte adhesion glycoprotein LFA-1 alpha subunit precun | 1.001 | 0.792 | 0.770 | 0.586 |
| transcription factor Sp1 (TSFP1) | 1.001 | 0.499 | 1.122 | 0.690 |
| neurogranin (NRGN); RC3 | 1.000 | 0.663 | 0.416 | 0.000 |
| interleukin-8 precursor (IL-8); monocyte-derived neutrophil c | 0.985 | 1.062 | 3.695 | 18.291 |
| tristetraproline (TTP); TIS11; ZFP36; growth factor-inducible | 0.984 | 0.959 | 0.723 | 0.415 |
| major prion protein precursor (PRP); PRP27-30; PRP33-35 [symbol] | 0.970 | 1.186 | 1.270 | 2.842 |
| stromal cell derived factor 1 receptor (SDF1 receptor); fusin; | 0.968 | 0.794 | 0.636 | 0.372 |
| mutL protein homolog; DNA mismatch repair protein MLH1; | 0.966 | 0.694 | 0.769 | 0.770 |
| transcriptional repressor protein yin & yang 1 (YY1); delta tr [symbol] | 0.962 | 0.445 | 1.180 | 0.293 |
| heme oxygenase 1 (HO1); HSOXYGR | 0.952 | 0.197 | 0.000 | 0.312 |
| epidermal growth factor receptor substrate 15 (EPS15); AF- [symbol] | 0.952 | 0.496 | 0.292 | 0.000 |
| casein kinase I gamma 2 (CKI-gamma 2) | 0.946 | 0.546 | 0.329 | 0.000 |
| CD27L antigen receptor precursor; T-cell activation CD27 ar | 0.945 | 0.161 | 0.463 | 0.764 |
| migration inhibitory factor-related protein 8 (MRP8); calgran [symbol] | 0.933 | 0.658 | 0.732 | 0.920 |
| transforming protein rhoA H12 (RHO12; ARH12; ARHA) | 0.928 | 0.737 | 0.802 | 0.716 |
| sodium/potassium-transporting ATPase beta 3 subunit (ATP | 0.927 | 0.000 | 3.423 | 0.000 |
| human immunodeficiency virus type I enhancer-binding prot [symbol] | 0.922 | 1.877 | 3.784 | 2.049 |
| transcriptional activator hSNF2-alpha | 0.900 | 0.864 | 1.179 | 0.401 |
| interleukin-4 receptor alpha subunit precursor (IL-4R-alpha; | 0.896 | 2.634 | 4.549 | 2.529 |
| interleukin-2 receptor alpha subunit precursor (IL-2 receptor | 0.896 | 1.232 | 2.373 | 1.590 |
| sodium/potassium-transporting ATPase alpha 1 subunit (Na | 0.894 | 0.640 | 2.611 | 0.678 |
| xeroderma pigmentosum group C repair complementing pro [symbol] | 0.893 | 0.444 | 0.923 | 1.773 |
| protein kinase C theta (PKC-theta) | 0.893 | 1.120 | 2.593 | 0.000 |
| placenta growth factors 1 + 2 (PLGF1 + PLGF2) | 0.891 | 0.869 | 1.737 | 5.059 |
| transmembrane 4 superfamily protein; SAS | 0.884 | 0.769 | 3.610 | 2.288 |
| ezrin; cytovillin 2; villin 2 (VIL2) | 0.883 | 0.676 | 0.470 | 0.673 |
| vascular endothelial growth factor precursor (VEGF); vascul [symbol] | 0.866 | 0.280 | 0.521 | 0.000 |
| EB1 protein | 0.865 | 0.734 | 0.854 | 0.936 |
| trans-acting T-cell specific transcription factor GATA3 | 0.861 | 0.743 | 1.021 | 0.455 |
| leukocyte adhesion glycoprotein p150, 95 alpha subunit pre [symbol] | 0.847 | 0.979 | 3.284 | 0.000 |
| transcriptional repressor NF-X1 | 0.838 | 0.495 | 1.717 | 1.803 |
| cellular nucleic acid binding protein (CNBP); sterol regulator: | 0.836 | 0.731 | 1.232 | 4.277 |
| ephrin A receptor 4 precursor; tyrosine-protein kinase recept | 0.822 | 3.121 | 5.124 | 1.801 |
| v-erbA related protein (EAR2) | 0.821 | 0.352 | 0.283 | 0.638 |
| retinoblastoma-like protein 2 (RBL2; RB2); 130-kDa retinobl [symbol] | 0.813 | 0.265 | 0.250 | 0.000 |
| cathepsin D precursor (CTSD) | 0.763 | 0.324 | 0.354 | 0.147 |
| basic transcription element-binding protein 2 (BTEB2); GC-b | 0.759 | 0.000 | 3.916 | 0.000 |
| erythrocyte glucose transporter 1 (GLUT1) | 0.759 | 0.185 | 1.200 | 1.418 |
| phospholipase C-gamma-2 (PLC-gamma-2; PLCG2); 1-pho [symbol] | 0.758 | 0.324 | 0.449 | 0.804 |
| IEX-1L anti-death protein; PRG-1; DIF-2 | 0.730 | 1.290 | 2.185 | 2.068 |
| deoxyribonuclease II (DNase II); acid DNase; lysosomal DN [symbol] | 0.728 | 0.355 | 0.357 | 0.146 |
| cell surface adhesion glycoproteins LFA-1/CR3/p150,95 beta | 0.700 | 0.529 | 0.317 | 0.343 |
| myelin-oligodendrocyte glycoprotein precursor (MOG) | 0.694 | 0.995 | 0.549 | 0.410 |
| glutathione S-transferase pi (GSTP1; GST3) | 0.693 | 0.850 | 0.944 | 0.814 |
| metalloproteinase inhibitor 1 precursor (TIMP1); erythroid pc | 0.691 | 0.761 | 0.271 | 0.200 |
| transforming protein rhoB; ARHB; ARH6 | 0.691 | 0.929 | 0.712 | 0.000 |
| erythroid differentiation protein (EDF); inhibin beta A subuni | 0.690 | 4.953 | 7.607 | 3.348 |
| nuclear factor kappa-B DNA binding subunit (NF-kappaB; NI | 0.680 | 0.595 | 3.578 | 0.000 |
| ZFM1 protein alternatively spliced product | 0.673 | 0.835 | 1.163 | 2.732 |
| neuroleukin (NLK); glucose-6-phosphate isomerase (GPI); p | 0.671 | 0.681 | 1.349 | 4.058 |
| ras-related C3 botulinum toxin substrate 1; p21-rac1; ras-like | 0.659 | 0.308 | 1.015 | 0.726 |
| jun-D | 0.652 | 0.272 | 0.000 | 0.000 |
| xeroderma plgmentosum group G complementing protein (X | 0.648 | 0.306 | 0.244 | 0.000 |
| macMARCKS; MARCKS-related protein (MRP); MLP | 0.640 | 0.163 | 0.000 | 0.946 |
| CD40 receptor-associated factor 1 (CRAF1) | 0.638 | 0.469 | 0.739 | 0.000 |
| c-rel proto-oncogene protein | 0.638 | 0.189 | 0.887 | 0.000 |
| urokinase-type plasminogen activator receptor GPI-anchorex | 0.627 | 0.780 | 1.131 | 1.590 |
| myb proto-oncogene; c-myb | 0.625 | 0.301 | 0.867 | 1.465 |
| proliferating cyclic nuclear antigen (PCNA); cyclin | 0.624 | 0.224 | 1.589 | 1.954 |
| cot proto-oncogene | 0.611 | 0.165 | 0.000 | 0.000 |
| phosphatidylinositol-4-phosphate 5-kinase II beta; 1-phospl [symbol] | 0.589 | 0.362 | 0.264 | 0.587 |
| cytochrome P450 reductase | 0.588 | 0.200 | 0.709 | 0.000 |
| extracellular signal-regulated kinase 2 (ERK2); mitogen-acti [symbol] | 0.578 | 0.630 | 1.211 | 0.610 |
| C5a anaphylatoxin receptor (C5AR); CD88 antigen | 0.570 | 0.142 | 0.335 | 0.468 |
| ribonuclease/angiogenin inhibitor (RAI); placental ribonuclea | 0.546 | 0.204 | 0.347 | 0.000 |
| adenomatous polyposis coli protein (APC protein); DP2.5 | 0.540 | 0.000 | 0.190 | 0.000 |
| MAPK/ERK kinase kinase 3 (MEK kinase 3; MEKK3) | 0.519 | 0.207 | 0.291 | 0.000 |
| estrogen receptor hSNF2b; global transcription activator SNI | 0.499 | 0.747 | 1.361 | 3.036 |
| N-ras; transforming p21 protein | 0.487 | 0.264 | 1.160 | 1.351 |
| thrombomodulin precursor (THBD; THRM); fetomodulin; CD | 0.486 | 0.312 | 0.000 | 0.000 |
| membrane-bound & soluble catechol-O-methyltransferase (C | 0.480 | 0.198 | 1.296 | 0.697 |
| B-raf proto-oncogene (RAFB1) | 0.470 | 0.149 | 0.618 | 0.000 |
| tyrosine-protein kinase receptor tyro3 precursor, rse; sky; dtl [symbol] | 0.468 | 0.274 | 0.927 | 0.000 |
| P2X purinoceptor 5 (P2X5) | 0.463 | 0.423 | 2.010 | 1.464 |
| MAD protein; MAX dimerizer | 0.460 | 0.704 | 0.679 | 0.000 |
| neutral amino acid transporter A (SATT); alanine/serine/cyst | 0.458 | 0.161 | 0.691 | 0.000 |
| zyxin + zyxin-2 | 0.438 | 0.630 | 0.357 | 0.356 |
| thioredoxin peroxidase 2 (TDPX2); thioredoxin-dependent p [symbol] | 0.409 | 0.363 | 1.771 | 0.851 |
| cell division protein kinase 9 (CDK9); serine/threonine protei [symbol] | 0.402 | 0.360 | 1.476 | 0.000 |
| sodium-& chloride-dependent taurine transporter | 0.393 | 0.425 | 0.283 | 0.195 |
| CDC-like kinase 3 (CLK3) | 0.392 | 0.451 | 0.000 | 0.000 |
| janus kinase 3 (JAK3); leukocyte janus kinase (L-JAK) | 0.387 | 0.805 | 0.920 | 0.000 |
| interleukin-1 receptor type II precursor (IL-1R2); IL-1R-beta | 0.384 | 0.000 | 0.000 | 0.000 |
| interleukin-18 precursor (IL-18); interferon-gamma-inducing | 0.371 | 0.488 | 0.993 | 0.679 |
| CDC25B; CDC25HU2; M-phase inducer phosphatase 2 | 0.364 | 0.210 | 0.371 | 0.295 |
| vascular endothelial growth factor receptor 1 (VEGFR1); tyr [symbol] | 0.360 | 0.255 | 7.255 | 2.796 |
| cytoplasmic antiproteinase 2 (CAP2); protease inhibitor 8 | 0.355 | 0.769 | 0.963 | 0.000 |
| TSG101 tumor susceptibility protein | 0.353 | 0.378 | 0.000 | 0.943 |
| mothers against dpp homolog 4 (SMAD4); MADR4; pancrea | 0.352 | 0.222 | 0.275 | 0.000 |
| alpha1 catenin (CTNNA1); cadherin-associated protein; alph [symbol] | 0.343 | 0.700 | 0.578 | 0.000 |
| cyclin-dependent kinase 4 inhibitor D (CDKN2D); p19-INK4 [symbol] | 0.340 | 0.203 | 0.989 | 0.000 |
| Von Hippel-Lindau tumor suppressor protein (VHL) | 0.334 | 0.161 | 0.388 | 0.000 |
| glutathione S-transferase theta 1 (GSTT1) | 0.321 | 0.327 | 1.055 | 0.000 |
| GA-binding protein beta-2 subunit (GABP-beta2); transcripti [symbol] | 0.320 | 0.541 | 1.258 | 2.051 |
| glutathione reductase (GRase; GSR; GR) | 0.308 | 0.299 | 0.645 | 0.944 |
| zinc finger X-chromosomal protein (ZFX) | 0.292 | 0.741 | 1.571 | 2.006 |
| monocarboxylate transporter 1 (MCT1) | 0.285 | 0.423 | 3.382 | 3.066 |
| G1/S-specific cyclin D2 (CCND2) + KIAK0002 | 0.285 | 2.747 | 10.960 | 8.701 |
| macrophage Inflammatory protein 2 alpha (MIP2-alpha); gro | 0.280 | 0.926 | 1.587 | 14.417 |
| guanine nucleotide-binding protein G-i/G-s/G-t beta subunit: | 0.272 | 0.350 | 0.000 | 0.000 |
| transducer of erbB2 (TOB) | 0.265 | 0.218 | 0.348 | 0.000 |
| fos-related-antigen 2 (FRA2) | 0.260 | 0.371 | 0.000 | 0.000 |
| erythroblastosis virus oncogene homolog 1 (ETS-1); p54 | 0.252 | 0.630 | 0.812 | 0.000 |
| C-src proto-oncogene (SRC1) | 0.231 | 0.311 | 0.000 | 0.000 |
| ADP-ribosylation factor 1 | 0.201 | 0.878 | 1.062 | 0.000 |
| glutathione peroxidase (GSHPX1; GPX1) | 0.197 | 0.099 | 0.434 | 0.150 |
| DNA polymerase gamma (POLG); mitochondrial DMA polyrr | 0.180 | 0.654 | 0.992 | 0.000 |
| macrophage colony stimulating factor I receptor precursor [symbol] | 0.164 | 0.127 | 0.049 | 0.110 |
| interleukin-2 precursor (IL-2); T-cell growth factor (TCGF) | 0.000 | 9.023 | 22.033 | 6.493 |
| CDW40 antigen; CD40L receptor precursor. nerve growth fa | 0.000 | 7.846 | 15.309 | 5.084 |
| Janus kinase 2 (JAK2); receptor-associated tyrosine kin [symbol] | 0.000 | 6.137 | 2.225 | 0.000 |
| cathepsin H precursor | 0.000 | 3.214 | 1.585 | 1.183 |
| interleukin-6 precursor (IL-6); B-cell stimulatory factor 2 (BSI | 0.000 | 3.036 | 5.932 | 2.607 |
| proteasome activator HPA28 subunit beta | 0.000 | 2.420 | 1.465 | 1.040 |
| interleukin-3 precursor (IL-3); multipotential colony-stimulati [symbol] | 0.000 | 2.373 | 5.295 | 0.000 |
| apoptosis regulator bcl-2 | 0.000 | 2.043 | 3.851 | 0.000 |
| cathepsin L precursor, major excreted protein (MEP) | 0.000 | 1.516 | 2.942 | 0.000 |
| signal transducer and activator of transcription 1 alpha/beta | 0.000 | 1.300 | 2.165 | 1.721 |
| proteasome component C5; macropain subunit C5; proteas [symbol] | 0.000 | 1.207 | 1.508 | 2.135 |
| integrin alpha 4 precursor (ITGA4); VLA4; CD49D antigen | 0.000 | 0.977 | 0.364 | 0.000 |
| interferon-inducible RNA-dependent protein kinase (P68 kin [symbol] | 0.000 | 0.913 | 3.394 | 0.000 |
| protein kinase C beta I (PKC-beta-1) | 0.000 | 0.901 | 0.436 | 0.000 |
| integrin alpha 6 precursor (ITGA6); VLA6; CD49F antigen | 0.000 | 0.885 | 2.982 | 0.000 |
| transcription factor RZR-alpha (RZRA); RAR-related orphan | 0.000 | 0.843 | 2.578 | 0.000 |
| p78 putative serine/threonine-protein kinase | 0.000 | 0.711 | 0.462 | 0.000 |
| Machado-Joseph disease protein 1 (MJD1) | 0.000 | 0.572 | 0.395 | 0.000 |
| caspase-8 precursor (CASP8); ICE-like apoptotic protease 5 | 0.000 | 0.546 | 0.223 | 0.000 |
| CACCC-box DNA-binding protein | 0.000 | 0.464 | 1.582 | 3.078 |
| microphthalmia-associated transcription factor (MITF) | 0.000 | 0.454 | 0.321 | 0.446 |
| xeroderma pigmentosum group B complementing protein (X | 0.000 | 0.426 | 0.595 | 0.000 |
| apoptotic protease activating factor 1 (APAF1) | 0.000 | 0.395 | 0.596 | 0.000 |
| ras-related protein RAB2 | 0.000 | 0.375 | 0.579 | 0.000 |
| cGMP-inhibited 3′,5′-cyclic phosphodiesterase B (CGI-PDE [symbol] | 0.000 | 0.372 | 0.268 | 0.000 |
| c-raf proto-oncogene | 0.000 | 0.369 | 0.328 | 0.000 |
| integrin-linked kinase (ILK) | 0.000 | 0.357 | 0.841 | 0.000 |
| vav oncogene | 0.000 | 0.353 | 0.451 | 0.000 |
| transforming growth factor beta receptor III precursor (TGF [symbol] | 0.000 | 0.340 | 0.669 | 0.000 |
| NCK melanoma cytoplasmic src homolog (HSNCK) | 0.000 | 0.333 | 0.482 | 0.000 |
| dual specificity mitogen-activated protein kinase kinase 5 (M | 0.000 | 0.280 | 0.563 | 0.000 |
| C-yes proto-oncogene (YES1) | 0.000 | 0.272 | 0.357 | 0.000 |
| MCM5 DNA replication licensing factor; CDC46 homolog | 0.000 | 0.252 | 0.500 | 2.372 |
| matrix metalloproteinase 9 (MMP9); gelatinase B; 92-kDa ty [symbol] | 0.000 | 0.157 | 0.000 | 7.207 |
| interleukin-1 alpha precursor (IL-1 alpha; IL1A); hematopoie [symbol] | 0.000 | 0.000 | 4.542 | 0.000 |
| leukemia Inhibitory factor precursor (LIF); differentiation-stirr | 0.000 | 0.000 | 4.513 | 4.934 |
| basic transcription factor 2 44-kDa subunit (BTF2p44) | 0.000 | 0.000 | 3.985 | 0.000 |
| neurotrophic tyrosine kinase receptor-related 3; TKT precurs | 0.000 | 0.000 | 3.636 | 0.000 |
| granzyme A precursor, cytotoxic T-lymphocyte proteinase 1; | 0.000 | 0.000 | 2.771 | 0.000 |
| homeobox protein HOX-A5; HOX-1C | 0.000 | 0.000 | 1.709 | 2.517 |
| calcitonin receptor (CTR; CALCR) | 0.000 | 0.000 | 0.000 | 3.255 |

TABLE 5
GENE CHANGES INDUCED BY ANTHRAX EXPOSURE IN VITRO
IN HUMAN LYMPHOID CELLS

| Gene Name | 2 h | 4 h | 8 h |
|---|---|---|---|
| Interferon regulatory factor 1 (IRF1) | 1.672 | 1.665 | 6.986 |
| ezrin; cytovillin 2, villin 2 (VIL2) | 1.33 | 1.832 | 6.444 |
| tumor necrosis factor precursor (TNF-alpha; TNFA); cac | 1.725 | 4.541 | 4.815 |
| adenomatous polyposis coil protein (APC protein); DP2. | 1.111 | 1.092 | 4.135 |
| mothers against dpp homolog 4 (SMAD4); MADR4; pan | 2.043 | 1.173 | 3.413 |
| leukocyte common antigen precursor (L-CA);CD45 anti [symbol] | 1.286 | 1.564 | 3.367 |
| Von Hippel-Lindau tumor suppressor protein (VHL) | 1.379 | 1.138 | 2.011 |
| HHR23A; UV excision repair protein protein RAD23A | −11.63 | 2.37 | 1.801 |
| 40S ribosomal protein S9 | 1.349 | 1.371 | 1.692 |
| Interleukin-1 beta precursor (IL-1; IL1B); catabolin | 3.053 | 1.432 | 1.667 |
| HLA class I histocompatibility antigen C-4 alpha subunit | −3.374 | 1.739 | 1.624 |
| ERBB-3 receptor protein-tyrosine kinase precursor, epid | 1.449 | 1.407 | 1.586 |
| Sp2 protein | −9 | −2.432 | −1.571 |
| stromal cell derived factor 1 receptor (SDF1 receptor); f [symbol] | 1.961 | 1.334 | 1.531 |
| jun-D | 1.676 | 3.444 | 1.512 |
| trans-acting T-cell specific transcription factor GATA3 | 1.635 | −1.656 | 1.475 |
| early growth response protein 1 (hEGR1); transcription 1 | −2.212 | 1.311 | 1.39 |
| lipid-activated protein kinase PRK1; PKN cell morpholog [symbol] | −2.4 | −2.696 | 1.33 |
| CD27L antigen receptor precursor. T-cell activation CD2 | 24 | −1.531 | 1.29 |
| special AT-rich sequence binding protein 1 (SATB1); M [symbol] | 1.452 | 1.46 | 1.279 |
| guanine nucleotide-binding protein G(I)/G(S)/G(T) beta [symbol] | 1.576 | 1.262 | 1.24 |
| glutaredoxin | −4 | −1.613 | 1.239 |
| rho GDP dissociation inihibitor 1 (RHO-GDI 1); RHO-G [symbol] | −1.23 | −6.564 | 1.18 |
| 40S ribosomal protein S19 (RPS19) | −2.888 | 1.11 | 1.177 |
| MAP kinase-activated protein kinase 2 (MAPKAP kinase | 1.852 | −1.457 | 1.172 |
| calcium-dependent protease small (regulatory) subunit; | 2.07 | 1.126 | 1.128 |
| helix-loop-helix protein; DNA-binding protein inhibitor Id- | 2.701 | 1.164 | 1.005 |
| granulocyte colony stimulating factor receptor precursor | −1.029 | −3.947 | 0 |
| fte-1; yeast mitochondrial protein import homolog; 40S [symbol] | −2.616 | −1.193 | −1.004 |
| TIS11B protein; EGF response factor 1 (ERF1) | 3.26 | −1.517 | −1.005 |
| cAMP-dependent transcription factor ATF-4; DNA-bindir | 1.55 | −1.254 | −1.014 |
| zinc finger protein 91 (ZNF92); HPF7; HTF10 | 1.202 | −1.58 | −1.021 |
| erythroblastosis virus oncogene homolog 1 (ETS-1); P5 [symbol] | −9 | 2.277 | −1.077 |
| transforming protein rhoA H12 (RHO12; ARH12; ARHA) | −2.404 | −1.155 | −1.079 |
| ets domain protein elk-3; NET; SRF accessory protein [symbol] | 1.636 | −1.373 | −1.08 |
| 60S ribosomal protein L6 (RPL6); TAX-responsive enha | 4.046 | −1.005 | −1.086 |
| induced myeloid leukemia cell differentiation protein MC | 1.849 | −1.033 | −1.099 |
| transmembrane 4 superfamily protein; SAS | −1.777 | −1.355 | −1.145 |
| purine-rich single-stranded DNA-binding protein alpha ( [symbol] | 2.077 | −1.372 | −1.153 |
| mutL protein homolog; DNA mismatch repair protein ML | 2.667 | −5.556 | −1.195 |
| microsomal glutathione S-transferase 12 (GST12; MGS | 1.103 | −1.273 | −1.205 |
| transcription initiation factor TFIID 31-KDa subunit; TAFI | 1.48 | −2.55 | −1.219 |
| calcium/calmodulin-dependent protein kinase type IV ca | −1.397 | −1.353 | −1.23 |
| acyl-CoA-binding protein (ACBP); diazepam binding inh | −1.486 | −2.278 | −1.285 |
| fli-1 oncogene; ergB transcription factor | 2.509 | −1.447 | −1.29 |
| cyclin-dependent kinase Inhibitor 1 (CDKN1A); melano [symbol] | 3.324 | −1.209 | −1.377 |
| CDC-like kinase 1 (CLK1) | 1.523 | −1.531 | −1.441 |
| signal transducer and activator of transcription 6 (STAT [symbol] | 2.321 | −1.257 | −1.457 |
| thymosin beta-10 (TMSB10; THYB10); PTMB10 | −3.071 | −1.185 | −1.564 |
| growth factor receptor-bound protein 2 (GRB2) Isoform; | 1.296 | −2.111 | −1.587 |
| c-jun proto-oncogene; transcription factor AP-1 | 1.427 | −3.192 | −1.615 |
| T-cell-specific rantes protein precurso [symbol] . sis delta; small i | −1.62 | −1.573 | −1.739 |
| ZFM1 protein alternatively spliced product | 1.329 | −2.538 | −1.749 |
| leukocyte adhesion glycoprotein LFA-1 alpha subunit pr [symbol] | −1.003 | −1.68 | −1.774 |
| glutathione S-transferase A1 (GTH1; GSTA1); HA subur | 2.033 | 1.234 | −2.072 |
| transducer of erbB2 (TOB) | −3 | −2.079 | −2.121 |
| caspase-10 precursor (CASP10); ICE-LIKE apoptotic pr [symbol] | −1.741 | −8.132 | −2.157 |
| c-myc oncogene | −1.5 | −2.621 | −2.37 |
| ribonuclease/angiogenin Inhibitor (RAI); placental ribon [symbol] | −1.158 | 0 | −2.395 |
| cation-Independent mannose-6-phosphate receptor prec | −1.725 | −1.423 | −2.583 |
| neuromedin B receptor (NMBR); neuromedin-B-preferrin | −1.725 | −1.574 | −4.199 |
| migration Inhibitory factor-related protein 8 (MRP8); cal [symbol] | −1.719 | −4.771 | −5.686 |
| NADH-ubiquinone oxidoreductase B18 subunit; complex | −1.63 | −3.444 | −6.769 |

TABLE 6
GENE CHANGES INDUCED BY ANTHRAX IN VIVO IN MONKEYS

| Change | Function | Gene Name | 24 h | 48 h | 72 h |
|---|---|---|---|---|---|
| AU | TUMOR SUPPRESS [symbol] | putative protein-tyrosine phosphatase [symbol] | 4.53 | 4.42 | 3.43 |
| AU | TRANSCRIPTlON FA | cAMP-response element binding prote [symbol] | 12.09 | 3.32 | 6.50 |
| AU | SIGNAL TRANSDUC | ephrin type-A receptor 1 precursor; tyr [symbol] | 4.93 | 2.05 | 4.66 |
| AU | RECEPTOR-ASSOC [symbol] | ink adaptor protein | 10.90 | 8.14 | 9.35 |
| AU | PROTEIN TURNOVE [symbol] | cathepsin H precursor | 41.72 | 7.85 | 19.14 |
| AU | PROTEIN TURNOVE [symbol] | proteasome component C3; macropair | 15.71 | 5.53 | 16.75 |
| AU | PROTEIN TURNOVE [symbol] | proteasome component C8; macropair | 13.33 | 3.83 | 10.40 |
| AU | PROTEIN TURNOVE [symbol] | methionine aminopeptidase 2 (METAP | 6.65 | 1.53 | 4.23 |
| AU | PROTEIN TURNOVE [symbol] | proteasome component C2; macropair | 4.36 | 6.59 | 13.46 |
| AU | PHOSPHOLIPASES [symbol] | phosphatidylinositol 3-kinase catalytic | 2.85 | 3.36 | 5.93 |
| AU | OTHER INTRACELL [symbol] | GAP-associated protein | 5.70 | 2.87 | 12.23 |
| AU | OTHER EXTRACELL [symbol] | parathymosin | 25.00 | 8.41 | 16.69 |
| AU | OTHER DNA SYNTHI [symbol] | translin; recombination hotspot binding | 9.21 | 1.74 | 3.84 |
| AU | NERVOUS SYSTEM-I | activated RNA polymerase II transcript | 32.48 | 3.09 | 39.28 |
| AU | NERVOUS SYSTEM-I | transcription initiation factor TFIID 31- [symbol] | 25.63 | 3.35 | 8.94 |
| AU | NERVOUS SYSTEM-I | GA-binding protein beta-2 subunit (GA | 24.63 | 1.66 | 13.87 |
| AU | NERVOUS SYSTEM-I | hypoxia-inducible factor 1 alpha (HIF1 | 24.40 | 7.29 | 28.03 |
| AU | NERVOUS SYSTEM-I | interferon regulatory factor 2 (IRF2) | 22.91 | 4.05 | 5.90 |
| AU | NERVOUS SYSTEM-I | cAMP-responsive element-binding prot [symbol] | 16.72 | 2.62 | 8.77 |
| AU | NERVOUS SYSTEM-I | FUSE binding protein | 7.75 | 1.24 | 3.04 |
| AU | NERVOUS SYSTEM-I | human immunodeficiency virus type I [symbol] | 5.97 | 4.18 | 3.87 |
| AU | NERVOUS SYSTEM-I | transcription factor ETR101 | 4.07 | 4.92 | 10.12 |
| AU | KINASE SUBSTRATE | hint protein; protein kinase C inhibitor | 8.39 | 8.56 | 15.51 |
| AU | INTRACELLULAR KI [symbol] | Janus kinase 2 (JAK2); receptor-assoc [symbol] | 17.30 | 8.82 | 13.58 |
| AU | INTRACELLULAR KI [symbol] | protein kinase C beta I (PKC-beta-1) | 8.45 | 7.32 | 10.78 |
| AU | INTRACELLULAR KI [symbol] | dual specificity mitogen-activated prot [symbol] | 6.17 | 3.25 | 3.70 |
| AU | INTRACELLULAR KI [symbol] | cAMP-dependent protein kinase alpha- | 3.98 | 3.18 | 2.64 |
| AU | INTERLEUKIN & INT [symbol] | interleukin-7 receptor alpha subunit pr [symbol] | 2.51 | 1.17 | 4.10 |
| AU | HOMEOSTASIS & DE | natural killer cell enhancing factor (NK [symbol] | 3.60 | 2.79 | 9.30 |
| AU | GROWTH FACTORS | macrophage-specific colony-stimulatin | 12.39 | 2.16 | 7.83 |
| AU | GROWTH FACTORS | placenta growth factors 1 + 2 (PLGF1 [symbol] | 4.30 | 4.58 | 9.38 |
| AU | GROWTH FACTORS | heparin-binding EGF-like growth factor | 3.48 | 7.21 | 2.97 |
| AU | GROWTH FACTORS | heregulin-beta3; glial growth factor; ne [symbol] | 3.26 | 6.12 | 4.91 |
| AU | GROWTH FACTORS | interleukin-8 precursor (IL-8); monocyt [symbol] | 2.93 | 2.41 | 5.48 |
| AU | GROWTH FACTOR [symbol] | granulocyte colony stimulating factor [symbol] | 30.03 | 1.57 | 25.51 |
| AU | G PROTEINS | guanine nucleotide regulatory protein [symbol] | 3.64 | 6.02 | 3.22 |
| AU | G PROTEIN-COUPLE | prostaglandin E2 (PGE) receptor EP4 [symbol] | 21.85 | 10.97 | 21.77 |
| AU | DNA POLYMERASES | proliferating cyclic nuclear antigen (PC [symbol] | 5.83 | 2.00 | 8.10 |
| AU | DISEASE-RELATED I | Alzheimer's disease amyloid A4 protei [symbol] | 24.28 | 1.72 | 17.68 |
| AU | DEATH RECEPTORS | tumor necrosis factor receptor 1 (TNF [symbol] | 9.76 | 3.64 | 17.37 |
| AU | DEATH RECEPTORS | adenosine A1 receptor (ADORA1) | 5.42 | 1.33 | 242 |
| AU | CYCLINS | cyclin K | 8.13 | 1.84 | 4.85 |
| AU | CYCLINS | cyclin T CDK9-associated | 6.69 | 2.21 | 4.89 |
| AU | CELL SURFACE ANT | fibronectin receptor beta subunit (FNRI [symbol] | 29.68 | 2.59 | 17.52 |
| AU | CELL SURFACE ANT | cadherin 3 (CDH3); placental cadherin | 22.80 | 2.24 | 19.62 |
| AU | CELL SURFACE ANT | B-lymphocyte CD19 antigen precursor; | 15.40 | 1.41 | 24.70 |
| AU | CELL CYLCLE REGU | cyclin G-associated kinase (GAK) | 10.78 | 3.33 | 3.86 |
| AU | CDK INHIBITORS | cyclin-dependent kinase 4 inhibitor D ( [symbol] | 7.01 | 3.50 | 10.90 |
| AU | CASPASES | caspase-4 precursor (CASP4); ICH-2 [symbol] | 19.09 | 1.73 | 25.68 |
| AU | CASPASES | caspase-2 precursor (CASP2); ICH-1L | 4.31 | 5.24 | 2.78 |
| AU | BCL FAMILY | apoptosis regulator bcl-x | 9.34 | 2.93 | 25.8 6 |
| AU | APOPTOSIS-ASSOC | growth arrest & DNA-damage-inducible | 24.34 | 1.35 | 29.25 |
| AU | APOPTOSIS-ASSOC | apoptotic protease activating factor 1 ( [symbol] | 22.13 | 2.28 | 14.00 |
| AD | TUMOR SUPPRESS [symbol] | LUCA15 putative tumor suppressor | −17.59 | −3.18 | −9.43 |
| AD | TRANSCRIPTION FA | signal transducer and activator of trans [symbol] | −5.15 | −2.51 | −6.89 |
| AD | TRANSCRIPTION FA | erythroblastosis virus oncogene homol [symbol] | −3.36 | −1.55 | −3.10 |
| AD | NERVOUS SYSTEM-I | B-cell lymphoma 6 protein (bcl-6); zinc [symbol] | −3.43 | −1.28 | −3.61 |
| AD | KINASE SUBSTRATE | 14-3-3 protein sigma; stratifin; epitheli [symbol] | −5.56 | −1.19 | −5.98 |
| AD | KINASE SUBSTRATE | 14-3-3n protein eta; protein AS1; YWH [symbol] | −18.29 | −2.77 | −15.46 |
| AD | INTRACELLULAR KI [symbol] | lipid-activated protein kinase PRK1; PK [symbol] | −4.33 | −1.77 | −1.42 |
| AD | G PROTEINS | ADP-ribosylation factor 1 | −32.07 | −8.70 | −24.39 |
| AD | CYCLINS | G1/S-specific cyclin D3(CCND3) | −4.78 | −1.07 | −5.75 |
| 72hU | PROTEIN TURNOVEI | matrix metalloproteinase 9 (MMP9); g [symbol] | 0.00 | 1.63 | 11.23 |
| 72hU | PROTEIN TURNOVEI | proteasome activator HPA28 subunit b [symbol] | 0.00 | 1.54 | 7.05 |
| 72hU | PROTEIN TURNOVEI | bikunin; hepatocyte growth factor activ [symbol] | 0.00 | 1.94 | 6.15 |
| 72hU | OTHER ONCOGENE: | insulin-like growth factor binding protei [symbol] | 0.00 | 1.17 | 18.60 |
| 72hU | OTHER EXTRACELL [symbol] | Wnt-13 | 0.00 | 0.00 | 5.93 |
| 72hU | OTHER DNA SYNTH [symbol] | growth arrest & DNA-damage-inducibl [symbol] | 1.24 | −2.42 | 7.59 |
| 72hU | OTHER CELL CYCLE | cyclin-D binding Myb-like protein (hDM [symbol] | 4.83 | 1.52 | 15.19 |
| 72hU | OTHER CELL CYCLE | DNA-binding protein inhibitor ID-1; Id-1 [symbol] | 0.00 | 2.64 | 49.00 |
| 72hU | OTHER CELL CYCLE | C-1 | −3.05 | 1.40 | 8.96 |
| 72hU | NEUROTRANSMITTE | membrane-bound & soluble catechol- [symbol] | 1.01 | −1.06 | 10.16 |
| 72hU | NEURONAL DEVELC | roundabout 1 (ROBO1) | 0.00 | 1.72 | 13.44 |
| 72hU | NERVOUS SYSTEM-I | ZFM1 protein alternatively spliced prod [symbol] | 4.22 | 1.48 | 19.61 |
| 72hU | NERVOUS SYSTEM-I | RBP2 retinoblastoma binding protein | 3.93 | 1.71 | 19.79 |
| 72hU | NERVOUS SYSTEM-I | HIV-1 TATA element modulatory facto [symbol] | 1.17 | 1.66 | 8.50 |
| 72hU | NERVOUS SYSTEM-I | interleukin enhancer-binding factor (ILF [symbol] | 1.02 | 1.23 | 8.19 |
| 72hU | NERVOUS SYSTEM-I | histone acetyltransferase B subunit 2; [symbol] | 0.00 | 2.31 | 6.13 |
| 72hU | NERVOUS SYSTEM-I | ets-related gene transforming protein ( [symbol] | 0.00 | 1.09 | 5.41 |
| 72hU | LIGAND-GATED ION | P2X purinoceptor 6 (P2X6); P2XM | 0.00 | 1.76 | 19.54 |
| 72hU | INTRACELLULAR KI [symbol] | dual-specificity mitogen-activated prot [symbol] | 1.19 | 2.21 | 7.82 |
| 72hU | INTERLEUKINS | interleukin-13 precursor (IL-13); NC30 | 0.00 | 1.59 | 7.97 |
| 72hU | INTERLEUKINS | interleron gamma precursor (IFN-gam [symbol] | 0.00 | −1.04 | 4.60 |
| 72hU | INTERLEUKINS | interleukin-5 precursor (IL-5); T-cell re [symbol] | 0.00 | 1.66 | 4.22 |
| 72hU | INTERLEUKIN & INT [symbol] | interleukin-1 receptor type I precursor ( [symbol] | 3.91 | 2.31 | 11.55 |
| 72hU | HORMONES | inhibin alpha subunit precursor (INHA) | 0.00 | 1.18 | 9.61 |
| 72hU | HORMONES | ribonuclease/angiogenin inhibitor (RA [symbol] | −6.28 | 1.38 | 8.77 |
| 72hU | GROWTH FACTORS | hepatocyte growth factor-like protein; [symbol] | 0.00 | 1.27 | 7.78 |
| 72hU | GROWTH FACTORS | endothelial-monocyte activating polyp [symbol] | 0.00 | 1.58 | 6.06 |
| 72hU | GROWTH FACTOR [symbol] | N-sam; fibroblast growth factor recept [symbol] | 0.00 | 2.47 | 7.30 |
| 72hU | FACILITATED DIFFU: | aquaporin 4; WCH4; mercurial-insens [symbol] | 0.00 | 1.60 | 7.08 |
| 72hU | EXTRACELLULAR T [symbol] | apolipoprotein E precursor (APOE) | 0.00 | 1.40 | 7.77 |
| 72hU | DNA POLYMERASES | DNA polymerase gamma (POLG); mit [symbol] | 2.37 | −2.28 | 11.44 |
| 72hU | DEATH RECEPTORS | adenosine A2A receptor (ADORA2A) | 0.00 | 2.09 | 22.32 |
| 72hU | DEATH LIGANDS | lymphotoxin-alpha precursor (LT-alpha | 0.00 | 4.35 | 13.40 |
| 72hU | DEATH LIGANDS | tumor necrosis factor precursor (TNF- [symbol] | −2.79 | 2.21 | 16.30 |
| 72hU | CELL SURFACE ANT | platelet membrane glycoprotein IIB pre | 1.42 | 1.42 | 12.59 |
| 72hU | CELL SURFACE ANT | integrin alpha 6 precursor (ITGA6): VL [symbol] | 0.00 | 1.21 | 9.31 |
| 72hU | CELL SURFACE ANT | CD44 antigen hematopoietic form prec | 0.00 | 1.65 | −7.99 |
| 72hU | CASPASES | cysteine protease ICE-LAP3 | 0.00 | 1.30 | 25.66 |
| 72hU | APOPTOSIS-ASSOC | cytoplasmic antiproteinase 3 (CAP3); [symbol] | 0.00 | −2.15 | 8.47 |
| 72hD | TUMOR SUPPRESS [symbol] | breast cancer type 2 susceptibility prot [symbol] | 0.00 | −1.45 | −4.44 |
| 72hD | TUMOR SUPPRESS [symbol] | neurofibromatosis protein type I (NF1); | 0.00 | 1.11 | −5.73 |
| 72hD | TUMOR SUPPRESS [symbol] | p53 cellular tumor antigen | 0.00 | 0.00 | −6.45 |
| 72hD | TUMOR SUPPRESS [symbol] | tumor suppressor protein DCC precurs | 0.00 | 2.33 | −6.89 |
| 72hD | TUMOR SUPPRESS [symbol] | moesin-ezrin-radbdn-like protein (MER [symbol] | 0.00 | −2.01 | −6.99 |
| 72hD | TRANSCRIPTION FA | signal transducer and activator of trans [symbol] | 0.00 | 1.81 | −5.56 |
| 72hD | TRANSCRIPTION FA | fos-related antigen 2 (FRA2) | 0.00 | −1.98 | −5.63 |
| 72hD | TRANSCRIPTION FA | c-rel proto-oncogene protein | 0.00 | −1.90 | −12.45 |
| 72hD | SIGNAL TRANSDUC⁻ | ephrin A4 precursor (EFNA4); EPH-rel; | 0.00 | −2.53 | −7.17 |
| 72hD | SIGNAL TRANSDUC⁻ | ephrin A3 precursor (EFNA3); EPH-rel; | 3.42 | 0.00 | −7.83 |
| 72hD | PHOSPHOLIPASES [symbol] | phospholipase C gamma 1 (PLC-gam [symbol] | 0.00 | 1.18 | −5.84 |
| 72hD | OTHER INTRACELL [symbol] | zyxin + zyxin-2 | 0.00 | 1.24 | −12.73 |
| 72hD | NERVOUS SYSTEM- [symbol] | transcription factor IIIC box B-binding [symbol] | 0.00 | −1.37 | −4.67 |
| 72hD | NERVOUS SYSTEM- [symbol] | nuclear factor NF90 | 1.34 | 1.69 | −6.87 |
| 72hD | NERVOUS SYSTEM- [symbol] | homeobox A1 protein (HOXA1); HOX1 | 0.00 | 1.54 | −9.09 |
| 72hD | G PROTEINS | transforming protein rhoB; ARHB; AR [symbol] | 0.00 | −1.07 | −11.14 |
| 72hD | DNA DAMAGE REPA | DNA ligase [symbol] ; polydeoxyribonucleotide [symbol] | 0.00 | −1.10 | 6.53 |
| 72hD | CELL CYLCLE REGU | CDC-like kinase 3 (CLK3) | 0.00 | 1.19 | −5.02 |
| 72hD | ADENYLYL/GUANYL [symbol] | cAMP-dependen 3′,5′-cyclic phosphod | 0.00 | −1.19 | −5.39 |
| 72hD | ADENYLYL/GUANYL [symbol] | adenylate cyclase VII; ATP pyrophosp [symbol] | 0.00 | −1.48 | −0.77 |
| 48hU | TYROSINE PHOSPH, | protein-tyrosine phosphatase 1B (PTP- [symbol] | 0.00 | 4.25 | 0.00 |
| 48hU | TYROSINE KINASE [symbol] | tyrosine-protein kinase receptor tyro3 [symbol] | 0.00 | 4.24 | 1.71 |
| 48hU | TUMOR SUPPRESS [symbol] | transforming growth factor beta recept [symbol] | 0.00 | 7.92 | 2.13 |
| 48hU | TUMOR SUPPRESS [symbol] | EB1 protein | 1.45 | 4.51 | 1.35 |
| 48hU | SIGNAL TRANSDUC⁻ | T-lymphocyte activation CD86 antigen | 0.00 | 7.45 | 0.00 |
| 48hU | SIGNAL TRANSDUC⁻ | Interfeukin-6 receptor beta subunit pre [symbol] | 0.00 | 4.51 | 0.00 |
| 48hU | RECEPTOR-ASSOC [symbol] | NCK melanoma cytoplasmic src homo [symbol] | 0.00 | 6.59 | 0.00 |
| 48hU | OTHER INTRACELL [symbol] | FRAP-related protein; protein kinase A | 0.00 | 5.05 | 0.00 |
| 48hU | OTHER DNA SYNTH [symbol] | deoxynbonuclease II (DNase II); acid [symbol] | 1.55 | 4.02 | 0.00 |
| 48hU | OTHER CELL CYCLE | p55CDC | 0.00 | 7.44 | −1.44 |
| 48hU | NERVOUS SYSTEM- [symbol] | zinc-finger DNA-binding protein | 0.00 | 6.70 | 2.39 |
| 48hU | NERVOUS SYSTEM- [symbol] | transcription factor TFIIB; GTF2B | 0.00 | 6.52 | 3.11 |
| 48hU | NERVOUS SYSTEM- [symbol] | metal-regulatory transcription factor | 0.00 | 5.61 | 0.00 |
| 48hU | NERVOUS SYSTEM- [symbol] | C-ets-2 | 0.00 | 4.93 | 0.00 |
| 48hU | NERVOUS SYSTEM- [symbol] | transcriptional regulator interferon-stirr [symbol] | 0.00 | 4.48 | −1.84 |
| 48hU | NERVOUS SYSTEM- [symbol] | ets transcription factor, NERF2 | 0.00 | 4.20 | 0.00 |
| 48hU | NERVOUS SYSTEM- [symbol] | TRAF-interacting protein (I-TRAP) + T [symbol] | 0.00 | 4.06 | 0.00 |
| 48hU | INTRACELLULAR KW [symbol] | MAPK/ERK kinase kinase 3 (MEK kina | 0.00 | 5.84 | 0.00 |
| 48hU | INTRACELLULAR KI [symbol] | casein kinase I gamma 2 (CKI-gamm [symbol] | 0.00 | 5.69 | −1.71 |
| 48hU | INTERLEUKIN & INT [symbol] | interferon-gamma receptor (IFNR-gam [symbol] | 0.00 | 6.38 | 1.13 |
| 48hU | GDP/GTP EXCHANG | GTPase-activating protein (GAP); ras [symbol] | 0.00 | 4.44 | 0.00 |
| 48hU | G PROTEINS | Ral A; GTP-binding protein | 0.00 | 4.91 | 1.22 |
| 48hU | DEATH RECEPTOR- [symbol] | FAN protein | 0.00 | 4.90 | 0.00 |
| 48hU | CALCIUM-BINDING [symbol] | recoverin; cancer-associated retinopat [symbol] | 2.52 | 7.62 | 0.00 |
| 48hU | ADENYLYL/GUANYL [symbol] | cGMP-Inhibited 3′,5′-cyciic phosphodie | 0.00 | 4.90 | 0.00 |
| 48hD | PROTEIN TURNOVE [symbol] | cathepsin D precursor (CTSD) | 1.72 | −4.65 | 3.52 |
| 48hD | PHOSPHOLIPASES [symbol] | phospholipase C beta 3 (PLC beta 3); | 0.00 | −4.09 | −1.13 |
| 48hD | NERVOUS SYSTEM- [symbol] | interferon consensus sequence-binding | 0.00 | −4.97 | 0.00 |
| 48hD | NERVOUS SYSTEM- [symbol] | CYCLIC-AMP-DEPENDENT TRANSC | 0.00 | −5.31 | 0.00 |
| 48hD | NERVOUS SYSTEM- [symbol] | PRB-binding protein E2F1; retinoblast [symbol] | 0.00 | −7.67 | 0.00 |
| 48hD | INTRACELLULAR KI [symbol] | tyk2 non-receptor protein tyrosine kina; | 0.00 | −4.03 | −1.33 |
| 48hD | INTERLEUKIN & INT [symbol] | interfeukin-1 receptor type II precursor | 0.00 | −4.07 | 0.00 |
| 48hD | HORMONES | corticotropin-releasing factor-binding p [symbol] | 0.00 | −4.02 | 0.00 |
| 48hD | HOMEOSTASIS & DE | growth arrest & DNA-damage-Inducible | 0.00 | −5.62 | 0.00 |
| 48hD | GROWTH FACTORS | interferon gamma-induced protein prec [symbol] | 0.00 | −4.93 | 0.00 |
| 48hD | GROWTH FACTOR [symbol] | platelet-activating factor receptor (PAF [symbol] | 0.00 | −4.13 | 0.00 |
| 48hD | G PROTEIN-COUPLE | metabotropic glutamate receptor 5 pre [symbol] | 0.00 | −4.07 | 0.00 |
| 48hD | G PROTEIN-COUPLE | somatostatin receptor type 2 (SS2R); [symbol] | 0.00 | −5.27 | 0.00 |
| 48hD | G PROTEIN-COUPLE | mu-type opioid receptor (MOR-1) | 0.00 | −5.86 | 0.00 |
| 48hD | DISEASE-RELATED [symbol] | atrophin-1; dentatorubral-pallidoluysiar [symbol] | 0.00 | −5.14 | 1.32 |
| 48hD | DISEASE-RELATED [symbol] | Kallmann syndrome protein precursor [symbol] | 0.00 | −6.09 | 0.00 |
| 48hD | DISEASE-RELATED I | FCMD; fukutin | 0.00 | −7.02 | 0.00 |
| 48hD | CELL SURFACE ANT | integrin alpha 4 precursor (ITGA4); VL | 0.00 | −4.55 | 0.00 |
| 48hD | CELL SURFACE ANT | semaphorin; CD100 | 0.00 | −6.41 | 0.00 |
| 48hD | CDK INHIBITORS | cyclin-dependent kinase 4 inhibitor B ( [symbol] | 0.00 | −6.43 | 0.00 |
| 48hD | BCL FAMILY | NIP1 (NIP1) | 0.00 | −4.55 | 0.00 |
| 48hD | BCL FAMILY | NIP3 (NIP3) | 0.00 | −5.63 | 0.00 |
| 48 72 [symbol] | SIGNAL TRANSDUC [symbol] | epithelial discoidin domain receptor 1 [symbol] | 0.00 | 2.95 | 5.23 |
| 48 72 [symbol] | SERINE/THREONINE | c-raf proto-oncogene | 1.44 | 3.75 | 5.54 |
| 48 72 [symbol] | PROTEIN TURNOVEI | proteasome component C5; macropair | 2.09 | 5.20 | 26.46 |
| 48 72 [symbol] | PHOSPHOLIPASES [symbol] | phosphatidylinositol 3-kinase regulator | 0.00 | 8.42 | 6.90 |
| 48 72 [symbol] | OTHER INTRACELL [symbol] | TRRAP protein | 2.07 | 9.09 | 16.73 |
| 48 72 [symbol] | NON-RECEPTOR TY | C-src proto-oncogene (SRC1) | 0.00 | 1.88 | 4.57 |
| 48 72 [symbol] | INTRACELLULAR SIC | transforming protein p21/K-ras 2B | −2.68 | 7.75 | 5.16 |
| 48 72 [symbol] | INTRACELLULAR KI [symbol] | cAMP-dependent protein kinase beta-c | 1.08 | 5.12 | 7.59 |
| 48 72 [symbol] | HORMONES | cellular retinoic acid-binding protein II [symbol] | 0.00 | 2.90 | 7.59 |
| 48 72 [symbol] | HOMEOSTASIS & DE | ferrochelatase precursor; protoheme f [symbol] | 0.00 | 7.15 | 13.20 |
| 48 72 [symbol] | DEATH RECEPTORS | protein-tyrosine phosphatase zeta prec | 0.00 | 3.91 | 4.82 |
| 48 72 [symbol] | ATPASE TRANSPOR | sodium/potassium-transporting ATPas | 0.00 | 3.61 | 4.17 |
| 48 72 [symbol] | INFLAMMATION | alpha-1-acid glycoprotein 1 precursor [symbol] | 0.00 | −3.63 | −4.17 |
| 48 72 [symbol] | INFLAMMATION | eosinophil granule major basic protein | 0.00 | −4.07 | −4.25 |
| 48 72 [symbol] | GROWTH FACTORS | transforming growth factor-alpha (TGF | 0.00 | −3.43 | −5.28 |
| 48 72 [symbol] | GROWTH FACTORS | osteoclast stimulating factor | 0.00 | −3.04 | −7.27 |
| 48 72 [symbol] | G PROTEINS | neuro epithelioma transforming gene 1 | 0.00 | −2.58 | −4.38 |
| 24hU | TYROSINE PHOSPH [symbol] | serine/threonine protein phosphatase [symbol] | 26.91 | 1.33 | 3.35 |
| 24hU | TYROSINE PHOSPH [symbol] | serine/threonine protein phosphatase 2 | 25.80 | 1.43 | 2.14 |
| 24hU | TYROSINE PHOSPH [symbol] | PTPCAAX1 nuclear tyrosine phosphata [symbol] | 24.98 | 2.03 | 2.04 |
| 24hU | TYROSINE PHOSPH [symbol] | protein phosphatase 2C alpha isoform | 16.20 | 2.77 | 3.16 |
| 24hU | TYROSINE KINASE [symbol] | macrophage colony stimulating factor | 48.94 | 3.74 | 1.42 |
| 24hU | TYROSINE KINASE [symbol] | ERBB-3 receptor protein-tyrosine kinas [symbol] | 9.64 | −1.14 | 1.27 |
| 24hU | TUMOR SUPPRESS [symbol] | c-myc purine-binding transcription fact | 31.41 | 1.20 | 2.27 |
| 24hU | TRANSCRIPTION FA | signal transducer and activator of trans [symbol] | 16.26 | 1.62 | 2.45 |
| 24hU | TRANSCRIPTION FA | c-myc oncogene | 41.72 | 1.63 | 1.63 |
| 24hU | TRANSCRIPTION FA | myb proto-oncogene; c-myb | 18.51 | 1.38 | −1.11 |
| 24hU | TRANSCRIPTION FA | ets-related protein tel; ets translocation | 10.22 | 2.24 | 2.74 |
| 24hU | TRANSCRIPTION FA | c-jun proto-oncogene; transcription fac | 6.31 | 1.29 | 2.21 |
| 24hU | SIGNAL TRANSDUC [symbol] | urokinase-type plasminogen activator [symbol] | 20.68 | 2.05 | 2.08 |
| 24hU | RECEPTOR-ASSOCI [symbol] | tyrosine-protein kinase lyn | 42.15 | 2.52 | 1.92 |
| 24hU | RECEPTOR-ASSOCI [symbol] | c-src kinase (CSK); protein-tyrosine ki [symbol] | 30.65 | 2.40 | 10.40 |
| 24hU | RECEPTOR-ASSOCI [symbol] | growth factor receptor-bound protein 2 | 14.02 | 1.50 | 1.84 |
| 24hU | RECEPTOR-ASSOCI [symbol] | epidermal growth factor receptor subst [symbol] | 12.12 | 2.04 | 2.92 |
| 24hU | PROTEIN TURNOVEI | alpha-1-antitrypsin precursor; alpha-1 [symbol] | 48.86 | 2.67 | 3.03 |
| 24hU | PROTEIN TURNOVEI | zinc finger X-chromosomal protein (ZF | 28.33 | −2.72 | 2.53 |
| 24hU | PROTEIN TURNOVEI | leukocyte elastase Inhibitor (LEI); mon | 5.33 | 2.10 | 0.00 |
| 24hU | PHOSPHOLIPASES [symbol] | phosphatidylinositol-4-phosphate 5-kin [symbol] | 15.82 | 2.51 | 1.30 |
| 24hU | PHOSPHOLIPASES [symbol] | phospholipase C-gamma-2 (PLC-gam [symbol] | 10.19 | 2.29 | 2.81 |
| 24hU | OTHER ONCOGENE: | matrix metalloproteinase 11 (MMP11); | 19.92 | 1.57 | 2.29 |
| 24hU | OTHER INTRACELLL | leucine-rich repeat protein SHOC-2; ra [symbol] | 27.50 | 3.48 | 3.81 |
| 24hU | OTHER INTRACELLL | tuberin; tuberous sclerosis 2 protein (T [symbol] | 14.69 | 2.69 | 3.01 |
| 24hU | NON-RECEPTOR TY | C-fgr proto-oncogene (p55-FGR); SRC [symbol] | 51.43 | 1.44 | 2.90 |
| 24hU | NON-RECEPTOR TY | C-yes proto-oncogene (YES1) | 12.39 | −1.45 | 0.00 |
| 24hU | NON-RECEPTOR TY | CBL-B | 10.10 | 2.05 | 1.68 |
| 24hU | NEURONAL DEVELC | glia maturation factor beta (GMF-beta) | 7.25 | −2.71 | 0.00 |
| 24hU | NEUROMEDIATORS | acyl-CoA-binding protein (ACBP); diaz [symbol] | 38.20 | 2.01 | 1.90 |
| 24hU | NERVOUS SYSTEM-I | CACCC-box DNA-binding protein | 31.42 | 2.41 | 0.00 |
| 24hU | NERVOUS SYSTEM-I | trans-acting T-cell specific transcription | 28.26 | 1.97 | 1.23 |
| 24hU | NERVOUS SYSTEM-I | nucleobindin precursor (NUC) | 27.48 | 1.69 | 1.70 |
| 24hU | NERVOUS SYSTEM-I | CCAAT-binding transaction factor sut [symbol] | 25.98 | 1.33 | 1.95 |
| 24hU | NERVOUS SYSTEM-I | cellular nucleic acid binding protein (CI [symbol] | 23.52 | 1.95 | 1.27 |
| 24hU | NERVOUS SYSTEM-I | putative transcription activator DB1 | 22.78 | 2.06 | 2.24 |
| 24hU | NERVOUS SYSTEM-I | estrogen receptor hSNF2b; global tran; | 20.64 | 1.68 | 2.20 |
| 24hU | NERVOUS SYSTEM-I | zinc finger protein 91 (ZNF92); HPF7; I [symbol] | 17.36 | 1.95 | 1.70 |
| 24hU | NERVOUS SYSTEM-I | transcriptional repressor protein yin & [symbol] | 15.89 | 2.69 | 3.57 |
| 24hU | NERVOUS SYSTEM-I | transactionfactor LSF | 12.12 | 1.36 | −1.49 |
| 24hU | NERVOUS SYSTEM-I | transcriptional activator hSNF2-alpha | 9.73 | 1.51 | 1.73 |
| 24hU | NERVOUS SYSTEM-I | heat shock factor protein 1 (HSF1): he [symbol] | 9.01 | 2.17 | 1.83 |
| 24hU | NERVOUS SYSTEM-I | CCAAT transcription binding factor ga [symbol] | 7.63 | 1.73 | 0.00 |
| 24hU | NERVOUS SYSTEM-I | paired box protein PAX-5; B-cell specif [symbol] | 6.40 | 1.22 | 1.19 |
| 24hU | NERVOUS SYSTEM-I | DNA-binding protein TAXRE8302; alb [symbol] | 5.00 | 1.55 | 1.90 |
| 24hU | NERVOUS SYSTEM-I | Sp3 protein | 3.69 | −1.64 | −3.75 |
| 24hU | NERVOUS SYSTEM-I | ets domain protein elk-3; NET; SRF a [symbol] | 3.51 | 1.41 | 1.60 |
| 24hU | KINASE SUBSTRATE | protein kinase C substrate 80-kDa pro [symbol] | 32.38 | 1.84 | 2.77 |
| 24hU | KINASE SUBSTRATE | 14-3-3 protein beta/alpha; protein kina: [symbol] | 26.20 | 1.87 | 2.39 |
| 24hU | INTRACELLULAR SI [symbol] | N-ras; transforming p21 protein | 4.72 | 1.80 | 0.00 |
| 24hU | INTRACELLULAR KI [symbol] | mitogen-activated protein kinase p38 ( [symbol] | 33.40 | 2.48 | 1.93 |
| 24hU | INTRACELLULAR KI [symbol] | protein kinase C delta (NPKC-delta) | 32.15 | 4.90 | 1.17 |
| 24hU | INTRACELLULAR KI [symbol] | protein kinase MLK-3; sprk | 23.72 | 2.51 | 1.02 |
| 24hU | INTRACELLULAR KI [symbol] | cAMP-dependent protein kinase I alpha | 22.75 | 2.06 | 3.36 |
| 24hU | INTRACELLULAR KI [symbol] | serine kinase | 19.02 | 1.53 | 1.51 |
| 24hU | INTRACELLULAR KI [symbol] | tyrosine-protein kinase ack | 17.33 | 1.56 | 3.37 |
| 24hU | INTRACELLULAR KI [symbol] | calcium/calmodulin-dependent protein | 8.94 | 1.56 | 1.71 |
| 24hU | INTRACELLULAR KI [symbol] | dual specificity mitogen-activated prote [symbol] | 7.31 | 4.15 | 2.41 |
| 24hU | INTRACELLULAR KI [symbol] | dual specificity mitogen-activated prote [symbol] | 7.27 | 1.48 | 1.76 |
| 24hU | INTRACELLULAR KI [symbol] | glycogen synthase kinase 3 beta (GSK | 4.70 | −1.34 | −1.19 |
| 24hU | INTERLEUKINS | interleukin-18 precursor (IL-18); interfe [symbol] | 17.01 | 3.04 | 0.00 |
| 24hU | INTERLEUKINS | interleukin-10 precursor (IL-10); cytoki [symbol] | 5.66 | −1.29 | −1.18 |
| 24hU | INTERLEUKIN & INTE [symbol] | interleukin-6 receptor alpha subunit pr [symbol] | 4.06 | 1.23 | 0.00 |
| 24hU | HORMONES | estrogen sulfotransferase (STE; EST1) | 6.96 | 1.22 | 2.19 |
| 24hU | HOMEOSTASIS & DE | heat shock 90-kDa protein A (HSP90A | 30.63 | 1.64 | 2.62 |
| 24hU | HOMEOSTASIS & DE | mitochondrial matrix protein P1 precur [symbol] | 15.40 | 1.74 | −1.70 |
| 24hU | HOMEOSTASIS & DE | heat-shock protein 40 (HSP40) | 11.13 | −1.04 | −1.45 |
| 24hU | HOMEOSTASIS & DE | glutathione S-transferase theta 1 (GST | 9.81 | 1.37 | 2.98 |
| 24hU | HOMEOSTASIS & DE | glutaredoxin | 8.50 | 1.13 | −5.25 |
| 24hU | HOMEOSTASIS & DE | glutathione S-transferase A1 (GTH1; [symbol] | 6.69 | −1.20 | 2.31 |
| 24hU | GROWTH FACTORS | migration inhibitory factor-related prote [symbol] | 51.68 | 1.34 | −1.77 |
| 24hU | GROWTH FACTORS | insulin-like growth factor binding prote [symbol] | 8.88 | 1.53 | 1.00 |
| 24hU | G PROTEINS | ras-related protein RAP-1B; GTP-bind [symbol] | 48.81 | 2.79 | 2.82 |
| 24hU | G PROTEINS | ras-related C3 botulinum toxin substra [symbol] | 31.24 | 1.57 | 7.23 |
| 24hU | G PROTEINS | guanine nucleotide-binding protein G( [symbol] | 7.64 | −1.12 | 1.52 |
| 24hU | G PROTEINS | ras-related protein RAB5A | 4.64 | 1.61 | −1.22 |
| 24hU | DNA DAMAGE REPA | xeroderma pigmentosum group C repa | 14.95 | 1.13 | 2.05 |
| 24hU | DISEASE-RELATED I | Machado-Joseph disease protein 1 (M [symbol] | 12.57 | −3.20 | 1.23 |
| 24hU | CYCLINS [symbol] | fte-1; yeast mitochondrial protein impo [symbol] | 34.47 | 1.56 | 2.98 |
| 24hU | CYCLINS | cation-independent mannose-6-phosp [symbol] | 9.32 | 1.01 | 1.02 |
| 24hU | CELL SURFACE ANT | cell surface glycoprotein mac-1 alpha [symbol] | 18.22 | 1.62 | 3.49 |
| 24hU | CELL SURFACE ANT | platelet membrane glycoprotein IIIA pr [symbol] | 8.45 | 1.21 | 2.31 |
| 24hU | CELL CYLCLE REGU | CDC-like kinase 1 (CLK1) | 15.99 | 1.81 | 2.03 |
| 24hU | CELL CYLCLE REGU | CDC2-related protein kinase CHED | 5.38 | 1.45 | 0.00 |
| 24hU | CDK INHIBITORS | cyclin-dependent kinase 4 inhibitor (C [symbol] | 5.31 | −1.30 | 0.00 |
| 24hU | CASPASES | caspase-8 precursor (CASP8); ICE-like | 30.83 | 1.58 | 2.79 |
| 24hU | CALPAINS | calcium-dependent protease small (reg [symbol] | 26.15 | 1.78 | 1.66 |
| 24hU | APOPTOSIS-ASSOC | defender against cell death 1 (DAD1) | 40.10 | 2.41 | 2.85 |
| 24hD | TUMOR SUPPRESS [symbol] | cadherin1 (CDH1); epithelial cadherin I | −4.93 | 0.00 | 0.00 |
| 24hD | TRANSCRIPTION FA | jun-D | −27.02 | −1.26 | −2.02 |
| 24hD | SIGNAL TRANSDUC⁻ | stromal cell derived factor 1 receptor ( [symbol] | −9.18 | −1.28 | 1.99 |
| 24hD | SERINE/THREONINE | A-raf proto-oncogene serine/threonine- | −12.57 | 1.32 | 0.00 |
| 24hD | NERVOUS SYSTEM-I | Sp2 protein | −7.45 | −1.16 | −1.39 |
| 24hD | NERVOUS SYSTEM-I | cAMP-dependent transcription factor A | −22.50 | −1.17 | −2.21 |
| 24hD | INTRACELLULAR SI [symbol] | C-cb [symbol] proto-oncogene | −4.62 | −1.34 | 0.00 |
| 24hD | DNA DAMAGE REPA | DNA-repair protein XRCC1 | −26.15 | 1.05 | 1.44 |
| 24hD | DEATH RECEPTORS | CD27L antigen receptor precursor; T-c | −13.56 | 1.23 | 1.55 |
| 24hD | CELL SURFACE ANT | leukocyte adhesion glycoprotein LFA-1 | −4.52 | 1.19 | 1.57 |
| 24hD | CELL CYLCLE REGU | BUBR1 protein kinase | −3.57 | 1.73 | 0.00 |
| 24hD | CASPASES | caspase-10 precursor (CASP10); ICE-I | −63.06 | 1.17 | 1.25 |
| 24hD | CALCIUM-BINDING F | S100 calcium-binding protein A7; psori | −6.06 | 0.00 | 0.00 |
| 24 48 [symbol] | TYROSINE PHOSPH, | protein phosphatase PP2A 55-kDa reg | 3.88 | 2.18 | 0.00 |
| 24 48 [symbol] | TUMOR SUPPRESS [symbol] | p78 putative SERINE/THREONINE-protein k | 20.97 | 5.12 | 1.67 |
| 24 48 [symbol] | TUMOR SUPPRESS [symbol] | p53-associated mdm2 protein | 2.96 | 5.33 | −1.83 |
| 24 48 [symbol] | SYMPORTERS & AN [symbol] | sodium/hydrogen exchanger 1 (Na+/H− | 5.73 | 10.90 | −1.67 |
| 24 48 [symbol] | SERINE/THREONINE | pim-1 proto-oncogene | 3.62 | 3.44 | 0.00 |
| 24 48 [symbol] | NEUROMEDIATORS | glial growth factor 2 precursor (GGFH | 17.72 | 10.16 | 2.05 |
| 24 48 [symbol] | NERVOUS SYSTEM-I | purine-rich single-stranded DNA-bindin | 18.65 | 3.80 | 1.03 |
| 24 48 [symbol] | NERVOUS SYSTEM-I | DNA-binding protein HIP116; ATPase; | 7.79 | 2.47 | −2.00 |
| 24 48 [symbol] | NERVOUS SYSTEM-I | transcription factor Sp1 (TSFP1) | 6.18 | 3.60 | 1.42 |
| 24 48 [symbol] | NERVOUS SYSTEM-I | RNA polymerase II elongation factor S | 5.57 | 4.31 | 0.00 |
| 24 48 [symbol] | NERVOUS SYSTEM-I | transcription elongation factor SII | 3.02 | 2.17 | −5.28 |
| 24 48 [symbol] | INTRACELLULAR KI [symbol] | ribosomal protein S6 kinase II alpha 3 | 19.73 | 5.10 | 2.16 |
| 24 48 [symbol] | INTRACELLULAR KI [symbol] | Janus kinase 1 (JAK1) | 4.75 | 3.70 | 1.33 |
| 24 48 [symbol] | INTRACELLULAR KI [symbol] | cAMP-dependent protein kinase type I | 4.65 | 3.47 | 1.67 |
| 24 48 [symbol] | INTRACELLULAR KI [symbol] | protein kinase C eta type (NPKC-eta); | 4.23 | 5.69 | −3.55 |
| 24 48 [symbol] | HOMEOSTASIS & DE | glutathione S-transferase mu1 (GSTM [symbol] | 3.52 | 2.87 | 1.33 |
| 24 48 [symbol] | G PROTEINS | ras-related protein RAB2 | 28.35 | 9.69 | 2.05 |
| 24 48 [symbol] | BCL FAMILY | BCL-2 binding athanogene-1 (BAG-1); | 11.31 | 3.89 | 0.00 |
| 24 48 [symbol] | OTHER CELL CYCLE | CDC25B; CDC25HU2; M-phase induce | −4.89 | −2.25 | 0.00 |
| 24 48 [symbol] | NON-RECEPTOR TY | proto-oncogene tyrosine-protein kinase | −3.61 | −1.79 | 0.00 |
| 24 48 [symbol] | LIGAND-GATED ION | 5-hydroxytryptamine 3 receptor precur [symbol] | −2.37 | −8.77 | 0.00 |
| 24 48 [symbol] | CELL CYLCLE REGU | aurora- & IPL1-like midbody-associate | −3.54 | −5.05 | 0.00 |

AU = up at all time point;
AD = down at all time point;
24hU = up in 24 hr;
48hU = up in 48 hr;
72hU = up in 72 hr

TABLE 7a
GENE CHANGES INDUCED BY VEE VIRUS IN VITRO IN HUMAN LYMPHOID CELLS -
ARRAY I

| Gene code | Gene Function | Protein/gene | VEE-1 h | VEE-4 h |
|---|---|---|---|---|
| A01a | Oncogenes & Tumor Suppressors | Von Hippel-Lindau tumor suppres [symbol] | 0.4 | −2.2 |
| A02b | G Proteins | ras-related protein RAP-1A; C21K | 3.5 | −1.9 |
| A02d | Oncogenes & Tumor Suppressors | erythroblastosis virus oncogene h [symbol] | 1.5 | 0.0 |
| A03c | Oncogenes & Tumor Suppressors | neogenin | 1.1 | −2.0 |
| A03e | Oncogenes & Tumor Suppressors | c-raf proto-oncogene | 1.5 | −2.9 |
| A03g | Oncogenes & Tumor Suppressors | N-ras; transforming p21 protein | 1.8 | 0.0 |
| A03l | Other Cell Cycle Proteins | prothymosin alpha (ProT-alpha; F | 0.0 | −2.4 |
| A04b | Oncogenes & Tumor Suppressors | ezrin; cytovillin 2; villin 2 (VIL2) | 1.7 | −2.2 |
| A04d | Oncogenes & Tumor Suppressors | jun-D | 2.9 | 0.0 |
| A04e | Oncogenes & Tumor Suppressors | A-raf proto-oncogene serine/threo [symbol] | 1.7 | −1.3 |
| A04g | Oncogenes & Tumor Suppressors | C-cbl proto-oncogene | 1.7 | 0.0 |
| A04l | Other Cell Cycle Proteins | DNA-binding protein inhibitor ID-1 | 1.8 | 0.2 |
| A04n | Ligand-Gated Ion Channels | ASIC3 proton gated cation chann [symbol] | 0.6 | −1.6 |
| A05j | Cell Cycle-Regulating Kinases | serine/threonine-protein kinase K [symbol] | 0.5 | −1.6 |
| A06c | Oncogenes & Tumor Suppressors | C-maf transcription factor | 1.4 | −2.1 |
| A06n | Ligand-Gated Ion Channels | ATP-sensitive inward rectifier pot [symbol] | 0.0 | −1.6 |
| A07c | Oncogenes & Tumor Suppressors | elk-1; ets-related proto-oncogene | 1.0 | −2.3 |
| A08a | Oncogenes & Tumor Suppressors | moesin-ezrin-radixin-like protein (I | −1.0 | −1.6 |
| A08d | Oncogenes & Tumor Suppressors | v-erbA related protein (EAR2) | 0.0 | −2.5 |
| A08h | Cyclins | cyclin H (CCNH); MO15-associate | 2.0 | −0.2 |
| A08m | Facilitated Diffusion Proteins | brain glucose transporter 3 (GTR3 | 2.1 | 0.1 |
| A09e | Oncogenes & Tumor Suppressors | papillary thyroid carcinoma-encod [symbol] | 0.0 | −1.5 |
| A09g | Oncogenes & Tumor Suppressors | insulin-like growth factor binding p | 5.0 | 0.0 |
| A10a | Oncogenes & Tumor Suppressors | p53 cellular tumor antigen | 0.0 | −1.9 |
| A10c | Oncogenes & Tumor Suppressors | c-jun proto-oncogene; transcriptio [symbol] | 2.2 | −2.1 |
| A10k | Other Cell Cycle Proteins | geminin | 0.0 | −1.6 |
| A11c | Oncogenes & Tumor Suppressors | myb proto-oncogene; c-myb | 2.2 | 0.0 |
| A11d | Oncogenes & Tumor Suppressors | v-erbA related protein (EAR3); CC | 0.2 | −1.6 |
| A11e | Oncogenes & Tumor Suppressors | ERBB2 receptor protein-tyrosine k | 0.5 | −2.4 |
| A11f | Oncogenes & Tumor Suppressors | ski oncogene | 1.5 | 0.0 |
| A12b | Oncogenes & Tumor Suppressors | maguk p55 subfamily member 2; I | 0.0 | −2.1 |
| A12c | Oncogenes & Tumor Suppressors | c-myc oncogene | 3.1 | −1.6 |
| A12f | Oncogenes & Tumor Suppressors | snoN oncogene | −2.3 | 0.0 |
| A13b | Oncogenes & Tumor Suppressors | tumor suppressor maspin; proteas | 0.7 | −1.8 |
| A13e | Oncogenes & Tumor Suppressors | ERBB4 receptor protein-tyrosine k | 1.5 | 0.0 |
| A13g | Cyclins | cyclin K | 2.3 | 0.0 |
| A13m | Facilitated Diffusion Proteins | putative renal organic anion trans [symbol] | 0.0 | −2.2 |
| A14a | Oncogenes & Tumor Suppressors | colorectal mutant cancer protein (I | 0.0 | −2.6 |
| A14c | Oncogenes & Tumor Suppressors | L-myc proto-oncogene (MYCL1) | 0.0 | −1.5 |
| A14d | Oncogenes & Tumor Suppressors | C-mos proto-oncogene serine/thre | 0.5 | −1.9 |
| A14g | Cyclins | cyclin E2 | 2.2 | 0.0 |
| A14l | Other Cell Cycle Proteins | RCL growth-related c-myc-respon [symbol] | 1.3 | −1.5 |
| B01g | Intracellular Adaptors & Receptor- | proto-oncogene tyrosine-protein ki | 1.7 | 0.2 |
| | Associated Proteins | | | |
| B01l | Intracellular Kinase Network Members | serine/threonin-protein kinase PA [symbol] | 1.3 | −2.0 |
| B02d | Intracellular Transducers, Effectors & | autocrine motility factor receptor ( [symbol] | 0.0 | −1.5 |
| | Modulators | | | |
| B03a | Voltage-Gated Ion Channels | KCNQ3 potassium channel | 0.4 | −2.1 |
| B03j | Intracellular Kinase Network Members | janus kinase 3 (JAK3); leukocyte j | 2.9 | 0.4 |
| B04c | Extracellular Transporters & Carrier | apolipoprotein E precursor (APOE | 1.7 | −2.0 |
| | Proteins | | | |
| B04g | Intracellular Adaptors & Receptor- | epidermal growth factor receptor s | 1.9 | 0.0 |
| | Associated Proteins | | | |
| B04h | Intracellular Kinase Network Members | tyrosine-protein kinase ack | 1.9 | 0.0 |
| B05b | Symporters & Antiporters | high-affinity glutamate transporter | 0.0 | −1.8 |
| B06a | Cell Signaling & Extracellular | sodium-dependent dopamine tran [symbol] | 0.2 | −2.0 |
| | Communication Proteins | | | |
| B06i | Intracellular Kinase Network Members | protein kinase C epsilon type (NPI | 1.6 | 1.1 |
| B06j | Intracellular Kinase Network Members | C-jun N-terminal kinase 3 alpha2 [symbol] | −2.3 | 0.8 |
| B07b | ATPase Transporters | copper-transporting ATPase 2; co [symbol] | 0.0 | −4.5 |
| | Intracellular Transducers, Effectors & | | | |
| B07d | Modulators | ephrin type-B receptor 2 precurso [symbol] | 0.0 | −1.9 |
| B07k | Intracellular Kinase Network Members | calcium/calmodulin-dependent pr [symbol] | 0.3 | 1.8 |
| | Intracellular Transducers, Effectors & | | | |
| B07l | Modulators | ephrin A3 precursor (EFNA3); EP [symbol] | 0.4 | 1.9 |
| B07m | G Proteins | Ral A; GTP-binding protein | 0.0 | 1.6 |
| B08h | Intracellular Kinase Network Members | LIM domain kinase 1 (LIMK-1) | 0.5 | 1.6 |
| B08i | Intracellular Kinase Network Members | protein kinase C gamma type (PK [symbol] | 0.0 | 1.6 |
| B08k | Intracellular Kinase Network Members | phosphorylase B kinase gamma c [symbol] | 0.7 | 1.9 |
| B09k | Intracellular Kinase Network Members | casein kinase I gamma 2 (CKI-ga | 0.0 | 1.8 |
| B10a | Symporters & Antiporters | sodium- & chloride-dependent tau | 0.5 | 1.8 |
| B10f | Intracellular Adaptors & Receptor- | c-src kinase (CSK); protein-tyrosin | 2.0 | 0.0 |
| | Associated Proteins | | | |
| B10h | lntracellular Kinase Network Members | dual-specificity mitogen-activated | 0.3 | 2.2 |
| | Intracellular Transducer, Effectors & | | | |
| B11d | Modulators | Interferon-gamma (IFN-gamma) r [symbol] | −1.6 | 0.9 |
| B12h | Intracellular Kinase Network Members | dual specificity mitogen-activated | 0.0 | 2.2 |
| B12i | Intracellular Kinase Network Members | calcium/calmodulin-dependent pr [symbol] | 1.8 | 0.9 |
| B13n | G Proteins | RalB GTP-binding protein | −1.8 | 1.6 |
| C01d | Transcription Activators & Repressors | signal transducer and activator of | 0.3 | 1.8 |
| C01h | Death Receptor-Associated Proteins & | caspase & rip adaptator with death | −1.6 | −1.2 |
| | Adaptors | | | |
| C01i | Calpains | calpain p94 large (catalytic) subun | −2.0 | 0.4 |
| C02e | Kinase Activators & Inhibitors | protein kinase C substrate 80-KDa | 0.5 | 1.8 |
| C02j | DNA Fragmentation Proteins | CAD; DNA fragmentation factor 4 [symbol] | −1.5 | 0.1 |
| C03e | Kinase Activators & Inhibitors | linker for activation of T-cells (LA [symbol] | 1.0 | 1.7 |
| | Other Intracellular Transducers, | | | |
| C03f | Effectors & Modulators | SH3P18 SH3 domain-containing [symbol] | 0.0 | 1.6 |
| C04g | Death Receptors | lymphocyte activation CD30 antig [symbol] | −2.8 | 0.0 |
| C04j | Death Kinases | rac-alpha serine/threonine kinase | 0.1 | −2.3 |
| C04k | Other Apoptosis-Associated Proteins | Inhibitor of apoptosis protein 3 (A [symbol] | 0.4 | −1.6 |
| C04l | DNA Polymerases, Replication Factors | DNA topoisomerase I (TOP1) | 0.0 | 1.7 |
| | & Topoisomerases | | | |
| C04n | DNA Damage Repair Proteins & Ligases | excision repair protein ERCC6; C [symbol] | 1.6 | 0.0 |
| C05a | Intracellular Protein Phosphatases | leukocyte antigen-related protein [symbol] | 0.0 | −2.3 |
| | Other Intracellular Transducers, | | | |
| C05f | Effectors & Modulators | connector enhancer of KSR-like p [symbol] | −2.3 | 0.3 |
| C05h | Caspases | caspase-3 (CASP3); apopain prec | −1.8 | 0.7 |
| C06e | Kinase Activators & Inhibitors | 14-3-3 protein sigma; stratifin; epi [symbol] | 2.9 | 0.9 |
| C06f | Death Receptor Ligands | CD40 ligand (CD40-L); tumor nec [symbol] | −2.4 | −1.4 |
| C07g | Death Receptors | tumor necrosis factor receptor (T [symbol] | 0.0 | 1.8 |
| C07j | Death Kinases | Fas-activated serine/threonine (F [symbol] | 0.4 | −2.0 |
| C07k | Other Apoptosis-Associated Proteins | cytoplasmic antiproteinase 3 (CAF | −1.7 | 1.7 |
| C09a | Intracellular Protein Phosphatases | protein phosphatase 2B regulatory | 0.0 | −1.5 |
| C09b | Adenylate/Guanylate Cyclases & | bone marrow stromal antigen 1 (B | −1.8 | 1.4 |
| | Diesterases | | | |
| C09g | Death Receptors | adenosine A1 receptor (ADORA1) | 2.9 | 0.0 |
| C09j | Other Apoptosis-Associated Proteins | IEX-1L anti-death protein; PRG-1; | −1.8 | 0.8 |
| C10b | Adenylate/Guanylate Cyclases & | calcium/calmodulin-dependent 3′, [symbol] | 0.9 | −1.7 |
| | Diesterases | | | |
| | GTP/GDP Exchangers & GTPase | | | |
| C10d | Activity Modulators | GTPase-activating protein (GAP); | 1.8 | 0.0 |
| C10i | Bcl Family Proteins | induced myeloid leukemia cell diff | −4.8 | 0.8 |
| C10m | DNA Damage Repair Proteins & Ligases | Ku 70-kDa subunit; ATP-depende [symbol] | 0.0 | −1.6 |
| C10n | DNA Damage Repair Proteins & Ligases | DNA mismatch repair protein PMS | −1.6 | 0.0 |
| | Other Intracellular Transducers, | | | |
| C11e | Effectors & Modulators | IkappaB kinase complex-associat [symbol] | 0.0 | −1.8 |
| C11h | Caspases | caspase-10 precursor (CASP10); I | 2.9 | 0.0 |
| C11i | Bcl Family Proteins | BCL-2-related protein A1 (BCL2A | −2.6 | 0.0 |
| C11l | DNA Polymerases, Replication Factors | DNA polymerase epsilon subunit [symbol] | 0.0 | −1.6 |
| | & Topoisomerases | | | |
| C11n | DNA Damage Repair Proteins & Ligases | Rad50 | −1.5 | 0.0 |
| C12a | Intracellular Protein Phosphatases | serine/threonine protein phosphat [symbol] | 2.3 | 0.9 |
| C12b | Intracellular Transducers, Effectors & | ephrin A4 precursor (EFNA4); EP [symbol] | 1.4 | −2.0 |
| | Modulators | | | |
| C12c | Transcription Activators & Repressors | NF-kappaB transcription factor p6: | 0.0 | −1.7 |
| C12f | Death Receptor Ligands | CD27 ligand (CD27LG); CD70 ant | −1.6 | −1.0 |
| C12i | Bcl Family Proteins | bcl-2 interacting killer (BIK); NBK: | −1.8 | −1.0 |
| C12j | Other Apoptosis-Associated Proteins | growth arrest & DNA-damage-ind [symbol] | −3.5 | 0.0 |
| C12l | DNA Polymerases, Replication Factors | replication factor C 36-kDa subuni [symbol] | −1.7 | 0.0 |
| | & Topoisomerases | | | |
| C13d | Kinase Activators & Inhibitors | muscle/brain cAMP-dependent pr [symbol] | 0.0 | −1.9 |
| C13e | Other Intracellular Transducers, | leukemia inhibitory factor receptor | −2.0 | 0.0 |
| | Effectors & Modulators | | | |
| C13f | Death Receptors | insulin-like growth factor I receptor | −1.6 | −1.2 |
| C13g | Death Receptor-Associated Proteins & | DAXX | −1.8 | 0.0 |
| | Adaptors | | | |
| C13i | Bcl Family Proteins | NIP1 (NIP1) | −3.2 | 0.1 |
| C13j | Other Apoptosis-Associated Proteins | clusterin precursor (CLU); comple [symbol] | −2.8 | −1.8 |
| C13k | DNA Polymerases, Replication Factors | MCM3 DNA replication licensing f [symbol] : | −2.5 | 0.0 |
| | & Topoisomerases | | | |
| C13m | DNA Damage Repair Proteins & Ligases | DNA ligase III (LIG3); polydeoxyril | −2.3 | 0.2 |
| C14a | Intracellular Protein Phosphatases | myotubularin | 2.5 | −1.9 |
| C14b | Adenylate/Guanylate Cyclases & | adenylate cyclase VII; ATP pyrop [symbol] | −1.0 | −1.5 |
| | Diesterases | | | |
| | Other Intracellular Transducers, | | | |
| C14e | Other Intracellular Transducers, | junction plakoglobin (JUP); desmc | 3.4 | −2.2 |
| | Effectors & Modulators | | | |
| C14f | Death Receptors | retinoic acid receptor epsilon (RA [symbol] | −2.4 | −1.4 |
| C14h | Calpains | calpain 2 large (catalytic) subunit; | −2.2 | 0.7 |
| C14i | Bcl Family Proteins | NIP3 (NIP3) | −1.7 | −2.2 |
| C14j | Other Apoptosis-Associated Proteins | early response protein NAK1; TR3 | −2.4 | 0.0 |
| C14l | DNA Polymerases, Replication Factors | activator 1 37-kDa subunit; replica | −2.2 | −1.3 |
| | & Topoisomerases | | | |
| C14m | DNA Damage Repair Proteins & Ligases | DNA ligase IV (LIG4); polydeoxyr [symbol] | −1.5 | 0.0 |
| C14n | DNA Damage Repair Proteins & Ligases | uracil-DNA glycosylase precursor | −1.1 | −2.1 |
| D01b | Apoptosis-Associated Proteins | growth arrest & DNA-damage-ind [symbol] | 2.0 | −1.3 |
| D01n | Basic Transcription Factors | CACCC-box DNA-binding protein | 1.9 | 0.8 |
| D02a | DNA Damage Repair Proteins & Ligases | DNA-dependent protein kinase (DI | 0.4 | 2.8 |
| D03c | Cell Signaling & Extracellular | prostaglandin E2 (PGE) receptor E | 0.0 | −1.6 |
| | Communication Proteins | | | |
| D03j | Basic Transcription Factors | CCAAT transcription binding facto | 0.0 | 1.6 |
| D03n | Basic Transcription Factors | cellular nucleic acid binding protei [symbol] | 1.9 | 1.3 |
| D04k | Transcription Activators & Repressors | metal-regulatory transcription fact [symbol] | 0.0 | 2.2 |
| D04n | Basic Transcription Factors | basic transcription factor 2 44-kDa | 1.7 | 0.6 |
| D05m | Transcription Activators & Repressors | nuclear factor NF-kappa-B p100 s | 1.7 | 1.9 |
| D06k | Transcription Activators & Repressors | transcription repressor protein PRI | 0.8 | 2.2 |
| D06m | Transcription Activators & Repressors | octamer-binding transcription fact [symbol] | 1.4 | 2.2 |
| D06n | Basic Transcription Factors | transcriptional repressor NF-X1 | 1.8 | 1.6 |
| D07j | Transcription Activators & Repressors | HIV-1 TATA element modulatory f [symbol] | 0.0 | 2.1 |
| D07k | Transcription Activators & Repressors | PCAF-associated factor 65 beta | 0.5 | 2.2 |
| D07l | Transcription Activators & Repressors | glucocorticoid receptor repression | 1.9 | 0.8 |
| D07n | Transcription Activators & Repressors | cAMP-responsive element-binding | 1.5 | 0.6 |
| D08i | Cell Signaling & Extracellular | | | |
| | Communication Proteins | major prion protein precursor (PRI | 0.0 | 2.3 |
| D08j | Basic Transcription Factors | hypoxia-inducible factor 1 alpha ( [symbol] | 0.0 | 3.9 |
| D09f | Cell Signaling & Extracellular | acyl-CoA-binding protein (ACBP); | 0.0 | 1.9 |
| | Communication Proteins | | | |
| D09j | Transcription Activators & Repressors | jun activation domain binding prot [symbol] | 0.0 | 1.7 |
| D09m | Basic Transcription Factors | basic transcription element-bindin [symbol] | 0.3 | 1.9 |
| D09n | Basic Transcription Factors | GA-binding protein beta-2 subunit | 0.9 | 2.7 |
| D10j | Transcription Activators & Repressors | ets domain protein elk-3; NET; S [symbol] | 1.4 | 2.2 |
| D10l | Transcription Activators & Repressors | interleukin enhancer-binding facto | 0.5 | 1.8 |
| D11j | Basic Transcription Factors | histone acetyltransferase B subun | 1.6 | 2.7 |
| D11m | Transcription Activators & Repressors | helix-loop-helix protein; DNA-bind | 0.7 | 2.5 |
| D11n | Basic Transcription Factors | transcription factor ZFM1 | 1.8 | 3.1 |
| D12e | Cell Signaling & Extracellular | histidine decarboxylase (HDC) | 1.8 | 0.1 |
| | Communication Proteins | | | |
| D12g | Cell Signaling & Extracellular | glia maturation factor beta (GMF-I | −1.0 | 2.6 |
| | Communication Proteins | | | |
| D12n | Basic Transcription Factors | ZFM1 protein alternatively spliced | 2.6 | 1.9 |
| D13k | Transcription Activators & Repressors | B-cell lymphoma 6 protein (bcl-6); | 0.6 | 1.5 |
| D13n | Basic Transcription Factors | transcription factor RZR-alpha (RZ [symbol] | 2.0 | 1.4 |
| D14d | Cell Signaling & Extracellular | leptin receptor precursor; obese re [symbol] | −2.5 | 0.1 |
| | Communication Proteins | | | |
| D14g | Cell Signaling & Extracellular | myelin-associated glycoprotein pr [symbol] | −2.1 | 0.0 |
| | Communication Proteins | | | |
| E01a | Transcription Activators & Repressors | brain-specific homeobox/POU dor | −1.5 | 0.0 |
| E01k | Growth Factor & Chemokine Receptors | granulocyte colony stimulating fac | −2.1 | 2.2 |
| E01l | Interleukin & Interferon Receptors | interleukin-2 receptor alpha subun | 1.2 | 1.5 |
| E02b | Basic Transcription Factors | transcription factor HTF4; transcri [symbol] | 0.9 | 1.8 |
| E02i | Cell-Cell Adhesion Receptors | integrin alpha 4 precursor (ITGA4) | 0.4 | 1.8 |
| E02j | Cell-Cell Adhesion Receptors | semaphorin; CD100 | 1.0 | 1.5 |
| E02l | Interleukin & Interferon Receptors | interleukin-6 receptor alpha subun | −2.4 | 2.4 |
| E02m | Interleukin & Interferon Receptors | interleukin-1 receptor type II precu | −1.6 | 1.8 |
| E02n | Xenobiotic Transporters | growth arrest & DNA-damage-ind [symbol] | 1.9 | 1.1 |
| E03e | Transcription Activators & Repressors | heat shock factor protein 1 (HSF1 [symbol] | 0.8 | 1.6 |
| E03k | Growth Factor & Chemokine Receptors | neuromedin B receptor (NMBR): n | 0.7 | 1.8 |
| E03l | Interleukin & Interferon Receptors | interferon-alpha/beta receptor alp [symbol] | 0.7 | 2.0 |
| E03n | Translation | 14.5-kDa translational inhibitor pr [symbol] | 0.9 | 2.0 |
| E04a | Transcription Activators & Repressors | homeobox protein HOXB7; HOX2 [symbol] | 0.0 | 1.8 |
| E04g | Cell-Cell Adhesion Receptors | cadherin 11 precursor (CDH11); o [symbol] | −2.3 | 0.0 |
| E05e | Transcription Activators & Repressors | putative transcription activator DB | −1.2 | 2.6 |
| E05i | Cell-Cell Adhesion Receptors | integrin beta 6 precursor (ITGB6) | −1.6 | 1.3 |
| E07b | Transcription Activators & Repressors | fli-1 oncogene; ergB transcription | 0.0 | 2.2 |
| E07e | Transcription Activators & Repressors | zinc finger protein 91 (ZNF92); HF | 0.0 | 2.2 |
| | Histone Acetyltransferases & | | | |
| E07f | Deacetylases | RPD3 protein; histone deacetylas [symbol] | −1.5 | 0.3 |
| E07i | Matrix Adhesion Receptors | leukocyte adhesion glycoprotein p | −1.8 | 2.2 |
| E07k | Growth Factor & Chemokine Receptors | activin type I receptor; serine/thre [symbol] | −1.6 | 0.0 |
| E08c | CDK Inhibitors | trans-acting T-cell specific transcri | 1.2 | 1.5 |
| E08f | Chromatin Proteins | high mobility group protein (HMG- | 0.0 | 2.0 |
| E08h | Cell-Cell Adhesion Receptors | CD44 antigen hematopoietic form | 0.0 | 2.1 |
| E08k | Growth Factor & Chemokine Receptors | granulocyte-macrophage colony-s | −2.0 | 1.3 |
| E08l | Interleukin & Interferon Receptors | interferon-alpha/beta receptor bet [symbol] | 1.0 | 1.6 |
| E09d | Transcription Activators & Repressors | zinc-finger DNA-binding protein | 0.5 | 2.7 |
| E09i | Cell-Cell Adhesion Receptors | fibronectin receptor beta subunit ( [symbol] | 0.0 | 2.5 |
| E11a | Basic Transcription Factors | transcription factor ETR101 | 2.1 | 2.2 |
| E11n | Xenobiotic Transporters | microsomal glutathione S-transfer | 0.0 | 2.7 |
| E12c | CDK Inhibitors | Sp3 protein | 0.0 | 1.6 |
| E12g | Cell-Cell Adhesion Receptors | vitronectin receptor alpha subunit | −1.0 | 2.9 |
| E12n | Xenobiotic Transporters | glutathione S-transferase pi (GSTI | 0.0 | 2.0 |
| E13k | Growth Factor & Chemokine Receptors | N-sam; fibroblast growth factor re [symbol] | 0.5 | 2.2 |
| E14b | Cell Cycle-Regulating Kinases | FUSE binding protein | 0.8 | 1.6 |
| E14g | Matrix Adhesion Receptors | intercellular adhesion molecule-1 [symbol] | −2.4 | 0.5 |
| E14i | Cell-Cell Adhesion Receptors | leukocyte adhesion glycoprotein L | 2.2 | 1.3 |
| F01e | Growth Factors, Cytokines & | thrombomodulin precursor (THBD | −1.5 | 0.0 |
| | Chemokines | | | |
| F02f | Growth Factors, Cytokines & | vascular endothelial growth factor | −1.8 | 0.6 |
| | Chemokines | | | |
| F02l | Proteosomal Proteins | proteasome component C3; macr [symbol] | 0.0 | 1.9 |
| F03l | Proteosomal Proteins | proteasome component C5; macr [symbol] | 0.0 | 2.2 |
| F04c | Other Extracellular Communication | B94 protein | −1.7 | 0.0 |
| | Proteins | | | |
| F05e | Growth Factors, Cytokines & | hepatoma-derived growth factor ( [symbol] | −1.8 | 0.1 |
| | Chemokines | | | |
| F05g | Growth Factors, Cytokines & | migration inhibitory factor-related [symbol] | −2.0 | 0.0 |
| | Chemokines | | | |
| F05j | Interleukins & Interferons | interleukin-14 precursor (IL-14); hi | 0.0 | −2.1 |
| F06c | Growth Factors, Cytokines & | eosinophil granule major basic prc | −1.9 | 0.5 |
| | Chemokines | | | |
| F06f | Growth Factors, Cytokines & | heparin-binding EGF-like growth f: [symbol] | −1.6 | 0.1 |
| | Chemokines | | | |
| F06g | Growth Factors, Cytokines & | migration inhibitory factor-related [symbol] | −2.1 | 0.0 |
| | Chemokines | | | |
| F06k | Amino-& Carboxypeptidases | carboxypeptidase H precursor (CF | 0.0 | −1.7 |
| F07b | Xenobiotic Metabolism | cytosolic superoxide dismutase 1 [symbol] | 1.5 | 1.4 |
| F07f | Growth Factors, Cytokines & | hepatocyte growth factor (HGF); s | −1.7 | 0.3 |
| | Chemokines | | | |
| F07g | Growth Factors, Cytokines & | platelet-derived growth factor A s [symbol] | −1.8 | 0.7 |
| | Chemokines | | | |
| F07n | Protease Inhibitors | tissue inhibtor of mettaloproteinas [symbol] | −1.6 | −1.1 |
| | Other Intracellular Transducers, | | | |
| F08g | Effectors & Modulators | leukemia inhibitory factor precurs [symbol] | −2.6 | 0.0 |
| F08k | Cysteine Proteases | cathepsin H precursor | −1.6 | −1.4 |
| F08m | Metalloproteinases | matrix metalloproteinase 12 (MMF [symbol] | 0.5 | −1.5 |
| F09a | Xenobiotic Metabolism | cytochrome P450 IIF1 (CYP2F1) | −2.9 | 0.0 |
| F09d | Growth Factors, Cytokines & | transforming growth factor-alpha ( | −1.7 | 0.1 |
| | Chemokines | | | |
| F09e | Growth Factors, Cytokines & | uromodulin; Tamm-Horsfall urinar | −1.8 | −1.1 |
| | Chemokines | | | |
| F09h | Hormones | glucagon precursor (GCG) | 0.0 | −1.6 |
| F09i | Interleukins & interferons | interleukin-1 alpha precursor (IL-1 | −1.6 | 0.9 |
| F10a | Xenobiotic Metabolism | dioxin-inducible cytochrome P450 | −2.7 | −1.2 |
| F10d | Growth Factors, Cytokines & | transforming growth factor-beta (T | 2.4 | 1.1 |
| | Chemokines | | | |
| F10e | Growth Factors, Cytokines & | T-cell-specific rantes protein prec [symbol] | 2.0 | 0.0 |
| | Chemokines | | | |
| F10g | Growth Factors, Cytokines & | macrophage inflammatory protein | −0.0 | 1.7 |
| | Chemokines | | | |
| F10j | Interleukins & Interferons | interleukin-9 precursor (IL-9); T-ce [symbol] | 0.3 | −1.8 |
| F10n | Amino- & Carboxypeptidases | dipeptidyl peptidase IV (DPP IV; [symbol] | −1.1 | −3.0 |
| F11a | Xenobiotic Metabolism | S-mephenytoin 4 hydroxylase; cyt | −2.4 | −1.0 |
| F11b | Other Stress Response Proteins | 25-hydroxy vitamin D3 1-alpha hy [symbol] | −2.0 | 0.3 |
| F11k | Other Enzymeslinvolved in Protein | insulin-degrading enzyme; insulys | −1.6 | 0.4 |
| | Turnover | | | |
| F12c | Growth Factors, Cytokines & | bone morphogenetic protein 1 (B [symbol] | −1.4 | −1.5 |
| | Chemokines | | | |
| F12e | Growth Factors, Cytokines & | monocyte chemotactic protein 1 p | −2.9 | −3.0 |
| | Chemokines | | | |
| F13d | Growth Factors, Cytokines & | kidney epidermal growth factor (E [symbol] | −1.7 | 0.0 |
| | Chemokines | | | |
| F13f | Growth Factors, Cytokines & | thymosin beta-10 (TMSB10; THY [symbol] | 0.0 | −2.1 |
| | Chemokines | | | |
| F13g | Growth Factors, Cytokines & | 0X40 ligand (OX4OL); GP34; tax- [symbol] | 0.0 | −2.3 |
| | Chemokines | | | |
| F13h | Hormones | cellular retinoic acid-binding protei | 3.9 | −1.0 |
| F13i | Interleukins & Interferons | interleukin-6 precursor (IL-6); B-c [symbol] | 2.0 | −2.5 |
| F13j | Other Extracellular Communication | thymosin beta 4; FX | −1.2 | −3.3 |
| | Proteins | | | |
| F14a | Drug-Resistance Proteins | serum paraoxonase/arylesterase 1 | −1.0 | −1.7 |
| F14c | Growth Factors, Cytokines & | bone morphogenetic protein 3 (B [symbol] | 2.8 | −2.2 |
| | Chemokines | | | |
| F14e | Growth Factors, Cytokines & | amphiregulin (AR); colorectum cel | 3.7 | −1.2 |
| | Chemokines | | | |
| F14g | Growth Factors, Cytokines & | interleukin-8 precursor (IL-8): mon | −3.2 | −1.3 |
| | Chemokines | | | |
| F14k | Proteosomal Proteins | proteasome inhibitor HPI31 subun | −1.1 | −3.4 |
| G11 | Housekeeping Genes | ubiquitin | 0.8 | −2.6 |
| G13 | Housekeeping Genes | phospholipase A2 | 4.0 | 1.6 |
| G29 | Housekeeping Genes | brain-specific tubulin alpha 1 subu | 1.8 | 0.0 |
| G43 | Housekeeping Genes | cytoplasmic beta-actin (ACTB) | 0.0 | −2.1 |
| G45 | Housekeeping Genes | 23-kDa highly basic protein; 60S r | 0.0 | −2.5 |
| G47 | Housekeeping Genes | 40S ribosomal protein S9 | 0.3 | −1.8 |

TABLE 7b
GENE CHANGES INDUCED BY VEE VIRUS IN VITRO IN HUMAN LYMPHOID CELLS -
ARRAY II

| Gene code | Classification#1 | Protein/gene | VEE-1 h | VEE-4 h |
|---|---|---|---|---|
| E05e | Growth Factors, Cytokines & Chemokines | FIBROBLAST GROWTH FAC | 6.6 | 3.3 |
| D13n | Hormone Receptors | somatostatin receptor type 4 ( | 4.5 | 0.6 |
| C14h | Complex Lipid Metabolism | famesyl pyrophosphate synth [symbol] | 4.4 | 0.0 |
| B12a | Xenobiotic Metabolism | cytochrome P450 IA1 (CYP1A | 4.3 | 0.2 |
| C04c | Exocytosis Proteins | syntaxin 1A (STX1A); neuron- | 4.2 | 2.8 |
| C13l | Extracellular Transporters & Carrier | apolipoprotein E precursor (A [symbol] | 4.1 | 2.7 |
| E11f | Intracellular Protein Phosphatases | serine/threonine protein phos [symbol] | 4.0 | 1.3 |
| B09d | Oncogenes & Tumor Suppressors | AF-17 protein | 3.8 | 1.5 |
| A01c | Cell Surface Antigens | leukocyte CD37 antigen | 3.7 | 0.3 |
| C02b | Other Membrane Channels & | GAP JUNCTION BETA-1 PR [symbol] | 3.7 | 0.0 |
| F07e | Calpains | calpain 1 large (catalytic) sub [symbol] | 3.5 | 1.2 |
| C03b | Extracellular Matrix Proteins | cartilage glycoprotein 39 prec [symbol] | 3.4 | 0.0 |
| A03j | Cell Surface Antigens | T-cell surface glycoprotein CD | 3.2 | 1.6 |
| D07e | Ribosomal Proteins | 60S ribosomal protein L22 (R [symbol] | 3.2 | 0.8 |
| A04k | Cell Surface Antigens | T-cell surface glycoprotein CD | 3.2 | 0.7 |
| E12l | Intracellular Protein Phosphatases | serine/threonine protein phos [symbol] | 3.0 | 1.1 |
| F09g | G Protein-Coupled Receptors | B2-Bradykinin receptor | 3.0 | 0.2 |
| C14j | Complex Lipid Metabolism | mevalonate kinase | 2.9 | −9.8 |
| E06k | Hormones | natriuretic peptide precursor B | 2.8 | 0.3 |
| C14e | Complex Lipid Metabolism | annexin III (ANX3); lipocortin l | 2.8 | 0.0 |
| A01i | Cell Surface Antigens | leukemia virus receptor 1 (GL | 2.7 | 1.3 |
| C06k | Other Trafficking & Targeting Proteins | syntaxin 3 (STX3) | 2.7 | 2.5 |
| A03i | Cell Surface Antigens | T-cell surface glycoprotein CD | 2.5 | 2.4 |
| C09d | Simple Carbohydrate Metabolism | long-chain-fatty-acid-CoA liga [symbol] | 2.5 | 0.6 |
| B11f | GTP/GDP Exchangers & GTPase Activity | GTPase-activating protein (G [symbol] | 2.5 | 0.1 |
| B08h | Oncogenes & Tumor Suppressors | zinc finger protein hrx; ALL-1; | 2.5 | 0.7 |
| F08g | Other Enzymeslinvolved in Protein | fibrinogen B beta polypeptide | 2.4 | 0.7 |
| B10a | Oncogenes & Tumor Suppressors | probable ATP-dependent RN [symbol] | 2.4 | 0.0 |
| A03h | Cell Surface Antigens | early activation CD69 antigen | 2.4 | 0.7 |
| C09n | Complex Carbohydrate Metabolism | LYSOSOMAL ALPHA-MANN [symbol] | 2.3 | −1.3 |
| B07h | Oncogenes & Tumor Suppressors | dek protein | 2.3 | 0.0 |
| B05k | Other Immune System Proteins | NEUTROPHIL DEFENSINS 1 | 2.2 | 0.0 |
| F12k | Other Cytoskeleton & Motility Proteins | brain variant 1 ankyrin (ankyri | 2.2 | 0.8 |
| A10i | Basic Transcription Factors | HOMEOBOX PROTEIN HOX [symbol] | 2.2 | 0.0 |
| E08e | Intracellular Kinase Network Members | protein-tyrosine phosphatase [symbol] | 2.1 | 0.2 |
| D09b | Growth Factor & Chemokine Receptors | bone morphogenetic protein t [symbol] | 2.1 | 0.1 |
| D13k | Hormone Receptors | PARATHYROID HORMONE [symbol] | 2.1 | 0.0 |
| C14i | Complex Lipid Metabolism | squalene synthetase | 2.1 | 0.0 |
| E13n | G Proteins | ras-related protein RAP-1B; G | 2.1 | 2.6 |
| E12m | Intracellular Protein Phosphatases | serine/threonine protein phos [symbol] | 2.0 | 3.0 |
| E07l | Intracellular Adaptors & Receptor- | hematopoletic lineage cell-sp [symbol] | 2.0 | 0.0 |
| B11e | Oncogenes & Tumor Suppressors | ras-related protein R-ras2; ras | 2.0 | 0.5 |
| C10d | Complex Carbohydrate Metabolism | alpha-galactosidase A precurs | −1.9 | 0.4 |
| B10f | Oncogenes & Tumor Suppressors | nucleolar phosphoprotein B23 | 1.9 | 1.4 |
| C14d | Complex Lipid Metabolism | phosphatidylethanolamine-bin [symbol] | 1.9 | −2.9 |
| A02k | Cell Surface Antigens | L-selectin precursor, lymph nc | 1.9 | 1.5 |
| A06j | Transcription Activators & Repressors | lkaros/LyF-1 homolog hlk-1 | 1.9 | 0.0 |
| A03m | Cell Surface Antigens | T-cell differentiation CD6 anti [symbol] | 1.8 | 1.1 |
| F02d | Adenylate/Guanylate Cyclases & | guanylate cyclase soluble alp [symbol] | 1.8 | 1.6 |
| F03h | Calcium-Binding Proteins | sorcin 22-kDa protein (SRI); C | 1.8 | 0.0 |
| C04b | Exocytosis Proteins | vesicle-membrane fusion prot | 1.8 | 1.9 |
| D10e | Hormone Receptors | gonadotropin-releasing hormo | 1.8 | 1.6 |
| F01e | Phospholipases & Phosphoinositol | phospholipase C gamma 1 (P [symbol] | 1.8 | 0.2 |
| F04b | Kinase Activators & Inhibitors | diacyglycerol kinase alpha (D | 1.8 | 0.0 |
| F03e | Calcium-Binding Proteins | neuron-specific calclum-bindir | 1.7 | 0.1 |
| A03k | Cell Surface Antigens | T-cell CD7 antigen precursor; | 1.7 | 1.3 |
| F01m | Phospholipases & Phosphoinositol | phosphatidytinositol 3 kinase [symbol] | 1.7 | 0.5 |
| D02e | Complex Lipid Metabolism | 3-ketoacyl-CoA thiolase perox | 1.6 | 2.9 |
| B14m | Voltage-Gated Ion Channels | Inward rectifier potassium cha | 1.6 | −1.1 |
| B12j | Xenoblotic Metabolism | cytochrome P450 VIIA1 (CYF | 1.6 | 0.0 |
| E11c | Intracellular Protein Phosphatases | serine/threonine protein phos [symbol] | 1.6 | 0.9 |
| E11b | Intracellular Protein Phosphatases | dual-specificity protein phosp [symbol] | 1.6 | 0.4 |
| A11h | Basic Transcription Factors | HOMEOBOX PROTEIN SIX1 | 1.6 | −1.4 |
| C10l | Energy Metabolism | pyruvate kinase R/L (PKLR); [symbol] | 1.6 | 0.2 |
| F05f | Other Intracellular Transducers, Effectors | myo-inositol 1(or 4) monophos | 1.6 | 0.0 |
| C08c | Other Trafficking & Targeting Proteins | RAB GDP dissociation Inihibit [symbol] | 1.5 | 1.1 |
| F08d | Protease Inhibitors | tissue factor pathway inhibitor | 1.5 | 0.4 |
| E09m | Intracellular Transducers, Effectors & | G protein-coupled receptor kir | −1.5 | 0.6 |
| A11b | Basic Transcription Factors | HOMEOBOX PROTEIN MOX | −1.5 | 0.0 |
| D14i | Hormone Receptors | neuron-derived orphan recept [symbol] | −1.5 | 3.1 |
| B04g | Cell-Cell Adhesion Receptors | SUSHI REPEAT-CONTAININ | −1.5 | 0.0 |
| B13j | Voltage-Gated Ion Channels | dihydropyridine-sensitive I-typ | −1.6 | 0.2 |
| C02m | Cell Signaling & Extracellular | myelin basic protein (MBP) | −1.6 | 0.1 |
| G11 | Housekeeping Genes | ubiquitin | −1.6 | −2.0 |
| E08c | Intracellular Kinase Network Members | bone marrow kinase X-linked; | −1.6 | 0.0 |
| B14a | Voltage-Gated Ion Channels | voltage-gated potassium chan | −1.6 | −1.4 |
| F12l | Other Cytoskeleton & Motility Proteins | dematin; erythrocyte membra [symbol] | −1.6 | 0.0 |
| A02d | Cell Surface Antigens | annexin V; lipocortin V; endon | −1.6 | 4.4 |
| A12d | Basic Transcription Factors | NEUROGENIC DIFFERENTI [symbol] | −1.6 | 0.0 |
| E13g | G Proteins | GUANINE NUCLEOTIDE-BIN | −1.6 | −1.2 |
| A09i | Basic Transcription Factors | FORKHEAD-RELATED TRA [symbol] | −1.7 | −1.1 |
| E02f | Neurotransmitter Receptors | neuronal acetylcholine recept [symbol] | −1.7 | 0.6 |
| E06f | Growth Factors. Cytokines & Chemokines | granulins precursor (GRN); a [symbol] | −1.7 | 0.9 |
| C01c | Symporters & Antiporters | sodium- & chloride-dependent | −1.7 | 0.2 |
| E14h | GTP/GDP Exchangers & GTPase Activity | REGULATOR OF G-PROTEII | −1.8 | 0.5 |
| A06g | Basic Transcription Factors | Runt domain-containing protei | −1.9 | −2.0 |
| F13j | Functionally Unclassified Proteins | EYES ABSENT HOMOLOG 1 | −2.0 | 0.0 |
| D14g | Hormone Receptors | estrogen receptor beta (ER-b [symbol] | −2.1 | 0.7 |
| B02g | Transcription Activators & Repressors | TBX2 PROTEIN (T-BOX PRO | −2.1 | 0.0 |
| A05l | Cell Surface Antigens | plasma-cell membrane glycop | −2.1 | 0.0 |
| D05d | Other Metabolism Enzymes | ferritin heavy chain (FTH1); F | −2.2 | 1.6 |
| G45 | Housekeeping Genes | 23-kDa highly basic protein; 6 | −2.2 | −1.7 |
| G43 | Housekeeplng Genes | cytoplasmic beta-actin (ACTB | −2.2 | −2.3 |
| F11m | Intermediate Filament Proteins | nestin | −2.3 | 0.0 |
| C02j | Extracellular Matrix Proteins | lumican precursor (LUM); ken [symbol] | −2.7 | 0.0 |
| A13d | Basic Transcription Factors | SOX-1 PROTEIN | −3.2 | 0.0 |
| E11a | Intracellular Protein Phosphatases | dual-specificity protein phosp [symbol] | −3.7 | 0.0 |
| B01c | Basic Transcription Factors | paired box protein PAX-6; ocu | −4.9 | 0.0 |
| E07k | Other Intracellular Transducers, Effectors | signal transducing adaptor mo | −5.1 | 0.6 |
| A02g | Cell Surface Antigens | LGALS3, MAC2 (Galectin-3, [symbol] | 0.4 | 5.0 |
| A02d | Cell Surface Antigens | annexin V; lipocortin V; endon | −1.6 | 4.4 |
| A03n | Cell Surface Antigens | lymphocyte function-associate | 0.2 | 3.8 |
| A01g | Cell Surface Antigens | leukocyte surface CD53 antig [symbol] | 0.3 | 3.8 |
| E05e | Growth Factors, Cytokines & Chermokines | FIBROBLAST GROWTH FA [symbol] | 6.6 | 3.3 |
| D14i | Hormone Receptors | neuron-derived orphan recept | −1.5 | 3.1 |
| E12m | Intracellular Protein Phosphatases | serine/threonine protein phosp | 2.0 | 3.0 |
| D02a | Complex Lipid Metabolism | slmiliar to sterol O-acylfransfe | 0.1 | 3.0 |
| A02j | Cell Surface Antigens | endogfin precursor (ENG; EN [symbol] | 0.8 | 3.0 |
| D02e | Complex Lipid Metabolism | 3-ketoacyl-CoA thiolase perox | 1.6 | 2.9 |
| C07e | G Proteins | ras-related protein RAB-7 | 1.1 | 2.8 |
| D08k | Growth Factor & Chemokine Receptors | c factor receptor beta (GDNF [symbol] | 0.0 | 2.8 |
| C04c | Exocytosis Proteins | syntaxin 1A (STX1A); neuron- | 4.2 | 2.8 |
| C13l | Extracellular Transporters & Carrier | apolipoprotein E precursor (A [symbol] | 4.1 | 2.7 |
| E13n | G Proteins | ras-retated protein RAP-1B; G | 2.1 | 2.6 |
| E14m | GTP/GDP Exchangers & GTPase Activity | calpactin l light chain | 0.5 | 2.5 |
| C06k | Other Trafficking & Targeting Proteins | syntaxin 3 (STX3) | 2.7 | 2.5 |
| A01n | Cell Surface Antigens | lysosome-associated membra | 0.6 | 2.5 |
| E10n | Intracellular Protein Phosphatases | dual-specificity protein phosp [symbol] | 0.8 | 2.4 |
| D01e | Complex Lipid Metabolism | corticosteroid 11-beta-dehyd [symbol] | 0.6 | 2.4 |
| D01b | Complex Lipid Metabolism | delta 7 sterol reductase | 0.0 | 2.4 |
| A03i | Cell Surface Antigens | T-cell surface glycoprotein CD | 2.5 | 2.4 |
| A03f | Cell Surface Antigens | platelet glycoprotein IB beta s [symbol] | 0.0 | 2.3 |
| D02b | Complex Lipid Metabolism | steroid 5-alpha reductase 1 (S | 0.0 | 2.3 |
| F03g | Calcium-Binding Proteins | calgizzarin; S100C protein; M [symbol] | 0.1 | 2.2 |
| D02c | Complex Lipid Metabolism | steroid 5-alpha reductase 2 (S | 0.9 | 2.0 |
| D01c | Complex Lipid Metabolism | C-4 methyl sterol oxidase | 0.5 | 2.0 |
| A03e | Cell Surface Antigens | platelet glycoprotein lb alpha [symbol] | 0.0 | 2.0 |
| C13m | Complex Lipid Metabolism | cholinephosphate cytidylyltran | 0.5 | 2.0 |
| F07c | Metalloproteinases | ADAM10 | 1.3 | 2.0 |
| A03a | Cell Surface Antigens | laminin alpha-3 subunit precu [symbol] | 0.0 | 2.0 |
| C04b | Exocytosis Proteins | vesicle-membrane fusion prot | 1.8 | 1.9 |
| C05b | Other Trafficking & Targeting Proteins | SEC13-related protein (SEC1: | 0.7 | 1.9 |
| C07g | Other Trafficking & Targeting Proteins | ras-related protein RAB-1A; Y | 0.0 | 1.9 |
| G27 | Housekeeping Genes | liver glyceraldehyde 3-phosph | 0.1 | 1.9 |
| C06c | Other Trafficking & Targeting Proteins | alpha-soluble NSF attachmen [symbol] | 1.3 | 1.9 |
| F05n | Other Intracellular Transducers, Effectors | amyloid-like protein 2 | 1.2 | 1.9 |
| F04d | Other Intracellular Transducers, Effectors | 14-3-3 protein tau; 14-3-3 prot | 1.4 | 1.9 |
| A01m | Cell Surface Antigens | lysosome-associated membra | 0.9 | 1.8 |
| A04c | Cell Surface Antigens | CD83 antigen precursor [symbol] cell [symbol] | −1.3 | 1.8 |
| A02c | Cell Surface Antigens | lysosome membrane protein [symbol] | 0.0 | 1.8 |
| E09g | Intracellular Kinase Network Members | calcium/calmodulin-dependen | −1.2 | 1.8 |
| A01h | Cell Surface Antigens | leukemia virus receptor 2 (GL | 1.1 | 1.8 |
| F14b | Cell Signaling & Extracellular | major prion protein precursor [symbol] | 1.3 | 1.8 |
| A02n | Cell Surface Antigens | laminin alpha-2 subunit precu [symbol] | 0.0 | 1.8 |
| C05d | Other Trafficking & Targeting Proteins | coatomer alpha subunit; alpha | 0.3 | 1.8 |
| F11e | Orphan Receptors | RAR-related orphan receptor [symbol] | 1.4 | 1.8 |
| A01k | Cell Surface Antigens | Syndecan2 (Fibroglycan) (He [symbol] | 0.5 | 1.7 |
| F10c | G Protein-Coupled Receptors | EBV-induced G-protein-coupl [symbol] | 1.1 | 1.7 |
| F07d | Cysteine Proteases | cathepsin B precursor (CTSB) | 0.0 | 1.7 |
| D01n | Complex Lipid Metabolism | cholesterol acyltransferase | 0.0 | 1.7 |
| D01a | Complex Lipid Metabolism | sterol C5 desaturase (C5D); [symbol] | 1.3 | 1.7 |
| E03f | Other Receptors (by Ligands) | protein kinase C iota type (NP | −1.1 | 1.6 |
| D03m | Cell Signaling & Extracellular | histidine decarboxylase(HDC [symbol] | 0.0 | 1.6 |
| B11g | Oncogenes & Tumor Suppressors | clathrin assembly protein lym [symbol] | 0.4 | 1.6 |
| D13l | Hormone Receptors | somatostatin receptor type 1 [symbol] | 0.3 | 1.6 |
| A03j | Cell Surface Antigens | T-cell surface glycoprotein CD | 3.2 | 1.6 |
| D05d | Other Metabolism Enzymes | ferritin heavy chain (FTH1); F | −2.2 | 1.6 |
| F02d | Adenylate/Guanylate Cyclases & | guanylate cyclase soluble alp [symbol] | 1.8 | 1.6 |
| A03d | Cell Signaling & Extracellular | Kallmann syndrome protein [symbol] | 0.0 | 1.6 |
| F05e | Cell Signaling & Extracellular | presynaptic density protein 95 | 1.1 | 1.6 |
| D11e | Hormone Receptors | BB2-Bombesin receptor | 0.6 | 1.6 |
| D10e | Hormone Receptors | gonadotropin-releasing hormo | 1.8 | 1.6 |
| A05c | Cell Surface Antigens | complement decay-accelerati [symbol] | 0.8 | 1.6 |
| A02l | Cell Surface Antigens | P-selectin precursor (SELP); [symbol] | 0.1 | 1.6 |
| A02f | Cell Surface Antigens | platelet glycoprotein IX | 0.0 | 1.5 |
| F05d | Cell Signaling & Extracellular | 43-kDa postsynaptic protein; [symbol] | 1.3 | 1.5 |
| B05m | Other Immune System Proteins | grancalcin | 0.0 | −1.5 |
| C04m | Other Trafficking & Targeting Proteins | ER-Golgi Intermediate compa | 0.8 | −1.5 |
| A11k | Basic Transcription Factors | MYELIN TRANSCRIPTION F, | 0.0 | −1.5 |
| A12j | Basic Transcription Factors | POD1 - MESODERM-SPEC | −1.1 | −1.5 |
| D04m | Metabolism of Cofactors, Vitamins & | peroxisomal acyl-coenzyme A | −1.3 | −1.5 |
| B05g | Other Immune System Proteins | myeloperoxidase precursor (IV | −1.1 | −1.5 |
| C06i | Cell Signaling & Extracellular | synaptosomal-associated prot | 0.0 | −1.5 |
| A13c | Basic Transcription Factors | SREBP-1 - BASIC-HELIX-LO [symbol] | 0.0 | −1.5 |
| D08j | Growth Factor & Chemokine Receptors | leukocyte platelet-activating f [symbol] | 0.2 | −1.6 |
| A07j | Basic Transcription Factors | interferon regulatory factor 4 [symbol] | 0.4 | −1.6 |
| A11e | Basic Transcription Factors | HOMEOBOX PROTEIN PKN [symbol] | 0.0 | −1.6 |
| B07l | Oncogenes & Tumor Suppressors | AF-6 protein | 0.0 | −1.6 |
| D06a | Other Metabolism Enzymes | corticosteroid 11-beta.dehyd [symbol] | 0.9 | −1.6 |
| B01k | Transcription Activators & Repressors | host cell factor C1 (HCF); VP1 | 0.5 | −1.6 |
| B06m | Extracellular Transporters & Carrier | phospholipid transfer protein [symbol] | 0.2 | −1.6 |
| G29 | Housekeeping Genes | brain-specific tubulin alpha 1 [symbol] | 0.2 | −1.7 |
| A08h | Transcription Activators & Repressors | human Immunodeficiency viru | 0.0 | −1.7 |
| G45 | Housekeeping Genes | 23-kDa highly basic protein; 6 | −2.2 | −1.7 |
| C03n | G Proteins | ADP-ribosylation factor 1 | 0.5 | −1.7 |
| A14h | Basic Transcription Factors | ENX-1 PUTATIVE TRANSCR | 0.5 | −1.7 |
| E07j | Other Extracellular Communication | chondromodulin I precursor ( [symbol] | 0.0 | −1.7 |
| A14f | Basic Transcription Factors | ZINC FINGER PROTEIN ZF [symbol] | 0.2 | −1.7 |
| E06c | Growth Factors, Cytokines & Chemokines | PS2 protein precurso [symbol] HP1.A | 1.2 | −1.7 |
| B01l | Transcription Activators & Repressors | gamma-interferon-inducible p [symbol] | 1.2 | −1.7 |
| B03f | Transcription Activators & Repressors | Interleukin enhancer-binding f [symbol] | 0.4 | −1.7 |
| E04n | Growth Factors, Cytokines & Chemokines | CC chemokine eotaxin precu [symbol] | −1.1 | −1.8 |
| A09j | Basic Transcription Factors | EARLY GROWTH RESPONS | 0.0 | −1.8 |
| B12n | Xenobiotic Metabolism | D-amino acid oxidase (DAMO | 0.0 | −1.8 |
| E12j | Intracellular Protein Phosphatases | tyrosine phosphatase | 0.0 | −1.8 |
| A09c | Basic Transcription Factors | HATH-1 - ATONAL HOMOLO | 0.0 | −1.8 |
| B01f | Basic Transcription Factors | ESE1, ERT, JEN, ELF3 (Epit [symbol] | 0.0 | −1.8 |
| A06h | Transcription Activators & Repressors | nuclear factor NF-kappa-B p [symbol] | −1.0 | −1.8 |
| C01l | Symporters & Antiporters | sodium-dependent glutamate/ | 0.0 | −1.9 |
| B10e | Oncogenes & Tumor Suppressors | nuclear pore complex protein | 0.4 | −1.9 |
| C13d | Complex Lipid Metabolism | 3-hydroxy-3-methylglutaryl-co | 0.0 | −2.0 |
| B03h | Other Transcription Proteins | ELL protein | 0.0 | −2.0 |
| G11 | Housekeeping Genes | ubiquitin | −1.6 | −2.0 |
| A08i | Basic Transacription Factors | NF-ATc | 0.5 | −2.0 |
| A06g | Basic Transcription Factors | Runt domain-containing protei | −1.9 | −2.0 |
| C14c | Complex Lipid Metabolism | membrane-associated phosph | 0.0 | −2.0 |
| A06l | Transcription Activators & Repressors | Interferon regulatory factor 5 ( | 0.1 | −2.1 |
| B09n | Oncogenes & Tumor Suppressors | RNA-binding protein fus/tls | 1.0 | −2.2 |
| G43 | Housekeeping Genes | cytoplasmic beta-actin (ACTB | −2.2 | −2.3 |
| E14k | GTP/GDP Exchangers & GTPase Activity | REGULATOR OF G-PROTEI [symbol] | 0.2 | −2.4 |
| A11n | Basic Transcription Factors | MYOCYTE-SPECIFIC ENHAI | 0.6 | −2.5 |
| C14d | Complex Lipid Metabolism | phosphatidylethanolamine-bin | 1.9 | −2.9 |
| B01h | Transcription Activators & Repressors | Interleukin enhancer binding f [symbol] | −1.0 | −3.8 |
| B01g | Basic Transcription Factors | ZINC FINGER PROTEIN UBI | −1.2 | −8.9 |
| C14j | Complex Lipid Metabolism | mevalonate kinase | 2.9 | −9.8 |

TABLE 7c
GENE CHANGES INDUCED BY VEE VIRUS IN VITRO IN HUMAN LYMPHOID CELLS -
CANCER ARRAY

| Gene code | Gene Function | Protein/gene | VEE-1h | VEE-4h |
|---|---|---|---|---|
| F10e | Nucleotide Metabolism | adenosine deaminase (ADA); adenosine | 7.3 | 0.6 |
| F11d | Nucleotide Metabolism | thioredoxin reductase | 7.0 | 0.0 |
| F09d | Nucleotide Metabolism | 5′-nucleotidase precurso (5′-NT)r, ecto-n | 6.8 | −4.2 |
| F09e | Nucleotide Metabolism | adenylosuccinate synthetase; IMP-aspar | 6.7 | 0.1 |
| F11e | Nucleotide Metabolism | DR-nm23 | 6.4 | 0.0 |
| F08d | Nucleotide Metabolism | multifunctional protein ADE2 | 6.2 | 0.7 |
| F10d | Nucleotide Metabolism | ribonucleoside-diphosphate reductase M | 6.1 | 0.0 |
| F11c | Nucleotide Metabolism | uridine diphosphoglucose pyrophosphory | 5.9 | 0.7 |
| F12d | Nucleotide Metabolism | uridine 5′-monophosphate synthase (UM | 5.8 | 0.0 |
| F12e | Nucleotide Metabolism | microsomal UDP-glucuronosyltransferase | 5.5 | 1.1 |
| F09f | Nucleotide Metabolism | type I cytoskeletal 19 keratin; cytokeratin | 5.3 | 0.0 |
| F10f | Nucleotide Metabolism | type II cytoskeletal 11 keratin (KRT11); c | 5.2 | 0.8 |
| F12c | Amino Acid Metabolism | bifunctional purine biosynthesis protein | 5.0 | −1.2 |
| F09c | Nucleotide Metabolism | uridine phosphorylase (UDRPase; UP) | 4.8 | −2.6 |
| F13e | Nucleotide Metabolism | 5,6-dihydroxyindole-2-carboxylic acid ox | 4.6 | 1.3 |
| F08e | Nucleotide Metabolism | adenylate kinase 3 (AK3); mitochondrial | 4.5 | 2.0 |
| F13d | Nucleotide Metabolism | adenylate kinase isoenzyme 1 (AK1); AT | 4.4 | 1.7 |
| F11f | Nucleotide Metabolism | type II cytoskeletal 2 oral keratin; cytoke | 4.3 | 0.9 |
| F14d | Nucleotide Metabolism | thymidylate kinase | 4.1 | 0.4 |
| A14a | Intracellular Transducers, | cadherin (CDH1); epithelial cadherin pr | 3.9 | 1.2 |
| | Effectors & Modulators | | | |
| F13c | Amino Acid Metabolism | adenine phosphoribosyltransferase (APF | 3.9 | −2.4 |
| F08c | Nucleotide Metabolism | GMP synthase; glutamine amidotransfer | 3.9 | 0.0 |
| F10c | Nucleotide Metabolism | inosine-5′-monophosphate dehydrogenase | 3.9 | −1.1 |
| B04b | Intracellular Adaptors & Receptor- | APS | 3.8. | −1.7 |
| | Associated Proteins | | | |
| F11b | Extracellular Matrix Proteins | phosphoribosyl pyrophosphate synthetas | 3.8 | 0.2 |
| F12f | Nucleotide Metabolism | type II cytoskeletal 2 epidermal keratin ( [symbol] | 3.3 | 1.7 |
| C11n | Transcription Activators & | transcription factor HGATA-6 | 3.1 | 0.0 |
| | Repressors | | | |
| B01c | Intracellular Kinase Network | dual-specificity mitogen-activated protein | 3.1 | 0.0 |
| | Members | | | |
| C10m | Transcription Activators & | TAX1-binding protein 151 (TXBP151) | 3.0 | 1.3 |
| | Repressors | | | |
| F14c | Amino Acid Metabolism | adenylosuccinate lyase; adenylosuccinase | 3.0 | 0.0 |
| F03i | Cytoskeleton & Motility Proteins | hemoglobin alpha subunit | 2.9 | 0.0 |
| F12b | Extracellular Matrix Proteins | dopachrome tautomerase; dopachrome [symbol] | 2.9 | 0.4 |
| C10n | Transcription Activators & | myeloid ELF1-like factor | 2.8 | 0.0 |
| | Repressors | | | |
| F13b | Extracellular Matrix Proteins | cytosolic serine hydroxylmethyltransferase | 2.7 | 0.2 |
| F14f | Microfilament Proteins | type II cytoskeletal 5 keratin(KRT5): cyt [symbol] | 2.4 | 0.0 |
| A01d | Oncogenes & Tumor Suppressors | jun-B | 2.4 | 1.7 |
| F13f | Other Metabolism Enzymes | type II cytoskeletal 4 keratin (KRT4); cyt [symbol] | 2.3 | 0.0 |
| D12c | Cell Surface Antigens | T-cell surface glycoprotein CD5 precurs [symbol] | 2.3 | 0.0 |
| B12l | Kinase Activators & Inhibitors | STAT-induced STAT inhibitor 3 | 2.3 | −1.0 |
| F14g | Intermediate Filament Proteins | microtubule-associated protein 1B | 2.2 | 0.0 |
| F11a | Functionally Unclassified Proteins | tenascin precursor (TN); hexabrachion ( [symbol] | 2.2 | 0.6 |
| C11m | Transcription Activators & | putative regulatory protein TGF-beta-stir | 2.2 | 0.0 |
| | Repressors | | | |
| B04e | Intracellular Kinase Network | DCHT | 2.1 | −1.8 |
| | Members | | | |
| A01h | Oncogenes & Tumor Suppressors | interferon-inducible protein 9-27 | 2.1 | −1.3 |
| A01c | Oncogenes & Tumor Suppressors | c-jun proto-oncogene; transcription facto | 1.9 | 0.0 |
| F10a | Functionally Unclassified Proteins | nidogen precursor (NID); entactin | 1.8 | 1.7 |
| F13g | Intermediate Filament Proteins | coronin-like protein P57 | 1.8 | 0.7 |
| B10m | Other Intracellular Transducers, | junction plakoglobin (JUP); desmoplakin | 1.8 | 1.2 |
| | Effectors & Modulators | | | |
| E14m | Immunoglobulins | IgC mu heavy chain constant region | 1.8 | 0.0 |
| F09n | Functionally Unclassified Proteins | mitogen-responsive phosphoprotein DO [symbol] | 1.8 | 0.2 |
| E09m | Other Enzymeslinvolved in | membrane-bound & secreted immunoglo | 1.8 | 0.5 |
| | Protein Turnover | | | |
| C01a | Death Receptors | WSL protein + TRAMP + Apo-3 + death | 1.7 | −1.1 |
| F10b | Extracellular Matrix Proteins | IMP dehydrogenase 1 | 1.7 | 0.0 |
| E13m | Major Histocompatibility Complex | immunoglobulin alpha 1 heavy chain co [symbol] | 1.7 | 1.1 |
| | Proteins | | | |
| F05a | Functionally Unclassified Proteins | laminin alpha 4 subunit precursor (lamini | 1.6 | 0.2 |
| A10n | Other Cell Cycle Proteins | btg protein precursor; NGF-inducible ant | 1.6 | 0.0 |
| E05m | Protease Inhibitors | immunoglobulin rearranged gamma chain | 1.6 | 0.0 |
| C12n | Transcription Activators & | checkpoint suppressor 1 | 1.5 | 0.0 |
| | Repressors | | | |
| D01k | Intracellular Transducers, | urokinase-type plasminogen activator re [symbol] | −1.5 | 0.0 |
| | Effectors & Modulators | | | |
| E07f | Growth Factors, Cytokines & | interleukin-1 beta precursor (IL-1; IL1B); | −1.5 | 0.6 |
| | Chemokines | | | |
| A05g | Oncogenes & Tumor Suppressors | gamma-interferon-inducible protein; IP-3 | −1.6 | 1.5 |
| D09m | Other Receptors (by Ligands) | glutathione-S-transferase (GST) homolog | −1.6 | 1.8 |
| B13b | Intracellular Adaptors & Receptor- | epidermal growth factor receptor kinase | −1.8 | 0.2 |
| | Associated Proteins | | | |
| D07d | Cell Surface Antigens | Integrin beta 8 precursor (ITGB8) | −1.8 | 2.1 |
| D01b | Other DNA-Binding & Chromatin | nuclear domain 10 protein 52 (NDP52) | −1.9 | −2.3 |
| | Proteins | | | |
| D04n | Xenobiotic Metabolism | cytochrome B-245 heavy chain; P22 pha | −2.0 | 0.0 |
| F13m | Functionally Unclassified Proteins | P18 protein | −2.5 | 1.9 |
| G13 | Housekeeping Genes | phospholipase A2 | 0.0 | 3.2 |
| C06a | Death Receptors | decoy receptor 2 | 0.5 | 3.1 |
| C12k | Transcription Proteins | FHF-1 | 0.0 | 3.0 |
| E13j | Metalloproteinases | glia-derived neurite-promoting factor (G [symbol] | 0.0 | 2.8 |
| E09b | Growth Factors, Cytokines & | vascular endothelial growth factor precur | −1.2 | 2.7 |
| | Chemokines | | | |
| B06d | Intracellular Kinase Network | lipid-activated protein kinase PRK1; PKN | 0.2 | 2.7 |
| | Members | | | |
| G27 | Housekeeping Genes | liver glyceraldehyde 3-phosphate dehyd [symbol] | 0.0 | 2.7 |
| E03j | Other Extracellular | cathepsin D precursor (CTSD) | 0.0 | 2.7 |
| | Communication Proteins | | | |
| E02j | Other Extracellular | microsomal aminopeptidase N; myeloid | 0.0 | 2.7 |
| | Communication Proteins | | | |
| F14b | Extracellutar Matrix Proteins | aminoacylase 1 (ACY1) | 1.2 | 2.6 |
| F14e | Nucleotide Metabolism | BIGH3 | 0.4 | 2.5 |
| E05e | Growth Factors, Cytokines & | interleukin-1 receptor antagonist protein | 0.0 | 2.5 |
| | Chemokines | | | |
| E10i | Other Extracellular | matrix metalloproteinase 9 (MMP9); gel [symbol] | 0.4 | 2.5 |
| | Communication Proteins | | | |
| D04j | Interleukin & Interferon Receptors | interferon-alpha/beta receptor alpha sub [symbol] | 1.1 | 2.4 |
| D14l | Other Receptors (by Ligands) | frizzled homolog (FZD3) | 0.9 | 2.4 |
| E10j | Metalloproteinases | metalloproteinase inhibitor 1 precursor [symbol] | 0.0 | 2.4 |
| A12i | Cyclins | cyclin G2 (CCNG2) | 0.0 | 2.3 |
| A10f | Oncogenes & Tumor Suppressors | matrix metalloproteinase 11 (MMP11); s [symbol] | 0.0 | 2.3 |
| F04k | Chaperones & Heat Shock | 60S ribosomal protein L5 | 0.5 | 2.3 |
| | Proteins | | | |
| F05m | Functionally Unclassified Proteins | menin | 0.5 | 2.3 |
| E14h | Intracellular Transducers, | Wnt-8B | 0.1 | 2.3 |
| | Effectors & Modulators | | | |
| E06a | Extracellular Matrix Proteins | bone morphogenetic protein 4 (BMP4) [symbol] | 0.0 | 2.2 |
| E07m | Other Enzymeslinvolved in | HLA-DR antigen-associated invariant sul | 0.3 | 2.2 |
| | Protein Turnover | | | |
| F03m | Functionally Unclassified Proteins | HEM45 | 0.5 | 2.2 |
| C12j | Transcription Activators & | early growth response protein 1 (hEGR1: | 0.5 | 2.1 |
| | Repressors | | | |
| G47 | Housekeeping Genes | 40S ribosomal protein S9 | −1.0 | 2.1 |
| B04a | ATPase Transporters | ATP synthase coupling factor 6 mitochor | 0.3 | 2.1 |
| E08m | Other Enzymeslinvolved in | glioma pathogenesis-related protein; RT | −1.1 | 2.0 |
| | Protein Turnover | | | |
| C13g | DNA Damage Repair Proteins & | DNA-repair protein complementing XP-A | 0.0 | 2.0 |
| | Ligases | | | |
| G31 | Housekeeping Genes | HLA class I histocompatibllity antigen C- | 0.0 | 2.0 |
| F03h | Intermediate Filament Proteins | cardiac ventricular myosin right chain 2 | 0.2 | 2.0 |
| E11i | Other Extracellular | matrix metalloproteinase 12 (MMP12); [symbol] | 0.0 | 2.0 |
| | Communication Proteins | | | |
| A11i | Cyclins | cyclin H (CCNH); MO15-assoclated prot [symbol] | 0.3 | 2.0 |
| F03g | Intermediate Filament Proteins | type II cytoskeletal 8 keratin (KRT8); cyt [symbol] | 0.0 | 2.0 |
| C12b | Caspases | caspase-10 precursor (CASP10); ICE-L [symbol] | 0.4 | 2.0 |
| A10i | Cyclins | G2/mitotic-specific cyclin G1 (CCNG1; [symbol] | 0.3 | 1.9 |
| G43 | Housekeeping Genes | cytoplasmic beta-actin (ACTB) | 0.0 | 1.9 |
| E01i | Intracellular Transducers, | Wnt-10B precursor, Wnt-12 | 0.0 | 1.9 |
| | Effectors & Modulators | | | |
| C10b | Caspases | caspase-8 precursor (CASP8); ICE-like [symbol] | 0.0 | 1.9 |
| A10k | Cell Cycle-Regulating Kinases | cyclin-dependent kinase regulatory subu | 0.0 | 1.9 |
| E02f | Growth Factors, Cytokines & | insulin-like growth factor-binding protein | 0.0 | 1.8 |
| | Chemokines | | | |
| E14n | Immunoglobulins | collagen 8 alpha 1 subunit (COL8A1) | 0.0 | 1.8 |
| E01n | Major Histocompatibility Complex | unrearranged immunoglobulin V(H)5 ps [symbol] | 0.0 | 1.8 |
| | Proteins | | | |
| E01e | Growth Factors, Cytokines & | glia-activating factor precursor (GAF); fit | 0.4 | 1.8 |
| | Chemokines | | | |
| D09n | Other Stress Response Proteins | glial cell line-derived neurotropec factor [symbol] | 0.0 | 1.8 |
| E02g | Hormones | interleukin-14 precursor (IL-14); high mo | 0.0 | 1.8 |
| D03n | Other Stress Response Proteins | (2′-5′)oligoadenylate synthetase 2 ((2-5′) [symbol] | 0.0 | 1.8 |
| C03f | DNA Polymerases, Replication | replication factor C 38-kDa subunit (RFC | 0.5 | 1.8 |
| | Factors & Topoisomerases | | | |
| E02m | Cysteine Proteases | MHC class I truncated HLA G lymphocyt | 0.0 | 1.8 |
| A12e | Oncogenes & Tumor Suppressors | shb proto-oncogene | −1.2 | 1.8 |
| F05g | Intermediate Filament Proteins | desmin (DES) | 0.0 | 1.8 |
| C11l | Transcription Activators & | major histocompatibility complex enhan [symbol] | 0.0 | 1.8 |
| | Repressors | | | |
| D11h | Cell-Cell Adhesion Receptors | frizzled | 0.0 | 1.7 |
| E05j | Metalloproteinases | plasminogen precursor (PLG) | 0.0 | 1.7 |
| E06h | Interleukins & Interferons | angiopoietin 1 | 0.4 | 1.7 |
| E07i | Other Extracellular | matrix metalloproteinase 3 (MMP3); stro | 0.0 | 1.7 |
| | Communication Proteins | | | |
| D14g | Cell-Cell Adhesion Receptors | desmocollin 3A/3B precursor (DSC3) + [symbol] | 0.0 | 1.7 |
| D02c | Cell Surface Antigens | lymphocyte antigen | 0.0 | 1.7 |
| C02j | Basic Transcription Factors | RBP2 retinoblastoma binding protein | 0.1 | 1.7 |
| C08e | Other Apoptosis-Associated | maleylacetoacetate isomerase (MAAI); [symbol] | 0.0 | 1.7 |
| | Proteins | | | |
| E07k | Protease Inhibitors | putative ATP-dependent CLP protease [symbol] | 0.1 | 1.7 |
| F01d | Metabolism or Cofactors, | xanthine dehydrogenase/oxidase | 0.0 | 1.7 |
| | Vitamins & Related Substances | | | |
| F08g | Intermediate Filament Proteins | tubulin gamma subunit | 0.0 | 1.7 |
| D08k | Intracellular Transducers, | DNAX activation protein 12 | 0.0 | 1.7 |
| | Effectors & Modulators | | | |
| E05h | Interleukins & Interferons | beta-adrenergic receptor kinase 1 (beta- | 0.1 | 1.6 |
| F02n | Functionally Unclassified Proteins | KIAA0137 | 0.2 | 1.6 |
| E12m | Immune System Proteins | class II histocompatibility antigen M alph | 0.0 | 1.6 |
| F06n | Functionally Unclassified Proteins | early growth response alpha (EGR alpha | −1.0 | 1.6 |
| F13l | Other Proteins involved in | reinoic acid- & interferon-inducible 58K [symbol] | 0.0 | 1.6 |
| | Translation | | | |
| D05i | Intracellular Transducers, | fibroblast growth factor receptor 3 precu [symbol] | 0.1 | 1.6 |
| | Effectors & Modulators | | | |
| C04l | Transcription Activators & | retinoic acid receptor alpha: retinoid X re | 0.0 | 1.6 |
| | Repressors | | | |
| D12l | Other Receptors (by Ligands) | frizzled-related FrzB (FRITZ) + FrzB pre | 0.1 | 1.6 |
| C14b | Caspases | caspase-7 precursor (CASP7); ICE-like [symbol] | 0.5 | 1.6 |
| F04m | Functionally Unclassified Proteins | polyhomeotic 2 homolog (HPH2) | 0.0 | 1.6 |
| E01k | Metalloproteinases | cathepsin L precursor; major excreted p [symbol] | 0.0 | 1.6 |
| E06e | Growth Factors, Cytokines & | SDF1A; pre-B cell stimulating factor hon | 0.0 | 1.6 |
| | Chemokines | | | |
| D13j | Growth Factor & Chemokine | oncostatin M-specific receptor beta subu | 0.0 | 1.6 |
| | Receptors | | | |
| D14f | Cell Adhesion Receptors & | transient axonal glycoprotein 1 (TAX1; T | 0.0 | 1.6 |
| | Proteins | | | |
| E04g | Interleukins & Interferons | Interleukin-12 beta subunit precursor (IL- | 0.0 | 1.5 |
| A14l | Other Cell Cycle Proteins | transcription factor DP2 (Humdp2); E2F | 0.0 | 1.5 |
| A12j | Cell Cycle-Regulating Kinases | cdc2-related protein kinase PISSLRE | 0.2 | −1.5 |
| A13h | Oncogenes & Tumor Suppressors | active breakpoint cluster region-related [symbol] | 0.0 | −1.5 |
| B08a | Intracellular Adaptors & Receptor- | c-src kinase (CSK); protein-tyrosine kina | 0.1 | −1.5 |
| | Associated Proteins | | | |
| B01f | Intracellular Kinase Network | KIAA0096 | 0.1 | −1.5 |
| | Members | | | |
| A03e | Oncogenes & Tumor Suppressors | vascular endothelial growth factor recept | 0.0 | −1.6 |
| A09a | Oncogenes & Tumor Suppressors | retinoblastoma-associated protein (RB1) | 0.0 | −1.6 |
| A12f | Oncogenes & Tumor Suppressors | synapse-associated protein 102 (SAP10: | 0.0 | −1.6 |
| B13h | Intracellular Protein | protein phosphatase with EF-hands-2 lor | 0.1 | −1.6 |
| | Phosphatases | | | |
| B09h | Intracellular Protein | protein phosphatase 2A B56-alpha | 0.0 | −1.6 |
| | Phosphatases | | | |
| A01n | Other Cell Cycle Proteins | RBQ1 retinoplastoma binding protein | 0.4 | −1.6 |
| C07i | Recombination Proteins | V(D)J recombination activating protein 1 | 0.0 | −1.6 |
| B12n | Death Receptors | insulin-like growth factor 1 receptor (IGF1 | 0.0 | −1.6 |
| C09h | DNA Damage Repair Proteins & | DNA mismatch repair protein MSH6; mu | 0.0 | −1.7 |
| | Ligases | | | |
| D05g | Cell-Cell Adhesion Receptors | cadherin 12 (CDH12); brain cadherin pre [symbol] | 0.0 | −1.7 |
| A11b | Oncogenes & Tumor Suppressors | TSG101 tumor susceptibility protein | −1.0 | −1.7 |
| F02b | Extracellular Matrix Proteins | vitronectin precursor (VTN); serum spre [symbol] | 0.0 | −1.7 |
| A11h | Oncogenes & Tumor Suppressors | box-dependent myc-interacting protein 1 | 0.2 | −1.7 |
| C06i | Recombination Proteins | recA-like protein HsRad51; DNA repair p | 0.2 | −1.7 |
| C01k | Transcription Activators & | homeobox protein HOX-D3; HOX-4A | 0.0 | −1.7 |
| | Repressors | | | |
| D06c | Cell Surface Antigens | mesothelin precursor; CAK1 antigen | 0.0 | −1.7 |
| A01i | Oncogenes & Tumor Suppressors | leukemia-associated gene 1 | 0.0 | −1.7 |
| C04h | DNA Damage Repair Proteins & | HHR23A; UV excision repair protein pro [symbol] | 0.9 | −1.8 |
| | Ligases | | | |
| A04e | Oncogenes & Tumor Suppressors | tyrosine-protein kinase receptor tyro3 p [symbol] | 0.0 | −1.8 |
| C06k | Transcription Activators & | interferon regulatory factor 1 (IRF1) | 0.6 | −1.8 |
| | Repressors | | | |
| A12h | Oncogenes & Tumor Suppressors | cullin homolog 2 (CUL2) | −1.2 | −1.8 |
| A13i | Hormone Receptors | thyrotropin-releasing hormone receptor | 0.0 | −1.8 |
| F10g | Intermediate Filament Proteins | myosin-IXB | 1.1 | −1.8 |
| D07e | Cell-Cell Adhesion Receptors | Integrin alpha 1 (ITGA1); laminin & colla | 0.0 | −1.8 |
| C03b | Death Receptor-Associated | MAP kinase-activating death domain pro | 0.0 | −1.8 |
| | Proteins & Adaptors | | | |
| C13l | Hormone Receptors | muscarinic acetylcholine receptor M4 (C | 0.0 | −1.8 |
| B03c | Intracellular Kinase Network | dual specificity mitogen-activated protein | 0.4 | −1.8 |
| | Members | | | |
| C13e | DNA Polymerases, Replication | proliferating cyclic nuclear antigen (PCN | 0.0 | −1.8 |
| | Factors & Topoisomerases | | | |
| F06g | intermediate Filament Proteins | hyaluronan receptor (RHAMM) | 0.0 | −1.9 |
| B04f | Intracellular Kinase Network | STE20-like kinase; MST2 | 0.0 | −1.9 |
| | Members | | | |
| B09d | Intracellular Kinase Network | casein kinase I gamma 2 (CKI-gamma 2 | 0.0 | −1.9 |
| | Members | | | |
| B04h | Intracellular Kinase Network | protein kinase DYRK2 | 0.0 | −1.9 |
| | Members | | | |
| B11a | Intracellular Adaptors & Receptor- | c-fer proto-oncogene | 0.0 | −1.9 |
| | Associated Proteins | | | |
| B12h | Intracellular Protein | protein phosphatase PP2A 61-kDa regul. | 0.0 | −1.9 |
| | Phosphatases | | | |
| E14e | Growth Factors, Cytokines & | dishevelled (DVL) + dishevelled 3 (DVL3 | 0.0 | −1.9 |
| | Chemokines | | | |
| B14d | Intracellular Kinase Network | ribosomal protein S6 kinase II alpha 2 (S | 0.5 | −2.0 |
| | Members | | | |
| F11i | Other Trafficking & Targeting | cleavage stimulation factor 77-kDa subu | 0.0 | −2.0 |
| | Proteins | | | |
| B10a | Intracellular Adaptors & Receptor- | 70-kDa zeta-associated protein (ZAP70) | 0.0 | −2.0 |
| | Associated Proteins | | | |
| C03h | DNA Damage Repair Proteins & | DNA damage repair & recombination pro | 0.0 | −2.0 |
| | Ligases | | | |
| B09c | Intracellular Kinase Network | c-jun N-terminal kinase 1 (JNK1); JNK46 | 0.1 | −2.1 |
| | Members | | | |
| B14j | GTP/GDP Exchangers & GTPase | rho GDP dissociation inihibitor 1 (RHO-G | 0.1 | −2.1 |
| | Activity Modulators | | | |
| B10d | Intracellular Kinase Network | cAMP-dependent protein kinase beta-ca [symbol] | 0.1 | −2.1 |
| | Members | | | |
| C11f | DNA Polymerases, Replication | DNA topoisomerase III (TOP3) | 0.0 | −2.2 |
| | Factors & Topoisomerases | | | |
| B11b | Oncogenes & Tumor Suppressors | GRB-IR/GRB10 | 0.0 | −2.2 |
| B10g | Intracellular Kinase Network | B-lymphocyte germinal center kinase (G | 0.0 | −2.2 |
| | Members | | | |
| A02e | Oncogenes & Tumor Suppressors | platelet-derived growth factor receptor al | 0.0 | −2.2 |
| CO3n | Transcription Activators & | E4BP4 | 0.2 | −2.2 |
| | Repressors | | | |
| C06m | Transcription Activators & | B4-2 protein | 0.6 | −2.3 |
| | Repressors | | | |
| F12i | General Trafficking Proteins | ribonuclease 6 precursor | 0.2 | −2.3 |
| A06c | Oncogenes & Tumor Suppressors | N-myc proto-oncogene | 0.0 | −2.4 |
| C08j | Transcription Activators & | nuclear factor NF-kappa-B p100 subunit; | 0.2 | −2.4 |
| | Repressors | | | |
| F14i | Other Trafficking & Targeting | RNA helicase | 0.0 | −2.4 |
| | Proteins | | | |
| B02e | Intracellular Kinase Network | kinase suppressor of ras-1 (KSR1) | 0.0 | −2.5 |
| | Members | | | |
| B08c | Intracellular Kinase Network | cAMP-dependent protein kinase I alpha [symbol] | 0.0 | −2.6 |
| | Members | | | |
| B02k | GTP/GDP Exchangers & GTPase | regulator of G protein signaling 14 (RGS | 0.5 | −2.8 |
| | Activity Modulators | | | |
| B10k | GTP/GDP Exchangers & GTPase | Interferon-Induced guanylate-binding pro | 0.7 | −2.9 |
| | Activity Modulators | | | |
| C13h | DNA Damage Repair Proteins & | ALKB homolog protein | 0.0 | −3.0 |
| | Ligases | | | |
| F09h | Cytoskeleton & Motility Proteins | TRAM protein | 0.2 | −3.7 |

TABLE 7d
GENE CHANGES INDUCED BY VEE VIRUS IN VITRO IN HUMAN LYMPHOID
CELLS - ARRAY I

| Gene Function | Protein/gene | VEE-1h | VEE-4h |
|---|---|---|---|
| Basic Transcription Factors | hypoxia-inducible factor 1 alp [symbol] | 0.0 | 3.9 |
| Growth Factors, Cytokines & Chemokines | FIBROBLAST GROWTH FAC | 6.6 | 3.3 |
| Housekeeping Genes | phospholipase A2 | 0.0 | 3.2 |
| Basic Transcription Factors | transcription factor ZFM1 | 1.8 | 3.1 |
| Death Receptors | decoy receptor 2 | 0.5 | 3.1 |
| Transcription Proteins | FHF-1 | 0.0 | 3.0 |
| Cell-Cell Adhesion Receptors | vitronectin receptor alpha sub [symbol] | −1.0 | 2.9 |
| DNA Damage Repair Proteins & Ligases | DNA-dependent protein Kinase | 0.4 | 2.8 |
| Metalloproteinases | glia-derived neurite-promoting | 0.0 | 2.8 |
| Growth Factors, Cytokines & Chemokines | vascular endothelial growth fa | −1.2 | 2.7 |
| Xenobiotic Transporters | microsomal glutathione S-tran | 0.0 | 2.7 |
| Intracellular Kinase Network Members | lipid-activated protein kinase [symbol] | 0.2 | 2.7 |
| Housekeeping Genes | liver glyceraldehyde 3-phosph | 0.0 | 2.7 |
| Other Extracellular Communication | cathepsin D precursor (CTSD) | 0.0 | 2.7 |
| Proteins | | | |
| Other Extracellular Communication | microsomal aminopeptidase [symbol] | 0.0 | 2.7 |
| Proteins | | | |
| Transcription Activators & Repressors | zinc-finger DNA-binding protei [symbol] | 0.5 | 2.7 |
| Basic Transcription Factors | GA-binding protein beta-2 sub | 0.9 | 2.7 |
| Basic Transcription Factors | histone acetyltransferase B su | 1.6 | 2.7 |
| Transcription Activators & Repressors | putative transcription activato [symbol] | −1.2 | 2.6 |
| Extracellular Matrix Proteins | aminoacylase 1 (ACY1) | 1.2 | 2.6 |
| Cell Signaling & Extracellular | glia maturation factor beta (GI | −1.0 | 2.6 |
| Communication Proteins | | | |
| Nucleotide Metabolism | BIGH3 | 0.4 | 2.5 |
| Cell-Cell Adhesion Receptors | fibronectin receptor beta subu | 0.0 | 2.5 |
| Transcription Activators & Repressors | helix-loop-helix protein; DNA- [symbol] | 0.7 | 2.5 |
| Growth Factors. Cytokines & Chemokines | interleukin-1 receptor antagon | 0.0 | 2.5 |
| Other Extracellular Communication | matrix metalloproteinase 9 (M | 0.4 | 2.5 |
| Proteins | | | |
| Interleukin & Interferon Receptors | interleukin-6 receptor alpha s [symbol] | −2.4 | 2.4 |
| Interleukin & Interferon Receptors | interferon-alpha/beta receptor | 1.1 | 2.4 |
| Other Receptors (by Ligands) | frizzled homolog (FZD3) | 0.9 | 2.4 |
| Metalloproteinases | metalloproteinase inhibitor 1 [symbol] | 0.0 | 2.4 |
| Cyclins | cyclin G2 (CCNG2) | 0.0 | 2.3 |
| Oncogenes & Tumor Suppressors | matrix metalloproteinase 11 ( [symbol] | 0.0 | 2.3 |
| Chaperones & Heat Shock Proteins | 60S ribosomal protein L5 | 0.5 | 2.3 |
| Functionally Unclassified Proteins | menin | 0.5 | 2.3 |
| Intracellular Transducers, Effectors & | Wnt-8B | 0.1 | 2.3 |
| Modulators | | | |
| Cell Signating & Extracellular | major prion protein precursor [symbol] | 0.0 | 2.3 |
| Communication Proteins | | | |
| Transcription Activators & Repressors | metal-regulatory transcription | 0.0 | 2.2 |
| Growth Factor & Chemokine Receptors | N-sam; fibroblast growth facto [symbol] | 0.5 | 2.2 |
| Growth Factor & Chemokine Receptors | granulocyte colony stimulating | −2.1 | 2.2 |
| Intracellular Kinase Network Members | dual-specificity mitogen-activ [symbol] | 0.3 | 2.2 |
| Transcription Activators & Repressors | fli-1 oncogene; ergB transcrip [symbol] | 0.0 | 2.2 |
| Basic Transcription Factors | transcription factor ETR101 | 2.1 | 2.2 |
| Extracellular Matrix Proteins | bone morphogenetic protein 4 | 0.0 | 2.2 |
| Other Enzymeslinvolved in Protein | HLA-DR antigen-associated in | 0.3 | 2.2 |
| Turnover | | | |
| Transcription Activators & Repressors | PCAF-associated factor 65 be | 0.5 | 2.2 |
| Matrix Adhesion Receptors | leukocyte adhesion glycoprot [symbol] | −1.8 | 2.2 |
| Intracellular Kinase Network Members | dual specificity mitogen-activ [symbol] | 0.0 | 2.2 |
| Functionally Unclassified Proteins | HEM45 | 0.5 | 2.2 |
| Transcription Activators & Repressors | transcription repressor protein | 0.8 | 2.2 |
| Transcription Activators & Repressors | zinc finger protein 91 (ZNF92) | 0.0 | 2.2 |
| Transcription Activators & Repressors | ets domain protein elk-3; NE [symbol] | 1.4 | 2.2 |
| Transcription Activators & Repressors | octamer-binding transcription | 1.4 | 2.2 |
| Proteosomal Proteins | proteasome component C5; [symbol] | 0.0 | 2.2 |
| Cell Surface Antigens | Integrin beta 8 precursor (ITG [symbol] | −1.8 | 2.1 |
| Transcription Activators & Repressors | early growth response protein | 0.5 | 2.1 |
| Housekeeping Genes | 40S ribosomal protein S9 | −1.0 | 2.1 |
| Transcription Activators & Repressors | HIV-1 TATA element modulat [symbol] | 0.0 | 2.1 |
| Cell-Cell Adhesion Receptors | CD44 antigen hematopoietic [symbol] | 0.0 | 2.1 |
| ATPase Transporters | ATP synthase coupling factor | 0.3 | 2.1 |
| Other Enzymeslinvolved in Protein | glioma pathogenesis-related [symbol] | −1.1 | 2.0 |
| Turnover | | | |
| DNA Damage Repair Proteins & Ligases | DNA-repair protein compleme | 0.0 | 2.0 |
| Housekeeping Genes | HLA class I histocompatibility | 0.0 | 2.0 |
| Intermediate Filament Proteins | cardiac ventricular myosin lig [symbol] | 0.2 | 2.0 |
| Complex Lipid Metabolism | C-4 methyl sterol oxidase | 0.5 | 2.0 |
| Xenobiotic Transporters | glutathione S-transferase pi ( [symbol] | 0.0 | 2.0 |
| Other Extracellular Communication | matrix metalloproteinase 12 ( [symbol] | 0.0 | 2.0 |
| Proteins | | | |
| Cyclins | cyclin H (CCNH); MO15-asso [symbol] | 0.3 | 2.0 |
| Cell Surface Antigens | platelet glycoprotein Ib alpha [symbol] | 0.0 | 2.0 |
| Complex Lipid Metabolism | cholinephosphate cytidylyltran | 0.5 | 2.0 |
| Metalloproteinases | ADAM10 | 1.3 | 2.0 |
| Translation | 14.5-kDa translational inhibito | 0.9 | 2.0 |
| Intermediate Filament Proteins | type II cytoskeletal 8 keratin ( [symbol] | 0.0 | 2.0 |
| Chromatin Proteins | high mobility group protein (H [symbol] | 0.0 | 2.0 |
| Caspases | caspase-10 precursor (CASP [symbol] | 0.4 | 2.0 |
| Interleukin & Interferon Receptors | interferon-alpha/beta receptor | 0.7 | 2.0 |
| Cell Surface Antigens | laminin alpha-3 subunit precu [symbol] | 0.0 | 2.0 |
| Nucleotide Metabolism | adenylate kinase 3 (AK3); mit [symbol] | 4.5 | 2.0 |
| Transcription Activators & Repressors | nuclear factor NF-kappa-B p1 [symbol] | 1.7 | 1.9 |
| Cyclins | G2/mitotic-specific cyclin G1 ( | 0.3 | 1.9 |
| Basic Transcription Factors | basic transcription element-bi [symbol] | 0.3 | 1.9 |
| Exocytosis Proteins | vesicle-membrane fusion prot | 1.8 | 1.9 |
| Housekeeping Genes | cytoplasmic beta-actin (ACTB | 0.0 | 1.9 |
| Other Trafficking & Targeting Proteins | SEC13-related protein (SEC1 [symbol] | 0.7 | 1.9 |
| Intracellular Transducers, Effectors & | ephrin A3 precursor (EFNA3) [symbol] | 0.4 | 1.9 |
| Modulators | | | |
| Other Trafficking & Targeting Proteins | ras-related protein RAB-1A; Y | 0.0 | 1.9 |
| Cell Signaling & Extracellular | acyl-CoA-binding protein (AC [symbol] | 0.0 | 1.9 |
| Communication Proteins | | | |
| Basic Transcription Factors | ZFM1 protein alternatively spl | 2.6 | 1.9 |
| Intracellular Kinase Network Members | phosphorylase B kinase gamn | 0.7 | 1.9 |
| Housekeeping Genes | liver glyceraldehyde 3-phosph | 0.1 | 1.9 |
| Intracellular Transducers, Effectors & | Wnt-10B precursor; Wnt-12 | 0.0 | 1.9 |
| Modulators | | | |
| Caspases | caspase-8 precursor (CASP8) | 0.0 | 1.9 |
| Other Trafficking & Targeting Proteins | alpha-soluble NSF attachmen [symbol] | 1.3 | 1.9 |
| Proteosomal Proteins | proteasome component C3; [symbol] | 0.0 | 1.9 |
| Other Intracellular Transducers, Effectors | amyloid-like protein 2 | 1.2 | 1.9 |
| & Modulators | | | |
| Cell Cycle-Regulating Kinases | cyclin-dependent kinase regul [symbol] | 0.0 | 1.9 |
| Other Intracellular Transducers, Effectors | 14-3-3 protein tau; 14-3-3 prot | 1.4 | 1.9 |
| & Modulators | | | |
| Functionally Unclassified Proteins | P18 protein | −2.5 | 1.9 |
| Interleukin & Interferon Receptors | interleukin-1 receptor type II p | −1.6 | 1.8 |
| Growth Factors, Cytokines & Chemokines | insulin-like growth factor-bindi [symbol] | 0.0 | 1.8 |
| Transcription Activators & Repressors | homeobox protein HOXB7; H [symbol] | 0.0 | 1.8 |
| Basic Transcription Factors | transcription factor HTF4; tran [symbol] | 0.9 | 1.8 |
| Cell Surface Antigens | lysosome-associated membra [symbol] | 0.9 | 1.8 |
| Cell Surface Antigens | CD83 antigen precursor; cell [symbol] | −1.3 | 1.8 |
| Cell Surface Antigens | lysosome membrane protein [symbol] | 0.0 | 1.8 |
| Immunoglobulins | collagen 8 alpha 1 subunit (C [symbol] | 0.0 | 1.8 |
| Major Histocompatibility Complex | unrearranged immunoglobulin | 0.0 | 1.8 |
| Proteins | | | |
| Intracellular Kinase Network Members | calcium/calmodulin-dependent | −1.2 | 1.8 |
| Symporters & Antiporters | sodium- & chloride-dependent | 0.5 | 1.8 |
| Cell Surface Antigens | leukemia virus receptor 2 (GL | 1.1 | 1.8 |
| Growth Factors, Cytokines & Chemokines | glia-activating factor precurso [symbol] | 0.4 | 1.8 |
| Cell Signaling & Extracellular | major prion protein precursor [symbol] | 1.3 | 1.8 |
| Communication Proteins | | | |
| Other Receptors (by Ligands) | glutathione-S-transferase (GS | −1.6 | 1.8 |
| Death Receptors | tumor necrosis factor recepto [symbol] | 0.0 | 1.8 |
| Cell-Cell Adhesion Receptors | integrin alpha 4 precursor (IT [symbol] | 0.4 | 1.8 |
| Other Stress Response Proteins | glial cell line-derived neurotro [symbol] | 0.0 | 1.8 |
| Cell Surface Antigens | laminin alpha-2 subunit precu [symbol] $ | 0.0 | 1.8 |
| Hormones | interleukin-14 precursor (IL-14 [symbol] | 0.0 | 1.8 |
| Transcription Activators & Repressors | interleukin enhancer-binding [symbol] | 0.5 | 1.8 |
| Growth Factor & Chemokine Receptors | neuromedin B receptor (NMB [symbol] | 0.7 | 1.8 |
| Other Trafficking & Targeting Proteins | coatomer alpha subunit; alpha | 0.3 | 1.8 |
| Other Stress Response Proteins | (2′-5′)oligoadenylate synthetas [symbol] | 0.0 | 1.8 |
| DNA Polymerases, Replication Factors & | replication factor C 38-kDa su | 0.5 | 1.8 |
| Topoisomerases | | | |
| Orphan Receptors | RAR-related orphan receptor [symbol] | 1.4 | 1.8 |
| Kinase Activators & Inhibitors | protein kinase C substrate 80- | 0.5 | 1.8 |
| Transcription Activators & Repressors | signal transducer and activato | 0.3 | 1.8 |
| Intracellular Kinase Network Members | casein kinase I gamma 2 (CK | 0.0 | 1.8 |
| Cysteine Proteases | MHC class I truncated HLA G | 0.0 | 1.8 |
| Oncogenes & Tumor Suppressors | shb proto-oncogene | −1.2 | 1.8 |
| Intermediate Filament Proteins | desmin (DES) | 0.0 | 1.8 |
| Intracellular Kinase Network Members | calcium/calmodulin-dependen | 0.3 | 1.8 |
| Transcription Activators & Repressors | major histocompatibility comp | 0.0 | 1.8 |
| Functionally Unclassified Proteins | nidogen precursor (NID); enta | 1.8 | 1.7 |
| Cell-Cell Adhesion Receptors | frizzled | 0.0 | 1.7 |
| Cell Surface Antigens | Syndecan2 (Fibroglycan) (Her | 0.5 | 1.7 |
| Metalloproteinases | plasminogen precursor (PLG) | 0.0 | 1.7 |
| Interleukins & Interferons | angiopoietin 1 | 0.4 | 1.7 |
| Other Extracellular Communication | matrix metalloproteinase 3 (M | 0.0 | 1.7 |
| Proteins | | | |
| G Protein-Coupled Receptors | EBV-induced G-protein-coupl [symbol] | 1.1 | 1.7 |
| Cysteine Proteases | cathepsin B precursor (CTSB) | 0.0 | 1.7 |
| Nucleotide Metabolism | adenylate kinase isoenzyme 1 | 4.4 | 1.7 |
| Complex Lipid Metabolism | cholesterol acyltransferase | 0.0 | 1.7 |
| Growth Factors, Cytokines & Chemokines | macrophage inflammatory pro | 0.0 | 1.7 |
| Cell-Cell Adhesion Receptors | desmocollin 3A/3B precursor [symbol] | 0.0 | 1.7 |
| Other Apoptosis-Associated Proteins | cytoplasmic antiproteinase 3 ( | −1.7 | 1.7 |
| Cell Surface Antigens | lymphocyte antigen | 0.0 | 1.7 |
| Basic Transcription Factors | RBP2 retinoblastoma binding | 0.1 | 1.7 |
| DNA Polymerases, Replication Factors & | DNA topoisomerase I (TOP1) | 0.0 | 1.7 |
| Topoisomerases | | | |
| Oncogenes & Tumor Suppressors | jun-B | 2.4 | 1.7 |
| Other Apoptosis-Associated Proteins | maleylacetoacetate isomerase | 0.0 | 1.7 |
| Kinase Activators & Inhibitors | linker for activation of T-cells [symbol] | 1.0 | 1.7 |
| Transcription Activators & Repressors | jun activation domain binding | 0.0 | 1.7 |
| Protease Inhibitors | putative ATP-dependent CLP | 0.1 | 1.7 |
| Complex Lipid Metabolism | sterol C5 desaturase (C5D); I [symbol] | 1.3 | 1.7 |
| Metabolism of Cofactors, Vitamins & | xanthine dehydrogenase/oxid [symbol] | 0.0 | 1.7 |
| Related Substances | | | |
| Intermediate Filament Proteins | tubulin gamma subunit | 0.0 | 1.7 |
| Nucleotide Metabolism | type II cytoskeletal 2 epiderm [symbol] | 3.3 | 1.7 |
| Intracellular Transducers, Effectors & | DNAX activation protein 12 | 0.0 | 1.7 |
| Modulators | | | |
| Other Receptors (by Ligands) | protein kinase C iota type (NP | −1.1 | 1.6 |
| Interleukins & Interferons | beta-adrenergic receptor kina [symbol] | 0.1 | 1.6 |
| Cell-Signaling & Extracellular | histidine decarboxylase (HDC [symbol] | 0.0 | 1.6 |
| Communication Proteins | | | |
| Functionally Unclassified Proteins | KIAA0137 | 0.2 | 1.6 |
| Intracellular Kinase Network Members | protein kinase C gamma type | 0.0 | 1.6 |
| Oncogenes & Tumor Suppressors | clathrin assembly protein lym [symbol] | 0.4 | 1.6 |
| Immune System Proteins | class II histocompatibility anti [symbol] | 0.0 | 1.6 |
| Hormone Receptors | somatostatin receptor type 1 ( | 0.3 | 1.6 |
| Cell Surface Antigens | T-cell surface glycoprotein CD | 3.2 | 1.6 |
| G Proteins | RaIB GTP-binding protein | −1.8 | 1.6 |
| G Proteins | Ral A; GTP-binding protein | 0.0 | 1.6 |
| Housekeeping Genes | phospholipase A2 | 4.0 | 1.6 |
| Other Metabolism Enzymes | ferritin heavy chain (FTH1); F | −2.2 | 1.6 |
| Other Intracellular Transducers, Effectors | SH3P18 SH3 domain-containi | 0.0 | 1.6 |
| & Modulators | | | |
| Adenylate/Guanylate Cyclases & | guanylate cyclase soluble alpt | 1.8 | 1.6 |
| Diesterases | | | |
| Functionally Unclassified Proteins | early growth response alpha (I | −1.0 | 1.6 |
| Cell Cycle-Regulating Kinases | FUSE binding protein | 0.8 | 1.6 |
| Cell Signaling & Extracellular | Kallmann syndrome protein pt | 0.0 | 1.6 |
| Communication Proteins | | | |
| Other Proteins Involved in Translation | reinoic acid- & interferon-indu [symbol] | 0.0 | 1.6 |
| Basic Transcription Factors | CCAAT transcription binding f | 0.0 | 1.6 |
| Cell Signaling & Extracellular | presynaptic density protein 95 | 1.1 | 1.6 |
| Communication Proteins | | | |
| Hormone Receptors | BB2-Bombesin receptor | 0.6 | 1.6 |
| Intracellular Kinase Network Members | LIM domain kinase 1 (LIMK-1) | 0.5 | 1.6 |
| Hormone Receptors | gonadotropin-releasing hormo | 1.8 | 1.6 |
| Interleukin & Interferon Receptors | Interferon-alpha/beta receptor | 1.0 | 1.6 |
| Intracellular Transducers, Effectors & | fibroblast growth factor recept | 0.1 | 1.6 |
| Modulators | | | |
| Transcription Activators & Repressors | retinoic acid receptor alpha; r [symbol] | 0.0 | 1.6 |
| Other Receptors (by Ligands) | frizzled-related FrzB (FRITZ) | 0.1 | 1.6 |
| Caspases | caspase-7 precursor (CASP7) | 0.5 | 1.6 |
| Functionally Unclassified Proteins | polyhomeotic 2 homolog (HP [symbol] | 0.0 | 1.6 |
| Metalloproteinases | cathepsin L precursor, major [symbol] | 0.0 | 1.6 |
| Basic Transcription Factors | transcriptional repressor NF-X | 1.8 | 1.6 |
| Growth Factors, Cytokines & Chemokines | SDF1A; pre-B cell stimulating | 0.0 | 1.6 |
| Growth Factor & Chemokine Receptors | oncostatin M-specific receptor | 0.0 | 1.6 |
| Transcription Activators & Repressors | heat shock factor protein 1 (H [symbol] | 0.8 | 1.6 |
| CDK Inhibitors | Sp3 protein | 0.0 | 1.6 |
| Cell Adhesion Receptors & Proteins | transient axonal glycoprotein | 0.0 | 1.6 |
| Cell Surface Antigens | complement decay-accelerati [symbol] | 0.8 | 1.6 |
| Cell Surface Antigens | P-selectin precursor (SELP); [symbol] | 0.1 | 1.6 |
| Cell Surface Antigens | platelet glycoprotein IX | 0.0 | 1.5 |
| CDK Inhibitors | trans-acting T-cell specific tra [symbol] | 1.2 | 1.5 |
| Interleukins & interferons | interleukin-12 beta subunit pr [symbol] | 0.0 | 1.5 |
| Interleukin & Interferon Receptors | interleukin-2 receptor alpha SL | 1.2 | 1.5 |
| Cell Signaling & Extracellular | 43-kDa postsynaptic protein; [symbol] | 1.3 | 1.5 |
| Communication Proteins | | | |
| Transcription Activators & Repressors | B-cell lymphoma 6 protein (bc | 0.6 | 1.5 |
| Cell-Cell Adhesion Receptors | semaphorin; CD100 | 1.0 | 1.5 |
| Other Cell Cycle Proteins | transcription factor DP2 (Hum | 0.0 | 1.5 |
| Oncegenes & Tumor Suppressors | gamma-interferon-inducible p [symbol] | −1.6 | 1.5 |
| Adenylate/Guanylate Cyclases & | bone marrow stromal antigen | −1.8 | 1.4 |
| Diesterases | | | |
| Basic Transcription Factors | transcription factor RZR-alpha | 2.0 | 1.4 |
| Xenobiotic Metabolism | cytosolic superoxide dismutas | 1.5 | 1.4 |
| Cell-Cell Adhesion Receptors | leukocyte adhesion glycoprotein | 2.2 | 1.3 |
| Cell-Cell Adhesion Receptors | integrin beta 6 precursor (ITGI | −1.6 | 1.3 |
| Transcription Activators & Repressors | TAX1-binding protein 151 (TX | 3.0 | 1.3 |
| Basic Transcription Factors | cellular nucleic acid binding p [symbol] | 1.9 | 1.3 |
| Nucleotide Metabolism | 5,6-dihydroxyindole-2-carboxy | 4.6 | 1.3 |
| Growth Factor & Chemokine Receptors | granulocyte-macrophage colo [symbol] | −2.0 | 1.3 |
| Other Intracellular Transducers, Effectors | junction plakoglobin (JUP); de | 1.8 | 1.2 |
| & Modulators | | | |
| Intracellular Transducers, Effectors & | cadherin1 (CDH1); epithelial [symbol] | 3.9 | 1.2 |
| Modulators | | | |
| Nucleotide Metabolism | microsomal UDP-glucuronosy | 5.5 | 1.1 |
| Xenobiotic Transporters | growth arrest & DNA-damage- | 1.9 | 1.1 |
| Intracellular Kinase Network Members | protein kinase C epsilon type [symbol] | 1.6 | 1.1 |
| Major Histocompatibility Complex | immunoglobulin alpha 1 heav [symbol] | 1.7 | 1.1 |
| Proteins | | | |
| Growth Factors, Cytokines & Chemokines | transforming growth factor-be [symbol] | 2.4 | 1.1 |
| Intracellular Transducers, Effectors & | interferon-gamma (IFN-gamm | −1.6 | 0.9 |
| Modulators | | | |
| Nucleotide Metabolism | type II cytoskeletal 2 oral kera | 4.3 | 0.9 |
| Interleukins & Interferons | interleukin-1 alpha precursor ( | −1.6 | 0.9 |
| Intracellular Protein Phosphatases | serine/threonine protein phos [symbol] | 2.3 | 0.9 |
| Kinase Activators & Inhibitors | 14-3-3 protein sigma; stratifin; | 2.9 | 0.9 |
| Intracellular Kinase Network Members | calcium/calmodulin-dependen | 1.8 | 0.9 |
| Nucleotide Metabolism | type II cytoskeletal 11 keratin | 5.2 | 0.8 |
| Other Apoptosis-Associated Proteins | IEX-1L anti-death protein; PR [symbol] | −1.8 | 0.8 |
| Transcription Activators & Repressors | glucocorticoid receptor repres [symbol] | 1.9 | 0.8 |
| Intracellular Kinase Network Members | C-jun N-terminal kinase 3 alp [symbol] | −2.3 | 0.8 |
| Basic Transcription Factors | CACCC-box DNA-binding pro [symbol] | 1.9 | 0.8 |
| Bcl Family Proteins | induced myeloid leukemia cell | −4.8 | 0.8 |
| Calpains | calpain 2 large (catalytic) sub [symbol] | −2.2 | 0.7 |
| Nucleotide Metabolism | multifunctional protein ADE2 | 6.2 | 0.7 |
| Intermediate Filament Proteins | coronin-like protein P57 | 1.8 | 0.7 |
| Growth Factors, Cytokines & Chemokines | platelet-derived growth factor | −1.8 | 0.7 |
| Nucleotide Metabolism | uridine diphosphoglucose pyr [symbol] | 5.9 | 0.7 |
| Caspases | caspase-3 (CASP3); apopain [symbol] | −1.8 | 0.7 |
| Growth Factors, Cytokines & Chemokines | vascular endothelial growth fa | −1.8 | 0.6 |
| Nucleotide Metabolism | adenosine deaminase (ADA); | 7.3 | 0.6 |
| Transcription Activators & Repressors | cAMP-responsive element-bin | 1.5 | 0.6 |
| Growth Factors, Cytokines & Chemokines | interleukin-1 beta precursor (II | −1.5 | 0.6 |
| Functionally Unclassified Proteins | tenascin precursor (TN); hexa | 2.2 | 0.6 |
| Basic Transcription Factors | basic transcription factor 2 44 [symbol] | 1.7 | 0.6 |
| Growth Factors, Cytokines & Chemokines | eosinophil granule major bask | −1.9 | 0.5 |
| Other Enzymeslinvolved in Protein | membrane-bound & secreted | 1.8 | 0.5 |
| Turnover | | | |
| Matrix Adhesion Receptors | intercellular adhesion molecul | −2.4 | 0.5 |
| Calpains | calpain p94 large (catalytic) s [symbol] | −2.0 | 0.4 |
| Nucleotide Metabolism | thymidylate kinase | 4.1 | 0.4 |
| Intracellular Kinase Network Members | janus kinase 3 (JAK3); leukoc [symbol] | 2.9 | 0.4 |
| Other Enzymeslinvolved in Protein | insulin-degrading enzyme; ins | −1.6 | 0.4 |
| Turnover | | | |
| Extracellular Matrix Proteins | dopachrome tautomerase; do [symbol] | 2.9 | 0.4 |
| Growth Factors, Cytokines & Chemokines | hepatocyte growth factor (HG [symbol] | −1.7 | 0.3 |
| Histone Acetyltransferases & | RPD3 protein: histone deace [symbol] | −1.5 | 0.3 |
| Deacetylases | | | |
| Other Stress Response Proteins | 25-hydroxy vitamin D3 1-alph; | −2.0 | 0.3 |
| Other Intracellular Transducers, Effectors | connector enhancer of KSR- [symbol] | −2.3 | 0.3 |
| & Modulators | | | |
| DNA Damage Repair Proteins & Ligases | DNA ligase III (LIG3); polydeo | −2.3 | 0.2 |
| Functionally Unclassified Proteins | laminin alpha 4 subunit precu [symbol] | 1.6 | 0.2 |
| Functionally Unclassified Proteins | mitogen-responsive phosphop | 1.8 | 0.2 |
| Intracellular Adaptors & Receptor- | proto-oncogene tyrosine-prote | 1.7 | 0.2 |
| Associated Proteins | | | |
| Extracellular Matrix Proteins | cytosolic serine hydroxylmeth [symbol] | 2.7 | 0.2 |
| Intracellular Adaptors & Receptor- | epidermal growth factor recep | −1.8 | 0.2 |
| Associated Proteins | | | |
| Extracellular Matrix Proteins | phosphoribosyl pyrophosphate | 3.8 | 0.2 |
| Other Cell Cycle Proteins | DNA-binding protein inhibitor [symbol] | 1.8 | 0.2 |
| Bcl Family Proteins | NIP1 (NIP1) | −3.2 | 0.1 |
| DNA Fragmentation Proteins | CAD; DNA fragmentation fact [symbol] | −1.5 | 0.1 |
| Cell Signaling & Extracellular | histidine decarboxylase (HDC [symbol] | 1.8 | 0.1 |
| Communication Proteins | | | |
| Cell Signaling & Extracellular | leptin receptor precursor; obe [symbol] | −2.5 | 0.1 |
| Communication Proteins | | | |
| Growth Factors, Cytokines & Chemokines | hepatoma-derived growth fact | −1.8 | 0.1 |
| Growth Factors, Cytokines & Chemokines | heparin-binding EGF-like gro [symbol] | −1.6 | 0.1 |
| Growth Factors, Cytokines & Chemokines | transforming growth factor-alp | −1.7 | 0.1 |
| Nucleotide Metabolism | adenylosuccinate synthetase [symbol] | 6.7 | 0.1 |
| Facilitated Diffusion Proteins | brain glucose transporter 3 (G | 2.1 | 0.1 |
| Intracellular Kinase Network Members | dual-specificity mitogen-activ [symbol] | 3.1 | 0.0 |
| Intermediate Filament Proteins | microtubule-associated protei [symbol] | 2.2 | 0.0 |
| Oncogenes & Tumor Suppressors | N-ras; transforming p21 protei [symbol] | 1.8 | 0.0 |
| Oncogenes & Tumor Suppressors | jun-D | 2.9 | 0.0 |
| Oncogenes & Tumor Suppressors | C-cbl proto-oncogene | 1.7 | 0.0 |
| Oncogenes & Tumor Suppressors | insulin-like growth factor bind [symbol] | 5.0 | 0.0 |
| Oncogenes & Tumor Suppressors | myb proto-oncogene; c-myb | 2.2 | 0.0 |
| Oncogenes & Tumor Suppressors | ski oncogene | 1.5 | 0.0 |
| Oncogenes & Tumor Suppressors | snoN oncogene | −2.3 | 0.0 |
| Oncogenes & Tumor Suppressors | ERBB4 receptor protein-tyros [symbol] | 1.5 | 0.0 |
| Cyclins | cyclin K | 2.3 | 0.0 |
| Cyclins | cyclin E2 | 2.2 | 0.0 |
| Intracellular Adaptors & Receptor- | epidermal growth factor recep | 1.9 | 0.0 |
| Associated Proteins | | | |
| Intracellular Kinase Network Members | tyrosine-protein kinase ack | 1.9 | 0.0 |
| Intracellular Adaptors & Receptor- | c-src kinase (CSK); protein-ty [symbol] | 2.0 | 0.0 |
| Associated Proteins | | | |
| Death Receptors | lymphocyte activation CD30 a | −2.8 | 0.0 |
| DNA Damage Repair Proteins & Ligases | excision repair protein ERCC [symbol] | 1.6 | 0.0 |
| Death Receptors | adenosine A1 receptor (ADO [symbol] | 2.9 | 0.0 |
| DNA Damage Repair Proteins & Ligases | DNA mismatch repair protein | −1.6 | 0.0 |
| Caspases | caspase-10 precursor (CASP [symbol] | 2.9 | 0.0 |
| Bcl Family Proteins | BCL-2-related protein A1 (BC [symbol] | −2.6 | 0.0 |
| DNA Damage Repair Proteins & Ligases | Rad50 | −1.5 | 0.0 |
| Other Apoptosis-Associated Proteins | growth arrest & DNA-damage- | −3.5 | 0.0 |
| DNA Polymerases, Replication Factors & | replication factor C 36-kDa su | −1.7 | 0.0 |
| Topoisomerases | | | |
| Other Intracellular Transducers, Effectors | leukemia inhibitory factor rec [symbol] | −2.0 | 0.0 |
| & Modulators | | | |
| DNA Polymerases, Replication Factors & | MCM3 DNA replication licens [symbol] | −2.5 | 0.0 |
| Topoisomerases | | | |
| Other Apoptosis-Associated Proteins | early response protein NAK1 [symbol] | −2.4 | 0.0 |
| DNA Damage Repair Proteins & Ligases | DNA ligase IV (LIG4); polyde [symbol] | −1.5 | 0.0 |
| Cell Signaling & Extracellular | | | |
| Communication Proteins | myelin-associated glycoprotei [symbol] | −2.1 | 0.0 |
| Transcription Activators & Repressors | brain-specific homeobox/POU | −1.5 | 0.0 |
| Cell-Cell Adhesion Receptors | cadherin 11 precursor (CDH1 [symbol] | −2.3 | 0.0 |
| Growth Factors, Cytokines & Chemokines | thrombomodulin precursor(T [symbol] | −1.5 | 0.0 |
| Other Extracellular Communication | B94 protein | −1.7 | 0.0 |
| Proteins | | | |
| Growth Factors, Cytokines & Chemokines | migration inhibitory factor-rela | −2.0 | 0.0 |
| Growth Factors, Cytokines & Chemokines | migration inhibitory factor-rela | −2.1 | 0.0 |
| Xenobiotic Metabolism | cytochrome P450 IIF1 (CYP2 [symbol] | −2.9 | 0.0 |
| Growth Factors, Cytokines & Chemokines | T-cell-specific rantes protein [symbol] | 2.0 | 0.0 |
| Growth Factors, Cytokines & Chemokines | kidney epidermal growth facto | −1.7 | 0.0 |
| Housekeeping Genes | brain-specific tubulin alpha 1 [symbol] | 1.8 | 0.0 |
| Nucleotide Metabolism | thioredoxin reductase | 7.0 | 0.0 |
| Nucleotide Metabolism | DR-nm23 | 6.4 | 0.0 |
| Nucleotide Metabolism | ribonucleoside-diphosphate re | 6.1 | 0.0 |
| Nucleotide Metabolism | uridine 5′-monophosphate syn | 5.8 | 0.0 |
| Nucleotide Metabolism | type I cytoskeletal 19 keratin [symbol] | 5.3 | 0.0 |
| Nucleotide Metabolism | GMP synthase; glutamine am [symbol] | 3.9 | 0.0 |
| Transcription Activators & Repressors | transcription factor HGATA-6 | 3.1 | 0.0 |
| Cytoskeleton & Motility Proteins | hemoglobin alpha subunit | 2.9 | 0.0 |
| Transcription Activators & Repressors | myeloid ELF1-like factor | 2.8 | 0.0 |
| Other Metabolism Enzymes | type II cytoskeletal 4 keratin ( [symbol] | 2.3 | 0.0 |
| Cell Surface Antigens | T-cell surface glycoprotein C [symbol] | 2.3 | 0.0 |
| Transcription Activators & Repressors | putative regulatory protein TG | 2.2 | 0.0 |
| Oncogenes & Tumor Suppressors | c-jun proto-oncogene; transcri | 1.9 | 0.0 |
| Immunoglobulins | IgC mu heavy chain constant | 1.8 | 0.0 |
| Extracellular Matrix Proteins | IMP dehydrogenase 1 | 1.7 | 0.0 |
| Other Cell Cycle Proteins | btg protein precursor; NGF-in [symbol] | 1.6 | 0.0 |
| Protease Inhibitors | immunoglobulin rearranged g [symbol] | 1.6 | 0.0 |
| Transcription Activators & Repressors | checkpoint suppressor 1 | 1.5 | 0.0 |
| Intracellular Transducers, Effectors & | urokinase-type plasminogen a | −1.5 | 0.0 |
| Modulators | | | |
| Xenobiotic Metabolism | cytochrome B-245 heavy chai | −2.0 | 0.0 |
| Growth Factor & Chemokine Receptors | activin type I receptor; serine/ [symbol] | −1.6 | 0.0 |
| Other Intracellular Transducers, Effectors | leukemia Inhibitory factor pre [symbol] | −2.6 | 0.0 |
| & Modulators | | | |
| Oncogenes & Tumor Suppressors | erythroblastosis virus oncoger | 1.5 | 0.0 |
| GTP/GDP Exchangers & GTPase Activity | GTPase-activating protein (G [symbol] | 1.8 | 0.0 |
| Modulators | | | |
| Death Receptor-Associated Proteins & | DAXX | −1.8 | 0.0 |
| Adaptors | | | |
| Amino Acid Metabolism | adenylosuccinate lyase; aden [symbol] | 3.0 | 0.0 |
| Microfilament Proteins | type II cytoskeletal 5 keratin ( [symbol] | 2.4 | 0.0 |
| Cyclins | cyclin H (CCNH); MO15-asso [symbol] | 2.0 | −0.2 |
| Death Receptor Ligands | CD27 ligand (CD27LG): CD7 [symbol] | −1.6 | −1.0 |
| Hormones | cellular retinoic acid-binding p | 3.9 | −1.0 |
| Bcl Family Proteins | bcl-2 interacting killer (BIK); N | −1.8 | −1.0 |
| Xenobiotic Metabolism | S-mephenytoin 4 hydroxylase | −2.4 | −1.0 |
| Kinase Activators & Inhibitors | STAT-induced STAT inhibitor | 2.3 | −1.0 |
| Nucleotide Metabolism | inosine-5′-monophosphate del | 3.9 | −1.1 |
| Growth Factors, Cytokines & Chemokines | uromodulin; Tamm-Horsfall ur | −1.8 | −1.1 |
| Death Receptors | WSL protein + TRAMP + Apo | 1.7 | −1.1 |
| Protease Inhibitors | tissue inhibtor of mettaloprote | −1.6 | −1.1 |
| Amino Acid Metabolism | bifunctional purine biosynthes | 5.0 | −1.2 |
| Death Receptor-Associated Proteins & | caspase & rip adaptator with [symbol] | −1.6 | −1.2 |
| Adaptors | | | |
| Growth Factors, Cytokines & Chemokines | amphiregulin (AR); colorectu [symbol] | 3.7 | −1.2 |
| Death Receptors | insulin-like growth factor I rec [symbol] | −1.6 | −1.2 |
| Xenobiotic Metabolism | dioxin-inducible cytochrome P | −2.7 | −1.2 |
| Oncogenes & Tumor Suppressors | A-raf proto-oncogene serine/t [symbol] | 1.7 | −1.3 |
| DNA Polymerases, Replication Factors & | activator 1 37-kDa subunit; re [symbol] | −2.2 | −1.3 |
| Topoisomerases | | | |
| Oncogenes & Tumor Suppressors | interferon-inducible protein 9- [symbol] | 2.1 | −1.3 |
| Growth Factors, Cytokines & Chemokines | interleukin-8 precursor (IL-8); | −3.2 | −1.3 |
| Apoptosis-Associated Proteins | growth arrest & DNA-damage- | 2.0 | −1.3 |
| Death Receptors | retinoic acid receptor epsilon ( | −2.4 | −1.4 |
| Cysteine Proteases | cathepsin H precursor | −1.6 | −1.4 |
| Death Receptor Ligands | CD40 ligand (CD40-L); tumor | −2.4 | −1.4 |
| Other Immune System Proteins | grancalcin | 0.0 | −1.5 |
| Cell Cycle-Regulating Kinases | cdc2-related protein kinase P [symbol] | 0.2 | −1.5 |
| Other Trafficking & Targeting Proteins | ER-Golgi intermediate compa | 0.8 | −1.5 |
| Growth Factors, Cytokines & Chemokines | bone morphogenetic protein 1 | −1.4 | −1.5 |
| Basic Transcription Factors | MYELIN TRANSCRIPTION F [symbol] | 0.0 | −1.5 |
| Other Cell Cycle Proteins | RCL growth-related c-myc-res | 1.3 | −1.5 |
| Adenylate/Guanylate Cyclases & | adenylate cyclase VII; ATP py | −1.0 | −1.5 |
| Diesterases | | | |
| Oncogenes & Tumor Suppressors | papillary thyroid carcinoma-en | 0.0 | −1.5 |
| Basic Transcription Factors | POD1 - MESODERM-SPEC | −1.1 | −1.5 |
| Oncogenes & Tumor Suppressors | active breakpoint cluster regio | 0.0 | −1.5 |
| Intracellular Adaptors & Receptor- | c-src kinase (CSK); protein-ty [symbol] | 0.1 | −1.5 |
| Associated Proteins | | | |
| Intracellular Protein Phosphatases | protein phosphatase 2B regul [symbol] | 0.0 | −1.5 |
| Oncogenes & Tumor Suppressors | L-myc proto-oncogene (MYCL | 0.0 | −1.5 |
| Intracellular Transducers, Effectors & | autocrine motility factor recep | 0.0 | −1.5 |
| Modulators | | | |
| Metalloproteinases | matrix metalloproteinase 12 ( [symbol] | 0.5 | −1.5 |
| Intracellular Kinase Network Members | KIAA0096 | 0.1 | −1.5 |
| Metabolism of Cofactors. Vitamins & | peroxisomal acyl-coenzyme A | −1.3 | −1.5 |
| Related Substances | | | |
| Other Immune System Proteins | myeloperoxidase precursor [symbol] | −1.1 | −1.5 |
| Cell Signaling & Extracellular | | | |
| Communication Proteins | synaptosomal-associated prot | 0.0 | −1.5 |
| Basic Transcription Factors | SREBP-1 - BASIC-HELIX-LO [symbol] | 0.0 | −1.5 |
| Ligand-Gated Ion Channels | ASIC3 proton gated cation ch [symbol] | 0.6 | −1.6 |
| Other Cell Cycle Proteins | geminin | 0.0 | −1.6 |
| Growth Factor & Chemokine Receptors | leukocyte platelet-activating [symbol] | 0.2 | −1.6 |
| Oncogenes & Tumor Suppressors | vascular endothelial growth fa | 0.0 | −1.6 |
| Oncogenes & Tumor Suppressors | moesin-ezrin-radixin-like prot [symbol] | −1.0 | −1.6 |
| Hormones | glucagon precursor (GCG) | 0.0 | −1.6 |
| Basic Transcription Factors | Interferon regulatory factor 4 [symbol] | 0.4 | −1.6 |
| DNA Damage Repair Proteins & Ligases | Ku 70-kDa subunit; ATP-depe | 0.0 | −1.6 |
| Cell Signaling & Extracellular | | | |
| Communication Proteins | prostaglandin E2 (PGE) recep | 0.0 | −1.6 |
| Basic Transcription Factors | HOMEOBOX PROTEIN PKN [symbol] | 0.0 | −1.6 |
| Oncogenes & Tumor Suppressors | retinoblastoma-associated pr [symbol] | 0.0 | −1.6 |
| Oncogenes & Tumor Suppressors | AF-6 protein | 0.0 | −1.6 |
| Other Metabolism Enzymes | corticosteroid 11-beta-dehyd [symbol] | 0.9 | −1.6 |
| Transcription Activators & Repressors | host cell factor C1 (HCF); VP1 | 0.5 | −1.6 |
| Oncogenes & Tumor Suppressors | v-erbA related protein (EAR3) | 0.2 | −1.6 |
| Oncogenes & Tumor Suppressors | synapse-associated protein 1 [symbol] | 0.0 | −1.6 |
| Intracellular Protein Phosphatases | protein phosphatase with EF- [symbol] | 0.1 | −1.6 |
| Oncogenes & Tumor Suppressors | c-myc oncogene | 3.1 | −1.6 |
| Intracellular Protein Phosphatases | protein phosphatase 2A B56- [symbol] | 0.0 | −1.6 |
| DNA Polymerases, Replication Factors & | DNA polymerase epsilon subu | 0.0 | −1.6 |
| Topoisomerases | | | |
| Other Cell Cycle Proteins | RBQ1 retinoplastoma binding | 0.4 | −1.6 |
| Cell Cycle-Regulating Kinases | serine/threonine-protein kinas [symbol] | 0.5 | −1.6 |
| Recombination Proteins | V(D)J recombination activatin [symbol] | 0.0 | −1.6 |
| Other Apoptosis-Associated Proteins | inhibitor of apoptosis protein 3 | 0.4 | −1.6 |
| Extracellular Transporters & Carrier | phospholipid transfer protein [symbol] | 0.2 | −1.6 |
| Proteins | | | |
| Ligand-Gated Ion Channels | ATP-sensitive inward rectifier | 0.0 | −1.6 |
| Death Receptors | Insulin-like growth factor I rec [symbol] | 0.0 | −1.6 |
| Adenylate/Guanylate Cyclases & | calcium/calmodulin-dependen | 0.9 | −1.7 |
| Diesterases | | | |
| DNA Damage Repair Proteins & Ligases | DNA mismatch repair protein | 0.0 | −1.7 |
| Cell-Cell Adhesion Receptors | cadherin 12 (CDH12); brain c [symbol] | 0.0 | −1.7 |
| Housekeeping Genes | brain-specific tubulin alpha 1 [symbol] | 0.2 | −1.7 |
| Oncogenes & Tumor Suppressors | TSG101 tumor susceptibility [symbol] | −1.0 | −1.7 |
| Intracellular Adaptors & Receptor- | APS | 3.8 | −1.7 |
| Associated Proteins | | | |
| Extracellular Matrix Proteins | vitronectin precursor (VTN); S [symbol] | 0.0 | −1.7 |
| Oncogenes & Tumor Suppressors | box-dependent myc-interactin [symbol] | 0.2 | −1.7 |
| Recombination Proteins | recA-like protein HsRad51; D [symbol] | 0.2 | −1.7 |
| Amino- & Carboxypeptidases | carboxypeptidase H precursor | 0.0 | −1.7 |
| Transcription Activators & Repressors | human immunodeficiency viru | 0.0 | −1.7 |
| Housekeeping Genes | 23-kDa highly basic protein; 6 [symbol] | −2.2 | −1.7 |
| G Proteins | ADP-ribosylation factor 1 | 0.5 | −1.7 |
| Basic Transcription Factors | ENX-1 PUTATIVE TRANSC [symbol] | 0.5 | −1.7 |
| Other Extracellular Communication | chondromodulin I precursor (C | 0.0 | −1.7 |
| Proteins | | | |
| Basic Transcription Factors | ZINC FINGER PROTEIN ZR [symbol] | 0.2 | −1.7 |
| Transcription Activators & Repressors | homeobox protein HOX-D3; H | 0.0 | −1.7 |
| Cell Surface Antigens | mesothelin precursor; CAK1 a | 0.0 | −1.7 |
| Drug-Resistance Proteins | serum paraoxonase/arylester [symbol] | −1.0 | −1.7 |
| Oncogenes & Tumor Suppressors | leukemia-associated gene 1 | 0.0 | −1.7 |
| Growth Factors, Cytokines & Chemokines | PS2 protein precursor; HP1-A | −1.2 | −1.7 |
| Transcription Activators & Repressors | NF-kappaB transcription facto | 0.0 | −1.7 |
| Transcription Activators & Repressors | gamma-interferon-inducible p [symbol] | 1.2 | −1.7 |
| Transcription Activators & Repressors | interleukin enhancer-binding f [symbol] | 0.4 | −1.7 |
| Oncogenes & Tumor Suppressors | tumor suppressor maspin; pro | 0.7 | −1.8 |
| DNA Damage Repair Proteins & Ligases | HHR23A; UV excision repair [symbol] | 0.9 | −1.8 |
| Symporters & Antiporters | high-affinity glutamate transp [symbol] | 0.0 | −1.8 |
| Interleukins & Interferons | interleukin-9 precursor (IL-9); | 0.3 | −1.8 |
| Oncogenes & Tumor Suppressors | tyrosine-protein kinase recept [symbol] | 0.0 | −1.8 |
| Other Apoptosis-Associated Proteins | clusterin precursor (CLU); con | −2.8 | −1.8 |
| Transcription Activators & Repressors | interferon regulatory factor 1 ( | 0.6 | −1.8 |
| Oncogenes & Tumor Suppressors | cullin homolog 2 (CUL2) | −1.2 | −1.8 |
| Hormone Receptors | thyrotropin-releasing hormone | 0.0 | −1.8 |
| Intermediate Filament Proteins | myosin-IXB | 1.1 | −1.8 |
| Growth Factors, Cytokines & Chemokines | CC chemokine eotaxin precur | −1.1 | −1.8 |
| Basic Transcription Factors | EARLY GROWTH RESPONS | 0.0 | −1.8 |
| Cell-Cell Adhesion Receptors | integrin alpha 1 (ITGA1); lami | 0.0 | −1.8 |
| Xenobiotic Metabolism | D-amino acid oxidase (DAMO | 0.0 | −1.8 |
| Intracellular Protein Phosphatases | tyrosine phosphatase | 0.0 | −1.8 |
| Housekeeping Genes | 40S ribosomal protein S9 | 0.3 | −1.8 |
| Other Intracellular Transducers, Effectors | IkappaB kinase complex-asso | 0.0 | −1.8 |
| & Modulators | | | |
| Death Receptor-Associated Proteins & | MAP kinase-activating death [symbol] | 0.0 | −1.8 |
| Adaptors | | | |
| Hormone Receptors | muscarlnic acetylcholine rece [symbol] | 0.0 | −1.8 |
| Basic Transcription Factors | HATH-1 - ATONAL HOMOLO | 0.0 | −1.8 |
| Intracellular Kinase Network Members | dual specificity mitogen-activ [symbol] | 0.4 | −1.8 |
| Basic Transcription Factors | ESE1, ERT, JEN, ELF3 (Epit [symbol] | 0.0 | −1.8 |
| Intracellular Kinase Network Members | DCHT | 2.1 | −1.8 |
| DNA Polymerases, Replication Factors & | proliferating cyclic nuclear ant | 0.0 | −1.8 |
| Topoisomerases | | | |
| Transcription Activators & Repressors | nuclear factor NF-kappa-B p1 [symbol] | −1.0 | −1.8 |
| Intermediate Filament Proteins | hyaluronan receptor (RHAMM | 0.0 | −1.9 |
| Intracellular Kinase Network Members | STE20-like Kinase; MST2 | 0.0 | −1.9 |
| Symporters & Antiporters | sodium-dependent glutamate/ | 0.0 | −1.9 |
| Intracellular Transducers, Effectors & | ephrin type-B receptor 2 prec [symbol] | 0.0 | −1.9 |
| Modulators | | | |
| Oncogenes & Tumor Suppressors | p53 cellular tumor antigen | 0.0 | −1.9 |
| Intracellular Kinase Network Members | casein kinase I gamma 2 (CK | 0.0 | −1.9 |
| Oncogenes & Tumor Suppressors | C-mos proto-oncogene serine [symbol] | 0.5 | −1.9 |
| Intracellular Kinase Network Members | protein kinase DYRK2 | 0.0 | −1.9 |
| Intracellular Adaptors & Receptor- | c-fer proto-oncogene | 0.0 | −1.9 |
| Associated Proteins | | | |
| G Proteins | ras-related protein RAP-1A; C | 3.5 | −1.9 |
| Intracellular Protein Phosphatases | protein phosphatase PP2A 61 | 0.0 | −1.9 |
| Oncogenes & Tumor Suppressors | nuclear pore complex protein | 0.4 | −1.9 |
| Growth Factors, Cytokines & Chemokines | dishevelled (DVL) + dishevel [symbol] | 0.0 | −1.9 |
| Intracellular Protein Phosphatases | myotubularin | 2.5 | −1.9 |
| Kinase Activators & Inhibitors | muscle/brain cAMP-dependen | 0.0 | −1.9 |
| Intracellular Kinase Network Members | ribosomal protein S6 kinase II | 0.5 | −2.0 |
| Other Trafficking & Targeting Proteins | cleavage stimulation factor 77 | 0.0 | −2.0 |
| Death Kinases | Fas-activated serine/threonin [symbol] | 0.4 | −2.0 |
| Intracellular Kinase Network Members | serine/threonin-protein kinase | 1.3 | −2.0 |
| Complex Lipid Metabolism | 3-hydroxy-3-methylglutaryl-co | 0.0 | −2.0 |
| Other Transcription Proteins | ELL protein | 0.0 | −2.0 |
| Housekeeping Genes | ubiquitin | −1.6 | −2.0 |
| Basic Transcription Factors | NF-ATc | 0.5 | −2.0 |
| Intracellular Transducers, Effectors & | ephrin A4 precursor (EFNA4); | 1.4 | −2.0 |
| Modulators | | | |
| Cell Signaling & Extracellular | sodium-dependent dopamine | 0.2 | −2.0 |
| Communication Proteins | | | |
| Oncogenes & Tumor Suppressors | neogenin | 1.1 | −2.0 |
| Intracellular Adaptors & Receptor- | 70-kDa zeta-associated protel | 0.0 | −2.0 |
| Associated Proteins | | | |
| Basic Transcription Factors | Runt domain-containing protel | −1.9 | −2.0 |
| Complex Lipid Metabolism | membrane-associated phosph | 0.0 | −2.0 |
| Extracellular Transporters & Carrier | apolipoprotein E precursor (A [symbol] | 1.7 | −2.0 |
| Proteins | | | |
| DNA Damage Repair Proteins & Ligases | DNA damage repair & recomt | 0.0 | −2.0 |
| Voltage-Gated Ion Channels | KCNQ3 potassium channel | 0.4 | −2.1 |
| Housekeeping Genes | cytoplasmic beta-actin (ACTB | 0.0 | −2.1 |
| Growth Factors, Cytokines & Chemokines | thymosin beta-10 (TMSB10; T | 0.0 | −2.1 |
| Oncogenes & Tumor Suppressors | c-jun proto-oncogene; transcri | 2.2 | −2.1 |
| Intracellular Kinase Network Members | c-jun N-terminal kinase 1 (JNI | 0.1 | −2.1 |
| GTP/GDP Exchangers & GTPase Activity | rho GDP dissociation inihibitor | 0.1 | −2.1 |
| Modulators | | | |
| Oncogenes & Tumor Suppressors | C-maf transcription factor | 1.4 | −2.1 |
| Intracellular Kinase Network Members | cAMP-dependent protein kina [symbol] | 0.1 | −2.1 |
| DNA Damage Repair Proteins & Ligases | uracil-DNA glycosylase precur | −1.1 | −2.1 |
| Oncogenes & Tumor Suppressors | maguk p55 subfamily member | 0.0 | −2.1 |
| Transcription Activators & Repressors | interferon regulatory factor 5 ( | 0.1 | −2.1 |
| Interleukins & Interferons | interleukin-14 precursor (IL-14 | 0.0 | −2.1 |
| Oncogenes & Tumor Suppressors | Von Hippel-Lindau tumor supp | 0.4 | −2.2 |
| Bcl Family Proteins | NIP3 (NIP3) | −1.7 | −2.2 |
| Oncogenes & Tumor Suppressors | ezrin; cytovillin 2; villin 2 (VIL: | 1.7 | −2.2 |
| DNA Polymerases, Replication Factors & | DNA topoisomerase III (TOP3 | 0.0 | −2.2 |
| Topoisomerases | | | |
| Oncogenes & Tumor Suppressors | RNA-binding protein fus/tls | 1.0 | −2.2 |
| Growth Factors, Cytokines & Chemokines | bone morphogenetic protein 3 | 2.8 | −2.2 |
| Oncogenes & Tumor Suppressors | GRB-IR/GRB10 | 0.0 | −2.2 |
| Intracellular Kinase Network Members | B-lymphocyte germinal center | 0.0 | −2.2 |
| Other Intracellular Transducers, Effectors | junction plakoglobin (JUP); de | 3.4 | −2.2 |
| & Modulators | | | |
| Oncogenes & Tumor Suppressors | platelet-derived growth factor | 0.0 | −2.2 |
| Transcription Activators & Repressors | E4BP4 | 0.2 | −2.2 |
| Facilitated Diffusion Proteins | putative renal organic anion tr | 0.0 | −2.2 |
| Intracellular Protein Phosphatases | leukocyte antigen-related prot [symbol] | 0.0 | −2.3 |
| Oncogenes & Tumor Suppressors | elk-1; ets-related proto-oncog [symbol] | 1.0 | −2.3 |
| Death Kinases | rac-alpha serine/threonine kin [symbol] | 0.1 | −2.3 |
| Transcription Activators & Repressors | B4-2 protein | 0.6 | −2.3 |
| Housekeeping Genes | cytoplasmic beta-actin (ACTB | −2.2 | −2.3 |
| General Trafficking Proteins | ribonuclease 6 precursor | 0.2 | −2.3 |
| Growth Factors, Cytokines & Chemokines | OX40 ligand (OX40L); GP34; | 0.0 | −2.3 |
| Other DNA-Binding & Chromatin Proteins | nuclear domain 10 protein 52 | −1.9 | −2.3 |
| Other Cell Cycle Proteins | prothymosin alpha (ProT-alp [symbol] | 0.0 | −2.4 |
| Oncogenes & Tumor Suppressors | N-myc proto-oncogene | 0.0 | −2.4 |
| Oncogenes & Tumor Suppressors | ERBB2 receptor protein-tyrosi | 0.5 | −2.4 |
| Transcription Activators & Repressors | nuclear factor NF-kappa-B p1 [symbol] | 0.2 | −2.4 |
| Amino Acid Metabolism | adenine phosphoribosyltransf [symbol] | 3.9 | −2.4 |
| GTP/GDP Exchangers & GTPase Activity | REGULATOR OF G-PROTEII | 0.2 | −2.4 |
| Modulators | | | |
| Other Trafficking & Targeting Proteins | RNA helicase | 0.0 | −2.4 |
| Basic Transcription Factors | MYOCYTE-SPECIFIC ENHA [symbol] | 0.6 | −2.5 |
| Oncogenes & Tumor Suppressors | v-erbA related protein (EAR2) | 0.0 | −2.5 |
| Interleukins & Interferons | interleukin-6 precursor (IL-6); | 2.0 | −2.5 |
| Intracellular Kinase Network Members | kinase suppressor of ras-1 (KS | 0.0 | −2.5 |
| Housekeeping Genes | 23-kDa highly basic protein; 6 [symbol] | 0.0 | −2.5 |
| Nucleotide Metabolism | uridine phosphorylase (UDRP [symbol] | 4.8 | −2.6 |
| Housekeeping Genes | ubiquitin | 0.8 | −2.6 |
| Oncogenes & Tumor Suppressors | colorectal mutant cancer prot [symbol] | 0.0 | −2.6 |
| Intracellular Kinase Network Members | cAMP-dependent protein kina [symbol] | 0.0 | −2.6 |
| GTP/GDP Exchangers & GTPase Activity | regulator of G protein signalin [symbol] | 0.5 | −2.8 |
| Modulators | | | |
| GTP/GDP Exchangers & GTPase Activity | interferon-Induced guanylate- [symbol] | 0.7 | −2.9 |
| Modulators | | | |
| Oncogenes & Tumor Suppressors | c-raf proto-oncogene | 1.5 | −2.9 |
| Complex Lipid Metabolism | phosphatidylethanolamine-bin | 1.9 | −2.9 |
| Growth Factors, Cytokines & Chemokines | monocyte chemotactic protein | −2.9 | −3.0 |
| DNA Damage Repair Proteins & Ligases | ALKB homolog protein | 0.0 | −3.0 |
| Amino- & Carboxypeptidases | dipeptidyl peptidase IV (DPP I | −1.1 | −3.0 |
| Other Extracellular Communication | thymosin beta 4; FX | −1.2 | −3.3 |
| Proteins | | | |
| Proteosomal Proteins | proteasome inhibitor HPI31 s [symbol] | −1.1 | −3.4 |
| Cytoskeleton & Motility Proteins | TRAM protein | 0.2 | −3.7 |
| Transcription Activators & Repressors | interleukin enhancer binding f [symbol] | −1.0 | −3.8 |
| Nucleotide Metabolism | 5′-nucleotidase precurso (5′N | 6.8 | −4.2 |
| ATPase Transporters | copper-transporting ATPase 2 | 0.0 | −4.5 |
| Basic Transcription Factors | ZINC FINGER PROTEIN UBl | −1.2 | −8.9 |
| Complex Lipid Metabolism | mevalonate kinase | 2.9 | −9.8 |

TABLE 8a
GENE CHANGES INDUCED BY DENGUE VIRUS IN VITRO IN HUMAN LYMPHOID CELLS-
ARRAY I

| Gene code | Gene Function | Protein/gene | DEN-4h | DEN-8h |
|---|---|---|---|---|
| A01g | Oncogenes & Tumor Suppressors | transforming protein rhoA H12 (RHO1; | 0.0 | 1.7 |
| A01i | Cell Cycle-Regulating Kinases | BUBR1 protein kinase | −2.2 | 0.0 |
| A02d | Oncogenes & Tumor Suppressors | erythroblastosis virus oncogene homol | 0.0 | 1.6 |
| A03b | Oncogenes & Tumor Suppressors | EB1 protein | 0.0 | 2.2 |
| A03g | Oncogenes & Tumor Suppressors | N-ras; transforming p21 protein | 0.5 | 1.7 |
| A03k | CDK Inhibitors | cyclin-dependent kinase 4 inhibitor B ( [symbol] | 0.0 | −1.8 |
| A04b | Oncogenes & Tumor Suppressors | ezrin; cytovillin 2; villin 2 (VIL2) | −1.8 | 1.3 |
| A04e | Oncogenes & Tumor Suppressors | A-raf proto-oncogene serine/threonine | −1.5 | −1.0 |
| A04f | Oncogenes & Tumor Suppressors | proto-oncogene tyrosine-protein kinase | 0.0 | −1.7 |
| A04l | Other Cell Cycle Proteins | DNA-binding protein inhibitor ID-1; Id-1 | −3.8 | −1.3 |
| A05b | Growth Factors, Cytokines & | transforming growth factor-beta 3 (TG [symbol] | 0.0 | 2.7 |
| A05c | Oncogenes & Tumor Suppressors | p78 putative serine/threonine-protein k | 0.0 | 4.8 |
| A05d | Oncogenes & Tumor Suppressors | B-myb | 0.6 | 5.3 |
| A05e | Oncogenes & Tumor Suppressors | tyrosine-protein kinase receptor UFO [symbol] | 0.0 | 5.0 |
| A05f | Oncogenes & Tumor Suppressors | tyrosine-protein kinase ABL2; tyrosine | 0.3 | 4.0 |
| A05g | Oncogenes & Tumor Suppressors | INT-2 proto-oncogene protein precursc | 0.0 | 5.8 |
| A05h | Cyclins | G1/S-specific cyclin D3 (CCND3) | −1.6 | 0.5 |
| A05i | Cell Cycle-Regulating Kinases | cell division protein kinase 6 (CDK6); [symbol] | 0.0 | 7.0 |
| A05j | Cell Cycle-Regulating Kinases | serine/threonine-protein kinase KKIAL [symbol] | 0.0 | 5.9 |
| A05k | CDK Inhibitors | cyclin-dependent kinase 4 inhibitor D ( | 0.7 | 5.8 |
| A06b | Growth Factor & Chemokine | transforming growth factor beta recept [symbol] | 0.2 | −2.0 |
| A06c | Oncogenes & Tumor Suppressors | C-maf transcription factor | −2.4 | −1.4 |
| A06g | Oncogenes & Tumor Suppressors | mas proto-oncogene | −1.8 | −1.2 |
| A06k | CDK Inhibitors | cyclin-dependent kinase inhibitor 1C ( [symbol] | −1.5 | 1.3 |
| A07a | Oncogenes & Tumor Suppressors | neurofibromatosis protein type I (NF1); | 0.0 | −4.0 |
| A07b | Oncogenes & Tumor Suppressors | prohibitin (PHB) | 0.0 | −2.5 |
| A07c | Oncogenes & Tumor Suppressors | elk-1; ets-related proto-oncogene | 0.0 | −2.0 |
| A07e | Oncogenes & Tumor Suppressors | c-kit proto-oncogene; mast/stem cell g | 0.0 | −1.8 |
| A07g | Oncogenes & Tumor Suppressors | thrombopoietin receptor precursor (TP | −1.6 | 0.0 |
| A07l | Other Cell Cycle Proteins | 40S ribosomal protein S19 (RPS19) | −2.9 | −1.6 |
| A08a | Oncogenes & Tumor Suppressors | moesin-ezrin-radixin-like protein (MER | −1.3 | −2.2 |
| A08b | Oncogenes & Tumor Suppressors | tight junction protein zonula occludens | −1.1 | −1.8 |
| A08f | Oncogenes & Tumor Suppressors | C-fes proto-oncogene | 0.0 | −1.8 |
| A10k | Other Cell Cycle Proteins | geminin | −1.5 | 0.0 |
| A10m | Facilitated Diffusion Proteins | aquaporin 4; WCH4; mercurial-insensi | −2.3 | 0.5 |
| A12c | Oncogenes & Tumor Suppressors | c-myc oncogene | 1.7 | 1.6 |
| A12l | Other Cell Cycle Proteins | transducer of erbB2 (TOB) | −1.3 | −1.8 |
| A13c | Oncogenes & Tumor Suppressors | c-rel proto-oncogene protein | 2.1 | 0.0 |
| A13g | Cyclins | cyclin K | 0.0 | −1.6 |
| A13k | Other Cell Cycle Proteins | sprouty 2 (SPRY2) | −2.3 | 1.0 |
| A14a | Oncogenes & Tumor Suppressors | colorectal mutant cancer protein (MCC | 0.0 | −2.3 |
| A14d | Oncogenes & Tumor Suppressors | C-mos proto-oncogene serine/threonin | −7.9 | 0.3 |
| A14e | Oncogenes & Tumor Suppressors | platelet-derived growth factor receptor | −1.9 | 0.0 |
| A14g | Cyclins | cyclin E2 | −1.6 | 0.0 |
| B01j | Intracellular Kinase Network | cAMP-dependent protein kinase type I | −1.7 | 0.0 |
| B01k | Intracellular Kinase Network | lipid-activated protein kinase PRK1; PK | −2.2 | 0.0 |
| B01n | G Proteins | ADP-ribosylation factor 1 | 0.0 | −1.9 |
| B02g | Intracellular Adaptors & Receptor- | tyrosine-protein kinase lyn | 1.3 | 1.6 |
| B02n | G Proteins | ras-related protein RAP-1B; GTP-bindi | 2.3 | 2.4 |
| B03l | Intracellular Kinase Network | ribosomal protein S6 kinase II alpha 1 | 1.5 | 0.9 |
| B03n | G Proteins | ras-related protein RAB2 | 1.8 | 1.0 |
| B05g | Intracellular Adaptors & Receptor- | cell division cycle protein 25 nucleotid [symbol] | −2.5 | 0.0 |
| B05h | Intracellular Kinase Network | tyk2 non-receptor protein tyrosine kina | 0.0 | −1.5 |
| B05i | Intracellular Kinase Network | protein kinase C delta (NPKC-delta) | 2.0 | 0.8 |
| B05j | Intracellular Kinase Network | c-jun N-terminal kinase 2 (JNK2); JNK | 0.7 | −2.4 |
| B05l | Intracellular Kinase Network | ribosomal protein S6 kinase II alpha 3 | 1.7 | 1.2 |
| B06n | G Proteins | ras-related protein RAB5A | 2.0 | 0.0 |
| B07g | Intracellular Adaptors & Receptor- | Ink adaptor protein | 2.0 | 0.7 |
| B07h | Intracellular Kinase Network | mitogen-activated protein kinase p38 ( | 3.3 | 1.8 |
| B07m | G Proteins | Ral A; GTP-binding protein | 3.1 | 0.0 |
| B08a | Cell Signaling & Extracellular | sodium-dependent serotonin transport [symbol] | −3.1 | 0.2 |
| B08j | Phospholipases & Phosphoinositol | phosphatidylinositol 3-kinase regulator | 2.7 | 0.3 |
| B08k | Intracellular Kinase Network | phosphorylase B kinase gamma cataly | −1.7 | 0.0 |
| B09a | Cell Signaling & Extracellular | sodium-dependent noradrenaline trans | −5.4 | 0.0 |
| B09h | Intracellular Kinase Network | MAP kinase-activated protein kinase 2 | 4.4 | 1.4 |
| B10f | Intracellular Adaptors & Receptor- | c-src kinase (CSK); protein-tyrosine ki [symbol] | 1.8 | 0.1 |
| B10k | Intracellular Kinase Network | cAMP-dependent protein kinase beta- [symbol] | 1.6 | 0.4 |
| B10l | Phospholipases & Phosphoinositol | phosphatidylinositol 3-kinase catalytic | 1.5 | 0.5 |
| B10m | G Proteins | ras-related protein RAB-7 | 0.6 | −2.1 |
| B11g | Intracellular Kinase Network | mitogen-activated protein kinase kinas | 1.2 | 1.6 |
| B11j | Intracellular Kinase Network | 5′-AMP-activated protein kinase cataly | 1.7 | 1.0 |
| B11l | Phospholipases & Phosphoinositol | phosphatidylinositol 4-kinase alpha (P [symbol] | 2.0 | 0.4 |
| B12h | Intracellular Kinase Network | dual specificity mitogen-activated prot [symbol] | 1.5 | 1.0 |
| B12i | Intracellular Kinase Network | calcium/calmodulin-dependent protein | 3.2 | 1.2 |
| B12l | Phospholipases & Phosphoinositol | phospholipase C beta 2 (PLC-beta 2; [symbol] | 0.0 | 1.5 |
| B12m | G Proteins | guanine nucleotide-binding protein G( [symbol] | 3.0 | 3.5 |
| B13m | G Proteins | ras-related C3 botulinum toxin substra [symbol] | 2.2 | 0.9 |
| B14g | Intracellular Kinase Network | cAMP-dependent protein kinase I alph [symbol] | 1.9 | 2.1 |
| C01b | Intracellular Protein Phosphatases | PTPCAAX1 nuclear tyrosine phosphat [symbol] | 2.0 | 0.8 |
| C01d | Transcription Activators & Repressors | signal transducer and activator of tran [symbol] | −1.3 | −1.5 |
| C01k | Other Apoptosis-Associated Proteins | poly(ADP-ribose) polymerase (PARP [symbol] | −2.0 | 0.0 |
| C02i | Calpains | calcium-dependent protease small (re [symbol] | 0.2 | 2.0 |
| C02m | DNA Polymerases, Replication | MCM4 DNA replication licensing facto [symbol] | −1.6 | 0.0 |
| C02n | DNA Damage Repair Proteins & | xeroderma pigmentosum group D com | −2.1 | 0.0 |
| C04b | Adenylate/Guanylate Cyclases & | 3′5′-cAMP phosphodiesterase HPDE4 [symbol] | 0.0 | −2.3 |
| C04e | Kinase Activators & Inhibitors | hint protein; protein kinase C inhibitor [symbol] | 3.1 | 0.6 |
| C04n | DNA Damage Repair Proteins & | excision repair protein ERCC6; Cocka [symbol] | −1.6 | 0.4 |
| C05a | Intracellular Protein Phosphatases | leukocyte antigen-related protein prec [symbol] | 0.0 | −2.6 |
| C05b | Adenylate/Guanylate Cyclases & | adenylate cyclase type I; ATP pyropho [symbol] | 0.0 | −2.8 |
| C06e | Kinase Activators & Inhibitors | 14-3-3 protein sigma; stratifin; epitheli [symbol] | −1.6 | −2.3 |
| C06j | Death Kinases | interferon-inducible RNA-dependent p [symbol] | 1.8 | 1.6 |
| C08b | Adenylate/Guanylate Cyclases & | guanylate cyclase soluble beta-1 subu [symbol] | −2.4 | 0.0 |
| C09e | Other Intracellular Transducers. | TRRAP protein | 1.8 | 0.8 |
| C09g | Death Receptors | adenosine A1 receptor (ADORA1) | −3.3 | −1.9 |
| C09j | Other Apoptosis-Associated Proteins | IEX-1L anti-death protein; PRG-1, DIF | −1.6 | 0.0 |
| C09k | Other Apoptosis-Associated Proteins | inhibitor of apoptosis protein1 (HIAP1 [symbol] | 0.1 | −1.8 |
| C11h | Caspases | caspase-10 precursor (CASP10); ICE- [symbol] | −3.6 | −3.0 |
| C12e | Other Intracellular Transducers, | zyxin + zyxin-2 | −1.0 | −1.8 |
| C14b | Adenylate/Guanylate Cyclases & | adenylate cyclase VII; ATP pyrophosp [symbol] | 0.0 | −1.6 |
| C14h | Calpains | calpain 2 large (catalytic) subunit; M-ty [symbol] | 0.9 | 1.6 |
| D01f | Cell Signaling & Extracellular | dopamine beta-hydroxylase (DBH); do | −2.0 | 0.1 |
| D02i | Cell Signaling & Extracellular | peripheral myelin protein 22 (PMP22) [symbol] | 0.7 | 1.6 |
| D03e | Cell Signaling & Extracellular | acetylcholinesterase precursor (ACHE [symbol] | −1.2 | −1.6 |
| D03f | Cell Signaling & Extracellular | secretogranin II precursor (SGII); chro [symbol] | −1.6 | 0.0 |
| D03n | Basic Transcription Factors | cellular nucleic acid binding protein (C [symbol] | 1.7 | 1.1 |
| D05g | Cell Signaling & Extracellular | neuronal pentraxin II precursor (NP2) | 0.0 | 3.4 |
| D05m | Transcription Activators & Repressors | nuclear factor NF-kappa-B p100 subur | 0.9 | 1.6 |
| D05n | Transcription Activators & Repressors | estrogen receptor hSNF2b; global tran | 1.4 | 4.5 |
| D06d | Cell Signaling & Extracellular | GABA-B receptor 2 subunit (GABA-BR [symbol] | 0.2 | 3.1 |
| D06i | Cell Signaling & Extracellular | parkin | −1.5 | 0.0 |
| D06n | Basic Transcription Factors | transcriptional repressor NF-X1 | 1.0 | 3.9 |
| D07a | Recombination Proteins | recA-like protein HsRad51; DNA repai [symbol] | −1.7 | 0.0 |
| D07d | Cell Signaling & Extracellular | glutamate receptor 5 precursor (GLUR | −1.5 | 0.0 |
| D07e | Cell Signaling & Extracellular | neuroendocrine convertase 1 precurso | −2.6 | 0.5 |
| D07f | Cell Signaling & Extracellular | proenkephalin A precursor | −2.2 | 0.0 |
| D08d | Cell Signaling & Extracellular | neuronal acetylcholine receptor proteir | −2.2 | 0.0 |
| D08e | Cell Signaling & Extracellular | neuroendocrine convertase 2 precurso | −1.9 | 0.0 |
| D08j | Basic Transcription Factors | hypoxia-inducible factor 1 alpha (HIF1 | 1.9 | 0.6 |
| D09e | Cell Signaling & Extracellular | membrane-bound & soluble catechol- [symbol] | −1.7 | 0.0 |
| D09l | Transcription Activators & Repressors | interferon regulatory factor 7 (IRF-7) | −1.7 | 0.0 |
| D11e | Cell Signaling & Extracellular | flavin-containing amine oxidase A; mo | −2.0 | 0.3 |
| D11k | Transcription Activators & Repressors | ADA2-like protein | −1.5 | 0.0 |
| D12f | Cell Signaling & Extracellular | neuropeptide Y precursor (NPY) | −1.6 | −1.0 |
| D14j | RNA Polymerase | activated RNA polymerase II transcrip [symbol] | 1.9 | 2.2 |
| E02d | Transcription Activators & Repressors | TRAF-interacting protein (I-TRAF) + T [symbol] | 0.3 | −1.6 |
| E02k | Growth Factor & Chemokine | C5a anaphylatoxin receptor (C5AR); C | −1.8 | −2.6 |
| E03d | RNA Polymerase | transcription initiation factor TFIID 31- [symbol] | 2.3 | −1.5 |
| E03n | Translation | 14.5-kDa translational inhibitor protein | −1.8 | 0.0 |
| E04d | Transcription Activators & Repressors | AP4 basic helix-loop-helix DNA-bindin [symbol] | 0.5 | −3.3 |
| E04n | Xenobiotic Transporters | beta-defensin 2 precursor (hBD2); skin | −3.4 | 0.3 |
| E05d | Transcription Activators & Repressors | C-ets-2 | 0.0 | −2.1 |
| E05h | Cell-Cell Adhesion Receptors | NADH-ubiquinone oxidoreductase B18 | −1.6 | 0.8 |
| E06d | Transcription Activators & Repressors | raf-responsive zinc finger protein | 0.0 | −3.0 |
| E7b | Transcription Activators & Repressors | fli-1 oncogene; ergB transcription fact [symbol] | 2.5 | 1.1 |
| E07c | Cell Cycle-Regulating Kinases | homeobox protein hLim1; LHX1 | 1.6 | 0.0 |
| E07d | Transcription Activators & Repressors | orphan hormone nuclear receptor | 0.6 | −2.8 |
| E08c | CDK Inhibitors | trans-acting T-cell specific transcriptio [symbol] | 2.7 | 0.7 |
| E08d | Transcription Activators & Repressors | nuclear factor kappa-B DNA binding s [symbol] | 1.7 | 0.0 |
| E08e | Basic Transcription Factors | guanine nucleotide-binding protein G- [symbol] | 2.3 | 1.2 |
| E081 | Interleukin & Interferon Receptors | interferon-alpha/beta receptor beta su [symbol] | 1.7 | 0.2 |
| E08n | Drug-Resistance Proteins | soluble epoxide hydrolase (SEH); epo [symbol] | 1.6 | 0.0 |
| E09c | Transcription Activators & Repressors | transcription factor Sp1 (TSFP1) | 1.9 | 1.0 |
| E09d | Transcription Activators & Repressors | zinc-finger DNA-binding protein | 3.1 | 0.4 |
| E09i | Cell-Cell Adhesion Receptors | fibronectin receptor beta subunit (FNR [symbol] | 0.7 | 2.4 |
| E09l | Interleukin & Interferon Receptors | interleukin-2 receptor gamma subunit [symbol] | 1.7 | 0.0 |
| E10a | Transcription Activators & Repressors | early growth response protein 1 (hEGF | −1.7 | −1.3 |
| E10d | Transcription Activators & Repressors | 26S protease regulatory subunit 6A; T [symbol] | 1.9 | 1.1 |
| E10k | Growth Factor & Chemokine | corticotropin releasing factor receptor [symbol] | 2.9 | 2.2 |
| E11c | CDK Inhibitors | Sp2 protein | 1.6 | −1.0 |
| E11d | Transcription Activators & Repressors | purine-rich single-stranded DNA-bindin [symbol] | 2.4 | 0.0 |
| E11e | Transcription Activators & Repressors | tristetraproline (TTP): TIS11; ZFP36; g | 1.4 | −2.3 |
| E12g | Cell-Cell Adhesion Receptors | vitronectin receptor alpha subunit (VN [symbol] | 1.6 | 1.2 |
| E12l | Interleukin & Interferon Receptors | interleukin 10 receptor (IL-10R) | 0.9 | −1.5 |
| E12m | Xenobiotic Transporters | selenium-binding protein | 1.7 | 0.8 |
| E13d | Basic Transcription Factors | CCAAT-binding transcription factor su [symbol] | 2.6 | 0.7 |
| E13k | Growth Factor & Chemokine | N-sam; fibroblast growth factor recept [symbol] | 1.6 | 0.0 |
| E13m | Xenobiotic Transporters | microsomal stress 70 protein ATPase [symbol] | 1.8 | 0.0 |
| E13n | Xenobiotic Transporters | glutathione S-transferase theta 1 (GS [symbol] | 1.6 | 0.3 |
| E14i | Cell-Cell Adhesion Receptors | leukocyte adhesion glycoprotein LFA-1 | −3.8 | −2.1 |
| E14k | Interleukin & Interferon Receptors | interleukin-7 receptor alpha subunit p [symbol] | 2.2 | −0.8 |
| E14m | Drug-Resistance Proteins | thiosulfate sulfurtransferase; rhodanes | 1.6 | 0.7 |
| F01g | Growth Factors, Cytokines & | teratocarcinoma-derived growth factor | 0.0 | −1.9 |
| F02f | Growth Factors, Cytokines & | vascular endothelial growth factor pre [symbol] | 0.1 | 2.1 |
| F02l | Proteosomal Proteins | proteasome component C3; macropair | 1.9 | 1.8 |
| F03f | Growth Factors, Cytokines & | pleiotrophin precursor (PTN) + osteobl | −1.6 | 0.0 |
| F03l | Proteosomal Proteins | proteasome component C5; macropair | 2.0 | 1.2 |
| F03n | Protease inhibitors | endothelial plasminogen activator inhit | 1.7 | 0.3 |
| F04b | Heat Shock Proteins | heat shock 90-kDa protein A (HSP90A | 2.9 | 0.1 |
| F04e | Growth Factors, Cytokines & | hepatocyte growth factor activator (HG | −1.7 | −2.8 |
| F04g | Growth Factors, Cytokines & | interferon gamma-induced protein pre [symbol] | −2.0 | 0.0 |
| F04l | Proteosomal Proteins | proteasome component C8; macropair | 1.7 | 1.0 |
| F04n | Protease inhibitors | placental plasminogen activator inhibit | 2.0 | 2.5 |
| F05a | Xenobiotic Transporters | glutathione peroxidase (GSHPX1; GP) | −1.5 | −1.0 |
| F05b | Heat Shock Proteins | 27-kDa heat-shock protein (HSP27); s [symbol] | 2.8 | 0.0 |
| F05g | Growth Factors, Cytokines & | migration inhibitory factor-related prot [symbol] | 0.0 | 1.7 |
| F05i | Interleukins & Interferons | interleukin-18 precursor (IL-18); interfe | 1.5 | 0.8 |
| F05k | Protease Inhibitors | alpha-1-antitrypsin precursor; alpha-1 [symbol] | 1.4 | 1.6 |
| F05m | Metalloproteinases | matrix metalloproteinase 7 (MMP7); m | 1.9 | 0.0 |
| F06b | Heat Shock Proteins | 70-kDa heat shock protein 1 (HSP70.1 | 2.2 | 0.3 |
| F06e | Growth Factors, Cytokines & | endothelin 3 (EDN3; ET3) | 0.0 | −2.2 |
| F06i | Interleukins & Interferons | interferon gamma precursor (IFN-gam [symbol] | 2.4 | 0.1 |
| F06m | Metalloproteinases | matrix metalloproteinase 8 (MMP8); n [symbol] | 1.6 | −1.0 |
| F08f | Growth Factors, Cytokines & | keratinocyte growth factor (KGF); fibro | 0.2 | −2.1 |
| F08k | Cysteine Proteases | cathepsin H precursor | 1.5 | 1.7 |
| F09e | Growth Factors, Cytokines & | uromodulin; Tamm-Horsfall urinary gly | −1.7 | −1.2 |
| F09f | Growth Factors, Cytokines & | brain-derived neurotrophic factor (BD [symbol] | 0.0 | −3.5 |
| F09i | Interleukins & Interferons | interleukin-1 alpha precursor (IL-1 alph | 1.9 | 0.4 |
| F09n | Amino—& Carboxypeptidases | tripeptidyl-peptidase I precursor, tripep | 0.0 | −2.0 |
| F10a | Xenobiotic Metabolism | dioxin-inducible cytochrome P450 1B1 | −2.7 | 0.8 |
| F10e | Growth Factors, Cytokines & | T-cell-specific rantes protein precursor | −4.0 | 0.0 |
| F10g | Growth Factors, Cytokines & | macrophage inflammatory protein 2 al [symbol] | 1.6 | 3.6 |
| F10i | Interleukins & Interferons | interleukin-1 beta precursor (IL-1; IL1 [symbol] | 1.2 | 2.3 |
| F11g | Growth Factors, Cytokines & | placenta growth factors 1 + 2 (PLGF1 [symbol] | 1.4 | 1.5 |
| F12f | Growth Factors, Cytokines & | hepatocyte growth factor-like protein; [symbol] | 0.0 | −3.7 |
| F12g | Growth Factors, Cytokines & | granulocyte chemotactic protein 2 (GC | −1.3 | 4.6 |
| F12n | Cysteine Proteases | cathepsin L precursor; major excreted | 1.8 | 3.7 |
| F13h | Hormones | cellular retinoic acid-binding protein II [symbol] | 0.2 | −1.6 |
| F13n | Other RNA Processing, Turnover & | activator of RNA decay (ARD-1) | 0.1 | 1.6 |
| F14e | Growth Factors, Cytokines & | amphiregulin (AR); colorectum cell-de [symbol] | 0.0 | −1.6 |
| F14g | Growth Factors, Cytokines & | interleukin-8 precursor (IL-8); monocyt | 1.8 | 4.0 |
| F14k | Proteosomal Proteins | proteasome inhibitor HPI31 subunit [symbol] | 0.5 | −2.5 |
| F14n | Other Receptors (by Activities) | zinc finger X-chromosomal protein (ZF | 1.6 | 1.4 |
| G29 | Housekeeping Genes | brain-specific tubulin alpha 1 subunit ( [symbol] | 0.0 | −2.4 |
| G31 | Housekeeping Genes | HLA class I histocompatibility antigen [symbol] | 0.0 | −1.7 |
| G43 | Housekeeping Genes | cytoplasmic beta-actin (ACTB) | −1.8 | 0.0 |

TABLE 8b
GENE CHANGES INDUCED BY DENGUE VIRUS IN VITRO IN
HUMAN LYMPHOID CELLS-ARRAY II

| Gene code | Classification#1 | Protein/gene | DEN-4h | DEN-8h |
|---|---|---|---|---|
| A02d | Cell Surface Antigens | annexin V; lipocortin V; endonexin II | 5.8 | 4.1 |
| B10f | Oncogenes & Tumor Suppressors | nucleolar phosphoprotein B23; nucle | 4.6 | 1.7 |
| B06k | Other Immune System Proteins | L-plastin; lymphocyte cytosolic prote | 3.9 | 1.2 |
| D06m | Protein Modification Enzymes | protein disulfide Isomerase | 3.7 | −1.9 |
| F04l | Other Intracellular Transducers, | phosphatidylinositol transfer protein | 3.6 | 0.9 |
| | Effectors & Modulators | | | |
| D05h | Other Metabolism Enzymes | mitochondrial 4-aminobutyrate amin | 3.6 | 0.1 |
| E05C | Growth Factors, Cytokines & | monocyte chemotactic protein 3 pre | 3.6 | 4.5 |
| | Chemokines | | | |
| A03n | Cell Surface Antigens | lymphocyte function-associated anti | 3.5 | 5.1 |
| D14j | Hormone Receptors | nuclear receptor-related 1 | 3.4 | 0.0 |
| D02m | Nucleotide Metabolism | thymidylate synthase (TYMS; TS) | 3.3 | 0.2 |
| C04f | Exocytosis Proteins | annexin I (ANX1) | 3.1 | 2.5 |
| F03m | Kinase Activators & Inhibitors | 14-3-3 protein beta/alpha; protein ki [symbol] | 3.0 | 2.9 |
| D08e | RNA Processing, Tumover & | ATAXIN-2 RELATED PROTEIN | 2.9 | 0.5 |
| | Transport Proteins | | | |
| A01n | Cell Surface Antigens | lysosome-associated membrane gly | 2.9 | 1.7 |
| E09c | Intracellular Kinase Network | casein kinase I delta isoform (CKI-d [symbol] | 2.9 | 0.8 |
| | Members | | | |
| B10d | Oncogenes & Tumor Suppressors | B-cell translocation gene 1 protein ( [symbol] | 2.9 | 0.2 |
| F11d | Orphan Receptors | orphan receptor TR4 | 2.6 | 0.0 |
| E13k | G Proteins | GUANINE NUCLEOTIDE-BINDING | 2.6 | 0.0 |
| A01c | Cell Surface Antigens | leukocyte CD37 antigen | 2.6 | −1.0 |
| F04a | Kinase Activators & Inhibitors | 14-3-3 PROTEIN ZETA/DELTA (PR | 2.5 | 1.1 |
| A01g | Cell Surface Antigens | leukocyte surface CD53 antigen; cel | 2.5 | −0.2 |
| F01c | GTP/GDP Exchangers & GTPase | ras GTPase-activating-like protein I( | 2.4 | 0.0 |
| | Activity Modulators | | | |
| C08d | Other Trafficking & Targeting | RAB GDP dissociation inihibitor beta | 2.4 | 0.0 |
| | Proteins | | | |
| F14b | Cell Signaling & Extracellular | major prion protein precursor (PRP) | 2.4 | 1.1 |
| | Communication Proteins | | | |
| A10i | Basic Transcription Factors | HOMEOBOX PROTEIN HOX-B1 = I | 2.3 | −1.4 |
| A04c | Cell Surface Antigens | CD83 antigen precursor; cell surface | 2.3 | 0.2 |
| D07n | Other Apoptosis-Associated Proteins | TIA-1 related protein; nucleolysin T [symbol] | 2.3 | 2.7 |
| D04n | Metabolism of Cofactors, Vitamins & | very-long-chain-specific acyl-CoA d [symbol] | 2.2 | 0.9 |
| | Related Substances | | | |
| A12l | Basic Transcription Factors | SEF2-1B PROTEIN; HELIX-LOOP- [symbol] | 2.2 | 0.0 |
| A08k | Basic Transcription Factors | CCAAT/enhancer binding protein ga | 2.2 | 1.2 |
| A06m | Basic Transcription Factors | human T-cell leukemia virus enhanc | 2.2 | 0.3 |
| D04e | Amino Acid Metabolism | GLCLC, GLCL (Glutamate-cysteine | 2.2 | 2.7 |
| C04g | Exocytosis Proteins | annexin II (ANX2); lipocortin II; calp [symbol] | 2.1 | 1.6 |
| D02e | Complex Lipid Metabolism | 3-ketoacyl-CoA thiolase peroxisoma | 2.1 | 0.9 |
| E13f | Hormone Receptors | guanine nucleotide-binding protein [symbol] | 2.0 | 0.4 |
| A01b | Cell Surface Antigens | CD81 antigen; 26-kDa cell surface [symbol] | 1.9 | 0.0 |
| B05m | Other Immune System Proteins | grancalcin | 1.9 | 0.9 |
| C07g | Other Trafficking & Targeting | ras-related protein RAB-1A; YPT1-r [symbol] | 1.9 | 3.7 |
| | Proteins | | | |
| D14g | Hormone Receptors | estrogen receptor beta (ER-beta) | 1.9 | 0.0 |
| E09m | Intracellular Transducers, Effectors & | G protein-coupled receptor kinase G [symbol] | 1.9 | 0.6 |
| | Modulators | | | |
| D06c | Other Metabolism Enzymes | aldehyde dehydrogenase 2 (ALDH2) | 1.9 | 0.0 |
| G11 | Housekeeping Genes | ubiquitin | 1.8 | −2.3 |
| F04i | Other Intracellular Transducers, | mothers against dpp homolog 7 (S [symbol] | 1.8 | 1.0 |
| | Effectors & Modulators | | | |
| B01l | Transcription Activators & | gamma-interferon-inducible protein | 1.8 | 0.0 |
| | Repressors | | | |
| C01e | Cell Signaling & Extracellular | sodium-dependent dopamine transp | 1.8 | 0.0 |
| | Communication Proteins | | | |
| B01e | Basic Transcription Factors | TGF-beta inducible early protein (TI | 1.8 | 0.6 |
| E14a | G Proteins | GUANINE NUCLEOTIDE-BINDING | 1.7 | 0.0 |
| E09a | Intracellular Kinase Network | RIBOSOMAL PROTEIN S6 KINASE | 1.7 | 0.0 |
| | Members | | | |
| F05n | Other Intracellular Transducers, | amyloid-like protein 2 | 1.7 | 4.6 |
| | Effectors & Modulators | | | |
| F03l | Kinase Activators & Inhibitors | muscle/brain cAMP-dependent prot [symbol] | 1.7 | 1.7 |
| B08e | Oncogenes & Tumor Suppressors | erythroblastosis virus oncogene hon | 1.7 | 0.9 |
| A02k | Cell Surface Antigens | L-selectin precursor; lymph node ho | 1.7 | 2.3 |
| D08n | Intracellular Transducers, Effectors & | serine/threonine-protein kinase rece | 1.7 | 0.0 |
| | Modulators | | | |
| D06d | Other Metabolism Enzymes | platelet-activating factor acetylhydr [symbol] | 1.7 | 0.8 |
| A02e | Cell Signaling & Extracellular | axonin-1 precursor; transient axonal | 1.6 | 0.0 |
| | Communication Proteins | | | |
| B11e | Oncogenes & Tumor Suppressors | ras-related protein R-ras2; ras-like p | 1.6 | 0.0 |
| A11c | Basic Transcription Factors | HOMEOBOX PROTEIN MSX-2 (HC | 1.6 | 0.0 |
| C03e | Extracellular Matrix Proteins | collagen 10 alpha 1 subunit (COL10 | 1.6 | −1.4 |
| A02l | Cell Surface Antigens | P-selectin precursor (SELP); granul [symbol] | 1.6 | 0.0 |
| A03i | Cell Surface Antigens | T-cell surface glycoprotein CD3 eps | 1.6 | 3.0 |
| E11m | Intracellular Protein Phosphatases | protein-tyrosine phosphatase MEG1 | 1.6 | 0.0 |
| D14i | Hormone Receptors | neuron-derived orphan receptor 1 ( [symbol] | 1.5 | −1.2 |
| C11g | Energy Metabolism | alcohol dehydrogenase 5 chi polype | 1.5 | −1.1 |
| A10a | Basic Transcription Factors | HOMEOBOX PROTEIN DLX-2 | 1.5 | 0.0 |
| E12a | Intracellular Protein Phosphatases | protein-tyrosine phosphatase MEG2 | 1.5 | 0.0 |
| B10j | Oncogenes & Tumor Suppressors | retinoic acid receptor alpha | 1.5 | 1.6 |
| A10d | Basic Transcription Factors | HOMEOBOX PROTEIN EMX2 | −1.5 | −1.2 |
| B04g | Cell-Cell Adhesion Receptors | SUSHI REPEAT-CONTAINING PR [symbol] | −1.5 | 0.2 |
| B05f | Other Immune System Proteins | granzyme M precursor (GZMM); me | −1.5 | −1.5 |
| F04k | Hormone Receptors | G-alpha Interacting protein (GAIP) | −1.5 | 0.0 |
| E07a | Other Extracellular Communication | FOLLISTATIN 1 AND 2 PRECURS [symbol] | −1.6 | 0.2 |
| | Proteins | | | |
| C06i | Cell Signaling & Extracellular | synaptosomal-associated protein 25 | −1.6 | 0.0 |
| | Communication Proteins | | | |
| C13g | Complex Lipid Metabolism | lysosomal acid lipase/cholesteryl es [symbol] | −1.6 | 0.0 |
| C05k | Other Trafficking & Targeting | Golgi SNARE; GS27 | −1.6 | −1.1 |
| | Proteins | | | |
| E05k | Growth Factors, Cytokines & | proliferation-inducing ligand (APRIL [symbol] | −1.6 | 0.0 |
| | Chemokines | | | |
| C05i | Other Trafficking & Targeting | golga2; golgin 95-kDa protein | −1.6 | 0.2 |
| | Proteins | | | |
| E12f | Intracellular Protein Phosphatases | protein-tyrosine phosphatase alpha I | −1.6 | 0.0 |
| C12f | Simple Lipid Metabolism | mitochondrial enoyl-CoA hydratase: | −1.6 | 0.9 |
| B07b | Calcium-Binding Proteins | S100 calcium-binding protein A7; ps | −1.6 | 0.1 |
| B10g | Oncogenes & Tumor Suppressors | nuclear pore complex protein 214 ( [symbol] | −1.6 | 0.4 |
| B07i | Oncogenes & Tumor Suppressors | C6.1A protein | −1.6 | 0.7 |
| C03b | Extracellular Matrix Proteins | cartilage glycoprotein 39 precursor ( | −1.6 | 0.2 |
| E04i | Growth Factors, Cytokines & | beta chemokine Exodus 2 | −1.6 | 0.0 |
| | Chemokines | | | |
| C06d | Other Trafficking & Targeting | gamma-soluble NSF attachment pr [symbol] | −1.6 | 0.0 |
| | Proteins | | | |
| B13k | Voltage-Gated Ion Channels | dihydropyridine-sensitive I-type cha [symbol] | −1.7 | 0.0 |
| B08a | Oncogenes & Tumor Suppressors | rhombotin-2 (RBTN2; RHOM2); cys [symbol] | −1.7 | 0.5 |
| C03n | G Proteins | ADP-ribosylation factor 1 | −1.7 | −1.0 |
| A13m | Basic Transcription Factors | TRANSCRIPTIONAL ENHANCER [symbol] | −1.7 | 0.0 |
| C14n | Complex Lipid Metabolism | lanosterol synthase (LSS); oxidosqu | −1.7 | 0.0 |
| A10n | Basic Transcription Factors | HOMEOBOX PROTEIN MEIS3 (ME | −1.7 | 0.0 |
| B05k | Other Immune System Proteins | NEUTROPHIL DEFENSINS 1,2 A [symbol] | −1.7 | 0.7 |
| A12j | Basic Transcription Factors | POD1 - MESODERM-SPECIFIC B | −1.8 | 0.0 |
| C13l | Extracellular Transporters & Carrier | apolipoprotein E precursor (APOE) | −1.8 | −1.2 |
| | Proteins | | | |
| C04k | Other Trafficking & Targeting | ER lumen protein retaining receptor | −1.8 | 0.0 |
| | Proteins | | | |
| E01f | Cell Signaling & Extracellular | gamma-aminobutyric-acid receptor [symbol] | −1.8 | 0.9 |
| | Communication Proteins | | | |
| A08a | Basic Transcription Factors | homeobox protein HOX-A4; HOX-1 [symbol] | −1.9 | 0.1 |
| A11g | Basic Transcription Factors | PITX2 OR RIEG OR RGS - PITUIT [symbol] | −1.9 | 0.0 |
| G29 | Housekeeping Genes | brain-specific tubulin alpha 1 subuni | −1.9 | −2.3 |
| F01d | Phospholipases & Phosphoinositol | phospholipase C beta 2 (PLC-beta 2 | −1.9 | 0.0 |
| | Kinases | | | |
| C08l | Other Trafficking & Targeting | syntaxin 5 (STX5) | −1.9 | −1.0 |
| | Proteins | | | |
| C01f | Cell Signaling & Extracellular | sodium- & chloride-dependent GAB [symbol] | −1.9 | 0.0 |
| | Communication Proteins | | | |
| A11k | Basic Transcription Factors | MYELIN TRANSCRIPTION FACTO | −1.9 | 0.5 |
| F06a | Other Intracellular Transducers, | diacylglycerol kinase gamma (DGK- | −2.0 | 1.8 |
| | Effectors & Modulators | | | |
| B02f | Transcription Activators & | SMOOTH MUSCLE CELL LIM PRO | −2.0 | 0.0 |
| | Repressors | | | |
| E13i | Hormone Receptors | guanine nucleotide-binding protein [symbol] | −2.0 | 0.0 |
| A05g | Cell Surface Antigens | CD40 | −2.1 | 0.0 |
| B04f | Cell-Cell Adhesion Receptors | GAP JUNCTION ALPHA-8 PROTEI | −2.1 | 0.7 |
| F09h | G Protein-Coupled Receptors | Mrg = mas-related | −2.1 | 0.5 |
| E05f | Growth Factors, Cytokines & | FIBROBLAST GROWTH FACTOR- | −2.2 | −1.1 |
| | Chemokines | | | |
| D13h | Hormone Receptors | melanocortin-4 receptor (MC4-R) | −2.2 | 0.3 |
| C02j | Extracellular Matrix Proteins | lumican precursor (LUM); keratan s [symbol] | −2.2 | −1.0 |
| D10c | Hormone Receptors | histamine H1 receptor (HRH1) | −2.3 | 0.0 |
| A09c | Basic Transcription Factors | EARLY GROWTH RESPONSE PR [symbol] | −2.3 | −1.8 |
| A12a | Basic Transcription Factors | MYOGENIC FACTOR MYF-5 | −2.3 | 0.0 |
| A12n | Basic Transcription Factors | SKELETAL MUSCLE LIM-PROTEI [symbol] | −2.3 | 0.0 |
| A07m | Transcription Activators & | interferon regulatory factor 7 (IRF-7) | −2.3 | −1.3 |
| | Repressors | | | |
| A10e | Basic Transcription Factors | HOMEOBOX PROTEIN HB9 = HLX | −2.3 | 0.3 |
| E01c | Neurotransmitter Receptors | gamma-aminobutyric-acid receptor; | −2.3 | 0.0 |
| F09l | G Protein-Coupled Receptors | extracellular calcium-sensing recept | −2.5 | 0.3 |
| F05d | Cell Signaling & Extracellular | 43-kDa postsynaptic protein; acetylc | −2.5 | 0.1 |
| | Communication Proteins | | | |
| F06g | Cell Signaling & Extracellular | neuroendocrine convertase 1 precu [symbol] | −2.6 | 0.6 |
| | Communication Proteins | | | |
| E06k | Hormones | natriuretic peptide precursor B | −2.7 | 0.2 |
| E13b | Intracellular Protein Phosphatases | serine/threonine phosphatase | −2.7 | 0.7 |
| C14a | Complex Lipid Metabolism | lipoprotein lipase precursor (LPL) | −3.3 | 0.0 |
| C07e | G Proteins | ras-related protein RAB-7 | −4.2 | 2.3 |
| B06h | Other Immune System Proteins | calgranulin C (CAGC) CGRP; neutr [symbol] | 0.2 | 3.8 |
| G27 | Housekeeping Genes | liver glyceraldehyde 3-phosphate d [symbol] | 0.8 | 3.6 |
| E12c | Intracellular Protein Phosphatases | protein-tyrosine phosphatase G1 (P [symbol] | 1.4 | 3.5 |
| A02g | Cell Surface Antigens | LGALS3, MAC2 (Galectin-3, MAC-2 | 1.0 | 3.4 |
| B08h | Oncogenes & Tumor Suppressors | zinc finger protein hrx; ALL-1; MLL | −1.0 | 3.1 |
| F09i | G Protein-Coupled Receptors | PUTATIVE RECEPTOR PROTEIN [symbol] | 1.3 | 2.9 |
| F13m | Functionally Unclassified Proteins | PROTEIN PHPS1-2 | 1.1 | 2.7 |
| F06c | Other Intracellular Transducers, | guanine nucleotide-binding protein [symbol] | −0.3 | 2.7 |
| | Effectors & Modulators | | | |
| C09d | Simple Carbohydrate Metabolism | long-chain-fatty-acid-CoA ligase 1 + | 0.0 | 2.7 |
| C03d | Extracellular Matrix Proteins | osteocalcin precursor; gamma-carb [symbol] | 0.0 | 2.6 |
| E09b | Intracellular Kinase Network | casein kinase I alpha isoform (CKI-ε | 1.0 | 2.4 |
| | Members | | | |
| F02i | Adenylate/Guanylate Cyclases & | adenylate cyclase type VIII; ATP py [symbol] | 0.0 | 2.4 |
| | Diesterases | | | |
| F07h | Calpains | calcium-dependent protease small ( | 0.5 | 2.4 |
| C04e | Exocytosis Proteins | annexin IV (ANX4); lipocortin I; calp | 0.5 | 2.3 |
| D05g | Other Metabolism Enzymes | mitochondrial aldehyde dehydrogen [symbol] | 0.1 | 2.3 |
| B10l | Oncogenes & Tumor Suppressors | EVI2B protein precursor; ectropic vi [symbol] | 0.2 | 2.2 |
| C05g | Other Trafficking & Targeting | coatomer delta subunit; delta-coat p | 0.1 | 2.2 |
| | Proteins | | | |
| F07i | Proteosomal Proteins | HUNTINGTIN INTERACTING PRO [symbol] | 0.2 | 2.2 |
| C03k | Exocytosis Proteins | synaptotagmin V | 0.0 | 2.1 |
| A08f | Basic Transcription Factors | NF-AT4c | 1.2 | 2.0 |
| D05i | Other Metabolism Enzymes | 5-aminolevulinic acid synthase mito [symbol] | 0.4 | 2.0 |
| E14g | GTP/GDP Exchangers & GTPase | REGULATOR OF G-PROTEIN SIG [symbol] | 0.9 | 2.0 |
| | Activity Modulators | | | |
| F03n | Kinase Activators & Inhibitors | 14-3-3 PROTEIN EPSILON (MITO [symbol] | 1.3 | 2.0 |
| B11f | GTP/GDP Exchangers & GTPase | GTPase-activating protein (GAP); r [symbol] | 1.3 | 2.0 |
| | Activity Modulators | | | |
| G43 | Housekeeping Genes | cytoplasmic beta-actin (ACTB) | 0.5 | 2.0 |
| E09e | Intracellular Kinase Network | casein kinase II alpha' subunit (CK I [symbol] | 1.1 | 1.9 |
| | Members | | | |
| A03j | Cell Surface Antigens | T-cell surface glycoprotein CD5 pre [symbol] | 0.0 | 1.9 |
| E11g | Intracellular Protein Phosphatases | protein phosphatase 2C alpha isofo [symbol] | 1.4 | 1.9 |
| F07g | Calpains | calpain p94 large (catalytic) subunit; | 0.0 | 1.9 |
| D07d | Other Post-Translational Modification | cyclophilin 3 protein (CYP3); mitoch | 0.0 | 1.9 |
| | Proteins | | | |
| D12j | Cell Signaling & Extracellular | neuromedin K receptor (NKR); neun | 0.1 | 1.9 |
| | Communication Proteins | | | |
| F10c | G Protein-Coupled Receptors | EBV-induced G-protein-coupled rec [symbol] | 1.2 | 1.9 |
| E06f | Growth Factors, Cytokines & | granulins precursor (GRN); acrograr | 0.8 | 1.9 |
| | Chemokines | | | |
| F02n | Calcium-Binding Proteins | calbindin; avian-type vitamin D-dep [symbol] | 0.4 | 1.8 |
| F09k | G Protein-Coupled Receptors | adenosine A2B receptor (ADORA2 [symbol] | 0.0 | 1.8 |
| C05l | Other Trafficking & Targeting | cation-dependent mannose-6-phosp | 0.0 | 1.8 |
| | Proteins | | | |
| D03j | Amino Acid Metabolism | glycine dehydrogenase (decarboxyl [symbol] | 0.0 | 1.8 |
| D06b | Other Metabolism Enzymes | cytochrome P450 VA1 (CYP5A1) | 0.3 | 1.8 |
| B13h | Voltage-Gated Ion Channels | voltage-dependent anion-selective [symbol] | 1.4 | 1.8 |
| F08j | Other Enzymeslinvolved in Protein | vitamin K-dependent protein S | 0.0 | 1.8 |
| | Turnover | | | |
| D10g | Hormone Receptors | CCKB-Cholecystokinin receptor | 0.6 | 1.8 |
| C02d | Other Membrane Channels & | kidney UT2 urea transporter; SLC14 | −1.1 | 1.8 |
| | Transporters | | | |
| F03g | Calcium-Binding Proteins | calgizzarin; S100C protein; MLN70 | 0.0 | 1.8 |
| F08g | Other Enzymeslinvolved in Protein | fibinogen B beta polypeptide | 0.0 | 1.8 |
| | Turnover | | | |
| E13n | G Proteins | ras-related protein RAP-1B; GTP-bi [symbol] | 0.9 | 1.8 |
| G13 | Housekeeping Genes | phospholipase A2 | 0.3 | 1.8 |
| B07h | Oncogenes & Tumor Suppressors | dek protein | 1.5 | 1.8 |
| D02n | Nucleotide Metabolism | cytosolic thymidine kinase (TK1) | 1.0 | 1.7 |
| E02k | Neurotransmitter Receptors | M5-Muscarinic acetylcholine recept [symbol] | −1.1 | 1.7 |
| E10n | Intracellular Protein Phosphatases | dual-specificity protein phosphatase | 1.2 | 1.7 |
| C05e | Other Trafficking & Targeting | coatomer beta' subunit; beta'-coat p [symbol] | 0.0 | 1.7 |
| | Proteins | | | |
| E12l | Intracellular Protein Phosphatases | serine/threonine protein phosphatas [symbol] | 1.3 | 1.7 |
| D04m | Metabolism of Cofactors, Vitamins & | peroxisomal acyl-coenzynie A oxida | 0.6 | 1.7 |
| | Related Substances | | | |
| E06b | Growth Factors, Cytokines & | growth/differentiation factor 5 precu [symbol] | 0.0 | 1.7 |
| | Chemokines | | | |
| D12i | Cell Signaling & Extracellular | substance-K receptor (SKR); neurok | 0.0 | 1.7 |
| | Communication Proteins | | | |
| E05h | Growth Factors, Cytokines & | FIBROBLAST GROWTH FACTOR- | 0.0 | 1.7 |
| | Chemokines | | | |
| E14l | GTP/GDP Exchangers & GTPase | REGULATOR OF G-PROTEIN SIG [symbol] | 1.0 | 1.7 |
| | Activity Modulators | | | |
| B09k | Transcription Activators & | B-cell lymphoma 3-encoded protein | 0.9 | 1.7 |
| | Repressors | | | |
| E06l | Hormones | STC (Stanniocalcin) | −1.2 | 1.6 |
| F06b | Other Intracellular Transducers, | calpain inhibitor; calpastatin (CAST) | 0.8 | 1.6 |
| | Effectors & Modulators | | | |
| F06n | Serine Proteases | coagulation factor XII | 0.6 | 1.6 |
| F14d | Functionally Unclassified Proteins | DXS6673E protein; X-linked mental - | 0.0 | 1.6 |
| E05a | Growth Factors, Cytokines & | macrophage-derived chemokine pre | 0.2 | 1.6 |
| | Chemokines | | | |
| F06m | Serine Proteases | coagulation factor IX | 0.0 | 1.6 |
| F12g | Other Cytoskeleton & Motility | COFILIN | 1.3 | 1.6 |
| | Proteins | | | |
| G15 | Housekeeping Genes | hypoxanthine-guanine phosphoribos | 0.0 | 1.5 |
| D07b | Protein Modification Enzymes | platelet-activating factor acetylhydr [symbol] | 0.0 | 1.5 |
| C13m | Complex Lipid Metabolism | cholinephosphate cytidylyltransferas | 0.4 | 1.5 |
| D08j | Growth Factor & Chemokine | leukocyte platelet-activating factor [symbol] | 0.0 | 1.5 |
| | Receptors | | | |
| B10i | Oncogenes & Tumor Suppressors | platelet-derived growth factor (PDGI | 1.1 | 1.5 |
| | Cell Signaling & Extracellular | | | |
| D04i | Communication Proteins | glutamate decarboxylase 65-kDa is [symbol] | 0.4 | 1.5 |
| | Other Intracellular Transducers, | | | |
| F05k | Effectors & Modulators | mothers against dpp homolog 2 (hM | 0.3 | −1.5 |
| D14k | Hormone Receptors | vitamin D3 receptor (VDR) | 0.2 | −1.5 |
| B11b | Oncogenes & Tumor Suppressors | ras-related protein RAB-8; oncogene | −1.3 | −1.5 |
| F10b | G Protein-Coupled Receptors | probable G-protein-coupled receptor | 0.0 | −1.5 |
| E01k | Neurotransmitter Receptors | gamma-aminobutyric-acid receptor [symbol] | 1.1 | −1.5 |
| | Cell Signaling & Extracellular | | | |
| D03k | Communication Proteins | tryptophan 5-hydroxylase (TRPH); t [symbol] | 0.7 | −1.6 |
| B07f | Oncogenes & Tumor Suppressors | breakpoint cluster region protein (B [symbol] | 0.0 | −1.6 |
| A09i | Basic Transcription Factors | FORKHEAD-RELATED TRANSCR [symbol] | 0.0 | −1.6 |
| F02c | Adenylate/Guanylate Cyclases & Diesterases | guanylate cyclase soluble beta-1 sul | 0.1 | −1.6 |
| F09d | Protein phosphatase Receptors | protein-tyrosine phosphatase X prec | 0.1 | −1.6 |
| | Intracellular Kinase Network | | | |
| E08e | Members | protein-tyrosine phosphatase LC-PT | 0.0 | −1.6 |
| | Other Intracellular Transducers. | | | |
| F05a | Effectors & Modulators | diacylglycerol kinase zeta (DAG kin [symbol] | 0.0 | −1.7 |
| A11h | Basic Transcription Factors | HOMEOBOX PROTEIN SIX1 | 1.0 | −1.7 |
| E01h | Neurotransmitter Receptors | gamma-aminobutyric-acid receptor [symbol] | 1.0 | −1.7 |
| A04l | Cell Surface Antigens | cytotoxic T-lymphocyte protein 4-1 [symbol] | 0.0 | −1.7 |
| E05e | Growth Factors, Cytokines & Chemokines | FIBROBLAST GROWTH FACTOR- | 0.9 | −1.7 |
| C08j | Other Trafficking & Targeting | clathrin coat assembly protein AP17 | 0.0 | −1.7 |
| | Proteins | | | |
| A12i | Basic Transcription Factors | PAIRED MESODERM HOMEOBOX | −1.0 | −1.7 |
| B07c | Calcium-Binding Proteins | S100 calcium-binding protein A1; S- | 0.4 | −1.8 |
| B14a | Voltage-Gated Ion Channels | voltage-gated potassium channel p [symbol] | 0.3 | −1.8 |
| F01b | GTP/GDP Exchangers & GTPase | rap1 GTPase-GDP dissociation stim | 0.1 | −1.8 |
| | Activity Modulators | | | |
| C09a | Simple Carbohydrate Metabolism | galactoside 2-I-fucosyltransferase 1; | 0.0 | −1.8 |
| B01h | Transcription Activators & | interleukin enhancer binding factor 2 | 0.0 | −1.8 |
| | Repressors | | | |
| B13e | Ligand-Gated Ion Channels | P2X purinoceptor 7 (P2X7); ATP rec | 0.0 | −1.8 |
| E06d | Growth Factors, Cytokines & | melanoma-derived growth regulator | 0.0 | −1.9 |
| | Chemokines | | | |
| E12i | Intracellular Protein Phosphatases | protein phosphatase 2A B'alpha1 re [symbol] | 0.3 | −1.9 |
| A04f | Cell Surface Antigens | T-cell surface glycoprotein CD1A p [symbol] | 0.0 | −1.9 |
| A13h | Basic Transcription Factors | SOX-9 PROTEIN | 0.0 | −1.9 |
| C05c | Other Trafficking & Targeting | microsomal triglyceride transfer pro [symbol] | 0.2 | −1.9 |
| | Proteins | | | |
| B06a | Other Immune System Proteins | ficolin 1 (FCN1) + FCN2; serum lec [symbol] | 0.6 | −1.9 |
| A01a | Cell Surface Antigens | cell surface glycoprotein A15; T-cell | 0.0 | −1.9 |
| B11k | Xenobiotic Metabolism | uridine diphosphate glycosyltransfer | 0.0 | −1.9 |
| A07d | Basic Transcription Factors | T-cell-specific transcription factor 1 | −1.2 | −2.0 |
| A05i | Cell Surface Antigens | complement receptor type 1 precurs | 0.0 | −2.0 |
| A03c | Cell Signaling & Extracellular | myelin-associated glycoprotein prec | 0.0 | −2.0 |
| | Communication Proteins | | | |
| A09j | Basic Transcription Factors | HATH-1 - ATONAL HOMOLOG | −1.1 | −2.0 |
| A08i | Basic Transcription Factors | NF-ATc | 0.4 | −2.0 |
| C10g | Energy Metabolism | mitochondrial trifunctional protein er | 0.6 | −2.0 |
| E14c | GTP/GDP Exchangers & GTPase | regulator of G-protein signalling 2 (F | 0.3 | −2.1 |
| | Activity Modulators | | | |
| A12m | Basic Transcription Factors | SINGLE-MINDED HOMOLOG 2 | 0.0 | −2.1 |
| D13d | Hormone Receptors | D(4) DOPAMINE RECEPTOR (D(2 [symbol] | 0.0 | −2.2 |
| A11j | Basic Transcription Factors | MAX - HELIX-LOOP-HELIX ZIPP [symbol] | 0.0 | −2.2 |
| G45 | Housekeeping Genes | 23-kDa highly basic protein; 60S rib [symbol] | 0.0 | −2.3 |
| D13n | Hormone Receptors | somatostatin receptor type 4 (SS4R | −1.3 | −2.3 |
| C09l | Complex Carbohydrate Metabolism | alpha-L-iduronidase precursor | 0.0 | −2.4 |
| B06d | Other Immune System Proteins | LDL-associated phospholipase A2 | 1.3 | −2.4 |
| A06g | Basic Transcription Factors | Runt domain-containing protein PEE | −1.3 | −2.7 |
| E11a | Intracellular Protein Phosphatases | dual-specificity protein phosphatase | 1.1 | −2.7 |
| A02f | Cell Surface Antigens | platelet glycoprotein IX | 1.3 | −2.8 |
| A04i | Cell Surface Antigens | T-cell surface glycoprotein CD1D pr | 0.2 | −2.8 |
| G31 | Housekeeping Genes | HLA class I histocompatibility antige | −1.2 | −3.6 |

TABLE 8c
GENE CHANGES INDUCED BY DENGUE VIRUS IN VITRO IN
HUMAN LYMPHOID CELLS - ARRAY I

| Gene Function | Protein/gene | DEN-4h | DEN-8h |
|---|---|---|---|
| Cell Cycle-Regulating Kinases | cell division protein kinase 6 (CDK6); [symbol] | 0.0 | 7.0 |
| Cell Cycle-Regulating Kinases | serine/threonine-protein kinase KKIAL [symbol] | 0.0 | 5.9 |
| Oncogenes & Tumor Suppressors | INT-2 proto-oncogene protein precurs [symbol] | 0.0 | 5.8 |
| CDK Inhibitors | cyclin-dependent kinase 4 inhibitor D ( | 0.7 | 5.8 |
| Oncogenes & Tumor Suppressors | B-myb | 0.6 | 5.3 |
| Oncogenes & Tumor Suppressors | tyrosine-protein kinase receptor UFO p | 0.0 | 5.0 |
| Oncogenes & Tumor Suppressors | p78 putative serine/threonine-protein k | 0.0 | 4.8 |
| Chemokines | granulocyte chemotactic protein 2 (GC | 1.3 | 4.6 |
| Effectors & Modulators | amyloid-like protein 2 | 1.7 | 4.6 |
| Transcription Activators & Repressors | estrogen receptor hSNF2b; global tran | 1.4 | 4.5 |
| Cell Surface Antigens | annexin V; lipocortin V; endonexin II; [symbol] | 5.8 | 4.1 |
| Oncogenes & Tumor Suppressors | tyrosine-protein kinase ABL2; tyrosine | 0.3 | 4.0 |
| Chemokines | interleukin-8 precursor (IL-8); monocyt | 1.8 | 4.0 |
| Basic Transcription Factors | transcriptional repressor NF-X1 | 1.0 | 3.9 |
| Other Immune System Proteins | calgranulin C (CAGC) CGRP; neutrop [symbol] | 0.2 | 3.8 |
| Cysteine Proteases | cathepsin L precursor; major excreted | 1.8 | 3.7 |
| Proteins | ras-related protein RAB-1A; YPT1-rela | 1.9 | 3.7 |
| Chemokines | macrophage inflammatory protein 2 al [symbol] | 1.6 | 3.6 |
| Housekeeping Genes | liver glyceraldehyde 3-phosphate dehy | 0.8 | 3.6 |
| G Proteins | guanine nucleotide-binding protein G( [symbol] | 3.0 | 3.5 |
| Intracellular Protein Phosphatases | protein-tyrosine phosphatase G1 (PTP | 1.4 | 3.5 |
| Cell Surface Antigens | LGALS3, MAC2 (Galectin-3, MAC-2 a [symbol] | 1.0 | 3.4 |
| Communication Proteins | neuronal pentraxin II precursor (NP2) | 0.0 | 3.4 |
| Oncogenes & Tumor Suppressors | zinc finger protein hrx; ALL-1; MLL | −1.0 | 3.1 |
| Communication Proteins | GABA-B receptor 2 subunit (GABA-BR | 0.2 | 3.1 |
| Cell Surface Antigens | T-cell surface glycoprotein CD3 epsilo [symbol] | 1.6 | 3.0 |
| G Protein-Coupled Receptors | PUTATIVE RECEPTOR PROTEIN (P [symbol] | 1.3 | 2.9 |
| Functionally Unclassified Proteins | PROTEIN PHPS1-2 | 1.1 | 2.7 |
| Chemokines | transforming growth factor-beta 3 (TG [symbol] | 0.0 | 2.7 |
| Effectors & Modulators | guanine nucleotide-binding protein bet | −0.3 | 2.7 |
| Amino Acid Metabolism | GLCLC, GLCL (Glutamate-cysteine lig | 2.2 | 2.7 |
| Other Apoptosis-Associated Proteins | TIA-1 related protein; nucleolysin TIA [symbol] | 2.3 | 2.7 |
| Simple Carbohydrate Metabolism | long-chain-fatty-acid-CoA ligase 1 + lo | 0.0 | 2.7 |
| Extracellular Matrix Proteins | osteocalcin precursor; gamma-carbox) | 0.0 | 2.6 |
| Protease Inhibitors | placental plasminogen activator inhibit | 2.0 | 2.5 |
| G Proteins | ras-related protein RAP-1B; GTP-bindi | 2.3 | 2.4 |
| Cell-Cell Adhesion Receptors | fibronectin receptor beta subunit (FNR | 0.7 | 2.4 |
| Members | casein kinase I alpha isoform (CKI-alpl | 1.0 | 2.4 |
| Diesterases | adenylate cyclase type VIII; ATP pyro [symbol] | 0.0 | 2.4 |
| Calpains | calcium-dependent protease small (re [symbol] | 0.5 | 2.4 |
| Interleukins & Interferons | interleukin-1 beta precursor (IL-1; IL1 [symbol] | 1.2 | 2.3 |
| Cell Surface Antigens | L-selectin precursor; lymph node hom [symbol] | 1.7 | 2.3 |
| Exocytosis Proteins | annexin IV (ANX4); lipocortin I; calpac [symbol] | 0.5 | 2.3 |
| G Proteins | ras-related protein RAB-7 | −4.2 | 2.3 |
| Other Metabolism Enzymes | mitochondrial aldehyde dehydrogenas [symbol] | 0.1 | 2.3 |
| Oncogenes & Tumor Suppressors | EVI2B protein precursor; ectropic viral | 0.2 | 2.2 |
| Receptors | corticotropin releasing factor receptor | 2.9 | 2.2 |
| Oncogenes & Tumor Suppressors | EB1 protein | 0.0 | 2.2 |
| RNA Polymerase | activated RNA polymerase II transcript | 1.9 | −2.2 |
| Proteins | coatomer delta subunit; delta-coat prot | 0.1 | 2.2 |
| Proteosomal Proteins | HUNTINGTIN INTERACTING PROTE | 0.2 | 2.2 |
| Chemokines | vascular endothelial growth factor prec | 0.1 | 2.1 |
| Members | cAMP-dependent protein kinase I alph [symbol] | 1.9 | 2.1 |
| Exocytosis Proteins | synaptotagmin V | 0.0 | 2.1 |
| Basic Transcription Factors | NF-AT4c | 1.2 | 2.0 |
| Other Metabolism Enzymes | 5-aminolevulinic acid synthase mitoch [symbol] | 0.4 | 2.0 |
| Activity Modulators | REGULATOR OF G-PROTEIN SIGNA | 0.9 | 2.0 |
| Calpains | calcium-dependent protease small (reg | 0.2 | 2.0 |
| Kinase Activators & Inhibitors | 14-3-3 PROTEIN EPSILON (MITOCH [symbol] | 1.3 | 2.0 |
| Activity Modulators | GTPase-activating protein (GAP); ras [symbol] | 1.3 | 2.0 |
| Housekeeping Genes | cytoplasmic beta-actin (ACTB) | 0.5 | 2.0 |
| Members | casein kinase II alpha' subunit (CK II); | 1.1 | 1.9 |
| Cell Surface Antigens | T-cell surface glycoprotein CD5 precur | 0.0 | 1.9 |
| Intracellular Protein Phosphatases | protein phosphatase 2C alpha isoform | 1.4 | 1.9 |
| Calpains | calpain p94 large (catalytic) subunit; c [symbol] | 0.0 | 1.9 |
| Proteins | cyclophilin 3 protein (CYP3); mitochon | 0.0 | 1.9 |
| Communication Proteins | neuromedin K receptor (NKR); neuroki | 0.1 | 1.9 |
| G Protein-Coupled Receptors | EBV-induced G-protein-coupled recept | 1.2 | 1.9 |
| Chemokines | granulins precursor (GRN); acrogranin | 0.8 | 1.9 |
| Calcium-Binding Proteins | calbindin; avian-type vitamin D-depen [symbol] | 0.4 | 1.8 |
| G Protein-Coupled Receptors | adenosine A2B receptor (ADORA2B) | 0.0 | 1.8 |
| Proteins | cation-dependent mannose-6-phospha | 0.0 | 1.8 |
| Amino Acid Metabolism | glycine dehydrogenase (decarboxytati [symbol] | 0.0 | 1.8 |
| Other Metabolism Enzymes | cytochrome P450 VA1 (CYP5A1) | 0.3 | 1.8 |
| Voltage-Gated Ion Channels | voltage-dependent anion-selective cha [symbol] | 1.4 | 1.8 |
| Tumover | vitamin K-dependent protein S | 0.0 | 1.8 |
| Proteosomal Proteins | proteasome component C3; macropair [symbol] | 1.9 | 1.8 |
| Hormone Receptors | CCKB-Cholecystokinin receptor | 0.6 | 1.8 |
| Effectors & Modulators | diacylglycerol kinase gamma (DGK-ga | −2.0 | 1.8 |
| Transporters | kidney UT2 urea transporter; SLC14A [symbol] | −1.1 | 1.8 |
| Calcium-Binding Proteins | calgizzarin; S100C protein; MLN70 | 0.0 | 1.8 |
| Tumover | fibrinogen B beta polypeptide | 0.0 | 1.8 |
| G Proteins | ras-related protein RAP-1B; GTP-bindi | 0.9 | 1.8 |
| Members | mitogen-activated protein kinase p38 ( | 3.3 | 1.8 |
| Housekeeping Genes | phospholipase A2 | 0.3 | 1.8 |
| Oncogenes & Tumor Suppressors | dek protein | 1.5 | 1.8 |
| Oncogenes & Tumor Suppressors | transforming protein rhoA H12 (RHO1 [symbol] | 0.0 | 1.7 |
| Nucleotide Metabolism | cytosolic thymidine kinase (TK1) | 1.0 | 1.7 |
| Neurotransmitter Receptors | M5-Muscarinic acetylcholine receptor | −1.1 | 1.7 |
| Chemokines | migration inhibitory factor-related prot [symbol] | 0.0 | 1.7 |
| Intracellular Protein Phosphatases | dual-specificity protein phosphatase 5; | 1.2 | 1.7 |
| Cysteine Proteases | cathepsin H precursor | 1.5 | 1.7 |
| Kinase Activators & Inhibitors | muscle/brain cAMP-dependent protein | 1.7 | 1.7 |
| Proteins | coatomer beta' subunit; beta'-coat prot | 0.0 | 1.7 |
| Oncogenes & Tumor Suppressors | N-ras; transforming p21 protein | 0.5 | 1.7 |
| Intracellular Protein Phosphatases | serine/threonine protein phosphatase [symbol] | 1.3 | 1.7 |
| Related Substances | peroxisomal acyl-coenzyme A oxidase | 0.6 | 1.7 |
| Chemokines | growth/differentiation factor 5 precurso | 0.0 | 1.7 |
| Communication Proteins | substance-K receptor (SKR); neurokini | 0.0 | 1.7 |
| Chemokines | FIBROBLAST GROWTH FACTOR-14 | 0.0 | 1.7 |
| Activity Modulators | REGULATOR OF G-PROTEIN SIGN [symbol] | 1.0 | 1.7 |
| Transcription Activators & Repressors | B-cell lymphoma 3-encoded protein (b | 0.9 | 1.7 |
| Oncogenes & Tumor Suppressors | c-myc oncogene | 1.7 | 1.6 |
| Oncogenes & Tumor Suppressors | retinoic acid receptor alpha | 1.5 | 1.6 |
| Hormones | STC (Stanniocalcin) | −1.2 | 1.6 |
| Communication Proteins | peripheral myelin protein 22 (PMP22); | 0.7 | 1.6 |
| Members | mitogen-activated protein kinase kinas | 1.2 | 1.6 |
| Effectors & Modulators | calpain inhibitor; calpastatin (CAST); s | 0.8 | 1.6 |
| Protease Inhibitors | alpha-1-antitrypsin precursor; alpha-1 [symbol] | 1.4 | 1.6 |
| Transcription Activators & Repressors | nuclear factor NF-kappa-B p100 subur | 0.9 | 1.6 |
| Transport Proteins | activator of RNA decay (ARD-1) | 0.1 | 1.6 |
| Serine Proteases | coagulation factor XII | 0.6 | 1.6 |
| Functionally Unclassified Proteins | DXS6673E protein; X-linked mental re [symbol] | 0.0 | 1.6 |
| Chemokines | macrophage-derived chemokine precu | 0.2 | 1.6 |
| Serine Proteases | coagulation factor IX | 0.0 | 1.6 |
| Calpains | calpain 2 large (catalytic) subunit; M-ty | 0.9 | 1.6 |
| Oncogenes & Tumor Suppressors | erythroblastosis virus oncogene homol | 0.0 | 1.6 |
| Proteins | COFILIN | 1.3 | 1.6 |
| Exocytosis Proteins | annexin II (ANX2); lipocortin II; calpac [symbol] | 2.1 | 1.6 |
| Death Kinases | interferon-inducible RNA-dependent p [symbol] | 1.8 | 1.6 |
| Associated Proteins | tyrosine-protein kinase lyn | 1.3 | 1.6 |
| Kinases | phospholipase C beta 2 (PLC-beta 2; [symbol] | 0.0 | 1.5 |
| Chemokines | placenta growth factors 1 + 2 (PLGF1 | 1.4 | 1.5 |
| Housekeeping Genes | hypoxanthine-guanine phosphoribosylt | 0.0 | 1.5 |
| Protein Modification Enzymes | platelet-activating factor acetylhydrola [symbol] | 0.0 | 1.5 |
| Complex Lipid Metabolism | cholinephosphate cytidylyltransferase; | 0.4 | 1.5 |
| Receptors | leukocyte platelet-activating factor rec [symbol] | 0.0 | 1.5 |
| Oncogenes & Tumor Suppressors | platelet-derived growth factor (PDGF) | 1.1 | 1.5 |
| Communication Proteins | glutamate decarboxylase 65-kDa isofo | 0.4 | 1.5 |
| Members | MAP kinase-activated protein kinase 2 | 4.4 | 1.4 |
| Other Receptors (by Activities) | zinc finger X-chromosomal protein (ZF | 1.6 | 1.4 |
| CDK Inhibitors | cyclin-dependent kinase inhibitor 1C ( [symbol] | −1.5 | 1.3 |
| Oncogenes & Tumor Suppressors | ezrin; cytovillin 2; villin 2 (VIL2) | −1.8 | 1.3 |
| Members | calcium/calmodulin-dependent protein | 3.2 | 1.2 |
| Basic Transcription Factors | guanine nucleotide-binding protein G- [symbol] | 2.3 | 1.2 |
| Members | ribosomal protein S6 kinase II alpha 3 | 1.7 | 1.2 |
| Basic Transcription Factors | CCAAT/enhancer binding protein gam [symbol] | 2.2 | 1.2 |
| Cell-Cell Adhesion Receptors | vitronectin receptor alpha subunit (VN [symbol] | 1.6 | 1.2 |
| Proteosomal Proteins | proteasome component C5; macropair | 2.0 | 1.2 |
| Transcription Activators & Repressors | 26S protease regulatory subunit 6A; T [symbol] | 1.9 | 1.1 |
| Communication Proteins | major prion protein precursor (PRP); P | 2.4 | 1.1 |
| Transcription Activators & Repressors | fli-1 oncogene; ergB transcription factc | 2.5 | 1.1 |
| Basic Transcription Factors | cellular nucleic acid binding protein (C [symbol] | 1.7 | 1.1 |
| Members | 5′-AMP-activated protein kinase cataly | 1.7 | 1.0 |
| G Proteins | ras-related protein RAB2 | 1.8 | 1.0 |
| Proteosomal Proteins | proteasome component C8; macropair | 1.7 | 1.0 |
| Effectors & Modulators | mothers against dpp homolog 7 (SMA [symbol] | 1.8 | 1.0 |
| Other Cell Cycle Proteins | sprouty 2 (SPRY2) | −2.3 | 1.0 |
| Members | dual specificity mitogen-activated prot [symbol] | 1.5 | 1.0 |
| Transcription Activators & Repressors | transcription factor Sp1 (TSFP1) | 1.9 | 1.0 |
| Related Substances | very-long-chain-specific acyl-CoA deh [symbol] | 2.2 | 0.9 |
| Members | ribosomal protein S6 kinase II alpha 1 | 1.5 | 0.9 |
| Communication Proteins | gamma-aminobutyric-acid receptor bel | −1.8 | 0.9 |
| Complex Lipid Metabolism | 3-ketoacyl-CoA thiolase peroxisomal p | 2.1 | 0.9 |
| G Proteins | ras-related C3 botulinum toxin substra [symbol] | 2.2 | 0.9 |
| Oncogenes & Tumor Suppressors | erythroblastosis virus oncogene homol | 1.7 | 0.9 |
| Other Immune System Proteins | grancalcin | 1.9 | 0.9 |
| Simple Lipid Metabolism | mitochondrial enoyl-CoA hydratase sh [symbol] | −1.6 | 0.9 |
| Other Metabolism Enzymes | platelet-activating factor acetylhydrola [symbol] | 1.7 | 0.8 |
| Intracellular Protein Phosphatases | PTPCAAX1 nuclear tyrosine phosphat [symbol] | 2.0 | 0.8 |
| Members | protein kinase C delta (NPKC-delta) | 2.0 | 0.8 |
| lnterleukins & Interferons | interleukin-18 precursor (IL-18); interfe | 1.5 | 0.8 |
| Cell-Cell Adhesion Receptors | NADH-ubiquinone oxidoreductase B18 | −1.6 | 0.8 |
| Xenobiotic Metabolism | dioxin-inducible cytochrome P450 1B1 | −2.7 | 0.8 |
| Effectors & Modulators | TRRAP protein | 1.8 | 0.8 |
| Xenobiotic Transporters | selenium-binding protein | 1.7 | 0.8 |
| Other Immune System Proteins | NEUTROPHIL DEFENSINS 1, 2 AND | −1.7 | 0.7 |
| Drug-Resistance Proteins | thiosulfate sulfurtransferase; rhodanes | 1.6 | 0.7 |
| Associated Proteins | Ink adaptor protein | 2.0 | 0.7 |
| Basic Transcription Factors | CCAAT-binding transcription factor sul | 2.6 | 0.7 |
| Oncogenes & Tumor Suppressors | C6.1A protein | −1.6 | 0.7 |
| Cell-Cell Adhesion Receptors | GAP JUNCTION ALPHA-8 PROTEIN | −2.1 | 0.7 |
| Intracellular Protein Phosphatases | serine/threonine phosphatase | −2.7 | 0.7 |
| CDK Inhibitors | trans-acting T-cell specific transcriptio [symbol] | 2.7 | 0.7 |
| Basic Transcription Factors | hypoxia-Inducible factor 1 alpha (HIF1 | 1.9 | 0.6 |
| Modulators | G protein-coupled receptor kinase GRI | 1.9 | 0.6 |
| Kinase Activators & Inhibitors | hint protein; protein kinase C inhibitor [symbol] | 3.1 | 0.6 |
| Communication Proteins | neuroendocurine convertase 1 precurso | −2.6 | 0.6 |
| Basic Transcription Factors | TGF-beta inducible early protein (TIE [symbol] | 1.8 | 0.6 |
| G Protein-Coupled Receptors | Mrg = mas-related | −2.1 | 0.5 |
| Basic Transcription Factors | MYELIN TRANSCRIPTION FACTOR | −1.9 | 0.5 |
| Kinases | phosphatidylinositol 3-kinase catalytic | 1.5 | 0.5 |
| Oncogenes & Tumor Suppressors | rhombotin-2 (RBTN2; RHOM2); cystei [symbol] | −1.7 | 0.5 |
| Communication Proteins | neuroendocrine convertase 1 precurso | −2.6 | 0.5 |
| Cyclins | G1/S-specific cyclin D3 (CCND3) | −1.6 | 0.5 |
| Facilitated Diffusion Proteins | aquaporin 4; WCH4; mercurial-insens [symbol] | −2.3 | 0.5 |
| Interleukins & Interferons | interleukin-1 alpha precursor (IL-1 alph | 1.9 | 0.4 |
| Hormone Receptors | guanine nucleotide-binding protein G( [symbol] | 2.0 | 0.4 |
| Transcription Activators & Repressors | zinc-finger DNA-binding protein | 3.1 | 0.4 |
| Kinases | phosphatidylinositol 4-kinase alpha (Pl | 2.0 | 0.4 |
| Ligases | excision repair protein ERCC6; Cocka [symbol] | −1.6 | 0.4 |
| Members | cAMP-dependent protein kinase beta- [symbol] | 1.6 | 0.4 |
| Oncogenes & Tumor Suppressors | nuclear pore complex protein 214 (NU [symbol] | −1.6 | 0.4 |
| Basic Transcription Factors | HOMEOBOX PROTEIN HB9 = HLXB [symbol] | −2.3 | 0.3 |
| Protease Inhibitors | endotheliai plasminogen activator inhil | 1.7 | 0.3 |
| Communication Proteins | flavin-containing amine oxidase A; mo | −2.0 | 0.3 |
| G Protein-Coupled Receptors | extracellular calcium-sensing receptor | −2.5 | 0.3 |
| Heat Shock Proteins | 70-kDa heat shock protein 1 (HSP70.1 | 2.2 | 0.3 |
| Xenobiotic Transporters | glutathione S-transferase theta 1 (GS [symbol] | 1.6 | 0.3 |
| Oncogenes & Tumor Suppressors | C-mos proto-oncogene serine/threonin | −7.9 | 0.3 |
| Basic Transcription Factors | human T-cell leukemia virus enhancer | 2.2 | 0.3 |
| Xenobiotic Transporters | beta-defensin 2 precursor (hBD2); skin | −3.4 | 0.3 |
| Hormone Receptors | melanocortin-4 receptor (MC4-R) | −2.2 | 0.3 |
| Kinases | phosphatidylinositol 3-kinase regulator | 2.7 | 0.3 |
| Interleukin & Interferon Receptors | interferon-alpha/beta receptor beta sut | 1.7 | 0.2 |
| Cell Surface Antigens | CD83 antigen precursor; cell surface p | 2.3 | 0.2 |
| Proteins | FOLLISTATIN 1 AND 2 PRECURSOR | −1.6 | 0.2 |
| Communication Proteins | sodium-dependent serotonin transport [symbol] | −3.1 | 0.2 |
| Extracellular Matrix Proteins | cartilage glycoprotein 39 precursor (G [symbol] | −1.6 | 0.2 |
| Cell-Cell Adhesion Receptors | SUSHI REPEAT-CONTAINING PROT | −1.5 | 0.2 |
| Proteins | golga2; golgin 95-kDa protein | −1.6 | 0.2 |
| Hormones | natriuretic peptide precursor B | −2.7 | 0.2 |
| Heat Shock Proteins | heat shock 90-kDa protein A (HSP90A | 2.9 | 0.1 |
| Calcium-Binding Proteins | S100 calcium-binding protein A7; psor [symbol] | −1.6 | 0.1 |
| Communication Proteins | dopamine beta-hydroxylase (DBH); do | −2.0 | 0.1 |
| Basic Transcription Factors | homeobox protein HOX-A4; HOX-1D; I | −1.9 | 0.1 |
| Associated Proteins | c-src kinase (CSK); protein-tyrosine ki [symbol] | 1.8 | 0.1 |
| Communication Proteins | 43-kDa postsynaptic protein; acetylcho | −2.5 | 0.1 |
| Interleukins & Interferons | interferon gamma precursor (IFN-gam [symbol] | 2.4 | 0.1 |
| Members | cAMP-dependent protein kinase type I | −1.7 | 0.0 |
| Housekeeping Genes | cytoplasmic beta-actin (ACTB) | −1.8 | 0.0 |
| Communication Proteins | parkin | −1.5 | 0.0 |
| Cell Cycle-Regulating Kinases | BUBR1 protein kinase | −2.2 | 0.0 |
| Oncogenes & Tumor Suppressors | thrombopoietin receptor precursor (TP | −1.6 | 0.0 |
| Other Cell Cycle Proteins | geminin | −1.5 | 0.0 |
| Oncogenes & Tumor Suppressors | c-rel proto-oncogene protein | 2.1 | 0.0 |
| Oncogenes & Tumor Suppressors | platelet-derived growth factor receptor | −1.9 | 0.0 |
| Cyclins | cyclin E2 | −1.6 | 0.0 |
| Members | lipid-activated protein kinase PRK1; P [symbol] | −2.2 | 0.0 |
| Associated Proteins | cell division cycle protein 25 nucleotide | −2.5 | 0.0 |
| G Proteins | ras-related protein RAB5A | 2.0 | 0.0 |
| Members | phosphorylase B kinase gamma cataly | −1.7 | 0.0 |
| Communication Proteins | sodium-dependent noradrenaline trans | −5.4 | 0.0 |
| Factors & Topoisomerases | MCM4 DNA replication licensing facto [symbol] | −1.6 | 0.0 |
| Ligases | xeroderma pigmentosum group D com | −2.1 | 0.0 |
| Diesterases | guanylate cyclase soluble beta-1 subu [symbol] | −2.4 | 0.0 |
| Other Apoptosis-Associated Proteins | IEX-1L anti-death protein; PRG-1; DIF | −1.6 | 0.0 |
| Recombination Proteins | recA-like protein HsRad51; DNA repai [symbol] | −1.7 | 0.0 |
| Communication Proteins | glutamate receptor 5 precursor (GLUR | −1.5 | 0.0 |
| Communication Proteins | proenkephalin A precursor | −2.2 | 0.0 |
| Communication Proteins | neuronal acetylcholine receptor protein | −2.2 | 0.0 |
| Communication Proteins | neuroendocrine convertase 2 precurso | −1.9 | 0.0 |
| Communication Proteins | membrane-bound & soluble catechol- [symbol] | −1.7 | 0.0 |
| Transcription Activators & Repressors | interferon regulatory factor 7 (IRF-7) | −1.7 | 0.0 |
| Transcription Activators & Repressors | ADA2-like protein | −1.5 | 0.0 |
| Translation | 14.5-kDa translational inhibitor protein | −1.8 | 0.0 |
| Cell Cycle-Regulating Kinases | homeobox protein hLim1: LHX1 | 1.6 | 0.0 |
| Transcription Activators & Repressors | nuclear factor kappa-B DNA binding s [symbol] | 1.7 | 0.0 |
| Drug-Resistance Proteins | soluble epoxide hydrolase (SEH); epo [symbol] | 1.6 | 0.0 |
| Interleukin & Interferon Receptors | interleukin-2 receptor gamma subunit [symbol] | 1.7 | 0.0 |
| Transcription Activators & Repressors | purine-rich single-stranded DNA-bindin | 2.4 | 0.0 |
| Receptors | N-sam; fibroblast growth factor recepto | 1.6 | 0.0 |
| Xenobiotic Transporters | microsomal stress 70 protein ATPase [symbol] | 1.8 | 0.0 |
| Chemokines | pleiotrophin precursor (PTN) + osteobl | −1.6 | 0.0 |
| Chemokines | Interferon gamma-induced protein pre [symbol] | −2.0 | 0.0 |
| Metalloproteinases | matrix metalloproteinase 7 (MMP7); m | 1.9 | 0.0 |
| Chemokines | T-cell-specific rantes protein precursor | −4.0 | 0.0 |
| Proteins | RAB GDP dissociation inihibitor beta ( [symbol] | 2.4 | 0.0 |
| Basic Transcription Factors | SEF2-1B PROTEIN; HELIX-LOOP-HE | 2.2 | 0.0 |
| Cell Surface Antigens | CD81 antigen; 26-kDa cell surface pro | 1.9 | 0.0 |
| Hormone Receptors | estrogen receptor beta (ER-beta) | 1.9 | 0.0 |
| Other Metabolism Enzymes | aldehyde dehydrogenase 2 (ALDH2) | 1.9 | 0.0 |
| Transcription Activators & Repressors | gamma-interferon-inducible protein IFI | 1.8 | 0.0 |
| Communication Proteins | sodium-dependent dopamine transport | 1.8 | 0.0 |
| G Proteins | GUANINE NUCLEOTIDE-BINDING P [symbol] | 1.7 | 0.0 |
| Members | RIBOSOMAL PROTEIN S6 KINASE ( [symbol] | 1.7 | 0.0 |
| Modulators | serine/threonine-protein kinase recept [symbol] | 1.7 | 0.0 |
| Communication Proteins | axonin-1 precursor; transient axonal gl | 1.6 | 0.0 |
| Oncogenes & Tumor Suppressors | ras-related protein R-ras2; ras-like prot | 1.6 | 0.0 |
| Basic Transcription Factors | HOMEOBOX PROTEIN MSX-2 (HOX- | 1.6 | 0.0 |
| Cell Surface Antigens | P-selectin precursor (SELP); granule n | 1.6 | 0.0 |
| Intracellular Protein Phosphatases | protein-tyrosine phosphatase MEG1 (F | 1.6 | 0.0 |
| Basic Transcription Factors | HOMEOBOX PROTEIN DLX-2 | 1.5 | 0.0 |
| Intracellular Protein Phosphatases | protein-tyrosine phosphatase MEG2 (F | 1.5 | 0.0 |
| Hormone Receptors | G-alpha interacting protein (GAIP) | −1.5 | 0.0 |
| Communication Proteins | synaptosomal-associated protein 25 (S [symbol] | −1.6 | 0.0 |
| Complex Lipid Metabolism | lysosomal acid lipase/cholesteryl ester | −1.6 | 0.0 |
| Chemokines | proliferation-inducing ligand (APRIL) | −1.6 | 0.0 |
| Intracellular Protein Phosphatases | protein-tyrosine phosphatase alpha pr [symbol] | −1.6 | 0.0 |
| Chemokines | beta chemokine Exodus 2 | −1.6 | 0.0 |
| Proteins | gamma-soluble NSF attachment prote | −1.6 | 0.0 |
| Voltage-Gated Ion Channels | dihydropyridine-sensitive I-type chann [symbol] | −1.7 | 0.0 |
| Basic Transcription Factors | TRANSCRIPTIONAL ENHANCER FA [symbol] | −1.7 | 0.0 |
| Complex Lipid Metabolism | lanosterol synthase (LSS); oxidosquale | −1.7 | 0.0 |
| Basic Transcription Factors | HOMEOBOX PROTEIN MEIS3 (MEIS | −1.7 | 0.0 |
| Basic Transcription Factors | POD1 - MESODERM-SPECIFIC BA [symbol] | −1.8 | 0.0 |
| Proteins | ER lumen protein retaining receptor 1; | −1.8 | 0.0 |
| Basic Transcription Factors | PITX2 OR RIEG OR RGS - PITUITAR | −1.9 | 0.0 |
| Communication Proteins | sodium- & chloride-dependent GABA t [symbol] | −1.9 | 0.0 |
| Transcription Activators & Repressors | SMOOTH MUSCLE CELL LIM PROTE [symbol] | −2.0 | 0.0 |
| Hormone Receptors | guanine nucleotide-binding protein G [symbol] | −2.0 | 0.0 |
| Cell Surface Antigens | CD4O | −2.1 | 0.0 |
| Basic Transcription Factors | MYOGENIC FACTOR MYF-5 | −2.3 | 0.0 |
| Basic Transcription Factors | SKELETAL MUSCLE LIM-PROTEIN 1 | −2.3 | 0.0 |
| Neurotransmitter Receptors | gamma-aminobutyric-acid receptor al [symbol] | −2.3 | 0.0 |
| Complex Lipid Metabolism | lipoprotein lipase precursor (LPL) | −3.3 | 0.0 |
| Heat-Shock Proteins | 27-kDa heat-shock protein (HSP27); s [symbol] | 2.8 | 0.0 |
| Kinases | phospholipase C beta 2 (PLC-beta 2; [symbol] | −1.9 | 0.0 |
| G Proteins | Ral A: GTP-binding protein | 3.1 | 0.0 |
| Other Apoptosis-Associated Proteins | poly(ADP-ribose) polymerase (PARP; | −2.0 | 0.0. |
| Hormone Receptors | histamine H1 receptor (HRH1) | −2.3 | 0.0 |
| Communication Proteins | secretogranin II precursor (SGII); chro [symbol] | −1.6 | 0.0 |
| Interleukin & Interferon Receptors | interleukin-7 receptor alpha subunit pr [symbol] | 2.2 | −0.8 |
| Extracellular Matrix Proteins | lumican precursor (LUM); keratan sulf [symbol] | −2.2 | −1.0 |
| G Proteins | ADP-ribosylation factor 1 | −1.7 | −1.0 |
| Metalloproteinases | matrix metalloproteinase 8 (MMP8); n [symbol] | 1.6 | −1.0 |
| Proteins | syntaxin 5 (STX5) | −1.9 | −1.0 |

TABLE 8
GENE CHANGES INDUCED BY DENGUE VIRUS IN VITRO IN
HUMAN LYMPHOID CELLS - ARRAY I

| | | | |
|---|---|---|---|
| Xenobiotic Transporters | glutathione peroxidase (GSHPX1; GP) | −1.5 | −1.0 |
| Communication Proteins | neuropeptide-Y precursor (NPY) | −1.6 | −1.0 |
| Oncogenes & Tumor Suppressors | A-raf proto-oncogene serine/threonine [symbol] | −1.5 | −1.0 |
| CDK Inhibitors | Sp2 protein | 1.6 | −1.0 |
| Chemokines | FIBROBLAST GROWTH FACTOR-10 | −2.2 | −1.1 |
| Energy Metabolism | alcohol dehydrogenase 5 chi polypepti [symbol] | 1.5 | −1.1 |
| Proteins | Golgi SNARE; GS27 | −1.6 | −1.1 |
| Basic Transcription Factors | HOMEOBOX PROTEIN EMX2 | −1.5 | −1.2 |
| Proteins | apolipoprotein E precursor (APOE) | −1.8 | −1.2 |
| Chemokines | uromodulin; Tamm-Horsfall urinary gly | −1.7 | −1.2 |
| Hormone Receptors | neuron-derived orphan receptor 1 (NO | 1.5 | −1.2 |
| Oncogenes & Tumor Suppressors | mas proto-oncogene | −1.8 | −1.2 |
| Transcription Activators & Repressors | early growth response protein 1 (hEGF [symbol] | −1.7 | −1.3 |
| Transcription Activators & Repressors | interferon regulatory factor 7 (IRF-7) | −2.3 | −1.3 |
| Other Cell Cycle Proteins | DNA-binding protein Inhibitor ID-1; Id-1 | −3.8 | −1.3 |
| Extracellular Matrix Proteins | collagen 10 alpha 1 subunit (COL10A1 | 1.6 | −1.4 |
| Oncogenes & Tumor Suppressors | C-maf transcription factor | −2.4 | −1.4 |
| Basic Transcription Factors | HOMEOBOX PROTEIN HOX-B1 = HC | 2.3 | −1.4 |
| Other Immune System Proteins | granzyme M precursor (GZMM); met-a | −1.5 | −1.5 |
| Effectors & Modulators | mothers against dpp homolog 2 (hMA [symbol] | 0.3 | −1.5 |
| Hormone Receptors | vitamin D3 receptor (VDR) | 0.2 | −1.5 |
| Oncogenes & Tumor Suppressors | ras-related protein RAB-8; oncogene c | −1.3 | −1.5 |
| Interleukin & Interferon Receptors | interleukin 10 receptor (IL-10R) | 0.9 | −1.5 |
| G Protein-Coupled Receptors | probable G-protein-coupled receptor 9 [symbol] | 0.0 | −1.5 |
| Members | tyk2 non-receptor protein tyrosine kina | 0.0 | −1.5 |
| Neurotransmitter Receptors | gamma-aminobutyric-acid receptor rh [symbol] | 1.1 | −1.5 |
| Transcription Activators & Repressors | signal transducer and activator of tran [symbol] | −1.3 | −1.5 |
| RNA Polymerase | transcription initiation factor TFIID 31- [symbol] | 2.3 | −1.5 |
| Communication Proteins | tryptophan 5-hydroxylase (TRPH); tryp | 0.7 | −1.6 |
| Hormones | cellular retinoic acid-binding protein II [symbol] | 0.2 | −1.6 |
| Cyclins | cyclin K | 0.0 | −1.6 |
| Oncogenes & Tumor Suppressors | breakpoint cluster region protein (BCR | 0.0 | −1.6 |
| Basic Transcription Factors | FORKHEAD-RELATED TRANSCRIPT | 0.0 | −1.6 |
| Transcription Activators & Repressors | TRAF-interacting protein (I-TRAF) + T [symbol] | 0.3 | −1.6 |
| Diesterases | adenylate cyclase VII; ATP pyrophospl | 0.0 | −1.6 |
| Other Cell Cycle Proteins | 40S ribosomal protein S19 (RPS19) | −2.9 | −1.6 |
| Diesterases | guanylate cyclase soluble beta-1 subu [symbol] | 0.1 | −1.6 |
| Protein phosphatase Receptors | protein-tyrosine phosphatase X precur [symbol] | 0.1 | −1.6 |
| Members | protein-tyrosine phosphatase LC-PTP; | 0.0 | −1.6 |
| Communication Proteins | acetylcholinesterase precursor (ACHE [symbol] | −1.2 | −1.6 |
| Chemokines | amphiregulin (AR); colorectum cell-de [symbol] | 0.0 | −1.6 |
| Oncogenes & Tumor Suppressors | proto-oncogene tyrosine-protein kinas [symbol] | 0.0 | −1.7 |
| Effectors & Modulators | diacylglycerol kinase zeta (DAG kinas [symbol] | 0.0 | −1.7 |
| Basic Transcription Factors | HOMEOBOX PROTEIN SIX1 | 1.0 | −1.7 |
| Neurotransmitter Receptors | gamma-aminobutyric-acid receptor bel | 1.0 | −1.7 |
| Cell Surface Antigens | cytotoxic T-lymphocyte protein 4-1 pre | 0.0 | −1.7 |
| Chemokines | FIBROBLAST GROWTH FACTOR-11 | 0.9 | −1.7 |
| Proteins | clathrin coat assembly protein AP17; p | 0.0 | −1.7 |
| Basic Transcription Factors | PAIRED MESODERM HOMEOBOX P | −1.0 | −1.7 |
| Housekeeping Genes | HLA class I histocompatibility antigen [symbol] | 0.0 | −1.7 |
| Other Cell Cycle Proteins | transducer of erbB2 (TOB) | −1.3 | −1.8 |
| Calcium-Binding Proteins | S100 calcium-binding protein A1; S-10 | 0.4 | −1.8 |
| Voltage-Gated Ion Channels | voltage-gated potassium channel prote | 0.3 | −1.8 |
| Activity Modulators | rap1 GTPase-GDP dissociation stimula [symbol] | 0.1 | −1.8 |
| Simple Carbohydrate Metabolism | galactoside 2-1-fucosyltransferase 1; G | 0.0 | −1.8 |
| Oncogenes & Tumor Suppressors | c-kit proto-oncogene; mas/stem cell g | 0.0 | −1.8 |
| CDK Inhibitors | cyclin-dependent kinase 4 inhibitor B ( [symbol] | 0.0 | −1.8 |
| Oncogenes & Tumor Suppressors | (tight junction protein zonula occiudens | −1.1 | −1.8 |
| Transcription Activators & Repressors | interleukin enhancer binding factor 2 ( [symbol] | 0.0 | −1.8 |
| Basic Transcription Factors | EARLY GROWTH RESPONSE PROT | −2.3 | −1.8 |
| Ligand-Gated Ion Channels | P2X purinoceptor 7 (P2X7); ATP rece [symbol] | 0.0 | −1.8 |
| Other Apoptosis-Associated Proteins | inhibitor of apoptosis protein1 (HIAP1; | 0.1 | −1.8 |
| Effectors & Modulators | zyxin + zyxin-2 | −1.0 | −1.8 |
| Oncogenes & Tumor Suppressors | C-fes proto-oncogene | 0.0 | −1.8 |
| Death Receptors | adenosine A1 receptor (ADORA1) | −3.3 | −1.9 |
| G Proteins | ADP-ribosylation factor 1 | 0.0 | −1.9 |
| Chemokines | melanoma-derived growth regulatory [symbol] | 0.0 | −1.9 |
| Chemokines | teratocarcinoma-derived growth factor | 0.0 | −1.9 |
| Intracellular Protein Phosphatases | protein phosphatase 2A B'alpha1 regul [symbol] | 0.3 | −1.9 |
| Cell Surface Antigens | T-cell surface glycoprotein CD1A prec [symbol] | 0.0 | −1.9 |
| Basic Transcription Factors | SOX-9 PROTEIN | 0.0 | −1.9 |
| Proteins | microsomal triglyceride transfer protein | 0.2 | −1.9 |
| Other Immune System Proteins | ficolin 1 (FCN1) + FCN2; serum lectin | 0.6 | −1.9 |
| Cell Surface Antigens | cell surface glycoprotein A15; T-cell a [symbol] | 0.0 | −1.9 |
| Xenobiotic Metabolism | uridine diphosphate glycosyltransferas [symbol] | 0.0 | −1.9 |
| Amino- & Carboxypeptidases | tripeptidyl-peptidase 1 precursor; tripep [symbol] | 0.0 | −2.0 |
| Basic Transcription Factors | T-cell-specific transcription factor 1 (T- | −1.2 | −2.0 |
| Cell Surface Antigens | complement receptor type 1 precursor | 0.0 | −2.0 |
| Oncogenes & Tumor Suppressors | elk-1; ets-related proto-oncogene | 0.0 | −2.0 |
| Communication Proteins | myelin-associated glycoprotein precurs [symbol] | 0.0 | −2.0 |
| Basic Transcription Factors | HATH-1 - ATONAL HOMOLOG | −1.1 | −2.0 |
| Receptors | transforming growth factor beta recept [symbol] | 0.2 | −2.0 |
| Basic Transcription Factors | NF-ATc | 0.4 | −2.0 |
| Energy Metabolism | mitochondrial trifunctional protein enoy | 0.6 | −2.0 |
| Transcription Activators & Repressors | C-ets-2 | 0.0 | −2.1 |
| Activity Modulators | regulator of G-protein signalling 2 (RG | 0.3 | −2.1 |
| Basic Transcription Factors | SINGLE-MINDED HOMOLOG 2 | 0.0 | −2.1 |
| Chemokines | keratinocyte growth factor (KGF); fibro | 0.2 | −2.1 |
| G Proteins | ras-related protein RAB-7 | 0.6 | −2.1 |
| Cell-Cell Adhesion Receptors | leukocyte adhesion glycoprotein LFA-1 | −3.8 | −2.1 |
| Oncogenes & Tumor Suppressors | moesin-ezrin-radixin-like protein (MER | −1.3 | −2.2 |
| Chemokines | endothelin 3 (EDN3; ET3) | 0.0 | −2.2 |
| Hormone Receptors | D(4) DOPAMINE RECEPTOR (D(2C) | 0.0 | −2.2 |
| Basic Transcription Factors | MAX—HELIX-LOOP-HELIX ZIPPER | 0.0 | −2.2 |
| Housekeeping Genes | 23-kDa highly basic protein; 603 ribos [symbol] | 0.0 | −2.3 |
| Kinase Activators & Inhibitors | 14-3-3 protein sigma; stratifin; epithelia | −1.6 | −2.3 |
| Diesterases | 3'5'-cAMP phosphodiesterase HPDE4A | 0.0 | −2.3 |
| Housekeeping Genes | brain-specific tubulin alpha 1 subunit [symbol] | −1.9 | −2.3 |
| Housekeeping Genes | ubiquitin | 1.8 | −2.3 |
| Hormone Receptors | somatostatin receptor type 4 (SS4R) | −1.3 | −2.3 |
| Transcription Activators & Repressors | tristetraproline (TTP); TIS11; ZFP36; g [symbol] | 1.4 | −2.3 |
| Oncogenes & Tumor Suppressors | colorectal mutant cancer protein (MCC | 0.0 | −2.3 |
| Complex Carbohydrate Metabolism | alpha-L-iduronidase precursor | 0.0 | −2.4 |
| Members | c-jun N-terminal kinase 2 (JNK2); JNK | 0.7 | −2.4 |
| Other Immune System Proteins | LDL-associated phospholipase A2 | 1.3 | −2.4 |
| Housekeeping Genes | brain-specific tubulin alpha 1 subunit [symbol] | 0.0 | −2.4 |
| Proteosomal Proteins | proteasome inhibitor HPI31 subunit | 0.5 | −2.5 |
| Oncogenes & Tumor Suppressors | prohibitin (PHB) | 0.0 | −2.5 |
| Intracellular Protein Phosphatases | leukocyte antigen-related protein prec [symbol] | 0.0 | −2.6 |
| Receptors | C5a anaphylatoxin receptor (C5AR); C | −1.8 | −2.6 |
| Basic Transcription Factors | Runt domain-containing protein PEBP; | −1.3 | −2.7 |
| Intracellular Protein Phosphatases | dual-specificity protein phosphatase 6; | 1.1 | −2.7 |
| Cell Surface Antigens | platelet glycoprotein IX | 1.3 | −2.8 |
| Chemokines | hepatocyte growth factor activator (HG | −1.7 | −2.8 |
| Diesterases | adenylate cyclase type I; ATP pyropho | 0.0 | −2.8 |
| Transcription Activators & Repressors | orphan hormone nuclear receptor | 0.6 | −2.8 |
| Cell Surface Antigens | T-cell surface glycoprotein CD1D prec [symbol] | 0.2 | −2.8 |
| Transcription Activators & Repressors | raf-responsive zinc finger protein | 0.0 | −3.0 |
| Caspases | caspase-10 precursor (CASP10); ICE- [symbol] | −3.6 | −3.0 |
| Transcription Activators & Repressors | AP4 basic helix-loop-helix DNA-binding | 0.5 | −3.3 |
| Chemokines | brain-derived neurotrophic factor (BDN | 0.0 | −3.5 |
| Housekeeping Genes | HLA class I histocompatibility antigen [symbol] | −1.2 | −3.6 |
| Chemokines | hepatocyte growth factor-like protein; r | 0.0 | −3.7 |
| Oncogenes & Tumor Suppressors | neurofibromatosis protein type I (NF1); | 0.0 | −4.0 |

TABLE 9
GENES NOT EXPRESSED IN UNTREATED PBMC BUT EXPRESSED
UPON TREATMENT WITH AGENTS

Gene name
angiopoietin 1 receptor precursor; tyrosine-protein kinase receptor TIE-2; tyrosine-protein kinase receptor TI
fibroblast growth factor receptor 3 precursor (FGFR3); JTK4 + fibroblast growth factor receptor 2 precursor
ephrin type-B receptor 2 precursor; tyrosine-protein kinase receptor EPH-3; DRT; HEK; ERK
tyrosine kinase receptor HEK; ephrin type-A receptor 3 precursor; tyrosine-protein kinase receptor ETK1
interferon-gamma (IFN-gamma) receptor beta subunit precursor; IFN-gamma accessory factor 1 (AF1); IFN
synaptic vesicle amine transporter (SVAT); monoamine transporter; vesicular amine transportert 2 (VAT2)
rap1 GTPase activating protein 1 (RAP1GAP)
melanotransferrin precursor; melanoma-associated antigen p97
lactotransferrin precursor; lactoferrin
sodium/glucose cotransporter 2; (Na+/glucose cotransporter 2); low-affinity sodium-glucose cotransporter
epidermal growth factor receptor kinase substrate EPS8
titin
inhibitor of apoptosis protein 3 (API3: IAP3); X-linked inhibitor of apotosis protein (X-linked IAP; XIAP); IAP-
thrombin receptor (TR); F2R; PAR1
Rad50
protein kinase C gamma type (PKC-gamma)
cGMP-inhibited 3'/5'-cyclic phosphodiesterase A (CGI-PDE A)
protein kinase C zeta type (NPKC-zeta)
neogenin
c-kit proto-oncogene; mast/stem cell growth factor receptor precursor (SCFR); CD117 antigen
sodium-dependent proline transporter
G protein-activated inward rectifier potassium channel 2 (GIRK2); KATP-2; BIR1; KIR32
DNA damage repair & recombination protein 52 (RAD52)
thrombopoietin receptor precursor (TPOR); myeloproliferative leukemia protein (MPL)
sodium-dependent noradrenaline transporter; norepinephrine transporter (NET)
alpha-fetoprotein precursor; alpha-fetoglobulin
L-myc proto-oncogene (MYCL1)
glutathione synthetase (GSH synthetase; GSH-S); glutathione synthase
vesicular acetylcholine transporter VAChT)
epidermal growth factor receptor (EGFR)
tyrosine kinase tnk1
C-mos proto-oncogene serine/threonine-protein kinase
breast cancer type 2 susceptibility protein (BRCA2)
p53 cellular tumor antigen
homeobox protein HOX-11; tcl-3 proto-oncogene
prohibitin (PHB)
moesin-ezrin-radixin-like protein (MERLIN); schwannomin (SCH); neurofibromatosis 2 (NF2)
Wilms' tumor protein (WT33; WT1)
transforming growth factor-beta signaling protein 1 (BSP1); mothers against dpp homolog (MAD); MADR1; l
calcium/calmodulin-dependent 3′,5′-cyclic nucleotide phosphodiesterase 1A (CAM-PDE1A); HCAM-1
tumor suppressor protein DCC precursor; colorectal cancer suppressor
ciliary neurotropic factor receptor (CNTFR)
ephrin A3 precursor (EFNA3); EPH-related receptor tyrosine kinase ligand 3 (EPLG3); LERK3; EHK1 ligand
transforming growth factor-beta 3 (TGF-beta3)
pim-1 proto-oncogene
mu-type opioid receptor (MOR-1)
matrix metalloproteinase 2 (MMP2); 72-kDa gelatinase A; 72-kDa type IV collagenase precursor (CLG4A);
P2X purinoceptor 6 (P2X6); P2XM
DNA cytosine-5-methyltransferase (DNA metase; MCMT)
sodium/hydrogen exchanger 3 (Na+/H+exchanger 3; NHE3)
neurofibromatosis protein type I (NF1); neurofibromin
serotransferrin precursor; siderophilin; beta-1-metal binding globulin
ephrin type-A receptor 2 precursor; epithelial cell kinase (ECK); tyrosine-protein kinase receptor ECK
bystin
NT-3 growth factor receptor precursor (NTRK3); C-trk tyrosine kinase (TRKC)
sulfate transporter; diastrophic dysplasia protein
aurora- & IPL1-like midbody-associated protein kinase 1 (AIM1); ARK2
myotonic dystrophy protein kinase-like protein
leukocyte tyrosine kinase receptor precursor (LTK)
c-jun N-terminal kinase 1 (JNK1); JNK46
helix-loop-helix protein HLH 1R21; DNA-binding protein inhibitor Id-3; HEIR-1
calcium/calmodulin-dependent protein kinase type II beta subunit (CAM-kinase II beta; CAMK-II beta)
5-hydroxytryptamine 1A receptor (5HT1A); serotonin receptor
focal adhesion kinase (FADK); proline-rich tyrosine kinase 2 (PYK2)
ribosomal protein kinase B (RSKB)
N-myc proto-oncogene
liver glucose transporter 2
tyrosine kinase receptor tie-1 precursor
serine/threonine-protein kinase NEK3; NIMA-related protein kinase 3; HSPK 36
5-hydroxytryptamine 1D receptor (5-HT-1D; HTR1D); serotonin receptor
wee1Hu CDK tyrosine 15-kinase; wee-1-like protein kinase
ras-related protein RAB4A
bullous pemphigoid antigen 1 (BPAG1; BPA); hemidesmosomal plaque protein
6-O-methylguanine-DNA methyltransferase (MGMT); methylated-DNA-protein-cysteine methyltransferase
tyrosine-protein kinase receptor UFO precursor; axi oncogene
cyclin-dependent protein kinase 2 (CDK2); p33 protein kinase
glutamate decarboxylase 65-kDa isoform; 65-kDa glutamic acid decarboxylase (GAD-65); GAD2
cytochrome P450 IVB1 (EC 1.14.14.1) (P450-HP)
neurotensin/neuromedin N precursor (NT/NMN)
neuromedin B precursor
glutamate decarboxylase 67-kDa isoform; 67-kDa glutamic acid decarboxylase (GAD-67); GAD1
CDC25C; M-phase inducer phosphatase 3
43-kDa postsynaptic protein; acetylcholine receptor-associated 43-kDa protein; RAPSYN
cytochrome P450 IA2 (P450-P3) (P450-4)
chroline O-acetyltransferase (CHAT); choactase; choline acetylase
leptin precursor; obesity factor; obese protein
phenylalanine-4-hydroxylase (PAH); phe-4-monooxygenase
glutamate receptor subunit epsilon 3 precursor (GRIN2C); N-methyl D-aspartate receptor subtype 2C (NMD
G protein-activated inward rectifier potassium channel 3 (GIRK3); KIR3.3
geminin
gamma-aminobutyric-acid receptor gamma-2 subunit precursor (GABA(A) receptor)
extracellular signal-regulated kinase 1 (ERK1; p44-ERK1); microtubule-associated protein 2 kinase; insulin-
E2F dimerization partner 1; DRTF1-polypeptide 1 (DP1)
cAMP-dependent protein kinase type II alpha regulatory subunit (PRKAR2A; PKR2)
glycine receptor beta subunit precursor (GLRB)
ataxia-telangiectasia group D-associated protein
brain-derived neurotrophic factor (BDNF)/NT-3 growth factors receptor precursor; TRKB tyrosine kinase rec
myelin proteolipid protein (PLP); lipophilin
cell division protein kinase 5 (CDK5); tau protein kinase II catalytic subunit (TPKII catalytic subunit); serine/l
S100 calcium-binding protein A1; S-100 protein alpha chain
glutamate receptor 1 precursor (GLUR-1); GLUR-A; GluH1; ionotropic glutamate receptor ampa1
gamma-aminobutyric-acid receptor beta-1 subunit precursor (GABA(A) receptor)
G protein-activated inward rectifier potassium channel 1 (GIRK1); KIR31
metabotropic glutamate receptor 1 precursor (GRM1; MGLUR1)
DNA fragmentation factor 45 (DFF45)
cell division protein kinase 4; cyclin-dependent kinase 4 (CDK4); PSK-J3
aurora-related kinase 1 (ARK1)
neurotrophic tyrosine kinase receptor-related 3; TKT precursor
thyroxine-binding globulin precursor; T4-binding globulin
cadherin1 (CDH1); epithelial cadherin precursor (E-cadherin; CDHE); uvomorulin (UVO); CAM 120/80
matrix metalloproteinase 7 (MMP7); matrilysin
nociceptin receptor; orphanin FQ receptor; kappa-type 3 opioid receptor (KOR-3)
cyclin-dependent kinase regulatory subunit 1 (CKS1)
INT-2 proto-oncogene protein precursor (fibroblast growth factor-3) (FGF-3) (HBGF-3)
neuroendocrine convertase 1 precursor (NEC 1); prohormone convertase 1 (PC1); proprotein convertase 1
matrix metalloproteinase 13 (MMP13); collagenase 3 precursor
neuronal acetylcholine receptor protein alpha 6 subunit precursor
growth arrest & DNA-damage-inducible protein 45 gamma (GADD45 gamma)
mas proto-oncogene
androgen receptor (AR)
early growth response protein 3 (EGR3); zinc finger protein pilot
activin type I receptor; serine/threonine-protein kinase receptor R2 (SKR2); activin receptor-like kinase 4 (A
hepatic leukemia factor (HLF)
GABA-B receptor 2 subunit (GABA-BR2)
pancreatitis-associated protein 1 precursor
cell surface glycoprotein MUC18; melanoma-associated antigen A32; CD146 antigen; melanoma adhesion
puromycin-sensitive aminopeptidase (PSA)
cyclin-dependent kinase 4 inhibitor B (CDKN2B); p14-INK4B; multiple tumor suppressor 2 (MTS2)
acrosin precursor
acrosin-trypsin inhibtor II precursor; HUSI II
ras-related protein RAB3B
proenkephalin A precursor
serine/threonin-protein kinase PAK-beta; p21-activated kinase 3
inhibin alpha subunit precursor (INHA)
NF-kappaB transcription factor p65 subunit; RELA; NFKB3
AP4 basic helix-loop-helix DNA-binding protein
met proto-oncogene; hepatocyte growth factor receptor precursor (HGF-SF receptor)
triiodothyronine receptor; thyroid hormone receptor (THRA1); v-erbA-related protein ear-1
serine/threonine-protein kinase KKIALRE
platelet membrane glycoprotein IIB precursor (GP2B); integrin alpha 2B (ITGA2B); CD41 antigen
high-affinity interleukin-8 receptor A (IL-8R A); IL-8 receptor type 1; CDW128
cyclin A1 (CCNA1)
follicle stimulating hormone receptor (FSHR); follitropin receptor
cadherin 11 precursor (CDH11); osteoblast-cadherin (OB-cadherin); OSF4
voltage-gated potassium channel protein KV12; HUKIV; HBK5; RBK2; NGK1
ADA2-like protein
integrin alpha 3 (ITGA3); galactoprotein B3 (GAPB3); VLA3 alpha subunit; CD49C antigen
interleukin-11 (IL-11); adipogenesis inhibitory factor (AGIF)
dopamine beta-hydroxylase (DBH); dopamine-beta-monooxygenase precursor
vascular endothelial growth factor receptor 2 precursor (VEGFR2); kinase Insert domain receptor (KDR); FL
autocrine motility factor receptor (AMF receptor; AMFR)
colon carcinoma kinase 4 precursor (CCK4) + transmembrane receptor PTK7
ran GTPase activating protein 1 (RANGAP1)
D2 dopamine receptor (DRD2)
oligophrenin 1
mitochondrial cytochrome P450 XIA1 precursor; P450(SCC); cholesterol side-chain cleavage enzyme; chol
matrix metalloproteinase 1 (MMP1); Interstitial collagenase precursor (CLG); fibroblast collagenase
kidney glomeruli chloride channel; CIC-5
recoverin; cancer-associated retinopathy protein (CAR protein)
gamma-aminobutyric-acid receptor pi subunit precursor (GABA(A) receptor)
myelin basic protein (MBP)
synaptosomal-associated protein 25 (SNAP-25); super protein (SUP)
parkin
global transcription activator SNF2L1
ephrin A4 precursor (EFNA4); EPH-related receptor tyrosine kinase ligand 4 (EPLG4); LERK4
cyclin-dependent kinase inhibitor 1C (CDKN1C); p57-KIP2
gamma-aminobutyric-acid receptor epsilon subunit precursor (GABA(A) receptor)
25-hydroxy vitamin D3 1-alpha hydroxylase mitochondrial precursor (VD3 1A hydroxylase); 25-OHD-1 alph [symbol]
nociceptin precursor; orphanin FQ; PPNOC
LYL-1 protein
lissencephalin X; doublecortin (DCX)
neuroglycan C precursor
keratinocyte growth factor (KGF); fibroblast growth factor 7 (FGF7)
PCAF-associated factor 65 alpha
neuroendocrine protein 7B2 precursor; secretory granule endocrine protein I; secretogranin V
transcription factor GATA-4; GATA binding factor-4
glutamate receptor 2 precursor (GLUR2); GLUR-B; GLUR-K2
neuronatin; brain-specific mammalian developmental gene
NAD(P)H dehydrogenase; quinone reductase; DT-diaphorase; azoreductase; phylloquinone reductase; men
polymorphic arylamine N-acetyltransferase (PNAT) + monomorphic (MNAT)
ERBB4 receptor protein-tyrosine kinase; Her4 tyrosine kinase-EGF receptor related
calcium-activated potassium channel beta subunit; maxi K channel beta subunit; BK channel beta subunit; [symbol]
integrin alpha 6 precursor (ITGA6); VLA6; CD49F antigen
glia maturation factor beta (GMF-beta)
cytokine humig; Interferon-gamma-induced monokine (MIG)
platelet-derived growth factor receptor alpha subunit (PDGFRA); CD140A antigen
macrophage-stimulating protein receptor precursor (MSP receptor); p185-RON; CD136 antigen
acetylcholinesterase precursor (ACHE)
hepatocyte growth factor (HGF); scatter factor (SF); hepatopoeitin A
guanine nucleotide regulatory protein tim1
microsomal glutathione S-transferase 12 (GST12; MGST1)
B-cell differentiation CD72 antigen; Lyb-2
MCM3 DNA replication licensing factor; DNA polymerase alpha holoenzyme-associated protein P1; RLF be
CXC chemokine precursor
phosphatidylinositol 3-kinase regulatory beta subunit (PI3-kinase p85-beta subunit; PTDINS-3-kinase p85-b [symbol]
neuropeptide Y receptor type 1 (NPY1R)
C-fes proto-oncogene
neuronal pentraxin II precursor (NP2)
neural-cadherin precursor (N-cadherin; NCAD); cadherin 2 (CDH2)
galanin receptor type 1 (GALNR1; GALR1)
B-myb
serine/threonine-protein kinase NEK2; NIMA-related protein kinase 2; NIMA-like protein kinase 1; HSPK 21
replication factor C 36-kDa subunit (RFC36); activator 1 36-kDa subunit
metabotropic glutamate receptor 5 precursor (GRM5; MGLUR5)
canalicular multispecific organic anion transporter; multidrug resistance-associated protein 2 (MRP2); canal
voltage-gated potassium channel protein KV14; HUKII; HBK4; HPCN2
extracellular signal-regulated kinase 4 (ERK4); MAP kinase 4 (MAPK4; p63-MAPK); PRKM4
dual-specificity protein phosphatase 9; mitogen-activated protein kinase phosphatase 4 (MAP kinase phosp
tumor suppressor LUCA1; hyaluronoglucosaminidase (HYAL1)
nuclear factor I (NFI); NFI-X
bone proteoglycan II precursor (PGS2); decorin (DCN)
CCAAT/enhancer binding protein alpha (C/EBP alpha)
bub1 mitotic checkpoint kinase
plasma membrane calcium-transporting ATPase isoform 2 (PMCA2); ATP2B2; calcium pump;
neuro epithelioma transforming gene 1 (NEP1; NET1): guanine nucleotide regulatory protein
V(D)J recombination activating protein 1 (RAG1)
5-hydroxytryptamine 2A receptor (5HT2A); serotonin receptor type 2
ets domain protein elk-3; NET; SRF accessory protein 2 (SAP2)
cyclin E2
growth arrest & DNA-damage-inducible protein 45 beta (GADD45 beta)
cellular retinoic acid-binding protein II (CRABP2)
thrombospondin 2 precursor (THBS2; TSP2)
beta-defensin 2 precursor (hBD2); skin-antimicrobial peptide 1 (SAP1)
soluble epoxide hydrolase (SEH); epoxide hydratase; cytosolic epoxide hydrolase (CEH); EPHX2
dimethylaniline monooxygenase (N-oxide forming) 1 (EC 1.14.13.8); fetal hepatic flavin-containing monoox
neuronal acetylcholine receptor protein beta 4 subunit precursor (CHRNB4; NACHRB4)
glutamate (NMDA) receptor subunit epsilon 2 precursor; N-methyl D-aspartate receptor subtype 2B (NMDAI
neuromedin K receptor (NKR); neurokinin B receptor NK-3 receptor (NK-3R)
amphiphysin (AMPH)
calcitonin receptor (CTR; CALCR)
transcription intermediary factor 1 beta (TIF1B); KRAB-associated protein 1 (KAP1)
activator of RNA decay (ARD-1)
integrin alpha 1 (ITGA1); laminin & collagen receptor; VLA1; CD49A antigen
adenylate cyclase type II; ATP pyrophosphate-lyase; adenylyl cyclase
CCAAT-BINDING FACTOR (CBF).
G2/mitotic-specific cyclin B1 (CCNB1)
14.5-kDa translational inhibitor protein (p14.5); UK114 antigen homolog
DNA-binding protein Inhibitor ID-1; Id-1H
TSG101 tumor susceptibility protein
guanine nucleotide-binding protein G-i/G-s/G-t beta subunit 2; transducin beta 2 subunit 2

While the preferred embodiments of the invention have been illustrated and described, it will be appreciated that various changes can be made therein without departing from the spirit and scope of the invention.

1. A method of stage appropriate treatment for lethal shock based on diagnostic gene change markers from a patient that has been exposed to a lethal shock inducing agent comprising:
a. diagnosing whether a patient has been exposed to said lethal shock inducing agent by observing gene changes in a sample from said patient that are associated with an onset of lethal shock;
b. determining when said patient has been exposed to said lethal shock inducing agent; and
c. administering a stage specific therapeutic agent to said patient to counteract said gene changes that lead to lethal shock.

2. The method of claim 1, wherein if said gene changes indicate that serotonin (5-HT) is upregulated, administering said stage specific therapeutic agent comprising Zofran® and said Zofran® is administered within 2 to 3 hours of exposure to said lethal shock inducing agent.

3. The method of claim 1, wherein if said gene changes indicate that serotonin (5-HT) is upregulated, administering said stage specific therapeutic agent comprising Kytril® and said Kytril® is administered within 2 hours of exposure to said lethal shock inducing agent.

4. The method of claim 1, wherein if said gene changes indicate that Interleukin-2 is upregulated after 24 hours of SEB challenge, said stage specific therapeutic agent is antisense for the gene for Interleukin-2.

5. The method of claim 1, wherein if said gene changes indicate that TNF-alpha is upregulated after 24 hours of SEB challenge, said stage specific therapeutic agent is antisense for the gene for TNF-alpha.

6. The method of claim 1, wherein if said gene changes indicate that Interleukin-6 is upregulated after 24 hours of SEB challenge, said stage specific therapeutic agent is antisense for the gene for Interleukin-6.

7. The method of claim 1, wherein if said gene changes indicate that Guanylate binding protein is upregulated after 24 hours of SEB challenge, said stage specific therapeutic agent is antisense for the gene for Guanylate binding protein.

8. The method of claim 1, wherein if said gene changes indicate that Interferon gamma is upregulated after 24 hours of SEB challenge, said stage specific therapeutic agent is antisense for the gene for Interferon-gamma.

9. The method of claim 1, wherein if said gene changes indicate that angiopoietin 2 is upregulated after 24 hours of SEB challenge, said stage specific therapeutic agent is antisense for the gene for angiopoietin 2.

10. The method of claim 1, wherein if said gene changes indicate that Tie2 is upregulated after 24 hours of SEB challenge, said stage specific therapeutic agent is antisense for the gene for Tie2.

11. The method of claim 1, wherein if said gene changes indicate that vascular endothelial growth factor is upregulated after 24 hours of SEB challenge, said stage specific therapeutic agent is antisense for the gene for vascular endothelial growth factor.

12. The method of claim 1, wherein if said gene changes indicate that iNOS is upregulated after 24 hours of SEB challenge, said stage specific therapeutic agent is antisense for the gene for iNOS or inhibitor of the enzyme.

13. The method of claim 1, wherein if said gene changes indicate that FLT1 is upregulated after 24 hours of SEB challenge, said stage specific therapeutic agent is antisense for the gene for FLT1.

14. The method of claim 1, wherein if said gene changes indicate that the gene for 5HT2A is upregulated after 24 hours of SEB challenge, said stage specific therapeutic agent is antisense for the gene for 5HT2A.

15. The method of claim 1, wherein if said gene changes indicate that the gene VEGF is upregulated after 24 hours of SEB challenge, said stage specific therapeutic agent is antisense for the gene for VEGF.

16. The method of claim 1, wherein if said gene changes indicate that Flt is upregulated after 24 hours of SEB challenge, said stage specific therapeutic agent is antisense for the gene Flt.

17. The method of claim 1, wherein if said gene change indicate that FLT1 is upregulated after 24 hours of SEB challenge, said stage specific therapeutic agent is antisense for the gene for FLT1.

18. The method of claim 1, wherein if said gene changed indicate that the gene for angiotensin binding protein is down regulated after 24 hours of SEB challenge, said stage specific therapeutic agent is angiotensin binding protein.

19. The method of claim 1, wherein if said gene changed indicate that the gene for arginine vasopressin receptor 1A is down regulated after 24 hours of SEB challenge, said stage specific therapeutic agent is arginine receptor 1A.

20. The method of claim 1, wherein if said gene changed indicate that the gene for the protein vasopressin is down regulated after 24 hours of SEB challenge, said stage specific therapeutic agent is vasopressin.

21. The method of claim 1, wherein said stage specific therapeutic agent is P-38 inhibitor, and is administered within 2 hours of SEB challenge.

22. The method of claim 1, wherein said stage specific therapeutic agent is HPA-Na and is administered within 2 to 3 hours of SEB challenge.

23. The method of claim 1, wherein said stage specific therapeutic agent is anti-thrombin and wherein said anti-thrombib is administered within 2 to 12 hours of exposure to said lethal shock inducing toxic agent to block the effect of inflammatory mediators, vascular leakage and ischemia.

24. The method of claim 23, wherein said anti-thrombin is antithrombin III.

25. The method of claim 1, wherein said stage specific therapeutic agent is Xigris® and wherein said Xigris® is administered within 2-6 hours of exposure to said lethal shock inducing agent.

26. The method of claim 1, wherein said stage specific therapeutic agent is Pentoxifylin, and wherein said Pentoxifyline is administered within 4 hours of said lethal shock inducing toxic agent.

27. The method of claim 26, wherein said Pentoxifylin blocks a cytokine comprising TNF-alpha.

28. The method of claim 1, where said stage specific therapeutic agent is erythropoietin and wherein said erythropoietin is administered at 2-12 hours of said lethal shock inducing toxic agent.

29. The method of claim 28, wherein said lethal shock inducing toxic agent is SEB.

30. The method of claim 1, wherein said gene changes are down-regulation of said marker genes, and for said genes that are down-regulated, said therapeutic agent that is administered is proteins coded for by said genes or their products.

31. The method of 30 wherein said genes are selected from the group that are downregulated by SEB.

32. The method of claim 1, wherein said gene changes are up-regulation of said marker genes, and for said genes that are up-regulated, said therapeutic agent that is administered is antisense to said genes to block there expression.

33. The method of claim 32, wherein said genes are selected from the group consisting of IL-6, Myosin 1, Hypoxia Inducible Factor-1, Guanylate Binding Protein Isoform I, Aminolevulinate delta synthase 2, AMP deaminase, IL-17, DNAJ-like 2 protein, Cathepsin L, Transcription factor-20, M31724, pyenylalkylamine binding protein; HEC, GA17, arylsulfatase D gene, arylaulfatase E gene, cyclin protein gene, pro-platelet basic protein gene, PDGFRA, human STS WI-12000, mannosidase, beta A, lysosomal MANBA gene, UBE2D3 gene, Human DNA for Ig gamma heavy-chain, STRL22, BHMT, homo sapiens Down syndrome critical region, FI5613 containing ZNF gene family member, IL8, ELFR, homo sapiens mRNA for dual specificity phosphatase MKP-5, homo sapiens regulator of G protein signaling 10 mRNA complete, Homo sapiens Wnt-13 Mma, homo sapiens N-terminal acetyltransferase complex ard1 subunit, ribosomal protein L15 mRNA, PCNA mRNA, ATRM gene exon 21, HR gene for hairless protein exon 2, N-terminal acetyltransferase complex ard 1 subunit, HSM801431 homo sapiens mRNA, CDNA DKFZp434N2072,RPL26, and HR gene for hairless protein, regulator of G protein signaling 10.

34. The method of claim 1, wherein said sample comprises peripheral blood lymphoid cells.

35. The method of claim 1, wherein said sample comprises mammalian tissue.

36. The method of claim 1, wherein said shock inducing agent is LPS.

37. The method of claim 1, wherein said shock inducing agent is Staphylococcal enterotoxin B.

38. The method of claim 1, wherein said shock inducing agent is anthrax.

39. The method of claim 1, wherein said shock inducing agent is cholera.

40. The method of claim 1, wherein said shock inducing agent is plague.

41. The method of claim 1, wherein said upregulation and said down regulation is time dependant.

42. The method of claim 1, further comprising determining time of exposure based on early gene and late gene changes.

43. A method of treating impending shock caused by possible exposure to SEB comprising:
A) observing gene changes that are associated with exposure to SEB, said gene changes comprising:
a) downregulation of HIF-1,
b) followed by subsequent downregulation of Epo,
c) followed by subsequent down regulation of ECE,
d) followed by subsequent down regulation of ET-1, said shock will occur;
B) determining when said patient has been exposed to said lethal shock inducing agent; and
C). administering a stage specific therapeutic agent to said patient to conteract said gene changes that lead to lethal shock.

44. A method of treating impending shock caused by possible exposure to SEB comprising:
A) observing gene changes that are associated with exposure to SEB, said gene changes comprising:
a) (i) upregulation of SOD-1, followed by subsequent upregulation of H₂O₂, or
(ii) upregulation of Ras,
b) followed by subsequent upregulation of PI3K,
c) followed by subsequent upregulation of Akt,
d) followed by subsequent down regulation of RhoE,
e) followed by
(i) subsequent upregulation of CSPV, or
(ii) subsequent upregulation of RhoA,
f) followed by subsequent apoptosis,
g) followed by downregulation of ET-1, said shock will occur;
B) determining when said patient has been exposed to said lethal shock inducing agent; and
C) administering a stage specific therapeutic agent to said patient to conteract said gene changes that lead to lethal shock.

45. A method of stage appropriate treatment for illness induced by toxic agents or biological threat agents based on diagnostic gene change markers from a patient that has been exposed to a toxic agents or biological threat agents comprising:
a. diagnosing whether a patient has been exposed to said toxic or biological threat agents by observing gene changes in a sample from said patient that are associated with an onset of illness caused by said agent;
b. determining when said patient has been exposed to toxic or biological threat agents; and
c. administering a stage specific therapeutic agent to said patient to prevent illness associated with said toxic or biological threat agent agents.

47. The method of claim 1, wherein said agent also induces a cytokine storm, and further administering an anti-cytokines.

48. A method of stage appropriate treatment for illness induced by toxic agents or biological threat agents based on diagnostic gene change markers from a patient that has been exposed to a toxic agent or biological threat agent comprising: administering a stage specific therapeutic agent to said patient to prevent illness associated with said agent.
